(19) United States
(12) Patent Application Publication
(10) Pub. No.: US 2015/0064149 A1
(43) Pub. Date: Mar. 5, 2015

(54) MATERIALS AND METHODS FOR CORRECTING RECESSIVE MUTATIONS IN ANIMALS

(71) Applicant: Mice With Horns, LLC., Delray Beach, FL (US)

(72) Inventors: James WEST, Nashville, TN (US); William C. LINDSTROM, Huntsville, AL (US)

(21) Appl. No.: 14/477,387

(22) Filed: Sep. 4, 2014

Related U.S. Application Data
(60) Provisional application No. 61/873,650, filed on Sep. 4, 2013.

(51) Int. Cl. C12N 15/85

(52) U.S. Cl. CPC C12N 15/85; A61K 48/00
USPC 424/93.21

(57) ABSTRACT

The present invention provides materials and methods for correcting an undesirable nucleic acid sequence (such as a deleterious recessive mutation) in the genome of animals (such as cattle) using site-specific nucleases to facilitate gene correction. In certain embodiments, the present invention can be used to correct mutations associated with a heritable disease selected from alpha-mannosidosis, beta-mannosidosis, arthrogryposis multiplex (AM), contractural arachnodactyly (CA), developmental duplication (DD), neuropathic hydrocephalus (NH), idiopathic epilepsy, osteopetrosis, protoporphyria, pulmonary hypoplasia and anasarca (PHA), titbial hemimelia (TH), Spider Lamb Syndrome (SLS), and Brisket Disease.

### CROSS-REFERENCE TO RELATED APPLICATION

This application claims the priority benefit of U.S. Provisional Application Ser. No. 61/873,650, filed Sep. 4, 2013, which is incorporated herein by reference in its entirety.

The Sequence Listing for this application is labeled SeqList-03Sept14-ST25.txt which was created on Sep. 4, 2014 and is 365 KB. The entire content of the sequence listing is incorporated herein by reference in its entirety.

### BACKGROUND OF THE INVENTION

Both founder effects and use of line breeding for improvement of performance traits has led to an increase in deleterious recessive mutations in cattle over the past decades. Because of improvements in molecular biology techniques, many of the specific genes and mutations responsible for these recessive traits have been identified and valuable stud lines tested for their presence. The presence of such a gene has not only lead to many formerly high-worth animals being rendered useless for breeding purposes, but breed associations have eliminated whole lines of cattle by revoking registration privileges. Both trends reduce the available gene pool and contribute to even more line breeding and future defects.

Existing methods for eliminating recessive mutations in animals (such as cattle) include the use of standard selective breeding techniques. This approach harms both the individual cattle producer by sharply decreasing the value of individual cattle and, in the long run, the breed as a whole, by even further reducing what is already genetically a small breeding pool. Accordingly, improved methods for correcting deleterious recessive mutations in commercially-valuable animals (such as cattle) are needed.

### BRIEF SUMMARY

The present invention provides methods for correcting an undesirable recessive mutation in animals. In preferred embodiments, a correction construct having an exogenous nuclei acid molecule is administered to a recipient cell, as described herein, in a manner such that an undesirable nucleic acid in the recipient cell is modified in an advantageous way.

In one embodiment, the method comprises obtaining one or more spermatogonial stem cells (SSCs) of a male animal that has an undesirable recessive mutation in an endogenous nucleic acid molecule; and, optionally, determining a contiguous nucleic acid sequence of said endogenous nucleic acid molecule, wherein said contiguous nucleic acid sequence contains the recessive mutation;

providing a correction construct comprising an exogenous nucleic acid molecule for correction of the recessive mutation; and

introducing the correction construct into at least one of the SSCs using a nuclease (such as a site-specific nuclease), thereby obtaining at least one corrected SSC comprising a corrected nucleic acid molecule that does not contain the recessive mutation; and, optionally,

introducing one or more corrected SSCs into a reproductive organ of a male recipient animal; and, optionally,

collecting the donor-derived, fertilization-competent, haploid male gametes produced by the male recipient.

The corrective methods of the subject invention can also be practiced using somatic-cell nuclear transfer (SCNT). Any somatic cell including, for example, skin fibroblasts, can be isolated from the target animal. The recessive mutation is corrected in that cell, by the same methods exemplified herein for SSC. Well-known somatic cell nuclear transfer technologies (cloning) can then be used to create an animal genetically identical to the target animal, but with recessive mutations corrected.

In one embodiment, the correction construct comprises an exogenous nucleic acid molecule for correction of the undesirable nucleic acid sequence (such as a recessive mutation). In one embodiment, the exogenous nucleic acid molecule comprises a nucleic acid sequence that is identical to a contiguous nucleic acid sequence of the endogenous nucleic acid molecule wherein said contiguous nucleic acid sequence contains the undesirable nucleic acid sequence (such as a recessive mutation), except that the nucleic acid sequence of the exogenous nucleic acid molecule has a corrected nucleic acid sequence at position(s) corresponding to the undesirable nucleic acid sequence (such as a recessive mutation).

In another embodiment, the exogenous nucleic acid molecule comprises a nucleic acid sequence encoding a protein of interest, or a fragment of a protein of interest.

In one embodiment, the correction construct is introduced into the recipient cell using a site-specific nuclease. Site-specific nucleases useful according to the present invention include, but are not limited to, transcription activator-like effector nucleases (TALENs), zinc-finger nucleases (ZFNs), and/or clustered regulatory interspaced short palindromic repeat (CRISPR)/Cas-based RNA-guided DNA endonucleases.

In one embodiment, the correction construct further comprises an exercisable selection marker. The selection marker can be excisable by any recombinase (e.g., piggyback™, Cre-Loxp recombinase, Flp recombinase).

The present invention can be used to correct any undesirable nucleic acid sequence (including dominant mutations and recessive mutations) in animals. Mutations that can be corrected in accordance with the present invention include, but are not limited to, mutations causing heritable diseases, such as, alpha-mannosidosis, beta-mannosidosis, arthrogryposis multiplex (AM), contractural arachnodactyly (CA), developmental duplication (DD), neuropathic hydrocephalus (NH), idiopathic epilepsy, osteopetrosis, protoporphyria, pulmonary hypoplasia and anasarca (PHA), titbial hemimelia (TH), Spider Lamb Syndrome (SLS), and Brisket Disease.

The animals whose undesirable nucleic acid sequence(s) can be corrected in accordance with the present invention can be of any species. In certain embodiments, the animals are from any family of Equidae, Bovidae, Canidae, Felidae, and Suidae. In one specific embodiment, the animal is a bovine animal. In a preferred embodiment, the bovine animal is of the black Angus breed.

### Brief Description of the Sequences

SEQ ID NO:1 is a wild-type bovine exostosin-2 (EXT2) DNA sequence.

SEQ ID NO:2 is a wild-type bovine Aristaless-like 4 (ALX4) DNA sequence.

SEQ ID NO:3 is a mutant bovine ALX4 DNA sequence (mutant TH--found in TH-expressing phenotype and heterozygous TH carriers).

SEQ ID NO:4 is a wild-type bovine sequence of a bovine animal without arthrogryposis multiplex (AM).

SEQ ID NO:5 is an AM mutant DNA sequence.

SEQ ID NO:6 is the DNA sequence of a mutant fibroblast growth factor receptor 3 gene having a nucleotide mutation of T1719 to A1719.

### DETAILED DISCLOSURE

The current invention provides materials and methods for correcting an undesirable nucleic acid sequence (such as a deleterious recessive mutation) in the genome of an animal (such as cattle), wherein the invention utilizes knowledge of the genetic nature of mutations in animals, targeted gene correction, and, in certain embodiment, improved methods of spermatogonial stem cell (SSC) transfer to allow production of mutation-corrected sperm. The corrective methods of the subject invention can also be practiced using somatic-cell nuclear transfer (SCNT).

In one embodiment, the present invention provides a method for correcting an undesirable nucleic acid sequence (such as dominant mutations and recessive mutations) in animals, wherein the method comprises:

obtaining one or more spermatogonial stem cells (SSCs) of a male animal that has an undesirable nucleic acid sequence in an endogenous nucleic acid molecule; and, optionally, determining a contiguous nucleic acid sequence of said endogenous nucleic acid molecule, wherein said contiguous nucleic acid sequence contains the undesirable nucleic acid sequence;

providing a correction construct comprising an exogenous nucleic acid molecule for correction of the undesirable nucleic acid sequence; and

introducing the correction construct into at least one of the SSCs using a nuclease (such as a site-specific nuclease), thereby obtaining at least one corrected SSC comprising a corrected nucleic acid molecule that does not contain the undesirable nucleic acid sequence; and, optionally,

introducing one or more corrected SSCs into a reproductive organ of a male recipient animal; and, optionally,

collecting the donor-derived, fertilization-competent, haploid male gametes produced by the male recipient.

In one embodiment, the endogenous nucleic acid molecule encodes a non-functional protein, and the corrected nucleic acid molecule encodes a functional protein.

In one embodiment, the endogenous nucleic acid molecule containing the undesirable mutation (such as a dominant mutation or a recessive mutation) is in the genome of the animal. In one embodiment, the genome of at least one corrected SSC comprises a corrected nucleic acid molecule that does not contain the undesirable nucleic acid sequence.

In a preferred embodiment, the undesirable nucleic acid sequence is a recessive mutation, such as a deleterious recessive mutation. In one embodiment, the present invention provides a method for correcting an undesirable recessive mutation in animals, wherein the method comprises:

obtaining one or more spermatogonial stem cells (SSCs) of a male animal that has an undesirable recessive mutation in an endogenous nucleic acid molecule; and, optionally, determining a contiguous nucleic acid sequence of said endogenous nucleic acid molecule, wherein said contiguous nucleic acid sequence contains the recessive mutation;

providing a correction construct comprising an exogenous nucleic acid molecule for correction of the recessive mutation; and

introducing the correction construct into at least one of the SSCs using a nuclease (such as a site-specific nuclease), thereby obtaining at least one corrected SSC comprising a corrected nucleic acid molecule that does not contain the recessive mutation; and, optionally,

introducing one or more corrected SSCs into a reproductive organ of a male recipient animal; and, optionally,

collecting the donor-derived, fertilization-competent, haploid male gametes produced by the male recipient.

The term “mutation,” as used herein, refers to its ordinary meaning that is a heritable alteration in a DNA sequence. Mutations include additions, deletions, and substitutions of nucleotides of a DNA sequence.

The term “recessive allele,” as used herein, refers to its ordinary meaning that is an allele whose phenotype is not expressed in a heterozygote.

The term “dominant allele,” as used herein, refers to its ordinary meaning that is an allele whose phenotype is expressed in a heterozygote.

In one embodiment, the method comprises the step of determining a contiguous nucleic acid sequence of the endogenous nucleic acid molecule, wherein said contiguous nucleic acid sequence contains the undesirable nucleic acid sequence (such as a recessive mutation).

In certain embodiments, the contiguous sequence has a length of at least 30 base pairs, including, but not limited to, at least 40 base pairs, at least 50 base pairs, at least 100 base pairs, at least 150 base pairs, at least 200 base pairs, at least 300 base pairs, at least 500 base pairs, at least 700 base pairs, at least 1000 base pairs, at least 1500 pair pairs, at least 2000 base pairs, at least 2500 base pairs, at least 3000 base pairs, at least 3500 base pairs, at least 4000 base pairs, at least 4500 base pairs, at least 5000 base pairs, at least 7000 base pairs, at least 10 k base pairs, at least 15 k base pairs, at least 20 k base pairs, at least 30 k base pairs, at least 40 k base pairs, at least 50 k base pairs, at least 80 k base pairs, and at least 100 k base pairs.

In certain embodiments, the contiguous sequence has a length of no more than 500 k base pairs, including, but not limited to, no more than 400 k base pairs, no more than 300 k base pairs, no more than 200 k base pairs, no more than 100 k base pairs, no more than 80 k base pairs, no more than 50 k base pairs, no more than 30 k base pairs, no more than 20 k base pairs, no more than 10 k base pairs, no more than 7000 pair pairs, no more than 5000 base pairs, no more than 3000 base pairs, no more than 1000 base pairs, no more than 700 base pairs, no more than 500 base pairs, no more than 300 base pairs, and no more than 100 base pairs.

In certain embodiments, the contiguous nucleic acid sequence comprises sequences immediately upstream and/or immediately downstream of the undesirable nucleic acid sequence (such as, a recessive mutation), wherein the upstream sequence and/or the downstream sequence has a length of at least 30 base pairs, including, but not limited to, at least 20 base pairs, at least 30 base pairs, at least 40 base pairs, at least 50 base pairs, at least 100 base pairs, at least 150 base pairs, at least 200 base pairs, at least 300 base pairs, at least 500 base pairs, at least 700 base pairs, at least 1000 base pairs, at least 1500 pair pairs, at least 2000 base pairs, at least 2500 base pairs, at least 3000 base pairs, at least 3500 base pairs, at least 4000 base pairs, at least 4500 base pairs, at least 5000 base pairs, at least 7000 base pairs, at least 10 k base pairs, at least 15 k base pairs, at least 20 k base pairs, at least 30 k base pairs, at least 40 k base pairs, at least 50 k base pairs, at least 80 k base pairs, and at least 100 k base pairs.

In certain embodiments, the contiguous nucleic acid sequence comprises sequences immediately upstream and/or immediately downstream of the undesirable nucleic acid sequence (such as, a recessive mutation), wherein the upstream sequence and/or the downstream sequence has a length of no more than 500 k base pairs, including, but not limited to, no more than 400 k base pairs, no more than 300 k base pairs, no more than 200 k base pairs, no more than 10 k base pairs, no more than 80 k base pairs, no more than 50 k base pairs, no more than 30 k base pairs, no more than 20 k base pairs, no more than 100 k base pairs, no more than 7000 pair pairs, no more than 5000 base pairs, no more than 3000 base pairs, no more than 1000 base pairs, no more than 700 base pairs, no more than 500 base pairs, no more than 300 base pairs, and no more than 100 base pairs.

In one embodiment, the correction construct comprises an exogenous nucleic acid molecule for correction of the undesirable nucleic acid sequence (such as a recessive mutation). In one embodiment, the exogenous nucleic acid molecule comprises a nucleic acid sequence that is identical to a contiguous nucleic acid sequence of the endogenous nucleic acid molecule wherein said contiguous nucleic acid sequence contains the undesirable nucleic acid sequence, except that the nucleic acid sequence of the exogenous nucleic acid molecule has a corrected nucleic acid sequence at position(s) corresponding to the undesirable nucleic acid sequence.

In another embodiment, the exogenous nucleic acid molecule comprises a nucleic acid sequence encoding a protein of interest, or a fragment of the protein of interest.

In one embodiment, the exogenous nucleic acid molecule comprises a nucleic acid sequence encoding a functional protein of interest. In another embodiment, the corrected nucleic acid molecule comprises a corrected nucleic acid sequence (that no longer contains the undesirable nucleic acid sequence such as a recessive mutation) encoding a functional protein of interest.

In certain embodiments, the exogenous nucleic acid molecule comprises a double-strand DNA sequence having a length of at least 30 base pairs, including, but not limited to, at least 40 base pairs, at least 50 base pairs, at least 100 base pairs, at least 150 base pairs, at least 200 base pairs, at least 300 base pairs, at least 500 base pairs, at least 700 base pairs, at least 1000 base pairs, at least 1500 pair pairs, at least 2000 base pairs, at least 2500 base pairs, at least 3000 base pairs, at least 3500 base pairs, at least 4000 base pairs, at least 4500 base pairs, at least 5000 base pairs, at least 7000 base pairs, at least 10 k base pairs, at least 15 k base pairs, at least 20 k base pairs, at least 30 k base pairs, at least 40 k base pairs, at least 50 k base pairs, at least 80 k base pairs, and at least 100 k base pairs.

In certain embodiments, the exogenous nucleic acid molecule comprises a double-strand DNA sequence having a length of no more than 500 k base pairs, including, but not limited to, no more than 400 k base pairs, no more than 300 k base pairs, no more than 200 k base pairs, no more than 100 k base pairs, no more than 80 k base pairs, no more than 50 k base pairs, no more than 30 k base pairs, no more than 20 k base pairs, no more than 10 k base pairs, no more than 7000 pair pairs, no more than 5000 base pairs, no more than 3000 base pairs, no more than 1000 base pairs, no more than 700 base pairs, no more than 500 base pairs, no more than 300 base pairs, and no more than 100 base pairs.

In certain embodiments, the exogenous nucleic acid molecule comprises a single-strand DNA sequence having a length of at least 30 nucleotides, including, but not limited to, at least 40 nucleotides, at least 50 nucleotides, at least 100 nucleotides, at least 150 nucleotides, at least 200 nucleotides, at least 300 nucleotides, at least 500 nucleotides, at least 700 nucleotides, at least 1000 nucleotides, at least 1500 nucleotides, at least 2000 nucleotides, at least 2500 nucleotides, at least 3000 nucleotides, at least 3500 nucleotides, at least 4000 nucleotides, at least 4500 nucleotides, at least 5000 nucleotides, at least 7000 nucleotides, at least 10 k nucleotides, at least 15 k nucleotides, at least 20 k nucleotides, at least 30 k nucleotides, at least 40 k nucleotides, at least 50 k nucleotides, at least 80 k nucleotides, and at least 100 k nucleotides.

In certain embodiments, the exogenous nucleic acid molecule comprises a single-strand DNA sequence having a length of no more than 500 k nucleotides, including, but not limited to, no more than 400 k nucleotides, no more than 300 k nucleotides, no more than 200 k nucleotides, no more than 100 k nucleotides, no more than 80 k nucleotides, no more than 50 k nucleotides, no more than 30 k nucleotides, no more than 20 k nucleotides, no more than 10 k nucleotides, no more than 7000 nucleotides, no more than 5000 nucleotides, no more than 3000 nucleotides, no more than 1000 nucleotides, no more than 700 nucleotides, no more than 500 nucleotides, no more than 300 nucleotides, and no more than 100 nucleotides.

In one embodiment, the correction construct further comprises an excisable selection marker. Examples of selection markers useful according to the present invention include, but are not limited to, antibiotic resistance, fluorescent cell sorting marker, magnetic cell sorting marker, and any combination thereof. Suitable selection marker genes are known in the art, including but not limited to, nucleic acid molecules encoding proteins that mediate antibiotic resistance (e.g., ampicillin resistance, neomycin resistance, G418 resistance, and puromycin resistance), nucleic acid molecules encoding colored or fluorescent or luminescent proteins (e.g., green fluorescent protein, enhanced green fluorescent protein, red fluorescent protein, and luciferase), and nucleic acid molecules encoding proteins which mediate enhanced cell growth and/or gene amplification (e.g., dihydrofolate reductase). Epitope tags include, for example, one or more copies of FLAG, His, myc, Tap, HA or any detectable amino acid sequence.

The selection marker can be excisable by any recombinase (e.g., piggyback™, Cre-Loxp recombinase, and Flp recombinase). Vector designs of piggyback™, Cre-Loxp recombinase, Flp recombinase for excision of nucleic acid sequences are known in the art.

In one embodiment, the correction construct is introduced into the recipient cell (e.g. SSC) using a site-specific nuclease. Site-specific nucleases useful according to the present invention include, but are not limited to, transcription activator-like effector nucleases (TALENs), zinc-finger nucleases (ZFNs), and/or clustered regulatory interspaced short palindromic repeat (CRISPR)/Cas-based RNA-guided DNA endonucleases. TAL-effector nucleases are a class of nucleases that allow sequence-specific DNA cleavage, making it possible to perform site-specific gene editing.

Site-specific genome-editing materials and methods are known in the art. In certain embodiment, a site-specific nuclease is introduced to the host cell that is capable of causing a double-strand break near or within a genomic target site, which greatly increases the frequency of homologous recombination at or near the cleavage site. In certain embodiments, the recognition sequence for the nuclease is present in the host cell genome only at the target site, thereby minimizing any off-target genomic binding and cleavage by the nuclease.

In one embodiment, the site-specific nuclease recognizes the undesirable nucleic acid sequence. In one embodiment, the site-specific nuclease is engineered to cleave a pre-determined nucleic acid sequence from the endogenous nucleic acid molecule, wherein the pre-determined sequence contains the undesirable nucleic acid sequence.

Any somatic cell including, for example, skin fibroblasts, can be isolated from the target animal. The recessive mutation is corrected in that cell by the same methods exemplified herein for SSC. Somatic cell nuclear transfer (SCNT) (cloning) can then be used to create an animal genetically identical to the target animal, but with recessive mutations corrected. Thus, although SSCs are specifically exemplified herein it should be understood that other cells can be used in, for example, an SCNT procedure.

Site-specific nucleases can be introduced into the SSCs, or other recipient cell, using any method known in the art. In one embodiment, the site-specific nuclease enzymes are introduced directly into the recipient cell. In another embodiment, the present invention involves administering a nucleic acid molecule encoding a site-specific nuclease into the cell. In one embodiment, the nucleic acid molecule is in an expression vector. In one embodiment, the correction vector comprises a nucleic acid molecule encoding a site-specific nuclease.

The site-specific nuclease can be introduced into the cells before, during (or simultaneously), and/or after the administration of the correction vector to the cell.

In a further embodiment, the method for correcting an undesirable nucleic acid sequence (such as dominant mutations and recessive mutations) further comprises:

introducing one or more corrected somatic cells into a reproductive organ of a male recipient animal; and optionally,

collecting the donor-derived, fertilization-competent, haploid male gametes produced by the male recipient.

In one embodiment, male gametes produced by the recipient animal are sperm.

The animals whose undesirable nucleic acid sequence(s) can be corrected in accordance with the present invention can be of any species, including, but not limited to, mammalian species including, but not limited to, domesticated and laboratory animals such as dogs, cats, mice, rats, guinea pigs, and hamsters; livestock such as horses, cattle, pigs, sheep, goats, ducks, geese, and chickens; primates such as apes, chimpanzees, orangutans, humans, and monkeys; fish; amphibians such as frogs and salamanders; reptiles such as snakes and lizards; and other animals such as fox, weasels, rabbits, mink, beavers, ermines, otters, sable, seals, coyotes, chinchillas, deer, muskrats, and possum.

In certain embodiments, the animals are from any family of Equidae, Bovidae, Canidae, Felidae, and Suidae. In one embodiment, the animal is not a human. In one specific embodiment, the animal is a bovine animal. In a preferred embodiment, the bovine animal is of the black Angus breed. In certain embodiments, bovine animals of the present invention can include, but are not limited to, domesticated cattle, bison, and buffalos (e.g., water buffalo and African buffalo).

As used herein, “Angus” refers to any bovine animal with any Angus ancestry.

In certain embodiments, the present invention can be used to correct a single mutation, or two or more mutations in animals by introducing one or more correction constructs.

The present invention can be used to correct any undesirable nucleic acid sequence (including dominant mutations and recessive mutations) in animals. Mutations that can be corrected in accordance with the present invention include, but are not limited to, mutations causing, or associated with, heritable diseases, such as, alpha-mannosidosis, beta-mannosidosis, arthrogryposis multiplex (AM), contractural arachnodactyly (CA), developmental duplication (DD), neuropathic hydrocephalus (NH), idiopathic epilepsy, osteopetrosis, protoporphyria, pulmonary hypoplasia and anasarca (PHA), titbial hemimelia (TH), Spider Lamb Syndrome (SLS), and Brisket Disease.

In one embodiment, the present invention further comprises identifying whether an animal has an undesirable nucleic acid sequence (such as, a dominant mutation, a recessive mutation) in animals. Methods for genetic defect testing and for detecting mutations (such as additions, deletions, substitutions, and translocations) are known in the art. See U.S. Pat. Nos 8,158,356; 8,431,346; and 6,306,591.

### Heritable Diseases in Animals

Mutations that can be corrected in accordance with the present invention include, but are not limited to, mutations causing heritable diseases such as alpha-mannosidosis, beta-mannosidosis, arthrogryposis multiplex (AM), contractural arachnodactyly (CA), developmental duplication (DD), neuropathic hydrocephalus (NH), idiopathic epilepsy, osteopetrosis, protoporphyria, pulmonary hypoplasia and anasarca (PHA), titbial hemimelia (TH), Spider Lamb Syndrome (SLS), and Brisket Disease.

### Tibial Hemimelia (TH)

Tibial Hemimelia (TH) is a lethal congenital disorder in cattle characterized by severe and lethal deformities in newborn calves, including multiple skeletal deformities such as twisted rear legs with fused joints, large abdominal hernias and/or skull deformities. Often, such a calf is born dead, or if it survives birth cannot stand to nurse and must be destroyed, resulting in economic loss for owners.

TH is associated with mutations within the region of the genome that encodes Aristaless-like 4 (ALX4). ALX4 sequences and mutations causing TH are known in the art (see U.S. Pat. No. 8,158,356, which is hereby incorporated by reference in its entirety).

In one embodiment, a wild-type bovine exostosin-2 (EXT2) DNA sequence is SEQ ID NO:1; a wild-type bovine ALX4 DNA sequence is SEQ ID NO:2; a mutant bovine ALX4 DNA sequence is SEQ ID NO:3.

### Arthrogryposis Multiplex (AM)

Arthrogryposis Multiplex (AM), commonly referred to as “Curly Calf Syndrome,” is a genetic defect that has recently been reported in Angus cattle. Based on pedigree examination of affected calves, this genetic defect is determined to have an autosomal recessive mode of inheritance. Due to this recessive inheritance pattern, only calves that are homozygous (i.e., receiving a chromosome with the mutation from both parents) for the mutation causing AM are affected with multiple abnormalities most often including arthrogryposis (contracted or extended limbs with stiffened joints), scoliosis and kyphosis (abnormal curvature of the spine), and muscular hypoplasia (reduced muscle development). Less commonly, the syndrome is associated with mild hydrocephalus caused by inflammation of the brain. Calves are born dead or fail to thrive and die shortly after birth.

AM is associated with mutations within the region of the genome that encodes ISG15 ubiquitin-like modifier (ISG15), enhancer split 4 (HES4), and/or agrin (AGRN). The wild-type ISG15, HES4, and ARGN sequences, and mutations associated with AM are known in the art (see U.S. Pat. No. 8,431,346, which is hereby incorporated by reference in its entirety). One mutation causing AM is identified as a deletion of about 23,363 base pairs. This deletion encompasses the 5′ regulatory region of the hairy and enhancer of split 4 (HES4) gene, the entirety of the ISG15 ubiquitin-like modifier (ISG15) gene, and the 5′ regulatory region and first two exons of the agrin (AGRN) gene. The mutation results in a complete loss-of-function of AGRN thus producing the disease phenotype when an animal is homozygous for the deletion-containing chromosome.

SEQ ID NO:4 is a wild-type bovine sequence and various genetic deletion mutations are described in U.S. Pat. No. 8,431,346. SEQ ID NO:5 is an AM mutant genetic sequence.

### Spider Lamb Syndrome (SLS)

“Spider Lamb Syndrome” or “hereditary chondrodysplasia” is a semi-lethal congenital disorder in sheep causing severe skeletal abnormalities. These abnormalities can include abnormally long, spider-like legs, humped and twisted spines, deformed ribs and sternebra, facial deformities, lack of body fat, and underdevelopment of muscle. The most severe lesions progress to compression fractures from mechanical stress due to abnormal limb angulation. Radiological evaluation of Spider lamb shoulders, elbows and sternum reveal multiple, irregular islands of ossification. Histologic examinations of the vertebrae and long bones indicate an increase in width of the zone of proliferation, as well as hypertrophy and unevenness of the growth cartilage. Chondrocytes appear vacuolated and disorganized, lining up in bent nonparallel columns.

The Spider Lamb Syndrome is associated with mutations in the fibroblast growth factor receptor 3 (FGFR3) gene. The wild-type FGFR3 and various mutations in FGFR3 are known in the art (see, U.S. Patent No. 6,306,591, which is hereby incorporated by reference in its entirety).

SEQ ID NO:6 is a mutant fibroblast growth factor receptor 3 gene having a nucleotide mutation of T1719 to A1719.

### Brisket Disease

High-altitude pulmonary hypertension (HAPH) is a consequence of chronic alveolar hypoxia, leading to hypoxic vasoconstriction and remodeling of the pulmonary circulation. Brisket disease in cattle is a naturally occurring animal model of hypoxic pulmonary hypertension. Genetically susceptible cattle develop severe pulmonary hypertension and right heart failure at altitudes >7,000 ft.

Genes and mutations associated with Brisket Disease can be identified by a person skilled in the art. See Newman et al., High-altitude pulmonary hypertension in cattle (brisket disease): Candidate genes and gene expression profiling of peripheral blood mononuclear cells, Pulm Circ. 2011 October-December; 1(4): 462-469, which is hereby incorporated by reference in its entirety.

### Expression Constructs

The present invention also provides expression constructs and vectors for correction of recessive mutations in animals.

As used herein, the term “expression construct” refers to a combination of nucleic acid sequences that provides for transcription of an operably linked nucleic acid sequence. Expression constructs of the invention also generally include regulatory elements that are functional in the intended host cell in which the expression construct is to be expressed. Regulatory elements include promoters, transcription termination sequences, translation termination sequences, enhancers, and polyadenylation elements.

An expression construct of the invention can comprise a promoter sequence operably linked to a polynucleotide sequence encoding a peptide of the invention. Promoters can be incorporated into a polynucleotide using standard techniques known in the art. Multiple copies of promoters or multiple promoters can be used in an expression construct of the invention. In a preferred embodiment, a promoter can be positioned about the same distance from the transcription start site as it is from the transcription start site in its natural genetic environment. Some variation in this distance is permitted without substantial decrease in promoter activity. A transcription start site is typically included in the expression construct.

As used herein, the term “operably linked” refers to a juxtaposition of the components described wherein the components are in a relationship that permits them to function in their intended manner. In general, operably linked components are in contiguous relation. Sequence(s) operably-linked to a coding sequence may be capable of effecting the replication, transcription and/or translation of the coding sequence. For example, a coding sequence is operably-linked to a promoter when the promoter is capable of directing transcription of that coding sequence.

A “coding sequence” or “coding region” is a polynucleotide sequence that is transcribed into mRNA and/or translated into a polypeptide. For example, a coding sequence may encode a polypeptide of interest. The boundaries of the coding sequence are determined by a translation start codon at the 5′-terminus and a translation stop codon at the 3′-terminus.

The term “promoter,” as used herein, refers to a DNA sequence operably linked to a nucleic acid sequence to be transcribed such as a nucleic acid sequence encoding a desired molecule. A promoter is generally positioned upstream of a nucleic acid sequence to be transcribed and provides a site for specific binding by RNA polymerase and other transcription factors. In specific embodiments, a promoter is generally positioned upstream of the nucleic acid sequence transcribed to produce the desired molecule, and provides a site for specific binding by RNA polymerase and other transcription factors.

In addition to a promoter, one or more enhancer sequences may be included such as, but not limited to, cytomegalovirus (CMV) early enhancer element and an SV40 enhancer element. Additional included sequences are an intron sequence such as the beta globin intron or a generic intron, a transcription termination sequence, and an mRNA polyadenylation (pA) sequence such as, but not limited to, SV40-pA, beta-globin-pA, the human growth hormone (hGH) pA and SCF-pA.

In one embodiment, the expression construct comprises polyadenylation s equences, such as polyadenylation sequences derived from bovine growth hormone (BGH) and SV40.

The term “polyA” or “p(A)” or “pA” refers to nucleic acid sequences that signal for transcription termination and mRNA polyadenylation. The polyA sequence is characterized by the hexanucleotide motif AAUAAA. Commonly used polyadenylation signals are the SV40 pA, the human growth hormone (hGH) pA, the beta-actin pA, and beta-globin pA. The sequences can range in length from 32 to 450 bp. Multiple pA signals may be used.

In one embodiment, the genetic construct comprises a nucleic acid molecule encoding a selection marker, such as neomycin resistance biomarker protein, which can be excised through PIGGYBAC™ transposons. In one embodiment, the construct is flanked by short homology arms.

The term “vector” is used to refer to any molecule (e.g., nucleic acid, plasmid, or virus) used to transfer coding information (e.g., a polynucleotide of the invention) to a host cell.

The terms “expression vector” and “transcription vector” are used interchangeably to refer to a vector that is suitable for use in a host cell (e.g., a subject's cell) and contains nucleic acid sequences that direct and/or control the expression of exogenous nucleic acid sequences. Expression includes, but is not limited to, processes such as transcription, translation, and RNA splicing, if introns are present. Vectors useful according to the present invention include plasmids, viruses, BACs, YACs, and the like. Particular viral vectors illustratively include those derived from adenovirus, adeno-associated virus and lentivirus.

As used herein, the term “isolated” molecule (e.g., isolated nucleic acid molecule) refers to molecules which are substantially free of other cellular material, or culture medium when produced by recombinant techniques, or substantially free of chemical precursors or other chemicals when chemically synthesized.

The term “recombinant” is used to indicate a nucleic acid construct in which two or more nucleic acids are linked and which are not found linked in nature.

The term “nucleic acid” as used herein refers to RNA or DNA molecules having more than one nucleotide in any form including single-stranded, double-stranded, oligonucleotide or polynucleotide.

The term “nucleotide sequence” is used to refer to the ordering of nucleotides in an oligonucleotide or polynucleotide in a single-stranded form of nucleic acid.

The term “expressed” refers to transcription of a nucleic acid sequence to produce a corresponding mRNA and/or translation of the mRNA to produce the corresponding protein. Expression constructs can be generated recombinantly or synthetically or by DNA synthesis using well-known methodology.

The term “regulatory element” as used herein refers to a nucleotide sequence which controls some aspect of the expression of an operably linked nucleic acid sequence. Exemplary regulatory elements illustratively include an enhancer, an internal ribosome entry site (IRES), an intron, an origin of replication, a polyadenylation signal (pA), a promoter, a transcription termination sequence, and an upstream regulatory domain, which contribute to the replication, transcription, post-transcriptional processing of a nucleic acid sequence. Those of ordinary skill in the art are capable of selecting and using these and other regulatory elements in an expression construct with no more than routine experimentation. In one embodiment, the construct of the present invention comprises an internal ribosome entry site (IRES). In one embodiment, the expression construct comprises kozak consensus sequences.

Optionally, a reporter gene is included in the transgene construct. The term “reporter gene” as used herein refers to a gene that is easily detectable when expressed, for example, via chemiluminescence, fluorescence, colorimetric reactions, antibody binding, inducible markers, ligand binding assays, and the like. Exemplary reporter genes include but are not limited to green fluorescent protein. The production of recombinant nucleic acids, vectors, transformed host cells, proteins and protein fragments by genetic engineering is well known.

If desired, the vector may optionally contain flanking nucleic sequences that direct site-specific homologous recombination. The use of flanking DNA sequences to permit homologous recombination into a desired genetic locus is known in the art. At present it is preferred that up to several kilobases or more of flanking DNA corresponding to the chromosomal insertion site be present in the vector on both sides of the encoding sequence (or any other sequence of this invention to be inserted into a chromosomal location by homologous recombination) to assure precise replacement of chromosomal sequences with the exogenous DNA. See e.g. Deng et al, 1993, Mol. Cell. Biol 13(4):2134-40; Deng et al, 1992, Mol Cell Biol 12(8):3365-71; and Thomas et al, 1992, Mol Cell Biol 12(7):2919-23. It should also be noted that the cell of this invention may contain multiple copies of the gene of interest.

A “gene” includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

A “target site” or “target sequence” is a nucleic acid sequence that defines a portion of a nucleic acid to which a binding molecule will bind, provided sufficient conditions for binding exist.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a coding sequence for any polypeptide or fragment thereof, a functioning version of a malfunctioning endogenous molecule or a malfunctioning version of a normally-functioning endogenous molecule. An exogenous molecule can also be the same type of molecule as an endogenous molecule but be derived from a different species than the species the endogenous molecule is derived from. For example, a human nucleic acid sequence may be introduced into a cell line originating from a hamster or mouse.

An “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins (for example, a fusion between a ZFP DNA-binding domain and a cleavage domain) and fusion nucleic acids (for example, a nucleic acid encoding the fusion protein described supra).

“Complement” or “complementary sequence” means a sequence of nucleotides which forms a hydrogen-bonded duplex with another sequence of nucleotides according to Watson-Crick base-pairing rules. For example, the complementary base sequence for 5′-AAGGCT-3′ is 3′-TTCCGA-5′. This invention encompasses complementary sequences to any of the nucleotide sequences claimed in this invention.

### Nuclease-Mediated Site-Specific Genome Editing

Methods of site-specific genome editing are known in the art. In certain embodiments, the present invention uses transcription activator-like effector nucleases (TALENs), zinc-finger nucleases (ZFNs), and/or clustered regulatory interspaced short palindromic repeat (CRISPR)/Cas-based RNA-guided DNA endonucleases for site-specific genome editing, all of which are known in the art. See Gaj et al., ZFN, TALEN, and CRISPR/Cas-Based Methods for Genome Engineering, Trends in Biotechnology, July 2013, Vol. 31, No. 7, which is hereby incorporated by reference in its entireties.

TALENs (transcription activator-like effector nucleases) are fusions of the nuclease (such as Fokl) cleavage domain and DNA-binding domains derived from TALE proteins. TALEs contain multiple 33-35-amino-acid repeat domains that each recognizes a single base pair. TALENs can induce double-strand breaks that activate DNA damage response pathways and enable custom alteration.

ZFNs (zinc-finger nucleases) are fusions of the nonspecific DNA cleavage domain from a restriction endonuclease (such as Fokl) with zinc-finger proteins. ZFN dimers induce target DNA double-strand breaks that stimulate DNA damage response pathways. The binding specificity of the designed zinc-finger domain directs the ZFN to a specific genomic site.

ZFNickases (zinc-finger nickases) are ZFNs that contain inactivating mutations in one of the two nuclease (such as Fokl) cleavage domains. ZFNickases make only single-stranded DNA breaks and induce HDR without activating the mutagenic NHEJ pathway.

CRISPR/Cas (CRISPR associated) (clustered regulatory interspaced short palindromic repeats) systems are loci that contain multiple short direct repeats, and provide acquired immunity to bacteria and archaea. CRISPR systems reply on crRNA and tracrRNA for sequence-specific silencing of invading foreign DNA. Three types of CRISPR systems exist: in type II systems, Cas9 serves as an RNA-guided DNA endonuclease that cleaves DNA upon crRNA-tracrRNA target recognition.

crRNA: CRISPR RNA base pairs with tracrRNA to form a two-RNA structure that guides the Cas9 endonuclease to complementary DNA sites for cleavage.

A double-stranded break (DSB) is a form of DNA damage that occurs when both DNA strands are cleaved. DSBs can be products of TALENs, ZFNs, and CRISPR)/Cas9 action.

Homology-directed repair (HDR) is a template-dependent pathway for DSB repair. By supplying a homology-containing donor template along with a site-specific nuclease, HDR faithfully inserts the donor molecule at the targeted locus. This approach enables the insertion of single or multiple transgenes, as well as single nucleotide substitutions.

NHEJ (nonhomologous end joining) is a DSB repair pathway that ligates or joins two broken ends together. NHEJ does not use a homologous template for repair and thus typically leads to the introduction of small insertions and deletions at the site of the break.

PAMs (protospacer adjacent motifs) are short nucleotide motifs that occur on crRNA and are specifically recognized and required by Cas9 for DNA cleavage.

tracrRNA (transactivating chimeric RNA) is noncoding RNA that promotes crRNA processing and is required for activating RNA-guided cleavage by Cas9.

In one embodiment, the site-specific genome-editing method comprises contacting the host cell with one or more integration polynucleotides comprising an exogenous nucleic acid to be integrated into the genomic target site, and one or more nucleases capable of causing a double-strand break near or within the genomic target site. Cleavage near or within the genomic target site greatly increases the frequency of homologous recombination at or near the cleavage site.

In certain embodiments, a site-specific nuclease cleaves DNA in cellular chromatin, and facilitates targeted integration of an exogenous sequence (donor polynucleotide). In certain embodiments for targeted integration, one or more zinc finger or TALE DNA binding domains are engineered to bind a target site at or near the predetermined cleavage site, and a fusion protein comprising the engineered zinc finger or TALE DNA binding domain and a cleavage domain is expressed in a cell. Upon binding of the zinc finger or TALE DNA binding portion of the fusion protein to the target site, the DNA is cleaved, preferably via a double stranded break, near the target site by the cleavage domain. The presence of a double-stranded break facilitates integration of exogenous sequences as described herein via NHEJ mechanisms.

The exogenous (donor) sequence can be introduced into the cell prior to, concurrently with, or subsequent to, expression of the fusion protein(s).

“Recombination” refers to a process of exchange of genetic information between two polynucleotides. As used herein, “homologous recombination (HR)” refers to the specialized form of such exchange that takes place, for example, during repair of double-strand breaks in cells. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. “Cleavage” refers to the breakage of the covalent backbone of a DNA molecule.

Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends:

A “cleavage domain” comprises one or more polypeptide sequences which possesses catalytic activity for DNA cleavage.

A “cleavage half-domain” is a polypeptide sequence which, in conjunction with a second polypeptide (either identical or different), forms a complex having cleavage activity (preferably double-strand cleavage activity).

TAL effectors of plant pathogenic bacteria in the genus Xanthomonas play important roles in disease, or trigger defense, by binding host DNA and activating effector-specific host genes. see, e.g., Gu et al. (2005) Nature 435:1122-5; Yang et al., (2006) Proc. Natl. Acad. Sci. USA 103:10503-8; Kay et al., (2007) Science 318:648-51; Sugio et al., (2007) Proc. Natl. Acad. Sci. USA 104:10720-5; Romer et al., (2007) Science 318:645-8; Boch et al., (2009) Science 326(5959):1509-12; and Moscou and Bogdanove, (2009) 326(5959):1501. A TAL effector comprises a DNA binding domain that interacts with DNA in a sequence-specific manner through one or more tandem repeat domains. The repeated sequence typically comprises 34 amino acids, and the repeats are typically 91-100% homologous with each other. Polymorphism of the repeats is usually located at positions 12 and 13, and there appears to be a one-to-one correspondence between the identity of repeat variable-diresidues at positions 12 and 13 with the identity of the contiguous nucleotides in the TAL-effector's target sequence.

The TAL-effector DNA binding domain can be engineered to bind to a desired target sequence, and fused to a nuclease domain, e.g., from a type II restriction endonuclease, typically a nonspecific cleavage domain from a type II restriction endonuclease such as Fokl (see e.g., Kim et al. (1996) Proc. Natl. Acad. Sci. USA 93:1156-1160). Other useful endonucleases may include, for example, HhaI, HindIII, Nod, BbvCI, EcoRI, BglI, and AlwI. In certain embodiments, the TALEN comprises a TAL effector domain comprising a plurality of TAL effector repeat sequences that, in combination, bind to a specific nucleotide sequence in the target DNA sequence, such that the TALEN cleaves the target DNA within or adjacent to the specific nucleotide sequence. TALENS useful for the methods provided herein include those described in WO10/079430 and U.S. Patent Application Publication No. 2011/0145940.

In some embodiments of the methods provided herein, one or more of the nucleases is a zinc-finger nuclease (ZFN). ZFNs are engineered double-strand break inducing agents comprised of a zinc finger DNA binding domain and a double strand break inducing agent domain. Engineered ZFNs consist of two zinc finger arrays (ZFAs), each of which is fused to a single subunit of a non-specific endonuclease, such as the nuclease domain from the FokI enzyme, which becomes active upon dimerization. Typically, a single ZFA consists of 3 or 4 zinc finger domains, each of which is designed to recognize a specific nucleotide triplet (GGC, GAT, etc.). In certain embodiments, ZFNs composed of two “3-finger” ZFAs are capable of recognizing an 18 base pair target site; an 18 base pair recognition sequence is generally unique, even within large genomes such as those of humans and plants. By directing the co-localization and dimerization of two Fokl nuclease monomers, ZFNs generate a functional site-specific endonuclease that creates a double-stranded break (DSB) in DNA at the targeted locus.

Useful zinc-finger nucleases include those that are known and those that are engineered to have specificity for one or more target sites (TS) described herein. Zinc finger domains are amenable for designing polypeptides which specifically bind a selected polynucleotide recognition sequence, for example, within the target site of the host cell genome. ZFNs consist of an engineered DNA-binding zinc finger domain linked to a non-specific endonuclease domain, for example, nuclease domain from a Type IIs endonuclease such as HO or FokI. Alternatively, engineered zinc finger DNA binding domains can be fused to other double-strand break inducing agents or derivatives thereof that retain DNA nicking/cleaving activity. For example, this type of fusion can be used to direct the double-strand break inducing agent to a different target site, to alter the location of the nick or cleavage site, to direct the inducing agent to a shorter target site, or to direct the inducing agent to a longer target site. In some examples a zinc finger DNA binding domain is fused to a site-specific recombinase, transposase, or a derivative thereof that retains DNA nicking and/or cleaving activity. Additional functionalities can be fused to the zinc-finger binding domain, including transcriptional activator domains, transcription repressor domains, and methylases. In some embodiments, dimerization of nuclease domain is required for cleavage activity.

Each zinc finger recognizes three consecutive base pairs in the target DNA. For example, a 3 finger domain recognized a sequence of 9 contiguous nucleotides, with a dimerization requirement of the nuclease, two sets of zinc finger triplets are used to bind a 18 nucleotide recognition sequence.

Zinc finger binding domains can be “engineered” to bind to a predetermined nucleotide sequence. Non-limiting examples of methods for engineering zinc finger proteins are design and selection. A designed zinc finger protein is a protein not occurring in nature whose design/composition results principally from rational criteria. Rational criteria for design include application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496.

In one embodiment, the present invention employs markerless genomic integration of an exogenous nucleic acid using a site-specific nuclease. In one embodiment, an exogenous donor polynucleotide is introduced to a host cell, wherein the polynucleotide comprises a nucleic acid of interest (D) flanked by a first homology region (HR1) and a second homology region (HR2). HR1 and HR2 share homology with 5′ and 3′ regions, respectively, of a genomic target site (TS). A site-specific nuclease (N) is also introduced to the host cell, wherein the nuclease is capable of recognizing and cleaving a unique sequence within the target site. Upon induction of a double-stranded break within the target site by the site-specific nuclease, endogenous homologous recombination machinery integrates the nucleic acid of interest at the cleaved target site at a higher frequency as compared to a target site not comprising a double-stranded break.

Various methods are available to identify those cells having an altered genome at or near the target site without the use of a selectable marker. In some embodiments, such methods seek to detect any change in the target site, and include but are not limited to PCR methods, sequencing methods, nuclease digestion, e.g., restriction mapping, Southern blots, and any combination thereof.

Cleavage domains useful according to the present invention can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., 51 Nuclease; mung bean nuclease; pancreatic DNase I; micrococcal nuclease; yeast HO endonuclease; see also Linn et al. (eds.) Nucleases, Cold Spring Harbor Laboratory Press,1993). Non limiting examples of homing endonucleases and meganucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII are known. See also U.S. Pat. No. 5,420,032; U.S. Pat. No. 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue. One or more of these enzymes (or functional fragments thereof) can be used as a source of cleavage domains and cleavage half-domains.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, in one embodiment, fusion proteins comprise the cleavage domain (or cleavage half-domain) from at least one Type IIS restriction enzyme and one or more zinc finger binding domains, which may or may not be engineered.

An exemplary Type IIS restriction enzyme, whose cleavage domain is separable from the binding domain, is Fok I. This particular enzyme is active as a dimer. Bitinaite et al. (1998) Proc. Natl. Acad. Sci. USA 95: 10,570-10,575. Accordingly, for the purposes of the present disclosure, the portion of the Fok I enzyme used in the disclosed fusion proteins is considered a cleavage half-domain. Thus, for targeted double-stranded cleavage and/or targeted replacement of cellular sequences using zinc finger-Fok I fusions, two fusion proteins, each comprising a FokI cleavage half-domain, can be used to reconstitute a catalytically active cleavage domain. Alternatively, a single polypeptide molecule containing a zinc finger binding domain and two Fok I cleavage half-domains can also be used. Parameters for targeted cleavage and targeted sequence alteration using zinc finger-Fok I fusions are provided elsewhere in this disclosure.

Exemplary Type IIS restriction enzymes are described in co-owned International Publication WO 2007/014275, incorporated by reference herein in its entirety. Additional restriction enzymes also contain separable binding and cleavage domains, and these are contemplated by the present disclosure. See, for example, Roberts et al. (2003) Nucleic Acids Res. 31:418-420.

In some embodiments of the methods described herein, a host cell genome is contacted with one or more nucleases capable of cleaving, i.e., causing a double-stranded break at a designated region within a selected target site. In some embodiments, a double-strand break inducing agent is any agent that recognizes and/or binds to a specific polynucleotide recognition sequence to produce a break at or near the recognition sequence. Examples of double-strand break inducing agents include, but are not limited to, endonucleases, site-specific recombinases, transposases, topoisomerases, and zinc finger nucleases.

In some embodiments, each of the one or more nucleases is capable of causing a double-strand break at a designated region within a selected target site (TS)x. In some embodiments, the nuclease is capable of causing a double-strand break at a region positioned between the 5′ and 3′ regions of (TS)x with which (HR1)x and (HR2)x share homology, respectively. In other embodiments, the nuclease is capable of causing a double-strand break at a region positioned upstream or downstream of the 5′ and 3′ regions of (TS)x.

A recognition sequence is any polynucleotide sequence that is specifically recognized and/or bound by a double-strand break inducing agent. The length of the recognition site sequence can vary, and includes, for example, sequences that are at least 10, 12, 14, 16, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or more nucleotides in length.

In some embodiments, the recognition sequence is palindromic, that is, the sequence on one strand reads the same in the opposite direction on the complementary strand. In some embodiments, the cleavage site is within the recognition sequence. In other embodiments, the cleavage site is outside of the recognition sequence. In some embodiments, cleavage produces blunt end termini. In other embodiments, cleavage produces single-stranded overhangs, i.e., “sticky ends,” which can be either 5′ overhangs, or 3′ overhangs.

In some embodiments, the recognition sequence within the selected target site can be endogenous or exogenous to the host cell genome. When the recognition site is an endogenous sequence, it may be a recognition sequence recognized by a naturally-occurring, or native double-strand break inducing agent. Alternatively, an endogenous recognition site could be recognized and/or bound by a modified or engineered double-strand break inducing agent designed or selected to specifically recognize the endogenous recognition sequence to produce a double-strand break. In some embodiments, the modified double-strand break inducing agent is derived from a native, naturally-occurring double-strand break inducing agent. In other embodiments, the modified double-strand break inducing agent is artificially created or synthesized. Methods for selecting such modified or engineered double-strand break inducing agents are known in the art. For example, amino acid sequence variants of the protein(s) can be prepared by mutations in the DNA. Methods for mutagenesis and nucleotide sequence alterations are known in the art.

In some embodiments of the methods provided herein, one or more of the nucleases is an endonuclease. Endonucleases are enzymes that cleave the phosphodiester bond within a polynucleotide chain, and include restriction endonucleases that cleave DNA at specific sites without damaging the bases. Restriction endonucleases include Type I, Type II, Type III, and Type IV endonucleases, which further include subtypes. Restriction endonucleases are further described and classified, for example, in the REBASE database (webpage at rebase.neb.com; Roberts, et al., (2003) Nucleic Acids Res 31:418-20), Roberts, et al., (2003) Nucleic Acids Res 31:1805-12, and Belfort, et al., (2002) in Mobile DNA II, pp. 761-783, Eds. Craigie, et al., ASM Press, Washington, D.C.

In one embodiment, endonucleases useful according to the present invention are homing endonucleases, which bind and cut at a specific recognition sequence. The recognition sites for homing endonucleases are typically about 18 bp or more. In some embodiments, the homing nuclease is selected from the group consisting of: H-Drel, I-SceI, I-SceII, I-SceIII, I-SceIV, I-SceV, I-SceVI, I-SceVII, I-CeuI, I-CeuAIIP, I-CreI, I-CrepsbIP, I-CrepsbIIP, I-CrepsbIIIP, I-CrepsblVP, I-TliI, I-PpoI, Pi-PspI, F-SceI, F-SceII, F-SuvI, F-CphI, F-TevI, F-TevII, I-AmaI, I-Anil, I-ChuI, I-Cmoel, I-CpaI, I-CpaII, I-CsmI, I-CvuI, I-CvuAIP, I-DdiI, I-DdiII, I-DirI, I-DmoI, I-HmuI, I-HmuII, I-HsNIP, I-LlaI, I-MsoI, I-NaaI, I-NanI, I-NclIP, I-NgrIP, I-NitI, I-NjaI, I-Nsp236IP, I-PakI, I-PboIP, I-PcuIP, I-PcuAI, I-PcuVI, I-PgrIP, I-PobIP, I-PorI, I-PorIIP, I-PbpIP, I-SpBetaIP, I-ScaI, I-SexIP, I-SneIP, I-SpomI, I-SpomCP, I-SpomIP, I-SpomIIP, I-SquIP, I-Ssp68031, I-SthPhiJP, I-SthPhiST3P, I-SthPhiSTe3bP, I-TdeIP, I-TevI, I-TevII, I-TevIII, I-UarAP, I-UarHGPAIP, I-UarHGPA13P, I-VinIP, I-ZbiIP, PI-MgaI, PI-MtuI, PI-MtuHIP PI-MtuHIIP, PI-PfuI, PI-PfuII, PI-PkoI, PI-PkoII, PI-Rma43812IP, PI-SpBetaIP, PI-Scel, PI-TfuI, PI-TfuII, PI-Thyl, PI-TliI, or PI-TliII, or any variant or derivative thereof.

In some embodiments of the methods provided herein, one or more of the nucleases is a site-specific recombinase. A site-specific recombinase, also referred to as a recombinase, is a polypeptide that catalyzes conservative site-specific recombination between its compatible recombination sites, and includes native polypeptides as well as derivatives, variants and/or fragments that retain activity, and native polynucleotides, derivatives, variants, and/or fragments that encode a recombinase that retains activity. The recognition sites range from about 30 nucleotide minimal sites to a few hundred nucleotides. Any recognition site for a recombinase can be used, including naturally occurring sites, and variants.

In some embodiments of the methods provided herein, one or more of the nucleases is a transposase. Transposases are polypeptides that mediate transposition of a transposon from one location in the genome to another. Transposases typically induce double strand breaks to excise the transposon, recognize subterminal repeats, and bring together the ends of the excised transposon. In some systems other proteins are also required to bring together the ends during transposition. Examples of transposons and transposases include, but are not limited to, the Ac/Ds, Dt/rdt, Mu-Ml/Mn, and Spm(En)/dSpm elements from maize, the Tam elements from snapdragon, the Mu transposon from bacteriophage, bacterial transposons (Tn) and insertion sequences (IS), Ty elements of yeast (retrotransposon), Tal elements from Arabidopsis (retrotransposon), the P element transposon from Drosophila (Gloor, et al., (1991) Science 253:1110-1117), the Copia, Mariner and Minos elements from Drosophila, the Hermes elements from the housefly, the PiggyBack™ elements from Trichplusia ni, Tc1 elements from C. elegans, and IAP elements from mice (retrotransposon).

The Cre-LoxP recombination system is a site-specific recombination technology useful for performing site-specific deletions, insertions, translocations, and inversions in the DNA of cells or transgenic animals. The Cre recombinase protein (encoded by the locus originally named as “causes recombination”) consists of four subunits and two domains: a larger carboxyl (C-terminal) domain and a smaller amino (N-terminal) domain. The loxP (locus of X-over P1) is a site on the Bacteriophage P1 and consists of 34 bp. The results of Cre-recombinase-mediated recombination depend on the location and orientation of the loxP sites, which can be located cis or trans. In case of cis-localization, the orientation of the loxP sites can be the same or opposite. In case of trans-localization, the DNA strands involved can be linear or circular. The results of Cre recombinase-mediated recombination can be excision (when the loxP sites are in the same orientation) or inversion (when the loxP sites are in the opposite orientation) of an intervening sequence in case of cis loxP sites, or insertion of one DNA into another or translocation between two molecules (chromosomes) in case of trans loxP sites. The Cre-LoxP recombination system is known in the art, see, for example, Andras Nagy, Cre recombinase: the universal reagent for genome tailoring, Genesis 26:99-109 (2000).

The Lox-Stop-Lox (LSL) cassette prevents expression of the transgene in the absence of Cre-mediated recombination. In the presence of Cre recombinase, the LoxP sites recombine, and the stop cassette is deleted. The Lox-Stop-Lox (LSL) cassette is known in the art. See, Allen Institute for Brain Science, Mouse Brain Connectivity Altas, Technical White Paper: Transgenic Characterization Overview (2012).

### Materials for Correction of Undesirable Nucleic Acid Sequences in Animals

The present invention also provides materials for correction of an undesirable nucleic acid sequence (such as a deleterious recessive mutation) in animals. In one embodiment, the present invention provides a composition comprising a correction construct, a site-specific nuclease, and, optionally, one or more recipient cells. The recipient cells may be, for example, SSCs of a male animal whose genome contains an undesirable nucleic acid sequence (such as a deleterious recessive mutation).

Optionally, the composition may also comprise any material useful for performing the correction method of the present invention. The kit may also comprise, e.g., vectors, culture media, preservatives, diluents, components necessary for detecting the detectable agent (e.g., a selectable marker).

### Delivery Methods

The nucleic acids (including nucleic acid molecules encoding a site-specific nuclease, the correction construct) as described herein can be introduced into a cell using any suitable method. Nucleases can also be introduced directly into the cells. For example, two polynucleotides, each comprising sequences encoding one of the aforementioned polypeptides, can be introduced into a cell, and when the polypeptides are expressed and each binds to its target sequence, cleavage occurs at or near the target sequence. Alternatively, a single polynucleotide comprising sequences encoding both fusion polypeptides, is introduced into a cell. Polynucleotides can be DNA, RNA or any modified forms or analogues or DNA and/or RNA.

In certain embodiments, one or more proteins can be cloned into a vector for transfection of cells. Any vector systems may be used including, but not limited to, plasmid vectors, retroviral vectors, lentiviral vectors, adenovirus vectors, poxvirus vectors; herpesvirus vectors and adeno-associated virus vectors, etc. See, also, U.S. Pat. Nos. 6,534,261; 6,607,882; 6,824,978; 6,933,113; 6,979,539; 7,013,219; and 7,163,824, incorporated by reference herein in their entireties.

In certain embodiments, the nucleases and exogenous sequences are delivered in vivo or ex vivo in cells. Non-viral vector delivery systems for delivering polynucleotides to cells include DNA plasmids, naked nucleic acid, and nucleic acid complexed with a delivery vehicle such as a liposome or poloxamer.

Methods of non-viral delivery of nucleic acids in vivo or ex vivo include electroporation, lipofection, microinjection, biolistics, virosomes, liposomes (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787), immunoliposomes, polycation or lipid:nucleic acid conjugates, naked DNA, artificial virions, viral vector systems (e.g., retroviral, lentivirus, adenoviral, adeno-associated, vaccinia and herpes simplex virus vectors as described in WO 2007/014275 for delivering proteins comprising ZFPs) and agent-enhanced uptake of DNA.

Additional exemplary nucleic acid delivery systems include those provided by Amaxa Biosystems (Cologne, Germany), Maxcyte, Inc. (Rockville, Md.) and BTX Molecular Delivery Systems (Holliston, Mass.) and Copernicus Therapeutics Inc., (see for example U.S. Pat. No. 6,008,336).

Lipofection is described in for example, U.S. Pat. No. 5,049,386; U.S. Pat. No. 4,946,787; and U.S. Pat. No. 4,897,355 and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

Conventional viral based systems for the delivery of nucleases and nucleic acid molecules include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression.

In certain embodiments, the nucleic acids are delivered using viral vectors such as lentiviral vectors. Viral vectors may be used to deliver the donor nucleic acids as well if the donor is flanked by the nuclease molecules or other nuclease target sites that would allow for the generation of a linear donor molecule with single stranded overhangs that are compatible with those at the integration site following nuclease cleavage. Lentiviral transfer vectors can be produced generally by methods well known in the art. See, e.g., U.S. Pat. Nos. 5,994,136; 6,165,782; and 6,428,953. Preferably, the lentivirus donor construct is an integrase deficient lentiviral vector (IDLV). IDLVs may be produced as described, for example using lentivirus vectors that include one or more mutations in the native lentivirus integrase gene, for instance as disclosed in Leavitt et al. (1996) J. Virol. 70(2):721-728; Philippe et al. (2006) Proc. Nat'l Acad. Sci. USA 103(47):17684-17689; and WO 06/010834. In certain embodiments, the IDLV is an HIV lentiviral vector comprising a mutation at position 64 of the integrase protein (D64V), as described in Leavitt et al. (1996) J. Virol. 70(2):721-728. Additional IDLV vectors suitable for use herein are described in U.S. Patent Publication No. 20090117617, incorporated by reference herein.

Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)).

Replication-deficient recombinant adenoviral vectors (Ad) can be produced at high titer and readily infect a number of different cell types. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including nondividing, differentiated cells such as those found in liver, kidney and muscle. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)).

Microinjection: Direct microinjection of DNA into various cells, including egg or embryo cells, has also been employed effectively for transforming many species. In the mouse, the existence of pluripotent embryonic stem (ES) cells that are culturable in vitro has been exploited to generate transformed mice. The ES cells can be transformed in culture, then micro-injected into mouse blastocysts, where they integrate into the developing embryo and ultimately generate germline chimeras. By interbreeding heterozygous siblings, homozygous animals carrying the desired gene can be obtained.

### Spermatogonial Stem Cell Transfer

In one embodiment, the SSC transfer method useful according to the present invention comprises:

providing spermatogonial stem cells (SSCs) from a male donor animal;

introducing the donor SSCs into a reproductive organ of a sterile male recipient animal, whereby the sterile male recipient produces donor-derived, fertilization-competent, haploid male gametes; and optionally,

collecting the donor-derived, fertilization-competent, haploid male gametes produced by the sterile male recipient.

In certain embodiments, the SSC transfer method uses sterile, hybrid male recipient animals or sterile male recipient animals that have been genetically modified to have heritable male sterility.

In one embodiment, the recipient male animal is genetically modified such that it has an intact spermatogenic compartment but cannot perform spermatogenesis.

In certain embodiments, the sterile recipient animal can be produced via deletion or inactivating mutations of genes including, but not limited to, Deleted-in-Azoospermia like (DAZL); protamine genes (e.g., PRM1, PRM2) associated with DNA packaging in the sperm nucleus; genes in the azoospermia factor (AZF) region of the Y chromosome (such genes include, but are not limited to, USP9Y); and genes associated with male meiosis (such genes include, but are not limited to, HORMA domain-containing protein 1 (HORMAD1)). In another embodiment, the sterile recipient animal can be produced via genetic mutation(s) associated with sertoli cell-only syndrome (such genetic mutation includes mutations in USP9Y). In one specific embodiment, the recipient male animal is genetically modified such that it does not express functional Deleted-in-Azoospermia like (DAZL) protein. In one specific embodiment, the recipient male animal is genetically modified such that the DAZL gene is deleted.

In one specific embodiment, the recipient male animal is genetically modified such that the DAZL gene does not encode functional DAZL protein.

As used herein, an inactivating mutation refers to any mutation (genetic alteration of a DNA molecule) that leads to an at least 30% reduction of function of the protein encoded by the DNA molecule. In one embodiment, a 100%-inactivating mutation is any mutation (genetic alteration of a DNA molecule) that leads to a complete loss of function of the protein encoded by the DNA molecule. In one embodiment, the present invention provides a method for effecting spermatogonial stem cell (SSC) transfer, wherein the method comprises:

providing spermatogonial stem cells (SSCs) from a male donor animal;

introducing the donor SSCs into a reproductive organ of a sterile, hybrid male recipient animal, whereby the sterile, hybrid male recipient produces donor-derived, fertilization-competent, haploid male gametes; and optionally,

collecting the donor-derived, fertilization-competent, haploid male gametes produced by the sterile, hybrid male recipient.

The term “hybrid animal,” as used herein, refers to a crossbred animal with parentage of two different species. Hybrid male animals are usually sterile and cannot produce fertilization-competent, haploid male gametes. Examples of hybrid animals include, but are limited to, mules (a cross between a horse and a donkey), ligers (a cross between a lion and a tiger), yattles (a cross between a yak and a buffalo), dzo (a cross between a yak and a bull), and hybrid animals that are crosses between servals and ocelots/domestic cats. Hybrid animals include animals with 50:50 mixtures of parentage, as well as animals with mixtures different from 50:50 parentage, provided that the hybrid offspring of such mixtures is sterile.

In another embodiment, the SSC transfer method useful according to the present invention comprises:

providing spermatogonial stem cells (SSCs) from a male donor animal;

introducing the donor SSCs into a reproductive organ of a genetically-modified, sterile male recipient animal, whereby the sterile male recipient produces donor-derived, fertilization-competent, haploid male gametes, and wherein the sterile male recipient animal is genetically modified such that it has an intact spermatogenic compartment but cannot perform spermatogenesis; and optionally,

collecting the donor-derived, fertilization-competent, haploid male gametes produced by the sterile male recipient.

In another embodiment, the present invention provides a method for effecting spermatogonial stem cell (SSC) transfer, wherein the method comprises:

providing spermatogonial stem cells (SSCs) from a male donor animal;

introducing the donor SSCs into a reproductive organ of a genetically-modified male recipient animal whereby the recipient produces donor-derived, fertilization-competent, haploid male gametes, wherein the recipient animal is genetically modified such that the native male gametes produced by the recipient animal express at least one detectable biomarker label; optionally,

distinguishing the native male gametes produced by the recipient animal from the donor-derived male gametes produced by the recipient animal based on the detectable biomarker label; and optionally,

collecting donor-derived, fertilization-competent, haploid male gametes produced by the recipient animal.

In one specific embodiment, the native male gametes produced by the recipient animal express at least one detectable cell surface biomarker (such as cell-surface antigen tag(s)).

In one embodiment, native male gametes produced by the recipient animal express luminescent proteins. In one embodiment, native male gametes produced by the recipient animal are distinguished from the donor-derived male gametes produced by the recipient animal by flow sorting, such as fluorescence activated cell sorting (FACS) and magnetic-activated cell sorting (MACS).

In one embodiment, the genetically-modified recipient male animal comprises a reporter gene for expression on the cell surface of native male gametes. In certain embodiments, the reporter gene encodes a luminescent protein.

The term “luminescent protein,” as used herein, refers to a protein that emits light. Luminescent proteins useful according to the present invention include, but are not limited to, fluorescent proteins including, but not limited to, green fluorescent protein, yellow fluorescent protein, cyan fluorescent protein, and red fluorescent protein; and phosphorescent proteins. Fluorescent proteins are members of a class of proteins that share the unique property of being self-sufficient to form a visible wavelength chromophore from a sequence of three amino acids within their own polypeptide sequence. A variety of luminescent proteins, including fluorescent proteins, are publicly known. Fluorescent proteins useful according to the present invention include, but are not limited to, the fluorescent proteins disclosed in U.S. Pat. No. 7,160,698, U.S. Application Publication Nos. 2009/0221799, 2009/0092960, 2007/0204355, 2007/0122851, 2006/0183133, 2005/0048609, 2012/0238726, 2012/0034643, 2011/0269945, 2011/0223636, 2011/0152502, 2011/0126305, 2011/0099646, 2010/0286370, 2010/0233726, 2010/0184116, 2010/0087006, 2010/0035287, 2007/0021598, 2005/0244921, 2005/0221338, 2004/0146972, and 2001/0003650, all of which are hereby incorporated by reference in their entireties.

In one embodiment, donor SSCs are introduced into the testis of the male recipient animal.

In one embodiment, male gametes produced by the recipient animal are sperm.

In one embodiment, the donor spermatogonial stem cells (SSCs) embody a genetic background of interest. In one specific embodiment, the donor animal is from the Genus of Bos, including but not limited to, Bos Taurus (domestic cattle).

In certain embodiments, the recipient animal can be adult animals or immature animals. In one embodiment, the recipient animal is in puberty.

In a further embodiment, the present invention further comprises the step of fertilizing an egg from an animal species of interest with the donor-derived, fertilization-competent, haploid male gamete produced by the recipient animal. Methods of fertilization of eggs are known in the art, and include, but are not limited to, intracytoplasmic sperm injection (ICSI) and round spermatid injection (ROSI).

Parentages of the recipient hybrid animal, the recipient animal, and/or the donor animal can be of any animal species including, but not limited to, species of cats; mice; rats; wolves; coyotes; dogs; chinchillas; deer; muskrats; lions; tigers; pigs; hamsters; horses; cattle; sheep;

goats; ducks; geese; chickens; primates such as apes, chimpanzees, orangutans, monkeys; and humans.

In certain embodiments, one or both parentages of the recipient hybrid animal, the recipient animal, and/or the donor animal can be of any vertebrates, including fish, amphibians, birds, and mammals. In certain embodiments, one or both parentages of the recipient hybrid animal, the recipient animal, and/or the donor animal are not a human.

In certain embodiments, one or both parentages of the recipient hybrid animal, the recipient animal, and/or the donor animal can be from any family of Equidae, Bovidae, Canidae, Felidae, and Suidae.

Parentages of the recipient hybrid animal can be of 50:50 percentage, or of any mixture of parentages (including but not limited to 60:40; 70:30; 80:20:90:10; and any mixture in between), provided that the mixture of parentages maintains the sterility of the hybrid animal.

Mammalian spermatogonial stem cells (SSCs) self-renew and produce daughter cells that commit to differentiate into spermatozoa throughout adult life of the male. SSCs can be identified by functional assays known in the art, such as transplantation techniques in which donor testis cells are injected into the seminiferous tubules of a sterile recipient.

In one embodiment, donor spermatogonial stem cells can be cryopreserved and/or cultured in vitro. Frozen spermatogonial stem cells can be grown in vitro and cryopreserved again during the preservation period.

SSCs can be cultured in serum-containing or serum-free medium. In one embodiment, the cell culture medium comprises Dulbecco's Modified Eagle Medium (DMEM), and optionally, fetal calf serum.

In certain embodiments, SSC culture medium can comprise one or more ingredients including, but not limited to, glial cell-derived neurotrophic factor (GDNF), fibroblast growth factor-2 (FGF2), leukemia inhibitory factor (LIF), insulin-like growth factor-I (IGF-I), epidermal growth factor (EGF), stem cell factor (SCF), B27-minus vitamin A, Ham's F12 nutrient mixture, 2-mercaptoethanol, and L-glutamine.

Methods for transplanting spermatogonial stem cells into recipient reproductive organs (such as, the testis) are known in the art. Transplantation can be performed by direct injection into seminiferous tubules through microinjection or by injection into efferent ducts through microinjection, thereby allowing SSCs to reach the rete testis of the recipient. The transplanted spermatogonial stem cells adhere to the tube wall of the recipient seminiferous tubules, and then differentiate and develop into spermatocytes, spermatids and spermatozoa, and finally mature following transfer to the epididymis.

Methods for the introduction of one or more SSCs into a recipient male also include injection into the vas deferens and epididymis or manipulations on fetal or juvenile testes, techniques to sever the seminiferous tubules inside the testicular covering, with minimal trauma, which allow injected cells to enter the cut ends of the tubules. Alternatively, neonatal testis (or testes), which are still undergoing development, can be used.

### EXAMPLES

Following are examples that illustrate procedures and embodiments for practicing the invention. These examples should not be construed as limiting.

### Example 1

### Correction of Deleterious Recessive Mutations in Animals

This Example illustrates a method for correcting recessive mutations in animals such as cattle. In one embodiment, the method comprises the following steps:

(1) Testicular tissue is obtained from a donor animal harboring a deleterious recessive mutation. The testicular tissue can be obtained through, for example, castration or biopsy.

(2) Spermatogonial stem cells (SSCs) are cultured from the testicular tissue.

(3) The genomic region surrounding the recessive mutation is sequenced. In one embodiment, it is necessary to sequence a minimum region of about 1 kb surrounding the recessive mutation. In a preferred embodiment, a genomic region of about 2 kb or more surrounding the recessive mutation is sequenced.

(4) A correction construct, which comprises arms that match the region sequenced in (3) and a nucleic acid molecule for correcting the recessive mutation. In one embodiment, the correction construct comprises an excisable selection marker. Examples of selection markers useful according to the present invention include, but are not limited to, antibiotic resistance, fluorescent cell sorting marker, magnetic cell sorting marker, and any combination thereof. The selection marker can be excisable by any recombinase (e.g., piggyback™, Cre-Loxp recombinase, Flp recombinase)

(5) SSCs cultured in (2) are co-transfected with a site-specific nuclease engineered that recognizes the mutation site, along with the correction construct described in (4). Suitable site-specific nucleases include, but are not limited to, TALEN, ZFN, and CRISPR.

(6) The corrected SSCs are selected by, for example, antibiotic selection or cell sorting marker. The selectable marker is excised. As a result, the corrected SSCs are genetically identical to the sire of the donor animal, except that the deleterious recessive mutation has been corrected.

(7) SSCs differentiation is confirmed. SSCs can sometimes de-differentiate into pluripotent stem cells in culture, but can be re-differentiated into SSCs through use of protein signaling molecules. In the event that SSCs de-differentiate into pluripotent stem cells in culture, the present invention involves re-differentiating the de-differentiated cells into SSCs.

(8) SSCs are introduced into the rete testis of an animal (such as, pubescent bull or bovid hybrid). After 4-5 months, modified sperm genetically identical to the sire, but with correction of the mutation, becomes available.

### Example 2

### Spermatogonial Stem Cell (SSC) Transfer with the Use of Sterile Hybrids as Recipeints

Many commercially valuable animal breeds can be bred to closely related species, resulting in hybrid offspring with male sterility. For instance, cow/yak hybrids result in sterile dzo; horse/donkey crosses result in sterile mules; serval or ocelot/domestic cat crosses produce sterile hybrids. Parentages of hybrid animals can be 50:50, as well as mixtures different from 50:50, including, but not limited to 60:40; 70:30; 80:20: or 90:10, respectively, provided the mixture of parentages maintains the sterility of the hybrid offspring In each of these cases, male sterility is caused by failure of spermatogenesis resulting from failure of meiosis as the parental chromosomes are different enough that they do not pair up correctly to allow production of spermatozoa. Nevertheless, all of the cellular machinery (e.g., Sertoli cells, androgen binding protein) needed to make spermatozoa is present and functional in the sterile, hybrid recipient animal. Also, spermatogenesis in the recipient animal can proceed using donor SSCs from closely related breeds.

In one embodiment in accordance with the present invention, with the use of sterile hybrids as recipients of SSCs, all of the sperms produced by the sterile recipient animal are from the donor animal (the sterile recipient is incapable of making functional sperms carrying its own genetic information).

In certain embodiments, the recipient animal and the donor animal are from the same taxonomic family, sub-family, genus, or sub-genus. In one embodiment, Bovid spermatozoa can be produced in the recipient Dzo. In another embodiment, spermatozoa from the genus of Bos can be produced in the recipient Dzo. One criterion for the selection of donor animal and the recipient animal is based on functional compatibility between the donor and recipient spermatogenesis physiology (e.g., number of division cycles, expected growth factors).

An example of SSC transfer using sterile hybrids as recipient animals is illustrated as follows. Briefly, a punch biopsy of the testis of a stud bull is obtained, flow-sorted for SSC markers on day 1 to enrich for the desired cell population, then cultured extensively to both expand the SSC population and to ensure that only cells capable of self-renewal remain in culture. Cells can be frozen and preserved at this stage. A recipient Dzo (yak/bull hybrid) in mid puberty is placed under general anesthesia. The rete testis is imaged with ultrasound, a catheter is placed in the rete testis, and donor SSCs from the stud bull are introduced into the recipient Dzo. As the recipient is in mid-puberty, cellular niches for SSC exist but have not been filled with the native (non-functional) SSCs. Four to five months after the SSC transfer, sperms of the recipient Dzo are collected. After the SSC transfer, all of the sperms collected from the Dzo are derived from the donor stud bull.

### Example 3

### Spermatogonial Stem Cell (SSC) Transfer with the Use of Recipient Animals Genetically Modified to Have Spermatozoa Identifiable by Flow-Sorting

In one embodiment, the SSC transfer is performed using recipient animals genetically modified such that their spermatozoa express markers that can be easily identified by flow sorting. Spermatozoa identifiable by flow sorting include spermatozoa expressing fluorescent proteins and spermatozoa expressing unique cell-surface markers that can be detected by antibody.

An example of SSC transfer using recipient animals genetically modified to have spermatozoa identifiable by flow-sorting is illustrated as follows. Briefly, a punch biopsy of the testis of a stud bull is obtained, flow-sorted for SSC markers on day 1 to enrich for the desired cell population, then cultured extensively to both expand the SSC population and to ensure that only cells capable of self-renewal remain in culture. Cells can be frozen and preserved at this stage. A recipient bull in mid puberty, genetically modified to express fluorescent proteins in the acrosome cap, is placed under general anesthesia. The rete testis of the recipient bull is imaged with ultrasound, a catheter is placed in the rete testis, and SSCs from the donor are introduced. As the recipient is in mid-puberty, cellular niches for SSC exist but have not been filled with the native SSCs. Four to five months after the SSC transfer, sperms from the recipient are collected and flow sorted; fluorescent sperms, which are native sperms carrying the genetic information of the recipient animal, are discarded. Non-fluorescent sperms include native sperms in which the acrosome reaction has initiated. 100% of the non-fluorescent viable sperms are derived from the donor stud bull. The sperms produced by the recipient animal need not be genetically modified.

### Example 4

### Spermatogonial Stem Cell (SSC) Transfer with the Use of Recipient Animals Genetically Modified for Male Sterility

In one embodiment, in order to allow for improved recovery of donor semen in SSC transfer, recipient animals are genetically modified to have heritable male sterility. Heritable male sterility can be caused by having an intact spermatogenic compartment with failure of spermatogenesis. In mice, over 100 genes can disrupt sperm development or function (Matzuk et al.). Rats with naturally occurring mutations in the Deleted-in-Azoospermia like (DAZL) gene are used for SSC transfer for experimental models. An example of SSC transfer using recipient animals genetically modified to have male sterility is illustrated as follows. Briefly, a genetic modification is introduced to the male recipient animal such that the modified recipient has an intact spermatogenic compartment, but cannot perform spermatogenesis. In one specific embodiment, the male recipient animal comprises a DAZL deletion. DAZL mutant or knockout cattle are created using any genetic modification technology, and maintained in the heterozygote state. Sterile males are created by crossing two heterozygote DAZL knockout parents.

Specifically, a punch biopsy of the testis of a valuable stud bull is obtained, flow-sorted for SSC markers on day 1 to enrich for the desired cell population, then cultured extensively to both expand the SSC population and to ensure that only cells capable of self renewal remain in culture. Cells can be frozen and preserved at this stage. A recipient bull in mid puberty, with homozygous DAZL knockout, is placed under general anesthesia. The rete testis of the recipient is imaged with ultrasound, a catheter is placed in the rete testis, and SSCs are introduced into the rete testis. As the recipient is in mid-puberty, cellular niches for SSC exist, but have not been filled with the native SSC. Four or five months after the SSC transfer, sperms are collected and flow sorted; all of the collected sperms are derived from the donor stud bull. The sperms produced by the recipient animal need not be genetically modified.

All references, including publications, patent applications and patents, cited herein are hereby incorporated by reference to the same extent as if each reference was individually and specifically indicated to be incorporated by reference and was set forth in its entirety herein.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

It should be understood that the examples and embodiments described herein are for illustrative purposes only and that various modifications or changes in light thereof will be suggested to persons skilled in the art and are to be included within the spirit and purview of this application.

### References

Matzuk M M, Lamb D J. The biology of infertility: research advances and clinical challenges. Nat Med. 2008; 14(11):1197-1213.
Newman et al., High-altitude pulmonary hypertension in cattle (brisket disease): Candidate genes and gene expression profiling of peripheral blood mononuclear cells, Pulm Circ. 2011 October-December; 1(4): 462-469
U.S. Pat. Nos. 8,158,356, 8,431,346, and 6,306,591.

We claim:

1. A method for correcting an undesirable nucleic acid sequence in an animal, wherein the method comprises:
obtaining one or more spermatogonial stem cells (SSCs) of a male animal that has an undesirable nucleic acid sequence in an endogenous nucleic acid molecule; and, optionally, determining a contiguous nucleic acid sequence of said endogenous nucleic acid molecule, wherein said contiguous nucleic acid sequence contains the undesirable nucleic acid sequence;
providing a correction construct comprising an exogenous nucleic acid molecule for correction of the undesirable nucleic acid sequence; and
introducing the correction construct into at least one of the SSCs using a site-specific nuclease, thereby obtaining at least one corrected SSC comprising a corrected nucleic acid molecule that does not contain the undesirable nucleic acid sequence; and, optionally,
introducing one or more corrected SSCs into a reproductive organ of a male recipient animal; and, optionally,
collecting the donor-derived, fertilization-competent, haploid male gametes produced by the male recipient.

2. The method, according to claim 1, wherein the undesirable nucleic acid sequence is a recessive mutation.

3. The method, according to claim 1, comprising determining a contiguous nucleic acid sequence of said endogenous nucleic acid molecule, wherein said contiguous nucleic acid sequence contains the undesirable nucleic acid sequence.

4. The method, according to claim 3, comprising the contiguous nucleic acid sequence has a length of at least 5,000 base pairs.

5. The method, according to claim 1, wherein the exogenous nucleic acid molecule comprises a nucleic acid sequence that is identical to a contiguous nucleic acid sequence of the endogenous nucleic acid molecule wherein said contiguous nucleic acid sequence contains the undesirable nucleic acid sequence, except that the nucleic acid sequence of the exogenous nucleic acid molecule has a corrected nucleic acid sequence at position(s) corresponding to the undesirable nucleic acid sequence.

6. The method, according to claim 1, wherein the exogenous nucleic acid molecule comprises a nucleic acid sequence encoding a protein of interest, or a fragment of the protein of interest.

7. The method, according to claim 1, wherein the correction construct further comprises an excisable selection marker.

8. The method, according to claim 7, wherein the selection marker is excisable by a recombinase.

9. The method, according to claim 1, wherein the site-specific nuclease is selected from transcription activator-like effector nucleases (TALENs), zinc-finger nucleases (ZFNs), and clustered regulatory interspaced short palindromic repeat (CRISPR)/Cas-based RNA-guided DNA endonucleases.

10. The method, according to claim 1, comprising introducing one or more corrected SSCs into a reproductive organ of a male recipient animal; and optionally,
collecting the donor-derived, fertilization-competent, haploid male gametes produced by the male recipient.

11. The method, according to claim 10, wherein the male gametes produced by the recipient animal are sperm.

12. The method, according to claim 1, wherein the male animal is from any family of Equidae, Bovidae, Canidae, Felidae, and Suidae.

13. The method, according to claim 1, wherein the male animal is a bovine animal.

14. The method, according to claim 1, wherein the male animal is of the black Angus breed.

15. The method, according to claim 10, wherein the male recipient animal is from any family of Equidae, Bovidae, Canidae, Felidae, and Suidae.

16. The method, according to claim 10, wherein the male recipient animal is a bovine animal.

17. The method, according to claim 10, wherein the male recipient animal is of the black Angus breed.

18. The method, according to claim 1, wherein the endogenous nucleic acid molecule contains a mutation associated with a heritable disease selected from alpha-mannosidosis, beta-mannosidosis, arthrogryposis multiplex (AM), contractural arachnodactyly (CA), developmental duplication (DD), neuropathic hydrocephalus (NH), idiopathic epilepsy, osteopetrosis, protoporphyria, pulmonary hypoplasia and anasarca (PHA), and titbial hemimelia (TH), and Brisket Disease.

19. The method, according to claim 18, wherein the corrected nucleic acid molecule does not contain a mutation associated with a heritable disease selected from alpha-mannosidosis, beta-mannosidosis, arthrogryposis multiplex (AM), contractural arachnodactyly (CA), developmental duplication (DD), neuropathic hydrocephalus (NH), idiopathic epilepsy, osteopetrosis, protoporphyria, pulmonary hypoplasia and anasarca (PHA), and titbial hemimelia (TH), and Brisket Disease.

20. A method for correcting an undesirable nucleic acid sequence in an animal, wherein the method comprises:
obtaining a recipient somatic cell of an animal that has an undesirable nucleic acid sequence in an endogenous nucleic acid molecule; and, optionally, determining a contiguous nucleic acid sequence of said endogenous nucleic acid molecule, wherein said contiguous nucleic acid sequence contains the undesirable nucleic acid sequence;
providing a correction construct comprising an exogenous nucleic acid molecule for correction of the undesirable nucleic acid sequence; and
introducing the correction construct into at least one of the recipient cells using a site-specific nuclease, thereby obtaining at least one corrected recipient cell comprising a corrected nucleic acid molecule that does not contain the undesirable nucleic acid sequence; and, optionally,
performing somatic-cell nuclear transfer.


SEQUENCE LISTING (6 sequences)

SEQ ID NO: 1 - DNA, 160958 bp, Bos taurus
atgaaatccttgctgggtacaataatctgggcggtaggttattttcttccatcattttaagtatgtcttgccattccctcctggcttgaagagtttctattgaaagatcagctgttatccttatgggaattcccttgcgtgttatttgttgtttttcccttgctgcttttaatatttgttctttgtgtttgatctttgttaatttgattaatatgtgtcttggggtgttttgccttgggtttatcctgtttgggattctctgggtttcttggacttgggtgattatttccttccccagtttagggaagttttcaactattatctcctcaagtattttctcatggtctttctttttgtcttcttcttctggaacccctatgattcgaatgttgtagcgtttaatattgtcctggaggtctctgagattgtcctcatttcttttaattcgtttttcttttatcctctctgattcatttatttctaccattctatcttctaattcactaatcctatcttctgcctctgttattctactatttgttgcctccagagtgtttttaatttcatttattgcattattcattatatattgactcttttttatttcttctaggtccttgttaaacctttcttgcatcttctcaatccttgtctccaagctatttatctgtgattccattttaatttcaagattttggatcaatttcactatcattattcggaattctttatcaggtagattccctatctcttcctcttttgtttggtttggtgggcatttatcctgttcctttatctgctggctattcctctgtctcttcatcttgtttaaattgctgagtttggggtgtcctttctgtattctggcagtttgtggagttctctttattgtggcttttcctcgctgtgtgtgggtttgtacaggtggcttgtcaaggtttcctggttagggaagcttgtgtcggtgttctgatgggtggagctgtatttcttctctttgttcagtcgcgctgtggggagggagggagggatgctgcaaacaaataacactggcgtgtgctcgcagtgcctcagccacactgggtctgcccccgctcacggcgcgtgtagcctccctgcccacactgctcgggctctaggttgttccgccgggaacaatccgaggctggccctgggttgcatgtacctcccaggtccaagccgctcaggttcaggcactcgggtagtcctcagaggcgcagactcagttgggcctgagttttgtgctcttcccaggtccgagcagctcaagtgatgaggtgtttggcgagcgccaatgctgcgacttatcgcctccccgccactcggttatctgggtgtaaaaccggcgcatcttctcaggcagatgttaaccgtccagacccccaagaagttttagttagcaaagaagcctgcttacagttttatagataatgtctctctggggctgcgattgcccccttccggctctggctgcctgtcaccggagggggaaggtctgcagccggctatctccgttcagtcctttgttccgtgcgcgggcctggcggtgtcttaggttggggctggcttttcgcgtggtagatatcccacagtctggtttgctagcccaaattatttcgctcagatagtgctcagggtattcaggccagattcttactctaagcgatgcagcccgcgccgcgcctccctgcccagcccccgcttgctaatggcgcgtgcaggcgtctgcgctgcttctccgctgagggagttaccgtagggctcgcaatcttcgagttttaattgtttatttattttttcttcctgttatgttgccctctgtgcttccaaagcttggcacagattcggcagtgagaaggtttcctggtgtttggaaacttctctctttttaagactcccttcccgggacggaactccgtccctccctcttttgtctctttttttgtcttttatattttttcctacctcctttcgaagagttggattgcttttctgggtgcctgacgtcctctgccggcattcagaagttgttttgtggaatttactcaacgtttaaatgctcttttgatgaatttgtgggggagaaagtgttctccccgtcctactcctacgccatcttggctcctcccaaatgtcaatttctttagtctttttctcttttaaaaatttagctttacagatcctgtttggtttttaatttgtgtttttttttaattacttaattacttctatgtttgaatttattttgttgtgataatatctttttttatgatatgtaactcattaaaatttgtaaagctcatatttggatacagcttttgttcccttagccccagttttgttatacatgaaaacagtatgttttcacactgtgggtaatagacaattgggtggtataatcattgtagtaggttatggccagtattttaaaaaacggtgataggataatacaagaatacagtgttcatcacatattctatggcaaatactgtgtactaaggttaaataagcgtcgttttgtgaaacttatttcaaatgcatctgtgtctatgttagatcacaatacagaatgttgttttattataggttttagtaaaaaaaaaaaaaagagcttaaaccactgtattaaaaggcagtttgtctaacatttagtaaagatttcaaacctggctgcctaaaataaacatgtgaggggcttgctattttgacacattctcttttttttttgacacattctgtgaccttattttaggcctgctgatacagccctccagtgctgggaccaaggactctgttttaacaaactctacctgtgtgattcttacagttcgggtttttgagaaccactgattcaggatatagatagaccttgagagcctgttgggctgttgtgacacggggcttgtggagagtacggctccttgcatgcatctgtcccaaggccgcctcttttctagctccgggggggacatggggaaggaaggaaagcgatcagatgggtccggtggtagtaggggtcggggaccgcctgtaaacatcttgttcgttgttaggatgacagaaaacatgaaatagctctaatgcctgcatttctgtcatggaatcgattagtgtatattgaatggctaatgtatataccccaaagatgattatctttaagcttaaaatgtataaactttcatacactagtccatgacaatcatgtttttttgatacatcccagaccatggtctcagatacagcagatacaagttgaaatcattaatgatcattcagcacattttacccccaaagctgataatgattgcaacattcagggcttgacaggaccctactaagctaggcaggaagcagttttaccatgtaggcatgattggagagaactccatttagcaaagtgaaaatggttcataaaccctaagatggtctatgagcttttcaattgttttcctttatattaacgtgtgctcggtcactcagttgtgtctgactctgcaaccccattgactgtagcccaccaggctcctctgtacatggaatttcccaggcaagaatactggagtgggttgccatttcctactccaggggatcttcccaacccagggatcaaactcgtgttgcttgcatctcctgcattggcaggtggattctttaccactagcgccacctggggtttgaataacttttctgaaagatcagagactagaatgagaccattttgaagattggaatcttgacacaaggaatgccgaagtcacttgttgcagtgacgtgtctgtgatagatgaaggaatcacgctcaggtgtgggtaggcatcaggtgttggggggcttgtttggtaaggatcttaatttgtggtctacaggggcactttgcattttcagctgagggctaaagatttctttttatgtctctgtccatgacgcattggctgtttcacttttttcttgctatgtagcaaaccaccacaaaccttacaaacagcagcaacaatcgtgtgctcgctcaggattctgccgtctctgcagggctcgttggggatggttcctcttggctccacgtagtctctgcagtcttcctgagcttcctcacagtgtggcccctggccaatggctacagggtcttttaaagcctggtctcaaaccccccagaatcttccctttgccactttctcttggtcaaagcaagtcccaagtctagcccggattcgggtggaagggaaagagattccacgccgtgaatggacaaggggcatgcacattcatcagtgggaagaatgtttagcgtctgtctttagagacagatctacctcgctggccttgcttttcctccatctctcctttcctccttccctgtttctttcttcatttctcccttccatcaaaatgtgccaactatccccttattataaaacatcaatatatctttatttaaagattagtctttttttctctaatacccttttcccctttcttgatattttcctagtggtatttttacattatacattttgttatagttttgggtatctcattagtatctaaattcctctaaggtagatctttgctttcttctttaggttctctatgaggtttgaatgaatgaatgaatgaaaactttagtatatcaaacaaaaggccagtttgtccagatgaagcatcagtttttgttaggcaagtatttaaaaaacaaaatgaaactgtcctttggctgttccacttctaattttccttgcttggtcagcgctcttcttcttaggcctgatattaaccagcttatgctttcagagaatttgtatttatagaatatattctatagtaaatatatagcatattttcacatgtacaaatatttgtttactttatcagttcagttcagtcattcagttgtgtccaactctttgtgaccccatggactacagcacaccaggcttccctgtccatcaccaattcccagagcttactcaaactcatgtccatcgaatcagtgatgccatccaaccatctcatcctctgtcattcccatctcctcccaccttcaacctttcccagcatcagggacttttcaaatgagtcagttcttcgcagggtcaaatgtactggtcctttcgggggtcatctgtcaccctgaaggcttcattttcctctcttctccgctggtaaccaggagtgtgaggaggaggctgtcggggtcattatgtgcgcgtcggtcaagtacaacatccggggtcccgccctcatcccgagaatgaagaccaagcaccgcatctactacatcaccctcttctccatcgtcctgctgggtctgatcgccacgggcatgtttcagttctggccgcactccatcgagtcctccggcgactggagcgtggagaagcgcagcgtccgagacgtgccgctggtcaggctgccggccgacagcccggtgcccgagcgcggcgacctcagctgcaggatgcacacgtgtttcgacgtctaccgctgcggcttcaaccccaagaacaagatcaaggtgtacatctacccgctgaagaagtacgtgggcgaggcgggtgtcccggtgagcagcaccatctcccgggagtacaacgagctgctcacggccatctcagacagcgactactacaccgacgacgtcacccgcgcctgcctgttcgtcccgtccatcgacctgctcaaccagaactcgctccgcgtgaaggagacggcgcaggcgctggcccagctctccaggtatccgcaggtctcccagaaactaggggcaggggagggcagagccggtttccagccggcggctcatctcctgtctcaggtgggcacgtgggtgagccgctgacttggaggaggacccgcgctgcgggcaggcagccggtctctgcaccaccctcttgggccactgctgtgggttcccaacttcttcttcttctttttttttttttaattttattttatttttaaactttacaaaattgtattagttttgccaaatatcgaaatgaatccgccacaggttcccaacttcttaacatttagaagctaaggagggctcatcttcccaatccttccttttaaatgtaaatgcctttccccacctcccctctttattactcacatatacatattcactataaaaagcttgaaaatacagttaagcaaaaagaagaaagttagagtcatcccataagcctgcttttgacataaccattaatattttgatgttgcttctcagtcgtttctgtataaaacatgtacaggggcacgcatagatgcattttagtatgtatatgcacatgtatatacacatatcggagaaggcaatggcaccccactccagtactcttgcctggaaaatcccatggatggaggagcctggtgggctgcagtccatggggtctcgaagggtcggacacgactgagcaacttcactttcacttttcactttcatgccctggagaaggaaatggcagcccactccagtgttcttgcctggagaatcccagggacgggggagcctggtgggctgccgtctctggggtcgcacagagtcagacacgactgacgcgacttagaagtagtatatacacatgtgtagttccctggtggctcagacagcaaagcatctgcctacaatgtgggagacctgagttcaatccctggatagggaagatctcctggaaaaggaaatggcaacccactccagtattcttgcctggaaagtcccatggacagagaagcctggtgggctacagtccatgaagtcgcaaaaagtcggacacgactgaacgacttcactttcactttcatacacatatgtgtatgtataacattttgctcacacagcaaagcgtattcattttgctgccttttccccttcatttaccatgcatgtgtttccaaataatttcatctttgtatgtcagtgctgtatgacatatacacagaatgtatatgtattcgtttttacagttctgtatgggactccattttgcacacattccctaatgtccttaattagccttctgtgtttctttgtgtttttgtaaccaaatggtgaacactgcagtagcgttcagattttcatcagcgtaagcagttctgcagtgcaccagcttggaggacacatgccctggctttcctttggaggagccaagccagtgcccggagcgctgttgcgatctccaggcatgcagtttgccttcctgagaagtgaccctgttacaggtcagggatgcttgctacttgttgtccagcttcctttgtggctcatatggtgaagaagctgcctaatgcaggaggcctgggtttgatccctgagtctggaagatcccctgcagaagagaatggcaacccactccagtattcttgcctggagaatccccatggccagaggagcctggtgggctgcagtccgtggggtcgcacagagtcagacatggctgagcaactaacactttgacttgtcatccagaaactgacttgcctgtttcttcacagttgatgcgtgaattctacatgtttactctcttattaggtgggatagaggtaccaatcacctcctgttcaacatgttgcctggaggccctccagattataacacggccctggatgtccccagagacaggtaggtgtatttggggttgtccacctgatgggtttctgaggattaagtcaatgtaggacccttttgttcatgtgaaatcatatttctaagcccactaagacataactttgtaatcagaattgtttgttcaagtgtaaaattgtcctcggtcttttcctccctgagacctggaagcattgaaacctgccagaatctgtcccgggaaactaatggagggttaggttccagtggagcatcttagaggtctgtcttgtcttcttagtccgggttatgtgctcctgagcacacggtctttcccctaaaaggcctggttcatatcactggcttgtgtcagctctctctacatttgggagcattaagtagagtctcagctcctgaggggagcaggcgtggccgtgtcattggagcctgtaacgtccgggaagtgaggcactctaagtaccgcttactctcttctttccttccttcctaatgaaacttccaaacaggcagaagtggcatggggtggccacgatggcggttcaagttcaggtgctgtgcatttggccctgttgtaccaactgcgtggcttcctggaaacctcagaagggaggtgcccctaaggttcctctcctagtgcttggcctgaggctccgaagagagcgggaagtgtgggcccagcatgagggtcctgggcagcagccacgggaggggattactgcaagttcagatatgaactcatttatacccagctttgttgcagttgatgtttaattcttgaaatcattgttactgaaagaggtgagaacagccttaggcccgctgtggtctctgcttacttgaggccaacatcgtcatttgtgaaaatctacacttagtactgcttttccccagaagattaagtctaagccctctttgcttttaagtgtttatctagagtcagtattgagtttctatctggaaagatgatcatcactagaaaagaccctagcgccatccctcagctttggtgtagaaaccattttggacggtagagggcagtcaggccacacaagttctgtagaaacccgctgaacacaaggcggtctctggtttggctcttcgcccagcgctccggagtgcgttactgtctatgcgtttgcttgttctgggaaagtgaaatgtgttaggggacagaaaagggaaggcagagaggtgctgggaatgcagtgtcttcacctgctgctctgggtttagtttgaggagggttcgtttctgtttgggacatgtcggttcttgctgacgctttggtatttgtatctgactgggagaaagatttcatttttatcgagtgcacatgttacaaggagtttcatttctctgcaggaaagtgtcaagtaatcaggagagagttgaagttcttgctggtgcttaggtgttaaggatcaggaagggtaaatcattttccctgtgcgtgtatttttggaagtgattataggcagtcctgtagcaagtaatgaatcatcaaatagtcctcctttatctttccacgtgggaaatttcattttgaagccgaggtggtcttcgactggttggagtaaggaatagtagatttgtttgttaatttttctgaccctttgtccttgctcctggattcagaggaggtaagaaccatgcgtgtaggatggttttagcagaagctgcatatctggttattcagaaattagaaaacaggctagatttagaccgtttctaagatgcccctgaaatctaaagtcataagtgtgatttttttttttttttttaagaattatctgactcagaaaaatgcaaggaccaggactaaagcagatgtcagctcagcaaatacatctttcatgctgtgtgaatctgtgtttctctttcagtttttgaaaatcctatgtgtggtacacatataccttctgggtacaagattgaaacagtgtgcaagtatacaaagagtaaagtgtaaaaatatgttttcaaattttcctctggttttactcttttactgagaggtaatcattgttctcaaatactgtgtgttttttgagtctttgatggatttctagtcaaacacccacatatattcacctatccattttaagtcaaatgatagcaaattatacctcttcaatgcctgactttactgatctttgttttctcagctatattctgtaattttattactactttagatttttattatcctttctttgcaagtatctctctcaaatttctacttttgtataggtgtatatttatttaaacttcttagggaaaattttaaaacctgtataaaagaagtgagccccacacaaccactgtccagctttgttaattgtcaacttgattcatctatgctcctaccttcttcccaccagattatctagaaacaaatctcagatgttgtatcattatgtccgtaaatatttccatttgagactgcagaagaagtttttgtgtgtaactcagtggctcagtcggtaaagaatctgcctgcaatgaaggagacccctggttctatccctgggttggaaagatcccctggagaagggaatggctacccactccagtattcttgcctggaaaatcccatggacagaggatcttggcgggctgtagtccatggggtcacaaggagttggacacaattgagtgactgatactttcaacttttttcaagggctttttaaaaaatataactacagcactaatatcatacctacagcaattaacacttagttctttaacatcactagatattcaccaaatatctagccgttgctcagatatatatgtctcattttctttttttacagtttgaatctgtaaacaaatgaggatagccagtagagttggtcagtttttttttttttgcaaaacctaaaacttgaatctttgtatttgagggattcgcaggcctgattaaaaggaagcaaaagatacacttgcagacaaaccgtgtttagctctgtggatactcagatgggaggaggcgagctgtgagggcatctggggcagacccagcgtagggacgagggtccacaagacagggaggaagagggctcagtgatgtccctgggtcttcgagtcccgggaagtgctgaagatgctggcggacactcccagcgacggagcacctggcctggggtgagtcgtggtgctcacagctatgctttcttaggtgcaccgttcccgttcagtctctctcagctttggaacagatctccgatttgggaaaacagaacgcgcttctcagcaattcacttttcagtcaatgtaaaacacatgtgctgtaaagcaagggaggcttacactattttctccttggtaatgttcaggaaagcttggcagaagaagatgcggtgatttggagtggaggtcttgtcagcagcagggccggtgtgaggcttgtttgactaattgcagcggtggctcctcctgagagctccagcgagattgcatgtgcgtgatctgtgacatgaggcatgcttccggttacctgcttgcctcctcccagggcttaaccccctccctcagccccggggctccagggagagagtaacagtgaaagttggtgtcatgtacacctgcttcctcaccacagatgctcgccccttcctctggggcgtgggtgtaattacatcacccaccacgccgcttacctgggccgcctgatgctggtatattggatctgtgtttcctcctctgcacatcgatcgtgggggcccctgcagtctgcttgccatcatggtcatggaacccacccaaattcatgttggacggagtcactgccgaatgctaatcaggaaaaatgagaccgtatgttggtactacggctagtcattaagttttacagagcggactggcagctggagaggtgttccagcctgcacattacctccctaaccagttcttggtttcataaagcttaaacaaacagattgggatttccaggggtggtttggaaatctttgaacttaaactggtcatgtaagaagggaatgtaggctgtcatcagggcagggaggggattggtagcaatcaagattgcaaaaaatgtggctttgcagttcatacattttggctcagactgtgccgcgcatggacctggcagcactctgccccagtaagtgatttgatgggctcgggggccccccgggacacaataaccataaacgttggtgtgatgtacacctgcttcctttctaatgaagtgtcactgtattacttgctggatccagttaaagcagcgtgttcatggttttctaattaaggagaataaaagctcgtgtgggtggacttgcaggagaagcactcgaggtcaagttctggattctgcttcctgtggaagcatggggttgataagactgagtagctggaatgggtggtgagagctggtagaggctacacagtgcacacgaagctttgggacgacagtgaaaagaactgatcttgtgggaactggagcgggctggggcgcaggcagcgtgagatggggtactttgccgtcaccgtggctttttttttttttagctcattgtcccctgagcttccgtcagtgggacggtaagtgactgccccagccagtcctgttgttattcgtattactgtaaatgtgtttaagagtcagggcctcctcggtggctcagatggtaaagattctacccgcaatgcgggagaccctggtttgatcgctaggttgggaagatcccctggagaagggaatggtaacccacttcagcgtccttgcctggacaactccgtggacagaggagccttgagggctacagctcatggggttgcaaagagttggacgtgacggagcgactaacactaccacgttcaccgtctgtgacttctcggagaggtgaatgggatccaaacaaggtagcagaggggccgttcgcaggctgtcctcctggctgtgctgtattttaaaatagggaaagtaaggacagactgcttaggaccccagaggaaggcgggtgactcaggacgaggatgcagcgagtgcccctgagatgctgtacaacgtttagcactgtgtctttctagaaaagtgacttggtaaacatcagctgtggctgatactgttatgaacatccagctgcccctttccacagagcttatcagaacgaagactgtctttctttctcaccgtttgacaaaaccctttgtttttcagggctctgttggctggtggtggcttctctacgtggacttatcggcaaggctacgatgtcagcattcctgtctatagtccgctgtcagccgaggtggaccttccggagaaggggcccgggtaaggcgcctcgggcacagccagcggtgctgagagatgccagtgagggcccggggggcgggttgcactgcagtcgaaccatcagtgacaggccggggtccttgagggtccgtcattcctgtcccacaccaaccttatttatccttcagttctctctctgtctgtctctctctcagtcagtttgcagcgctgtctaccaagctccccaaatcagaaatctagttgctgtccataagtctttcctctcccttacttcctgtgcggaattgcaggcttccagaaggaaagcaggtgttcagcatcagttcagttcagttcagtcactcactcgtgtccgactctttgtgaccccatgaatcgcagcacgccagacctccctgtccatcaccaactcccggagttcactcagactcacggccatagagtcagtgatgccatccagccatctcatcctctgtcgtccccttctcctcctgcccccaatccctcccagcatcagagtcttttccattgagtcaactcttcgcatgaggtggccaaagtactggagtttcagctttagcatcattccttccaaagaaatcccagggctgatctccttcagaatggactggttggatctccttgcagtccaagggactctcaagagtcttctccaacaccacagttcaaaagcatcaattcttcggtgctcagccttcttaacggtccaactctcacatccatacataaccacaggaaaaaccatagccttgactagatgaacctttgttggcaaagtaatgtctctgcttttgaatatactatctaggttggtcataactttccttccaaggagtaagcgtcttttaatttcatggctgcagtcaccatctgcagtgattttggagcccccaaaaataaagtctgacactatttccactgtttccccatctatttcccatgaagtgatgagaccagatgccatgatcttcgttttctgaatgttgagctttaagccaactttttcactctgctctttcactttcatcaagaggcttttgagttcctcttcactttctgccataagggtggtgttatctgaatatctgaggttattgatatttctcccagcaatcttgattccagcttgtgtttcttccagtccagcgtttctcacgatgtactctgcatagaagttaaataagcagggtgacaatatacagcattcataaatcctgtcatttgtactgttttcccctgaaatcgaagttcccatgcaccagccaagggccagcctttcaaagaagagagccgccaggcccactgagttaagtctgttctgcacggggcactgtgggcagctaacaggcggaggttggtgatgccctaaacttctgccatgcccagggcggcccctcaccacaaagcatcattgggtccaaatgtcagcagtgctgtggctggagaaagcctgggggacacagggtgagcagtcttaagatggctcagactaaactctctttatcatctactctcccgagtgtaatctaggtctctaatgttggttgaggactggcacgtcccttaagatttaatccagggaagaaaacaggctgaaaggaaaagactgagagagattgaaacgaaggagagcaaaccactctgaacagtacaaattaccttcaaataagtttccaagaattacaagcttgctctgttggtggtattttcttggtaacttgatctctaatttttatgtgtaactagactgaaatgactatccttagatggtttaaggttgctgttgatctttagagtcctgagactctagaaactagagaatcttaagcagtctgtcttccagagcagcagcaggatgctcaagacacaaggggatggtcctccccgtggtggacaagcggtggatttctgtggcccgatgcggacccaccccactccgtttcccctccagggtttgcagttgggccctgtagttgtgctgtttcctttacaaataacactcctctgcctcaggagagaaaaaagaaaagcaaagacaggctctaggctcatcacactttccagggatctgggaggaggcagtctgggtgacctcacggcagcatttggaatctgttgctctttgtgagagttgatttttaagggaattcccacatctcttgagggctgcgtagagtagcaagggctttgtttttatttttattttaaatgtctctcaatattgtccaatgggaagctgaggattgttgctattgatagcttcttctcacagatgtgttggcctcagctggaattctcgtcatttctctctctaaaaggaactaaattgggagtttacaacggttactatggagggaaacttggagttctcagcatgattttgctgaaccttcgatatcagctctgctgggaagaaacatgaaagtcatagctggtcattttagtcctcgaaagaaaaacaaatccaccttaagattctgtttgcttaaaactaaaatgggcctactccaccagcccaccatctgccaggtcagagcgagccagcatgcaggcagggggaacatttttagactcttgttcacatctgcacatttatgtagaaaatggtggtgttgggtggggacccactgagcgtggtgactaagtgtctacaatagaactgtgtttcgtaacagcaaaatcagcccagagtctttctttactccagagtttctttgagcaccacgtgttagcatctgtacttgtttctccgtcttacgtagatggctccctctctctctgtctatgtgtattgaaaaccatgccttcccgtcgctctctcctattccatccagtgctgcaggggattctggctctctctctttccgtagctgtaatttccttctcggacagtgaggagtctagctcccgttatcccctgtatcttgcttgatcaaccctgtcgtgcgtgaccagagccctgtggtagcctctgcccctcaccgcgcaggtgtctatgcgccccacttgggctctgaccccctgtgccgagctcctgcttcccgtcgtcaccccggggcaggccccctcctcacatatcctgggctctgacccccccacactggaggggtgccgcccgtgccatcaccctccccgtgcgccttcagcctgggctgccctcctgccctgacttggcctccgcatcgcaggtggggaggccatgactccctctgtcccgtccccgctccctctttcagggatctcacagtctgggccaggccacctccctgctcttcccccaagaagttctcccccagatgaggactagagcactcccagtgggccagccccaccgcacggaaacgctgcctcccctccaggacgccagcactgcacacccgcctgcagggaccccctcggacaccagcgtggccgctcctgttcgaggcagcctggagtgccagttttgtgttagcggctctacttaaataggaagtctccagggacccagcaagtcccagcgctctgtggcagctgccgtagagccagcgtcttggggactggaaaggtctttagaggccgtttagctctcactttggagaaggaaatggcaacccactccagtattcttgcctggaaaatcccacagatggagaagcctggagggctacggtccatggggttgcaaagagtctgacacgactgagcaactaacactagttctgacttaatgtataccttgttactgtgatttcttcctgaaaagttttctgatttgtggtgaaaaagactcaccacctcacaagacagctcatcttcaaatggcctgagttgttgaggtggtttccgtgcttgcgttgagagtttgactgtccatcttcctcctgttctaagcagacgagcttgatcacagaagtcctccctttgggatgcctgaagaagactccctatatgtgatcttcccatcatacctctcccctctgaaaactgatgattttacgaaagaaaaaatgattgaggctgagggctgggggtagaattcaagagtctgtttcagtggctagcaggtatggggtcacacacagtcggacacgactgaagtgacttagcagcagcagcaacagcagcagcagccaatgtattgttcagcaaaatgcaattgaaggagaagttgtccaggatgagagaagccagtctgtacggctgatccaaactgtctcccgacgggctgctgttcggtgtcttttttacgtctgctcgcctttgccctgggctccgggtctgcactgctgtctggggcagctctctcaagcaggactttcagtgaggaaatgatctgtgtttagtggggtgccgacttccacgtatagctgttgagcccttgaaatggggctaaggtgaccgaggaactggattttaaaactttaatagatttaagtggtctcacgtggctaattgctattgaacagtgacgttctagggggtctgcttgtccctgtcttccctgcctgcctttgctttcagttcgactgtagagagaaatgaccggaagagatggttccggggacttgagggccatcagctgtggtgtgcgctgctccatttgctggagtcgagctgatctgcagctgggagagtgttcatttcctcactccctgcttgctctgtgtcttttgggcgtgacgcttaaagcttgggaaagataacactctctcagagagtgatttctttttggccacgggtagatgtgtaaggtgttctccagctggaccctccaattaagcctgagaaacgaaaggaaaataaagcaatttccctttctttctctccaaagcaagaagaaaagaaggtatggggttgggttcagactagagggtttgagtcctttctttctcactgcttcttgttagctcccctggtggccttgcctagagatggctgtcttgggtgttatccttggtctgtacagtttgaggaagatttgagtcctgcttctgggtttgatgggggtttcatttcttctggagcctgctttctatcattcctttcttcttcagtgatccctgctggttcctttgctgaggtcgtgtttattgcccctgtaacctctctgccttgtgggaatcttgccttctcggagagaattacttaccttctggtctccccagtgagttcacattttgtttctcgtggagatctgaatggtgctagacccttggaaatcctttcttcagtctgttctctctcacgcatccttgctatccgatgttaagactggttccatccattgacaaagccagttttttccacttgttctccacgaccaagtttggtcttcctgaatccatctggccttgaggatgggggctgcctttccctggagctcccccatagcatagggtgcccgtcagcagccggaagtaggctgctttcacggtcagtgctgtctgttaagaccccttgttttcctctttgaaggttgcagcctgctctcagctgcttttcagagcagagaactcagatcgaactaatgctaacgttggatgttcttccaaataatcaaccagtcagtcagtttctctctttacacacacacacacacacacacacacatacacactcacactcttaaagcatgctccctaagcttggcctattataagcatttcctcaaaacgttaagaacttttcctaaatgaaccctttaacaacagcatgatgcttcagcatgtgaatgagtcattgcttgccttctcagcatgtataatagtttaaatattcagcagtgctgagtgtagtttccccagaagtcaaggacgtgagtgaagatgggtgagaaagcactcgtagccctaagctctagacccgaagttggcctcaccgtgactcatgcagcggcggccagggtcccagtgccccccgttttctgcttcgcaggccgcggcggtacttcctgctgtcgtcccaggtggccctgcatccagagtacagagaggacctggccgccctccaggccagacacggcgaggcggtgctggtgctggacaagtgcagcaacctctccgagggcgtccctgccgcccggaggcgctgccaccagcagcaggcctttgactacccgcaggtgctgcaggtgagtgctccgcccgcccctctcagggcccgggctggggctgagcccagccgacaggcgctccgctccaagctgcactcgtcaagctccctgagcggagagcaaaaccggactgctctccactgctgccccgtggacttctgggagcttcagggtctgcgtgccgttagtgcaaagccgcttggcccctgagcgttccggctccttccacgaagctcatcttactgcaaaaagacacaccccgtagatgtacagaggagccgtgtagtcaaagcaggcacgtgtattttgcttttattcctgtattccttatccagcccagaggagcttgcttctttaagctgcagcttgtatcctccaactgagccagctcgccacggccacagttagttcttcctgggagtgggaggtctttattttttgttaccgttttttctcttggtgtttttgcctccctacatgtgtcctcctggtgtcgtgggagtgccagcacttcgtaactgcaggcagagtagtcctgtattatacttctgtattataagcaggctgtttgatactccaagagccagggggctgggaaagcagaaattgagttttgttcactctgctgttgtccagtcactcagtcgtatctgactgtctgtgatcccatggactgcagcacgcaaggcttccctgtccttcatcatctcctagagtttactcaaactcattgctatccaaccatctcatcctctgtcgtccccttctcctcccaccttcaatctttcccaacatcagggacttttcaaatgagtcagttcttcatatcaggtggccaaagtattggagcttcagcttctgcatcagcccttccaatgaatattcaagactggtttcctttaggattgactggtttgatctctttgcagtccaagggactctcgagagccccaacagttcaaaagcatcaggtctttggcactcagctttccttatattttaaatcttttgttcgcttaagtatttaaaacacaaatagttcagttcaaggagagatcttaaggagctggtgtgattgtgtgtgatttttattttttcaaagtctccgaagagaaccggcctgtgctgggattgttaaaataccttcaaaaagtgctgcatctcctctggaatgtctgactgataacagtggtgatgtgagcaggagtatttatagtaacctttgtctagacaggaaaaatggttatcagcacacatgcccacatctggacgccgactcttcggggccggctgagcgtccacctgcccctctgggcgccatgaccggtgccccttctccaccccctgccctttctcatcgctttctcctttatccccagaggaatttggcgtattgagttacttcatatttcttctgaactctccttttctctagctttaaagattaagtatttttgctttaaaatttttttttctgtttttctcctggagaggcaaaaactggaagaaacttgagtattagagacgggagctttccttatttttttcggttgacctattacaaccacctttttttcttgtgcatctctccttttcccaaacaggcccacatccagtgggttcgagggagtgaatgaggaagacctttagtgaggtcttgctttttgttcagttgtatctaaatagagcctatctgccttaatgtgttttcgaaggcatagcattcatgagatgaatgtcttctgtatacaaagcacactgctggatgctgtgggggtaggaagatgaaaggagatttgatgctttctgtgtagggggtcctctcaagagcccgatatgcccagtccaaataattctagtacaaggcaggcagaagtaagtattctgagccctgggaagccacggtggatatacacagcaccgcagggctgaaagaatccagtccgtgggaagtcatgctgttgagtttccagaggaagacctggaggtgggttgtgaacattttcagaatccaagcagcggaggcattgcagtgacactcaagagtggaaatccagagcattgcaacctcttcagcttttcagtctcacccgggtgagctgttatcttttagcattttctgtgttgcctggcttcagcccttgagagaactttgtggtctgccaggatcagagtaagtggatcagcggaaccggtttgtaatcccctcctctgcctgtgttctgcaggaggctactttctgcatggtccttcgtggagctcggctgggccaggcggtactgagtgatgtgttacgggccggctgcgtcccagtgatcatcgcagactcctacgttttgcctttctctgaagtccttgactggaagaggtgagtgttaccttctgatgaacctttatcccagggttcctgcatggattatttttaaagtcccccagcagttgtaatgcatagctaggtctgagaactgctccttcagattttttttgctccattttccatcaggagagttcgtccatgaacctgcagcagaggttggtcacccgaggcctttgggccgcatgtggcatgctgccagtgtctgtaaataaagtgttattgcatcacagcacacccattcattgacacataatctttggcggtttttgagctccagtggcagttgagtcctgcagtttaaaatgacagagattgtgtggccttgcatgtagtagggcctccataaatacatgctaaataaatgaatggattggggaatacagccgaaggagaagtcattgttaatttagagtggacagtgatgagtgaagacgagtgtacctggcaggcagaactggacacacagaggccctgtggtcgggcagagtgtggtgtattcaggaggctgagagggtacctgtgtggctggagcacagtggggagtggacggaggacaggagaggcatggagtggtgacggggaggtgagagatggagctcgggggagctcctttgcgctggtcggtcccaaggaagggagaggaaagagggactgctcactgcgaggaccgtggggctgagggaggcgcggggcattgttgcttctgctcgtgagacccgagcgggacacacatgcagccgtgctttgtatgcagggctgtccatacatccgctgtctccctctcgtctctagagcatctgtagttgtgccagaagaaaagatgtcagatgtctacagtattctgcagagcatcccccgaagacagattgaagaaatgcagagacaggtaaggggccaggcagccctgcgggggagggggcgagggggaggtgaaaggtggccttgactgcttggacgcagagcagtatccatgccgctgagagcgggcatgggattgatatcctccactgagtcggtcagccagacttaaaagaggaaaacagcgttagggagttgcatcagagaggcttggaataataactgtcaaagattcagaaacgctcctgaagcaagcctgctgtgacagtaatgtcacacctctgctatgtgttcaggatgcagagctgcccatctctggaaggccgtttacacagcaggtgagggctctggtgactgagacggacatggaagctggtgtgtttcagggtgatgcttctcaggagctttgaccttgagtgttgtgtatcagctagccagggtccccacagagtactgagcagagtttcctgtgctatgcagtagggccttattaattgtctattttatgtatagtagtgtgtatacatcagtcccaatctcccagtttattcctctctcccttccaccctggtaaccctgagtttgtcttctacatctgtgactctgcttctgttttgtaaataggttcatttgtactatcattttagattccacatataagcaacatcatctggtatttgtctttctctgtctgacctcactcacctctaggtctgtcacggtgctgccaatggcactgtttcatttgtgtggctaaaaccccatcgtatatgtgtacggcgtcttctttacccgttcctctgtccgtgggcatttaggttgcttccttgtcttggctgatgtaaacagggctactgcgaacattgaggcggtgcctgcgtcctttcagatcatggttttctccagttctttttcttcttttaatgggtttttacaagtttttattctcatcaaaattatctgtgtaggtgatgagccggagaagaggtttggagggttagggttaggctgtagccgcctccacacacccctgtgtctaacttggtcgcaggaatgagcctcaggtctctgggagagggcagggccgtcacccggtggtgtggaagtggggagggactctggcttctgacggctgcttacacacgttgtccgtctgtcctcctgccccgagctccgcctctccccccgagtgtgcgcagcggaacgcgggggtttctcggctcctctcgggtgctgtcttgggcctgtcgcgtcagccaccacctgctttcctgcttccacactttggtggtgttagctccttccccgtcctctttatctttggttatgccttcattttagggttttaggagggagttagtggatgtgttcagtccacctctttattttggacccagctccttagaccaaacagatggtacatcacacagtggttgagtctgtaggttccgaccctagactgcctgtggcgaatgccgccttggccggttactgactctgtgcccttggccaaggctctgccttctgtggatccccatgacctcatctgtcaagcattttgacattccgatttgaaaacaaatcacatgttaagtttagcctccccgtagtctatttcctagcacacccatgttcaggtttctgggcttccccatttgttgcttaactgctgacctcatttgagttgtagtgaattagaaaatcagcctagagatctctatagtcttgatctgaacttggggttcttacaactcatgggaagaaaaagtactccttgaagggacctggtgtttatgctctttgtgtttcattttatggcatcttgatttgttaacatagtaaatagggaatcatctgctaaatcaggtatcatcttttatagatttataaaaggctaagaaaacaccagggaggtgaaaaggagcaaattctgcccagttatcttccccactaccttcctgttgggaagagaatcacttagtttttagagcagagcacccagggccagctcacagaatgtactttttccctgggaacttggcttaggaaccacagatctgagacgagccttgccagtcttttcaaatgacaggttcagaatcagaaccattgaagcagagtgactaagggttacttaaaacagtgtacttttaggttatttgggctctgagagacacatgtggggtctttaaaagactctggcttgagccctgtgtcagtctctgttaattgtgtgactgtgacacatctgtgaccaaagctcctcagacttcctcatttatggaatcaagggtgggctggatcagctttcatgttccattcagctgttaaaaaacactgtgaaactcctgtccttgacagctttaataactgagtgatcatctctttcccatgaccacctcctgtcatgagggcctgttactgtctctggtggtgaataacctgtattatgtccactggcagaataaaggggatctcagtactttcctgggcttccctggtggcttagatggcaaagaatccgcctgccatgcaggagacccgggttcgatccattagttgagaagatctcccagagaagggactagcaacccactccagtgttcttgcctggagaattccacggacacacgagcctggtgggctacagtccatggggtcgcaaagagtcggatacgactgagcgactcgcacttgcagtactttcttccctcccctaaatcccccttctgcttgatggtgctcttctctctgctactttcagatgttctgagatgcttcgctgccgtggcccatttagtcctgctccctttctctgaatagtctgcatccccaaaatgacctcttttcttctcccaatgtcagatttctagactgaactgatctcagccagaggtgagattatccggttgaagcacttcccagagagttcgaaaggatgactaggttccactcccagattccagtggcacacctgactcccagattgtgacaccttgaaacggctgcacatgttgccagatgcctcccaaggcggcagaagtgctgtgcttgggagccgccttcctgttgataccgtggccgtctctctaagattagagacagctcttttgttctttgcagaatgtgcttgtcctttatctcctgtatcccctccattgattcagtcagaccttggcaggagtgactgaaaaaatagcacacgcttggttcttggaagttattacacctcagtgaattctctttagggaggtccaatttgagtctttgtaagacagggataaacttttaggggaaaacctgttaacatcagtagttgataccttagaaccctgggttgcttatgttagaatgagctgagactgaggagcagagggtcagattttcttgctcggcaactcaggaccggcccgaggggctgcacgcacagttgtaaccagcagagggctccatccactttgaagtgactcattttccttctgagtagccgtgtccaaggagtcctgagtgccatgagctatgctgacaccgttcctcagagccgctctctagagagacccgcttcttcctgtgctttttagcacaggattctgctctcctgtgatggatcagagccagactttctgaatttgacccctgagatacgggtgggatcttggtgaggacggtgatgcagcttcgagttcaggggctataatctgatcgatggatacgtggtgacagcccttaacatgacccaggagttaggacgcacccagagaggagggtgttctgcctgacaaggaagcgtaacttttcctcctcacctgagatgcgacagtgaagcagccgttaaagtcatatgcagggctagaacctgaagtaagagctggatgtaagctaagacttttgaagtacagcccgcatctgtgttatctacccctgaagaccaatagctggctgtgttggcgtaggtggcattggaagagctggggtggcctatagggctttctgtggtttagttagaaatcagttcctttcattaatagaaattaatagttgcttttgttgttgtccagtcggtaagtcatgtccgactctttgcaaccccttagactgtagcacgccaggcttccctgtccttcagtgtctcccagagtctgctcaaactcctgtccattgagtcagtgatgccgtccaaccatctcatcctctgctgcctccttctcctgccctcaatctttcccagcatcagggtcttttccaatgagtctgctctttgcatcaggtggccaaagtgttggagcttcagcttcagcatcagtcagaatattcataatattcagggttgatttccttcaggattgactggtttgattttcttgctatccaagggactctcaagaatcttctccaacattactgttcagaaacaccaattcttcggtgctcagccttctttatggtccacctctcacatctgtacatgactactgtgacccctacccacacatgcctaatagccatcctggggatatgtaggtataaggccaaagaaagctgagaagatggtacagattttccaaagcatgttagtggtctgaaatgggaggaagtctgggttaaagctgttagggacagatttattggggaggtagtacttgatggcagccttaaagatggagggttccatccactatctttctcgtgttggaagccaggaagaagtatgcaactaagtattttcctatagtctcatggaacattcacgtccccagtctcctttggaatcacaatgaaaaagagggcaacccaggatattgaaaggctgactagacactggactgtatggttggcagtgtttgtgtggatagggaggaaggtgtaaatggatcctcagtttatctgttgagtggagtttccaaacattcttttattggtcagtggctccgccagcagtgacctctaacctcagccacactaggaggtggaagtcaggagcctgccttgcctttgaactcttctttgtactcatttgactgggaacctttgaggtggcagaaacattcagcatcttctttgctctctctccttgaatgcttggccaacttaaaataccagtttcttttctattactgacccatctcagcctgccttcatattttccctattatactaagcataccaattatttgttctccggaggaggacgtgaagatataatgggctgattaggaaaaagaacatgtctgtcgaagaatatgtggtcccctctcctggcaggactgttgtcaggcctggctgtggtcgagcagagagcagaagagaccacagaacaggaagtgtgtgtgtccaccagttcatgcactttattcagggcagagaccccacaggaggctgctcttttatcttgcatgtgatttcagcacatccaactgtttgctttccatagacatggtagtttcgaagttacatctttgaaggacagaaagactgtaagtgaggggctgtgacatggggcatctgccagtcaggcatgaatagagaagacagaaaggctgttttaaaaatagaacttggatggaatgctctgtgaataataaatcctggagagggtgtggagagaagggaatcctcctgcactgttggtgggaatgtaaatcggtatagccgttatgagaacagtatgggggttccttaaaaaattaaagatagagctgcaatatgacctggcagtcccactcctgagcatatatttggagggaaacatgatccaaaaggatacatgtgccccagtgttcactgcagcactgtttacaatagccaagacaacctaaatgtctgtcgacagaggagtggataaaggtgtacagatatacaatggaatattactcaaccgttacaaagaatgaaataatgccatctgcagcaacgtggattgacctagagattgtcatactgagtgacataagtcagagaaggagaagtatcgtatgatatcccttatatatggaatctaaagagaactggtaccaatgaacttatgaaacagaaaaggacccgcagactcggagggtaaacctagggttgccaagatgagagatgcgggaaagaatagctggagagtttgggatggacctgtacacgctgctgtgattagaatggttagccaaggatggccagcgaggacctactgtatcgcaggaactctgctcagtgttgtgtggtggcctggatgggaggggagtttaacggagaatggatgcgtgtgtatgtatggccgggtccctgtgctgttcacctgaaactgccctaacattgtgtgttgattggctataccccaacacaagataaaaggttaaaaaaaaatagaacttggggcttcttggcactccagtggttagcacttcaccttccagtacaggctgtgtgggtttgatcctctgactgaggaactaaggtctcgcatgcgtcatggccaaaaatccagaacataaacaacagaagcaccattgtaacaaattcagtaaagacttttaaaaaatggtccacatcaaaaaaaacacaaaacttaaaaaatgaaaacagaactctcagcactctggatacatggcagacggcagaacgtgcttctcgatatcctgggtgtgtcctcgtaagacggtagaattccttggttcaaagaaatatgtacgtgtttgttttgcatattttgggtttctggcatccaaatgagtcctaccatcactttgattgttgaatatagtttctgttttgtcgccttgaagctgtttctgacggagcatcttaaaatcgtggaaatacagagatggctgtcctgagaacgccctgcagtcttttcagggaaggtttacgtttcgcgtatataaacgtgtttttaaaatactggctttcgcgtaaagtattttcgaaagttttctatctaaaataatttgcttttatttttttaaaagtgttttgtgatccgcctaaaggcgtcggccctcggatgtctaggacccccaggaacctggatggatgtgttctctgagggtcccctgcgcttggctgttgtgtccgttgacggctaggtagcagtctgtgtccatttctccctccctccctgcccccaaacacacacaatttacaaagcaaacttgttaaacagagcagtgagactaggcagtgagaggagcgatctgaattttttttaacgtgaggaaacggaagaagagccgtgaagcgatgctccggtgatcgcacgacctgagagggacagacggggaacatactgttttgctgcccacagcttgattcaaatcctagagctctgtggcctggattgaggttaaacgtcaggatgagcttcccagcaattcaggttagctgcttattggatcgtctctcctaaaaagtggttgcctgagaccctggagactggacacgtctctggaaatatgaagaacgtggaaaagacttcacttctgatttccaagatttgacattgtaacgtctgtcactctttcccactgcccgcccccccatactgtggaaattgaaaaaaactttaatatgcataaactgaattctcagggctttgaaaaaaatgccaagtattcccttcctccccctgctctctagtcccagcatgccactccaagctctgtttgccgatctctgctttcatactcgcccttaaaaaagctactggatcacttacccgtctacgaaacacgttgaccttgatccttggcgttctcctggtgaaacagacaaccgtggttctgatacaaagccaggctctttggccagtgggagagccttggggaagagtgtgattatgggatattttgagtttcataattttatttcaattttgttactctgttggagttttcctgaatgtataaatccatattgtgaaggagatcagtcaacttatgtttaaagcaggataaaataagggctcatttaattggaactcttaaggaagagcatttttatgtgtttaacttttttttttttcaagtttagcattaagaaccttattggaaaaagacttttgccaagtatctgatgggaagccaagaaaagcattttaatagaacggtctcaaatttatagtacaccagggcctgtaaataaatttccctgtctttaaggttggggctgatttgcagttattttgtatagcttctgagatttaaatcctcacagtccagatggggagaccataaagctgctttctttggcaaccctggcatacattgtccacggcgagctctgtctgtaaatgctcacttaccgtattgtgatgccagtttgaattggtttagcgacttttcctagagcagctccccccaccccaaatgttctcaaaatcttggagaattaagagtccttgatttcataatgcagactagtcaaataagtatgttttaagtaccggcttttttgctagaattgtataggtatattaaaggagatttttacaaagggggaggggaggaacacagatcctgtcctcagggtgctagggagagcactttcagcaatagtgaacgaagagggcggaagtgctggccctcagtcgtgctgcggaatggatcaggaggcgagagcaggcttcgtgaccgggtggcctcgaatagggtgagaggatgctctgagaggtgccgagctccgcccagaggttggcttctgtgggaaggactcggtaggtgaggtggaacagcctaccagcctgagggggaacttgaaataccagcaagagagaactttctaggaagctggaaggacgggaaagtcagtgacaagtggagagtaatgaaaaaatgcgcgaggaaaggggaaagcagtatcaattaaggagacaggggtaaaaaaaaaaaaatcaaagagctggaaggaaccttagaaattattcagccctttcttcatattttgtaaatgaggtggaagccctagcgttcaagtgatcttgaggatgaattgttagggcctgagcctcgcctcgcctggaacccaggtctgctgactccagctgatggattctgggtggaagggggaccttgtgtactacagtatttttcaatttagggagtcttgagtggcattttattttttaaaaaatgaaggataataggaaggcatcagagtattacctagcaagggtaaatctttcctgaactcttttttttgatatgtatatgtgcagtgtcattatttaatgtatttgtatgtttcttggtgtgggccccagtccagaaagtttgagggatgttgatagtgagtggcaagcacacaggcttttgagttgaggagacctgaatttgcatcccaggctgtgccttctgtctgccagctttgtgagcatgggcaagtcactttgccaagcatcaattttcttatcttagacctggggataatagtccgactctttgtgagcccacggactagaacctgccaggctcctctgttcatggaattctccaggcaataatactggagtgggttgccattcccttctggggatcttcctgacccagggatcgaacccaggtctcctgcactgcaagcagattctttaccatctgagctacagggacgcccccaataatacaatagagcagttgtgttgtttaaatgaaatccttgacttaaagcatctagaggccggtctgcctgctctgtgttggctgtcgtctgggagtgctccgcctgggtctaggtttgaacactggcaggagcctcctggtttggagcttggacttcaggaaccacagtaagttaccaacgcggatgtattatgcatttgcttctgggcttggaaacttgaaacagaacttattcctgtgggcctggtgtgctttctacatcttggaataaagggaggttcccctctctggttttcagaatggcttatgaaaatcagtggttctttagctggccttaaaaaaaattcattggcacgtttgagtgccagaattcataaagcaacgtttgggtcaagtactattactttttcccctttccagtgagaacgattttcatactgagtctttttaccccgtcttttaatgtatcctctgctggatcttgattttactcattttttttgagccagaagattaaaaaataagcataaagagaaaagtcaaaatcagaattgacatttgggtggtgaacacttggagagttggcattttactaggtatgtactaaaaataaagcaactttttatgtgaatttaagagggctggtgagatacctcttaggaatagctgttgttcgcctcctactgaaagggtaatgagttgtatcatctggttttcctggttctctgctgttcccctaacccctgctgcccttccccccacccctgctgcccttcccccacccctgtccagccagctccatcccatgtttgaaatcccgctggagagcgagtggagcgcttcatccctagatgatttgggccaggggctaaaacactgccataaatctgcagctcattttaatgccacgttaaaacagtgtgagcggctggtttccgtggcccctgccctctttctttatgggcttctttggctcttctttataaagaggccctgtggagtcattttaaagttcttatcagtttgctgacagctatatgggcctttctgctgagtccaggagctcaggcaggcgtgtgaaacttctgatattaaaattgaagaaaaatgccaggcagaagggattggggagcgcagcggtgatttggaggtgaaagactagcgaggtaattaacctctgcaggtccggaactgcgcccttggctggataagttgctcctggttggtgggggcagggcagcaagggttaggccagggagggctttccaagggcttttatttttatccccgatgagaaatgccgttcagctggcagcctgtgctttcaggcttcagcaagtgcgttgggcagtcctgaaaggctaagggagccaggtttacaatgcagtttttttttctttttcctaaaggccgtctgactggctagacctcggcttgtgagtctctggcagctccagctcactgtctctgtccacagcatcgctgttatcgaatttaaaggggctttcatcagtctgctccgcagccccttttagaccagcttgctaactcccagtgatcctcgcagagtgcttcgtggaagcgccaggccctctataaatgtcagcctgcagcttagcccttcttaccctctcagttacggagaggtagcctggaagccgctcctccctctcctccaccgcttttcctttctctctttgctggaaaactctagcagcagacagctgaggggaaggaaaaaccattacctggatcaaggccaggggcttgatggagcctcaaaatctctggcatctcctgaccagagtgtttttccaggcatttcaaaaagtacaccctcaccctccgccagctttcagctttccagccttgattccaggtgggctggcttgggggcatcatgccctcagaatctgtatctgtctgctcctcttctcctaactaaagcgtttaggaaaaggatagtagagataagcaaaatccagtggacatgtatttttaactgccttgcacccttttggcgcaaacacattatttttcctttttcaaatagttttggacatgagtacttttgcaagtgtattcaatttaaaatagtcatttaacttgaggttgaagcaaaagcaaagaatttttgtagttagtcaaattatatagctgttgatctaggatcaatgattttcattgagcaaacaataaaaaataccggttcgttttactacttgtgtggaagtagctcctgtggggggagtaattaggtatgaaaactgaccaagctataaatcaaaataaaataccccaacctccaaagcaatcaagctacttttttgaacatttctgacaatatcaatagatattttggtattctgaacaatatcagtagaataaattgatattctataattagttaagtttactgaatcagaagtttttgcttgtggtataattttaataaatttactaaaaatattttacaaataatgtactgaaagttctcagtaagtatatgattatgagcagtcttcattgtacagcctttgcaaggggtgtggttatatcatatgaccagtttcctctttgggcattggttgcagagctggctctagagttagccatgggttaatcccttgtaacaagtccccaatatggggcattgctttaggaaatggcctctctaataaaagtgttaaacattaatttgcagcctatataaggattgtctgactctttggggctggtttcctacagagtggaactatttgtgaagcatttctgagaaataattgctcattacaggaagtgtagtgcaggctttgtgaattcattatatatctacttagaaaaatatctaggtttgtttacaaaagaataacttataaattaccaagtgtcgatacaagcatttggtaaagaattcactattgtcaggtcttcagtaagagtcagactgatccattagatttctgtctatatttatgattattttgtatttgaatgtatttttctgtctccctcagcattgaagaggctgagagagtttggagttgcctgactactttgtaataacaccatcttgtaattccatgctactgttcttgggaatgtaagacaacaattgtgtatttatttcattttatttgactagatgacctttaatactgaatcagaccagaattggtacagaaatagcttcataaagtcatttctggcaatgtagttctctctctctccccttatagtttaaaggttaatttgtatttaaatgtgcacttatttcccattttgataagatttcagtagtaagcaagcaggtcatttgttatctttcagaattcagtactttttattaaaaggagcaagcggtttgtcttcaagtgtttgaagcccatatatgtatatatatatattgaagtgttctgaattctctgaatatatattggccaggtcttgatgattttatatatatatatatatatatatatatataaaatttgtattttagagagatctttgagtttttgtttgaagctacagactgagggttgaattctctcaactctcttgctctcactcagattcttagatgctttctggagtggggaatggggacataaatttctcttgagaatcctcgccgtgatgaacctctgctatgcttactcataggtaagaaagggcgcggggtaaagatgagaaccactcacagagcaagcatagctctttcaaacttctccctctgttcagagtcccaatttagggaacgcacattggggtttccactcagatcaacaggagagcgtgtcttaatacctgccacgtgatcagaccaaggcacttcccagctgcaagcggcttagggagggctcccgtggctccggtcagctggaaccggcattggacccacactattctcaacttctctgtttctgggaatgtgtggggacagtggagagggctcccatgcgacctgactgcgtctgtataatatccacagccagaaaaataaataaataaataaataacctgctaaagaatagctccccagccttctttagaattgttactacctgggaagcagatggaggggggaacatggccctgatgtggcaggatgtgggatgaaacgagccctcaagttctgtcctcggtcccttcacacgtgctatctgcgtgtgtgtcttaattaaaaggggtcggcctttcaagtgtgcccgctgcagtgagacttgttttatatttaccttttctgatcaaacaagcatacagctctctccctgttttggtatagcaaacatggggaatctcaccccaggcggattcctttacaacgaactgacccaagttgtgctcttatttatttatttttgatgacagatcctgattaaaggtttccagtccaaaccttttagctggtccatgaacccagccaggagagagggctggtcagctgctgattgccaaagaagctccccgacctgaccgagggaacagtgtaattcagaggtgtgggtaactggggaaccgggcacagcagaaatgaatctctcgtctctttgctgagaaggtttgaccaaacacagttttattggtcagtggctcctgctttagtaacctttcacccccatggctcttggaggtgtaagccactggccggccttgcctttgaactcctcatggctcctgccacctcaaagactcacagtcggagcttgtgttttgcttgggatctctcctcacagcagggcatcagcagttgggaaaactgatccacccttagagccttgacatatttgaaggtgatacccctgagacttgaaacctatggctttctatttcaggagctgcctgtcacatgtcaagaaacacgacacaggctgcccactttgctcctctgtccctcctggcttcccataggacgggagatagtaacatctgagcacaccctgcaagtattcaccgagtcctcccctcatcttcaagctcttttacccatttttcttctgacttgtcccaagtagagtctattttgaaataagttttcttttaaataaggctaaaacgtgcaaatctagccctctttggtgtctgggaacttgatgcaggtgaagatgatgcagtgaaagtgtcacagggcttacattgacacgttcagtcccagtgcgtccaggtcagatgagccctacttttaagatggcccagcttccttttaagatgggacactctcccctgtcttcttgagtcattctacgttctggaaaaactagatcttgcaagaagagatagaaatagcatggattgctctcccacatctgcttggcttccagcctacggaacaagagtgagagctttagtgttgagaggcgaatccagggacccatggttattgggtgacttcagtaaagcttctttggcaagaaatcaaacctcatattcaaagaatattgagttgaaaaatgccttctacttatgatgccgttagcttagcaaaggagtaaatgatctccattgagttcctctgctttcctagaattcaggtagaacattactgataatccttagcagaaagaatactctggcagggtctttttctgcttcagagtaaaggttgagtttttagcaggtacccaggtgtcagaaatggtagcccactccagtattcttacctggaaaatcccatggacggcagagcctggtaggctaccgtccatggggtcgcaaagagtcagacacgactgagggacttcacttttctcttcaggtgtcagaagaggcaggcctggtttttctgctgtcttattctgtggtcgtgagtgtggtcatgagcttgagctcgacaaagggaaggttgacaaagggatgagaagtagcatttatgttaactcccatgtctgataactgacctcatgaaaatctgatgaggcagttgtagttcttctcatttttacaaagagggaaggtgaacttagaggctaatggctggtctgtagttacaaaactttagtggcaggcttgggactgggaagaatttgtagcacatgattaaaaagttcacttttctcacctttcctctgggggcatttgccttttacactgaggtaagtatgtctttgagatgagtataaaccatcatcaatacattaattgctgtattgatgatggtttataggttgcttctccttgccactattttctgtttttttccacctactgggaaatttagttctcccatttctaaatcctattgcatgacttagaggaaggaatttgaaatcccctttcttcacttgcctctctttgtcccatgttccagttctttctctgtatccttgccctgatgataactcagatttttcaactctttatgggaatcagccgtgttatatgatgttatatgatgccatgtaagctgataaggaacctgtacatatttgattagcctttgggaaaaaaacccacgtcgtctaattaaacactgtgcacttaactgagcacaccgccttgctgtcagaaagtattgctctgggcctcatcctcctgacctcttccttacatttttgtcattcttcttctaaaaacatgtttacttctctatctggctttgctgggccttggttgcagcacgtgggctctcctagttgaggcatctgagctcgcagtcatgccttgtgggatccagttccatttgaacatgggcccccttcacggggagccactggaccaccagggaagtccctcgtgattcttaacaaatatatgctggaaccgttttctctggtaactataggcggaactaactgcatgtccattctttaatcactccccataaatataaatatggaggctggagatgaacagaaggacatctttggttctaaatggatgacatgcgcttacatataggcatttttggaattttctatgcaactctgtatcaaaataggatttgaatgctggtccacatacttgcttgttttctaacctttgaatgactcagggttttatgtgtaagagcaaggtctagaactcctagaaataagcacaagtgggtgtgctcagctcttcctacgaggcttgggctgcggctggtgttgagaagcctttagcggttggtgggagcaggctaggggggagcttggcctggaagactgtgtaaagacctcttcctctcagcaccaggaaaaggggattacattgagggaagttaagggatggttaacaaaaatggtacgcggggagagagagaaaactagatttaaaggctgagtactctgcagccagccaaggggaaaggctgtgagcaagttctcagagtcttcaccaaagggtgagaaggggaagaaagggcgccttttgcatggcagaggttcataaccatttgtcattgtttatttgctaagtcgtgtatgactcttgcgactccatggactgtagcccgctaggctcctctgtctatgggttttcccaggcaggagtacgggcaggaagggattgccatttgcttctccaggggatctccccagtccagggattgaacccatatctcctgctttgacaggtagattctttaccactgagctaccagggaagcgttcataatgatagtcgctattattaaggtgattttatgtttctcttcactacctatgtgccaactgggtagagattattttaaacaagacacaactacaacaaacctactgtggattctgaattctgatatgtttttagaaggctgggaatgcggaagaatgtaaaaataaaaataaaatagagattaaatcctaggagagattaaagtcctaacaagaatcatcaggttctatatgagatccgaggagaagagcaaaaatgtgttcattaggtgtgatacattagatctcgcactgggtagtggctgcatttcacatttaatatggttctcagctgttcccctgcaatgacacagctgaaaatagaggccttgctggttaagccaagtctagtcagtcctgcttgctgctgctgcaggggaaaatgagacattgagaatggcgctcagtccccttagtctgctccagtttctcgacactcgggcatgactctagaggaactgtaggataccagcgtaaagaagccacgagaacccgcggaaacctactgccccaactctgtagtacttatgggccaagttttcaaatccttgtccttgagtggggcccagcagattttggtttttactgagggccaccagagagttttcagtctaactaggaagataagtattttcaccccatgagataacagtagggcagagaatagatgctgactcttgtgtagaaattgtcagatgaattggaaaagggcctcagtatgctttcaaggagataaaacttgaagcccaggtaggattttttgttttttaattactaaattttaaaaaggtatttatttaatttatttagttgcccctcgtcttggctgcagctcgtggaatctttagttgtaactcagatcgtaatgcaagctcggttcgcgttactaagagtcgcagcgtgtgagctcttagtttcagcacgtgggatctagttcactggccagggatcgaacccaggcgccctgcactgggagctcggaggcttagccactggaccaccagggaaggcccttaatcactaagttttatttttcagagcagttttaggttcacagcaaaatcgagcaggaggtagggagagttcccatacaccctgtgtccccacagacgcatagcctcccacactaacagtgagccacagtggttcattgttagagtggagcctacactgacacagccttctctctcagagtctgtagtccttgttagggtttgctcttggggttgtctattctgtgggtttttaacaaacgtataatgtttcctccatgtcactatttctaatttaattccattgcggtctgggagcagacatcatatgacttcttttcatggttgttcaagtatgctttatggcccagaatgtagtctgtctcggtgaatgttacatgtgagcttgagaagaatgtgtccaatctgccgctgttggatgaaatagtctacagatgtcaattatttggtgctgtgaatacattcaactatgtcccttactgattttctgccttctggatctgtgtgcttctgacagagggcagctctgggagaggattcatctgtttctccttgaatttctaccagttcttgcctcatgtgttttgatgttcttttgtcggttgccttgaggagtgttatgttttcttggagtattgacctctttattattgtgtatgctactgtttatgtgttgtaattttcctcgttctgaagtcttctttgtctgaaattaacatagctacctatactgtcttcagattagtgttagcatgctctatctttctccgtctcgtttaatttatgtgtatctgtttatttaaagtgggtttctcatagacaagatatagttgggtcttgttttttcatccaccctgacaagcttttaattgtagtatttaagccattagcatttaaagtaatgattgagatacttgaattgacattgaccatatttgtcactgttttctatttgttggctttgttcttttttcctgttaatatttttgtcttctacacattttatgtcttttgtggttttaattagtagtttatatgattttattctctgtgttagcataccaattacactcctttttaaatactttttttagtggttgccctagagtttgcactttacatctatatcaaatccaaatccattttcaaatattcctgcttcagtgatagtgcaagtaccttccttatgttaacacagtaatcctggttctcttgtcccttgtatcgttgctgtcactcatttcacatatacatatatatatatatatacacacacacataatcaaatttgttgcttttatagattattatatgttagatcaattaagaataaggaaaatgacgattttaattttaccttcacttagtccctctctgatgcttttcctttctttatgtacatctgtttctgactacatcattttccttctctctgaagaacttcttttaacatttcctgtaagcagaaatactggcaacaaattttctcgatttttgcttgagaaattatttatttctcctttacttctgaagggttttccagggtgtaaaattctgtgttggtgcgttttttctcttaaccctttatagctgtcattccactttcttcttgcttgtatggtttctgagaagtcagatgtaattgttacctttgctcctctatagctaaggtgtttttttcccccctgtggcttctttcagaaaatttttaaatatttgcttttctaaagtttcactatgatatgcacaggtgtagtgtttttggtatgtatcctacccagtgttctctgagcttcctggatctgtggtttggtgtttggtattaatttgaggaaattctcagtcattaagtttcctctcccattgtattcttttcttttggtgtttgtattacgtgtatgttaccccttttatagtagtcccatggcccttgggtattttgttctttccctgccttttctctctttgcaattttggaagtttctgttgcaatattgccaagctcagattttatctttttagttatgaaaactcacaaatccagggactgcacgcaaactgtgtcacatagtgtccgttgtcacatagcctccattgctacaagaggctgggtaatatagtattttaagttgattttgttagtaaagatgaacgagataccagatactggggaggcagcttaatgtctgtctggaggactaagaataatctggatgtgagaagttagggtagtgagatgagctttgtctttttttgattgtgctgggtcttccttgctttgcgagggttttctctagtggcagtgagtccgggctactcttaggatgcaggagctcctcactgtgacggcttctcttgctgcagagcacgggctctaggcacatgggcttcagtaattgcagtgcttgagctcagtagttgtggctcgtggaccctagagcacacggctccaggagttggggcacaaggcatggggaatcttgcccaaccagggatcgaacccacgtcccctgcattggcagggggattcttttccactgtacgaccagggaagtccagtagaacgagctttgccactaactctgtgtgtgtgtgtgtgtgtgtgtgtgtgtgtgtgtgtgtgtgtgtatttgtgtgtaaaatctcactgggaattgctggcatagaagagtcttgttttttgttttatttattttacaaattattttgtagctggccactttattaaacttccttattagctttgatggatttttagttgattttctaggattttcttaatagaaaatcaatggaaagctcttggatttcttttcaaatactcttcctttgtctgttttcttattaattactgctactagattccttaggttttttttcctttctttcccaaattatcagattggggtgcttactttatttttggatgataattctgtgtatatatcagagtaaaattaaagtatgagattagaagcagttgaaatttagaaatatttgtggtctcagtagatttcatctctaagatttttagtaaaaactggctacatgcttttctcttttcagtagctaggatatgttatcaggcttcaacttaacatttaaggcatatgtcagatttttttgtttagtggaattattcaagaaagaaaatgttttatacttccctaagccacaaatacctagaggcttaaggattttaatagaatcctgttctgtatatacgtagactagctgtgtgtgtgtgtatgcacacgtgcgtgcacgctactcagtgaatggaatttcatagtttttaattcttttaaacaacactaaaaacagtatgctatttctctgatactacctcctccgggatgtaaagcttaattatcttctctttgaatatggtctgaacatagtgacttggttctaatgcataggctatgagatggggatggtaactttaccatggagaaacctggaagacctcacctttaactaggtgattaaggtgaacttcccctgtaataagtcacgttgatacccaccatgtgtcccagttacgaaggaaagagaaaggcacgtcatctgtgtagtattctccaaaatccataacctcagtcttaacagttaaagaacctcagaccaaccagacagagggacattctacaaaattcctgaccacatttggcaaggtgttaaggtcatgaactatagggaaagattgaaactaaggagccatgacagccagaagcaatgcagtgtcctggataaggtgccggatcagaaaaatgaccttagaagaaaactaggaaaatccaaaaagaaaacaaaagtctttagctgagttaatagaattaacctaacattaacatcttagttttgatcattgcaccatggagatgaaagagactaacattaggagaactagtgagggtattcagagctctctgttctactgttgcaactattctctaagtcaaaaactgtttcaaaatgaaaagttcaaaagaaagcgattacaagtcggcctggtaaataaaaacattttttatatttatcaaaaattgtaaagtgaagtctgctactgttaccagaacagatgtctgtggctttgtggaagggtcagtgaatggtgggtgtgggcgcctcaggtgccaggatcaagtaattgagataacgggactgggaagaccgtggtaggtgccggagaagtgcttccgggggcaccagggacaggtcctggtgcaggatgctggtgtggcgagggcgagtgaagtcactttgtgagtacagaagtgaagaaaagcactctcatgttttacggaggaaggcatttcagttcagttcagtcactcaattatgtccgactctttgtaaccccatgaaccgcagtacgccaggcctccctgtccatcaccaactcccggagtttactcaaacttacctccatcgagttggtgatgccatccaaccatctcatcctctgtcgtccccttctcctccagccttcagtctttcccaggatcagggtctttgctaatgagtcagttcttcacatcaggtggccaaagtattgcaacttcagcttcaacatcactccttccaatgaacacccaggactgatctcctttaggatggactggttggatctccttgcagtccaagggactttcaagagtcttctccaacaccacagttcaaaagcatcaattctttggtgctcagctttccttatagtccaactctcacattcaaacatgaccactggaaaaatcatagccttgacaagatggacctttgttgacaacgtaatgtctctgctttttaatatgctgtctaggttggtcataactttccttccaaggagtaagtgtcttttaatttcatggctgcagtcactatctgcagtgattttggagcccccaaaaataaagtcagccactgtttccccatctatttgccatgaagtgatgggaccggatgccatgagcttagttttctgaatgttgagctttaagccaactttttcactctcttctttcaccttcatcaagaggcttttgagttcctcttcactttctgtcataagggtggtgtcatctgcatatctgaggttattgatatttttcccggcaatcttgattccagcttgtgcttcttccagcccagcgtttctcatgatgtactctgcacagaagttaaataagcagggtgacaatatacagccttgacgtactccttttcctatttggaaccagtctgttgttccatgtccagttctaactgttgcttcctgacctgcatagagatttctcaagaggtaggtcaggtggtctggtattcccatctcttaaagaattttccacagtttattgtgatccacacagtcaaaggctttggcatagtcaataaagcagaaatagatgtttttctggaactctcttgctttttgatgatccagcagcacttaaatttaattaaattaacaaatgatttaagtaaagaaaaaaatggtgtgctgataaatcctcgatatgttgacctcttttgcttcagaaatctcttattttaaagcagataaaccctatttatgattgttcgttggaccactgagtaaaagtatcaaatgtcagtgagcttctcctttagaaaagctgacagcctggggctagtacatttttataaaaccttcagtccttgaggattgccccgaggggatgttgttgatttttgttttttttcctctccctgtttctgttgtcagttaaccctctggtttataaaaatggagtcagcgaaagaataaacaagaagccagtcttgcagtaatgctccactgggaatcaggacaccctgattatccctcgacctcaccattgtgtgtgttttcagctgccaggcggaggctgtccatgcctgtctccattgcctccaggctggccatggggttagccacaccccggaagtgaggtcagcaagaagggcactcggctccctgctcgggccctgtcacctgcccaggaaggtggcggggaagagagcccgtgtgaccctgcagagcttgccgtgtaggaggctcaggggccgtcacaggcatgctcactggctccatttgtcacagcaaagaaacgaaaaggagctcgtctgccggtggtacgttaagtgcttggaaagcctcggaatccctttggaagttttcagattcaccttcagacacagctttgaaaaattaatccatgaggcgtggaaatattttccagcccttcacctgagataaatgttctgtcagtgcagtgagctgagagaaaatctcctggcctcagatcccccggaatctcccaggttgctgctcagaggcagaagggcttctgcggctcgaacggtgcagctgtttctcaaacttcctgagggagaaggcaaatgctttatttaatctcccttggttctctgtgcttattcagtccgttttattgtttttcattaatttgggaaaactcttagccactatttctctaaatgtatctgttgccttgtttctgaaccccctctctggaactctggctccaggtactgttaggacatttccatgtggttctacagttcttggacgctctattttcttgtactctttttctctttgcatttcggtttggatcatttcttttttatttccaagtttactaccgtttttctttcctctgctctattgtgataagcacacttgaagaaattctatgatggtatgctttttatttctagcattttcatttggctctttgtttacagatagttttcatctgtctactgaataagcccatctgttcatgcaggttgtccactttttcactagattctttcacatgttaattatagttattttgaagtctccatctggtagttccaaacatcttggtcatctttcagcctagttcttttgacgggtttatgttatgacagtggattgttttctctcgtattttgtttctgataaactttgactgagtgatggactgtgttagaaaaacaggagacctaaagtaaatgtcgtgtccagcagtgggcagaccttctgtgagccattcgcatgggaggttgactcaacctagcatgtaactgggatgggtctgagtttttctgtcgctggacttaccttcagcaccgctgactccagatgctgccagtggtctcctcgttcttgtgtggggcacagggtactggggcttttctcagagttccagctccgccctccgcttccagcaggccctgcccccctgcactacacagggagccgtgctcccgtcggcgggtaggtagacttctgttgcttgtatgtggtgccagacctgtggtgggccaggagggttccattcccctggtccagcctcagtcttgcaggcttgtttgctggggccgtggaaggggtgctttctcagtgttcctggggcagctggagtctgccctttgtctgtgggggtctggggttggagggagtctcctacccctcccttgggagcagaggcttttgcttcgaccccagccacagctgagccttctaggagctggaagggttcctgccctccgagtgcggggcaggctgcctgtctccccctctcccagaagccgaggatctgcatccgcctttgggggtgagggcttttcctgctaacccccaagcaggtagagatttggctttggatgaaaaaaaaaagatttgagaaagggacagggctttctccctgtgtgccattgaagggaatgtctctggtttcttctcctgccctcctctttctccagaacatccagcagaaaagggctcgaaagtgagtgcagattctgcttgtgtccagaactcccaaactgccctactagcccacactcagtttgaagaatttgttaaaatgttagctggttttggtgactgcctttatagcagccacccccgccttctctgctcttcccgagctggatttgagtgtcccatggctcctggatggcgctcttagaaatcgggtgtccctgattgccttctgaccgcagctctcccacgcgtccagggcaagttacagtgttggtggtgtatctggtggctttttctcatggttactgtagattccatgtttctttgcgactttctacatcccaagcagaggcagatcttttattctgtggccaaccagtgggtgggggtggggaagtagagtaatattggggtacctactgatccaccgttacgtgtttggacagcattgcctgatgaggtttgacacagaggcatcaccggctggtatatttttttattttgtggttttcattagaaatgtccttttaatatattaaaacgaagtctgttggcatgggtcacattcaggagattctgttggaaagggcagtgcgcagttggttgccgatgaaagggtgttggagactcgtcactgtgcttgggatgcagtcgcccttggactactttggggtcctggggttcttctgtgtaatttcttcatggttagatatcctggacacttccaggtcagacttgcaaggcatagttctaaccactgattctctctcgtggtctatacacacacaatataagggacacccatggtcccatttctgccttgccatttcacctagaaacacgttttccttttttttcccccctcaaatccactccctgccccatcttttcccttttaaaattctgcatgcacgaggtgatcctttttatgaactgacggttttcctcctttaaggtcatgggctggtgcttagaagatctggagagttctgtgagatgcctgtggtgcctgtgagggagatggatttgttctttcaacaaacattcgactgtccagtccttgacaggcgctgtgctaggggctgcaagaccagtgatgagcaaaaccagatgcgtccccactctcacggtctgcagagaagtgctggcctctgccttggaagcttagagattcaggtagaagagacatgttagtgggtgagatattttccagtattaaggtctgtcatccttgtatttatttttattatacctacctcccttctctgaccagttggattcttttcctggcttcaagaattgaaatgaatggagtgtaaccttagaatcccttctgtctctcctcttcccctgtctccctccccgcccccaaatcacgcagaatgtagtatatactgtaagtgacctcgaggggcaggatgggtgcaggtgtggaggagatgttctgattatctgctctgcacagggcgaggagctttacaaacctcatctcacggaacctgcagctccaagagaaaggtggtgtggtgtccatcccctagattcaaaaaatagagagggttaagctgcttaacagagaaggcaatggcaccccactccagtactcttgcctggaaaatctcatggacggaagagcctggtgggctgcagtccatggggtcgatagagtcggacatgactgagcagcttcactttcacttttcactttcatgcgttggagaaggaaatggcaacccactccagcgttcttgcctggagaatcccagggacggaagggcctggtgggctgccgtctatggggttgcacagagtcggacatgactgaagcgacttagcagcagcagcagtaagctgcttaaaatcgtgcaagacactcatcaccatgactctttctcccatattgtacttgcttcctttaaacacttgttattgctagtcctgttggctttatgttactgaaaatcaaagaaattaaaccacagtagcagttttagtttgaagtttgtgtttgcttgagatagttttaagttggcccaatccttgccttttgcttcatgattctaatgtttgaaagtgtcataggaactttagttcctgtgctttgaaatctgcttttacaaaattactgtggtatcagtttcatgaagaaaagttaaatgctttgtcggaggttgttggtatgaccttttcatatatgacttagctttcagctgtgtagatgcatagaatagagcgtgtggggaatctgcaggtttgtggaagaatccctcatttcaaattttcagatagagataagtgtggcccatgttgatggggggaaaaatctcaaaacaaaataagtgtttgcatttagctcactggatgggaatggtggtgctttcatgtatatcacaacagagctgaaaactccttcctctctgccaaaaagtggcctatgctgagtcctgggggggctgtaaattatgccttctaaatgctgcaaggtgttaaagaggtcctgcagaaataattaagcctgtgaagttcacttatcctcaggcggagacacttgagctgtgattattggcaaggcttgcttcctttccaggagaactgcaatttggtggacaggatgagatgatttgagacatcttaaatcagagtctctgtcctacacttgtgcctctggctggtattctaggtggatttgttcccagtcattgtttcatgtaaatcccaaacgctgctgtctccactgtgattaaatgtgaatgcaaagctttggaatgctcgagtttgagtgtggtgtgatttgaaattaaaacaaacaaacaaacatatgacgatgaacccatttgcgtagaaaaggaggtaaatatttaagctgtgtttatgtgtgggctcatgcatcttggtgccttggtagaggaccatttccttactctgtgctggtactcttcatattctagaaggaggtagaaggtctgactttagagttagtattcacgggttggattggtgactttgccacttaatagctcagtttctcacatataaaatgaaatttgtatctgccacccgacccccaaaagttaacaaacttggttataaagagcagatgagatcatgggtacagtagtgctttgcagcaggcaaaacattctaacgtttgtaaggatactaatgaggaaaaaaaaaatgccagtttccacagccaagaaattcacagtgtagctggagagagaaaagtggtcatctgaaatgccagtaaagcagtgttagaacggtgtatcagagaactaagccataaggtaaaggctgtgaatgcggtgggatgtcttagtaagggaagattgttgatgttgggggtgctcagtaaaggtttcacagagaaggtaggttctcttaggcctcatgaactgtagatgctttggctggatgatgggaggggaagcagcagcatggctgagcaggaaagagactagagtgagtagagtgtatctacgaagcagcagcaggcagccaagaggccccccctggctgtacccagatggcaaaccgcaggacggctcaagtcagatgcccctggaagctaaatctgtttttgattttctctgaataggagaaattctcattgtgatgctttgccttctccttattgttagtggaaacaatagtcgtcatataactcagaagttatccaaggctttaactaatagaggacttgtccaggaaacagacccaaagtcagtaagtctagactagaaagtgtctttttttcctctccctattttggttatgcttccagtttctcatgctaaacttggatatgtttggcccccttgcataaatgcaaacagcctctgattttgagtgggatcgtaggctgaatgaagctggagaggtcatcggcgtcagcatcctgctttctttaggaaccgagtttaaactctgcttggataagaaactgtcttctttcagaagactctgacagagtggctggtttgggagtaaccctcctgtcaataaaagaaaaagaagtacagaagctcattataatagatgaaacaacagttcagagagcagccaggacttgagggataattcctgagagacaggagcaccctgagaatagctgcctgttcactctgatgccatcccccgaggacttctaattcacagtcatgggtatggggacttcacaggaaacagcagccccacagggctgacaggctggggttagggggttcccagagtatctgggatttgaaggacaaaagtctcaggagaaagggaatcatggggaaataaacaaaaaacgagcatgcagtcttccattaaggtctttgccaactcctaaggtgcacacgatcagagcaagactcctggagtccctgcagaaagaagcggctgggagcctaaggagctgtgcagctatttcagtaaccatatatagtgctaatgagacaaaggtccatgttcagggctggccaggatggaggggtcctgggaaataccccaggctttagttgaaggcccagaaggccacaccttcagactatgggcaagctgaaaaaagtcctgacaaaagttgaagctgataggattaaagtgttctgactgcattctctgtgcctgcaagcagaatgtgttatattcttaagagaaagataatctggattgtccagaatctctataatttttaatgcagaatattggtatctaataaaaaattaagatgcaagttagaaattgaaaccaaatgaccaaaatgaagaataaaaagagatggcagaaagagacttaaagtttattcagatattggagttatcagtcaggaactaacaatactcaataatacagaagtgaaggaggatttcaacaagatacaaaatcttcttagccaagtggaattctggaaatgaaaaatataagaagtgaaattaacagttcaatacatggatttaatagcagtttatagacttacacacacaaaggataaaggattattggaaaaggataaaggattattggaccagaaggaatattagtagaaaaatacacagattgaagcacacagagaaaaaatacagaaaagagaataagagacatacaggacatggtgaaaacattttacatacacgtgtgtctggagttgtagaagaggagagtgtgatagaaccaacattttaaaaaatactgacagagttttctagaagcatcagaagacataaagtcacagattagagaagctatacagttctcagtaggataaatacaaagagctacactgaggtacatctgtggacctctgtggacaaatagaatgagcaaacacttaaactcaactgtatcagaaattacattaagtataagtggactgtatgtacaactgaaagccaaagatttttaaatgggataaaatattaaaatccatttatatctttttatgagagataatctttagacaaactttaagggcagaaatttgaaagtaaaaattttggaaataaacacacatacacgcgtgcatacaggcagagagcaatcaataaaggtatagaatatttagatagtactaaataatcaacttactctgcttgacaagtgtaaaacattacactcatcaacagcaagatgctctttttaaagtgcacgtagatcattgacaaaaataaataatgtgcctggccataaatcaagtttaaataaattgcaaggttttgaaataacttgtatgttccctgactacagtgcaaagtagaaatcagtaacagataaatataaaatctgagagtgtttggaaattaaacagtatacttctattaataaatgccctttgggccaaagaaagtgttacaatggaaattaattttttttacctaaattgtaatgaaaatgatatgtaaatgtgtgggatgcatttaaaatagtgcatagaaataaatttacagctttgtatttaggtattaagaaagaagaaagattgaaatatctaaacttcaatctcaagaatctaaatgcagtatggattaaagtcccccaaaaccaaagaaagtaaaaataagagcagaaaggagcaaaatagaaaaaaagttaggtgaagaattaagtaagtaagataccacatagttgcactcatctcacatgccagcaaagcagtgctcaaaattcttcaagcggggcttcaacagtatgtgaactgagaacttacagatattcaagctgtatttagaaaaggcagaggaaccagaaatcaaattgccaacatctgttgcagagagttccagaagaacatctacttctgcttcattgactatgctaagcctttgactgtgtggatcacagcaaactggaaaattcttaaagagatgggaataccagaccaccttgcctgcctcatgagaaatctgtatgcaggtcaagaagcagcagttagaactggacatggaacaatggactggttccaaattgggaaaggagtacatcaacgctgtattttgtcaccctgcctgtttaacttatatgcagagtacatcatgcaaaatgccatactggatgaagcacaagctggaatcaagattgccgggagaaatatcaataacttcagatatgcagatgacaccacccttatgacagaaagcgaagaggaactaaagagcctcttgatgaaggtgaaagaggagagtgaaaaagctggctcaaaacttaacattcaaaaaattaaaatcatgccatctggtcccatcacttcatggcaaatagatggggagacaatggaaacagtgacagactttattttcttgggctccaaaatcactttagatggtaaatgcagccatgaaattaaaagatgtttgctccttggaagaaaagctgtgacaaacttagcatattaaaaagtagagacattactttgtcaacaaaggtccatctagtcaagtctgtggtttttccagtggtcatgtatggatgtgagagttggaccatgaagaaagctgagcgccaaagaattgatgcttttgaactgtgttgttggagaagactcttgagagttagtcagtcctaaaggaaatcagtcctgaatattcattggaaagactgatactgcagctgaaactcccatactttgtccacctgatgcgaagagccaactcattagaaaagaccctgatgctgggaaagattgaaggcaggaggagaaggggacgacagaggatgagatgattggatgacatggctgactcgatggatgagtttgagtaagctctgggagtcagtgatgcacagggatgcctgcctgctgtagtccatggcgtcgcaaagagtcagacacgactgagcgactgaactgaactgaagatgaagaaaatcaacaaaatgaaatggaaaagattaataaaattgataaacatcagtaagactgatcaagtacaaagggaaaacacagatgatcaatatcaggaatgaaaagagagatattactacagattttataggcattaaatagacatacatttagtccaaacatttaacagtttaaagtgaacatattcgttgaaaaataccacctacaaaactgacatgaaaatatgcagaatcttatgtctgtggaagaaaattcatgatttaaaatcgttacccaccccaccctcccaaaaaaaacacaaaaccttagatggcttttttgatgacttctcagtctttcaaggcagaaataatgccaaacttggctaacatttgtcaaagaatagaactttttctactcacttttcaactccttttatgaggccagcataacctgacactgaaatccaattttaacattacagtgttcagcaaaagaggtccgacacaaaagagtaaatactgtttggttccattaatatgaaattccttagtgggctaacagaaatcagagtagggattactcttggaggggtgagatactggctgggtgcagcagagaggactctcctggggtgctggaatgttctatatctttatctggatagtgattataggagtactgaatgtactttatgtgcactttagtagtaacattaaaaaaaaaagactataaacttgatacttcattcccatgtagtttcttttctgtctaaattgggttccgtgtgcattgcctgcgctcatgtcctgtttgactgccttggatgaatgcccattctgatggagttgccttgcacagcaggcaggcttttgccttgggcttgctcttctttgagaataaggaaatgaccccgacgcagaggtgcctgtgatggagcctctagttcttccctcactgctcatttcacgcagtgtggttttcttcacaggcacggtggttttgggaagcatacttccagtcaatcaaggccatcgccctggccaccctgcagatcatcaatgatcggatctatccatacgctgccatctcctatgaagactggaacgaccctcctgctgtggtgagtaaatgctggggtgagccacgtcaggtataggccagaggccaggacgatggaacatgtactggatttgtaaaggtgatttaagtttgagctttctaagatataagagtgtgctagaatatgtgggacccaataggaccagttttggtcttccctgttggctcagatggtaaggaatctacctgcagtgcgggagacctaggttcaatccctgggttgggaggattccctggaagagggcatggccacccactccagtatttttgcctggagaatccccatggacagaggagcctggcgggctacagtccacggggtaaggaaagagtaggacacgaatgaagcgacttagcacacacgcacgcaggaccagtttaattttgaaatgaccaaagtaacaaatactgaagcagcagtttgttttaaatgtgcttaggaaggcagtgtgtttgtttattgcagaaataaattgggaggcctgggtcttgttggaaatggaagacttcctgatgacttttgcaatggtggtgctttgaactcttgaaaagattctctaagcaattagtgatggcttatattttgagccctgcctcttgtgctttatgctaactaagtgctttgcatggatgtcaattaacattcaggcccctgctaggggacatcatcagcaaaaatgatagagaggcattagggattgctcagggtggccgagacccctcggtccttccagcagcaagatggggtgggcagatgctgctctcagagcttcgtccttaattgcaggagtcatgaggtacctgtggcctgggagggacaagtacctgaagctgtggggggcacagtctctgctctggggcacacctgctctctggctcacagaccccgaggcctctgcgtgagagagctagactcctctccgctgaatcattgctcttctctttgtggccagccgtccttgtctcctgactctacccgctcttaacagtgctgtatgctgccacctggtcggtcttctttttccctccccatctccctcctgcctttcttaagggcttattgtgtgccagtgctccaccaggtgcttagaaagcagaggaaaccaggccctgccctcagaggaactcagtgctaacttgggaagcccggaaggaagggtattgtatgagttgccctgcagtggagctgagggtggaattcccagggagccccgggcaagggcgggcagtgctcctgaaccgggagtgcccgagaggaagtaaaaccgtcaaggcaagtgaaacagtagcaaacacctcctttctgttttccttgagggaagatttccttctgtccatctcttcccttgacttccttgccaaaggtaaccttccagaatgtttgtgttcctgtatagacatactgtgtaattttagaatacaaatggggtcacacgatgcatattattctcaaccttgccttttttactttaaaactagatcatgggcttacttccatttcattctgtatagggatacttcagttttaaggactgctgtaatcatgttttggccttttggctaagatcacgtgttggactgctgtatgtgatcccatcgcttaggtctatcttacaggcaaatctgttttcaacagtaataggggggatgcatattattgcatccacacagcctttcaggtgtgtatgcacataaatgtgtgaatgtatctgtagaattagtaatagcagagaaataactagaagaataggcacactttaagtgttgatcagtcaatcagtctgttcaattgctcagccccgtctgactcttttcaaccccatcgacttgagtgttgatagattttaacaaattgctctttcaaaagactacaccagtttataattccaccaataacattttggttattctgcttcttcatgccttcagtaaccctgagtacttgaatttttgcttacttggaaggttgatatctctctctgttgctcccagaatacaaatataaatcctatttgaactgtgattgataaccctgactttttagattgtttttggcatgtattcattcagcgggtatttcctgagtgcctattttgttccaggcagtgttcttggtacttgagatatgtcagtcaacaaagcagagaaaaatccacgccactggggagtttacagtgcagtaggtcttgctatctggaagcaaggtttgtctttctagtaaaattttaaagctttctgtatactggtcctttacaagtcttgcaaaattttttcttaggtgttttatcattcttacactccgataaaggaattcatgctttctgacattttagttcctaactgattacttttgtatgaagacatattgttggcttttatgtataaacttgtaatactctgagttctgtaattgtttgacttttttcaagttgattctcttgtgtcttctagatgtacagtgatacctgcaaatgctaattattcttttttccaacacttgaaccttttgttatcttttatctaattgccttggctgctgcttccagaacaatgttaaatgaccatcatttaatgttgttaagtgttaaaagagaagggcgggtgttgtgtgagaaagggggaagttagatttccccctcccacagcttacctggttctctggaggacagagactcaggcaagtgagcgttagatacagggagggggtgggccaagaatgatgaattttggggtttctggttccatccagaaggagaagcaaattcaggggtcagagggacaagagaacacggcggcttcaggaactttgagggggtcagtgtagcaggagtgaagggtttgtgagggagatgcagaaggtggagccttctgagggcccagattgccaaacccctggcatcttaagggaggtgctggaatgtttccggtgggtgatgggagatcagcagagcacctccacagaagtcatgtgctgagattgcatttcgggtcattctgacatcagtgtggagggtgcttctgttgtaggaggacagggccaaaggcaggagactgtttcaggaaccaggtgagaattaatggaggataaatgtgagtgatggggcttccctggtggctcagtcggtaaagaatctgcctgcaatcctggagactgcctgcaagacaggtgacctgggtttgatccctgggtcaggaagttcccctggagaaggaaatggcaacccagtccagtattcttgcctggaaaatcccatggacagaggagcctggcagacaacagtccacagggtcgcaaagagttggacacaacttagcgactagaccaccacccaaatgtgagtgataccagggaccacctgccgtgctaactgggaaggcgtttaggaagagtgaggaggatggtgttgtctattgagtgcgagtacatgggcatcgcgggccaggtggtgttggagtccaggcatggtctggcagcagggccttccaccgtccagcgccaggcctcctttccagccttatttccttctgttcccttttggggagcactaggtttctggctccacaatgttgtccttcctgtgtttgctctctgcttttccttcctgtgcccttgtgaccattgcttctttccctggacgccgttcttcaccatcacctctggtcttatgcattttattcaggggccctggccatgtacccctttctccatgagatttttcctggaccccttacaaaaagtaatctttccttcttgtaaatcttaaagcgtattatctatttctttcctgtcatttaacatacatagaagtacaatttcctgtaaaattttaagtttcttaagggaatttctgagttatcttggtattccttataatatttagagcaatttaattcaggtaggaagccctttatacataaagaaggaaggagggattgaggagggtggaatttccaggacttgaaccatccaaagctcagtcgcatgctgacgtgacagcctttattttatttgaccttcaccctattgggaaggtgagaaaagtgaaagattccctctcatgtctaaagcagacctaatgaggctcacagagcctgatgactcgtccaaggtcgcataatgggcaagttgtgtaaacaagcaggaacccgggtcttctacccacagagtgtcttgactggcttatgcagtattatgggagccattgagcctcctccaggctgaaacatttccactcttctgagcatatggtacaagcttgagacaaagaagatcacatctgttgctctgcaggagaattcatacaggtgggataatcatgacccagctagagaatgtggagacagtgggcgctttcctgtacttttatctaaagctccaagaaaagtgtttgaggataaatggttgggattctggtctctgtctttcataagaatacagactaggagaaaatagtatatgaagttttatagttttgaaataggcatattttaattgaggacaaagcagaatgctggactcaatttgggaaagaagatgagtataaaaagcatagatctgaataagcaactggaaactatattatttatttatttatttatttttctttcttaaggggaatttagtgtttaagttctaggagctcaggtgatgagagccctgatttaaagctgtaggttgtacatttctgcttcttcagtgaaagcaactgacagaaagcctgaatagcattgatttatttatagttacctcccccgccccccgccccttcagaaatacttaacctgtcctagatgtggttactatgagcccagcctgcaggggagtctcccctctagattccatcaaatttaccatttcaggcaccccgaggtgggagttggtgtggatctcagtcagtaaatcaagggttggcctggctttttcaaagatctgcagtcagggcctccagccatggctctgagcttgctcagctggagaggagggccgagagcagagagcccttttttggccagatgccactgcctgagtttaacgttaaaaaaaagaagaggcttgtgtgtggatggtgaaaacagggaggggtggtggccggccgtgcagaaacccggtgcctgggccaccagatgctctgccgtcctcatttgtaaccggagcggggctgattgcattatccaccttttgctacactctcaagatggtgaaagcgtgtgagtaggtcattctggagcacaactttaaagtggcacagtggtccagtctggagagatgggccctttgtcttgaatcttctctttggaaggaatttctcaccaaatggaagtttctttgtgagagatgacacatttgttcttaatcattaaattttttagctcctcttgtcaaggatgcctgcattttacggtctcttgaggttttcatttctccccccccccctttttttttttttgcaagtctcagcaaaactttaccatctccccaaacactccccgcctgtgtcgcttctcaggcgtgtgccatttctggtctcccccttggcgggcctggtgcctggatctgggcctggcctgggtggctgggggtctcttcagacagcctcccccagcttacctctggtcgcctgcagggcacgagcttgtcgggaggacacagactgaggcccgggagcattgagaatgccgatatggagcgggccaagagccgtgaggttttggatttctggcctgtgccaacaaagccatatcctctcggtaaaatcacgtagagtggagtattttcccatattatttttgtacagcgtgattagaaggattatctccaccttatcgtgtgctaacgtgattgagtagtggtggtagtgtcagtcgctcagtcgtgtccgtctctgtggactgcagcccgccaggctcttccatccgtggaattctccaggaaagaatactggagtgggttgccatgcccttctccaggggatcttctcaacccggggatcaaacccaggtctcctgcatttaaggtggagtctttaccatttgagctacccaagaagtaaactttatttacctagttccttctggcagtgtttttcagatttgttgtcatagataacactagtatagataacccaggttttttttttttttaacactgactatactggacctagaacaacagaaaagggagacagcaggggtcccatagctattgacaaaaaacctgacaaactgacgaggaaagtggggaagtgacttaaaacggattgttgcgcccagcataagaataaggctgacagcaaaaacttgttcctatgaaatctagcccttgtggcagcttcgctgcaagccaggcctcaggtgcgggttatatgcactcacctaggcatcctgtggagaaggcaatggcaccccgctccagtactcttgcctggaatatcccacagatggaggagcctggtgggctacagtccatggggttgctaagagttggacacgactgagcaacttcactttcacttttcactttcatgcattggagaaggaaatggcaacctgctccagtgttcttgcctggagaatcccagggacggggaagccttgtgcgctcccgtctccagggtcgcatagagtcggacacgactgaagccacttagcagcagcagcagcaggcatcctgtaagcctgagtccagcttttgaagatagagggggaaggctcagcaggtgggaggcctgtagaagagcacttgcttgcatggcagtcctcacccacgaggggcagggtcagagctgtggactcggtgcagcagagcctgctgctgtccctagttcttggggaagcggttctccaatgtcaggaggttctccaatgtcaggacgtgtcagaagctcctgaaaggctcgtgaaaatgcagattgctgccactccacccctagtctcagatggtgttggtctgggtgaggtcctcggatttgcatttctagcaagttcccaagtagtgtcgatgcagctagtccaggggctgtgctttgatgtgggattggttcactgtgtgtgtcctgactcactgcctggagaaggcactccctctccccccatccagtgcctcttctgcatccctggaacttgattagaagcttccagagcctgggctaagcccacatctgtctcagctacaagaagaactcagggctaccggtacttagatgtccagacggcagccctcctcattcacagaggttgtggttgtataagctgctgaaaatatcactcagctgtaaatactaacaagctataaatcactctgcaggccgaaggagcgccagctcagatgcctggagcagaaccccgagtctgtttaactccactccctgccctttaacatccaggttgtggaatgtgtctctggaatttgaacacctcccctgctcaccccctcccccaggaggcctttttggcttattttctcccctttttcctcccactgcccagatatggctgaagtatgtatggaattcagaaattttagcttgaattttaagtaagtttaagcgttttggagtcttttgtttttagaggtgccacatttacagggtcacagttgcacagaaggtccgccttcctgcccccaatgccaggcctttccttgagttgccttcgccattgaactgtttcgtagctgtacttgcttctgtggaaacaagccggacagtctcaggtcatggagtaaagaaagccgagttgggtctgtggagcgagagacgcaggaagatcccaggagcttgacccggggccccatgccaagaattctgatggtcagatagtaacgtgggaaagatgaggtcgcaggtcagatccaggtggggaggataagtaaagcacaggaagggccagactcgggggtgctgcggagggggtcctgtcaatcagagcatccttcctggagagggagactcgtgtacccgggttgccatgtgtgcatggcgactccagtggtggcggaggtcgagccgcacgtgcagtttctctggatgcagaccttcctcacccacccgggcttttgtagagttcacttttatgactctggcatttatgagtcaacccctgtctgcttccttacgtcccagagaggagggcagcgagtggagtaggtcctcggggtccatggagcttggtctggcagcagatgaattgaggacaccttgggtattttggcaaagccatccttcacacaccagctgtcactcaccggcacatccaattccctttccccttgcttcagggcccactttctttctccctcgagtcagtttaagaggttcagtagcagaggttactgggaaaatgagttgtcgggtatcccaacatcaggggtaatgaattctatttctcgtatcctatactgtttacatagacatatcaattaatatttgagtgtaatttttctaatgagagggtcattacagctgcagagtagaaaaatttccacattgctccagctggagtttgagtcttgatttttggttctgtcttatctttcagctatatttcttaatttcatgtttaagaaaaaaatagaatggcttaacacatgaatggtttaaaaagtaaaatcataaaataacgcctgtaaattaaggcccattcccgaggggcagctgctctcagctctcctctgatatttattttcctaccttctcataaaatgctgatagtgctgtttttttttaatttctctatctcagacattctctctttacttccatagagttagtgctttgtttcctgttcctgcctcttcctctgcacgtttctcttccctctcatcttaccagtggggcagtgtcatcatttttcattagagcaatgtttattgttgcgtcattaggactatataaatgttggtgtcagtgggaccaagtaataaaacatggttatgtttcctttcttgcataactttttttattttccagggagtaaatgcatagctgtcttaaaaacaaaacaaaacaaaaacactctcttagctttccatgttcctatcactgctttttactaacttctccaacgaagagtgaaatggtcaagcgcaagtgaactatgggctgtcccccgccttcgctctctttccctccctcccacccttctcgtacatagcccgcacctgcagctccagtgtggattgactgctctctaggtctcctgtacagaggtcagcctcaattctctgttcaccatgattcttgcaattctcttcttttctcttctttgttggatttttgttttcaagttctaacatcctctttctttttgggtttttttttttttttaatccagtagcttccccagaaaagggactgttggggggtaaatttgctgtcaccttgtgtctctgaaaatgcatctattctctttcaccttgatgaatagctgtgtgcagtgctgtgcttgggctggtataaaattctggtccacgtggcttttcttcaaaatttcaaaggcattgcttcattgtctgctgatcttcctgttttaatctcaagagatgtcattctaattcctgatccttttgtgtgaaacttttaatcttcctgaaaaatcctgtgttctgaaactaatgatgatacaccctggtatgagtgtttttttgttcactggatactgatttgggccctttataagggaattttcacattttctatctcccccgccccgcctcgtttgctctctttttcgcttggtggaacttgggttaccttgatgatgaatgtgctagactgacccttatttttcttatcttttctttctgtcttaatggactctttgttttaatttcttagatattttctcaattttatcttctaatttttcattttaaaatttttgttcacttttaatttctaagagttttttcttgtcttctacctaatcttttttatagcttcctattcttgttttaaggatacagtactttctctctctgaagatatttgtggttttgtttttattttttattgtttttatataaatttattttaattggaggctaattactttacaatattgtattggttttgccatacagaaacatgaatccgccacgggtgtacatgtgttccccatcctgaacccccctcccacctccctccctgtaccatccctctgggtcatcccagtgcaccagccccaagcatcctgtatcctgcatcaaacctggactggtgattcgtttcatatatgatattatacatgtttcaatgccattctcccaaatcatcccaccctcgccctctcccacagagtccaaaagactgttctatacatctgtgtctcttttgctgtctcgcatacagggttatcgttaccctctttctaaattccatatatatgcattagtatactgtattggtgtttttctttctgtttttatttgtcttaagctccgtgtattgtttgtttcctttgaattccatcatcagggctttattttctgtctttcacattggaaactttcttcaaatacctgatgtttttcagcagtctattcttatttgggtgaggcactgaaaactgatgggaagccctgtgtgcatggtttggcttgtcaacttttgggctttactgaggtttatggatggggactcaactgtttaccccgttgttggtactcttcagttcagttcagtcgctcagtcgtgtccgattctttgcgaccccatgaactgcagcacgccaggcctccctgttcatcaccatctcccggagttcactcaaactcacgtccatcgagtcagtgatgccatccagccatctcatcctctgtcgtccccttctcctcctgcccccaatcactcccagcatcagagtcttttccaatgagtcaactcttcgcatgaggtggccaaaagtacttttaggttatttctgttggattggttcactgacccaaaggataatctttctgcccttgggagtagggcgaggatggatagtaagtctgggctgcaagtctttggggaggagaacgggggaaggagactggttcttatccttcatacagtagatgatcactgaatctgccgtatttggtgtggcgcctcatcctgctggccgtccgttgtgtctctggagtgtggggtctctctagttcattttctccagagaataaacctcctgctaagatgcagtgggtggagaggacagggagggcattgacctgcctgtgcacgtaggtttaagggatctcagaaccaacttttcttattcctgttttcagagtccgtcctccctagacacctaggccttcccatttctcagtctgctaaccatcatccttggaaggattagcttcccatgctttgctaggcagtttatgttcctttatctgccttttgtttgtttgcttaaacagacctacaattctgtatattttttaattttctattttgacataattttagacttaaatttccaaaaccagtgcatcgtttctgcacacccttcactcatagtcatcaaacactaatattttgccacctttcttttattcttttctgtgtgtgtgtaagcacacacaaacatatacattcatatttatgtatttttttcctgaaccatttgcaagttacagatatgaggcacctttactcctagatctttctcagaaaatcacccaaatgatcgctagtggtaaagaacccacttgccagtttaggagacataagggatgcaggttcaatctctaggtcgggaagatgccctggaggagggcatggcaacccattccagtattcttgcctggaggatccccatggacagaggagtctggccggctacagtccatggggtcacaaagagctggacaccgctgaagccgctgagcgcacacatacagaccttatttagattttgtcagttatcttcattatgtccattacagcagaagattgcaggcagtcattgtgttcagttgtcatgtctctgtagcttctactagaacagtttctcgggtccttctttgtctggcatgacagtggcctttttgaagagaacatgtcagttattttggaaggtatcctttgatttggatttgtctggtgcttttgtgttaaattatgcattccatttcaaattattgaaattcaggttaagtatttttggcaggaatgtcacaaaagtaatgttggttcttggtgtatcacgctggaggacacgtgatgttggtctgtcccattggtcgtgacgataacttcactcagagctttctccagtgggaggttacttccttcttttagtgtgtcttgttcggacgtactttgagactgtgtaaatatcctgttactcctcaaacctttactagggttagcatccaccaatgatttcttgtctgaatcaattgttataatggttgccaaatggtaattttaattcccatgctctatctgcttttgagagtgatttttattctttaacctgagggttcatgtcttttaatcagtgttggaatattctgcgtcattttcttgttgaatattatgtatctgtcattctcactgatcttggaatcctattaaatgcatgttagacctctttaattctgttgttgtgtctctcaagtttgtttttttttttccccttatgttttacacatttttaactctttgtgctgcatgttggaaaattttttctcatctattttgctctttcctttggccatgtccaatgtgtttttacccatccagtgggattaaaatttcagtagttatttctagaagctctgactatttttcaaatctgtctgcctgatcttttttcatagtatctttttttccctatgtattttcacttccctcttttgtgtgttaattatttgagatactcactttatattctccagtaattcaattttaaaatttctcatagcttctaaccctgtcgtttgcctcctgcctgttgctcttgttgcattatttccttatatgttttgcaatcttggaatgtgagtttcatctctggtgaggttttctgtggaaatcttcgccacctgagtgtaagaaggtatttctccagagagtttgttcttgttttgtggcttctgccagatgttccaggaggtattgcagagataaatcactttttaacattagtttctcggtcagaggtttccatactgcacagggagggtccatttaaaccccacaccagtgcgagggctagcctctgttggaagcatcccccgtccaaactcaggttgaaaaggacagatgattttctgtatgtccatgttctaggagtgactttcgggaccacctttttactgattgtacaggccttcaagggttcaggcttggtatgtaagtcccaccgcctgccccctctgcccccgtgcccaaagctgtgctcctcgtccaaagtgggtgtgagtgtccacgccctgcgccgctgggcccagtgacagcctccccgcggcgccttcagttctttgcagtttgctcatcgatttgggttcctggcaactttccatttcctgaatatttttttttaattttgttatattttatcctacgtttctgtctgttgtccagcaagagacgctgtctttttcgcctccccagatacgttgagctcctgtccactgttgtttccttgcctgttctttgtgccctgtgggtttatgctccccctcttctccccccaggaggaagcagggctaaaacacagtagacaaaacatacaccttattggaattccggagaagttaagaaatgattgcattttagaggttgttctttccactttgtattgaccttaatactcctgtcagctcaaaactgtacaaaagctttgattttcttggtcataactgtgtaggtattttgagcttggaggccacaagctataacttaatctttcccaagagtgtctgtttaccaagctagtgatgtcttccttcataccatagctgacctctgaaatatagtcagcgggccgccagcaaacatttttatgcccaggccaaagaaggagcctgtctttcgaactgagtatttcaggtgtgggatagccagatgaaactataaatttgtttcacctctcatgagaagtagtgctttgagccaccattagcattcttgaacactttggattatagatcagcctgtttgcttgaatcagagaaattactagctttttcactgaagtttttttttgttgtttttttttttaaagtgttttcagtatactttttaagtaacagatccagggatgaggatggtgaatttgaagactgtgtcttaagaataatccacattttttactatcaaacgactgaactgttatttttaacatacaaagttaagaaccagtaaaatcaacaacatgttgtatgtgctcctactttattgtattcacccatttaattgattagacagtatcttaaaccattttaccacagatgtatttagtgaaatttattacttgcagactgatataactcatgaattattaaaagcatcagcatatggaaccttctctttctcatctagattctttgaatacggcttaggttgagccatttaactggcttctgtccatcccactgatggaagccacagtcagatgagggttcttataaatgattggcttcctttccgagcaagaagaggataaaatgtcagctttttcctctactacctgaaaagcaatcaggagcagccctcccactcttcttgacagtagtttggctttatagaagaagggcaggcattgagccaaatgtgaatgtcagtgctatataaagaaaaaagttaagtagctaaaactccgatagtaactggaatttttagagcagtattcagatgggtttaccctgtaagctttcagaaagctaacaataactcagcctgaactgcaaggtttaatcccttctttcctggtgatgatcccagtaaattgaagttacttaccgaatgtctcttcacagcgatttgctcatgtgtaggactgggtttagggggtaataatagtttcagaagaatagggtagtggttagtgactgattaccaattactaatcaaagccattgaaaaggacattttggaatcaaccggagttccagaccatttaggagcctgacattgtgctttcttatctctttaaaatttgttatccttacctgtaaaatgaactattacttatttcataggattattatgagacagtagtgaagtaatgtatgtaggagcattttgtaagtggtattgataaggctgctagtgtaacgttctatcacatggaaacatacactctagaattgggtgggactctggagaacaaaactgaaatttacagagggcacagttttatacccagataggctctgttgtgtattttctgttgttgaatccttgcatccccaaagtagcattctgctagctccattcctacagatgaagaaggggagtcttgtttatttttaagatctgcgtctccatcatcccatttcatacatgaggaaatggaggtctgcagaagtgtggtgacttgtctgacgcagagattctcagtccatcattccatctgttataataggttatgatgttgtcgcagtaattgtttttgtattgcacacagacttggccctgaaattaatcaggaaatacaatagcacctaattcaattattagcgtaaatatagaaagacttgtgtcactggcttcttggtggattaggtatgctaaatgaccttcccaactgaacaacaaaaatattgaatgaagtatgataaagcctgttgctaagttgagtgaaaagtaagatacttgttgaccctaaacaaagtgaagccaggtgtcctgggaggtgctttcactgcagacatttgcagagttcggtggcgtagagcctcagcctggttgactgtctgaagcagggagcaggggagcgggagagaaagtgtgtggcctgagcaagttggagaatctatcgggagcttcctgggtgaagccagggagctaaaagagcctcttgagtgaaggggtgaagaagcgcaagtctgtattgaccctgctgttgagcactgcattcataagccagcttcacatggtttgtggtccatgttcgtactacaggtatagtccaaacaaaccgtaagaggagaatttcctcggaggtggccatgagttggtaggggccagcagaagtggacacggaccctctctgtaggggttcgttttcagtccagctctcaaatgatgaacgtgtctgtggaaaatgagcagctcactgtaaaaaacaaaacaaaaaccccacgatcagaaataaggcaaaccctatcaagaaataaggcagtatcacgagcgagaaccagcagaaacacagctgcaaggcttgggcaagctccagaagatagtggaggacagcgaggcctggcatgctgcagtccgtggggttgcaaagagttagacgtgacatagagactgaatgacactttagatgattaaattcaaagaaatggtttggaatgcagcccaaagagacagaaaatattaacgaattgaagttacgtggagagcagagggacaaggtccaacatatgtctagtcagagttccaggacgaagagagacaatggagaagaggcccatttttgaaagaataatagctgacagttttccagagctgttgaatgatagtagatctcagagctaggaagaccagcaaaccgcaagcaggacagtcgtagtgaaactgcagtgcacggaagaaaaaggagcttttaaacacatctagatgttcaagctcgttttagaaaaggcagaggaaccagagatcaaattgccaacatccgctgggtcatggaaaaagcaagcgagttccagaaaaacatctatttctgctttattgactatgccaaagcctttgactgtgtggatcacaataaactgtggaaaattcttcaagagatggaaataccagaccacctgacctgcctcttaagaaacctgtatgcaggtcaggaagcaacattagaattggacatggaacaacagactggttccaaataggaaaaggaatacatcaaagatgtatattgtcaccctgcttatttaacttatatgcatagtacatcatgagaaatgctgggctggaagaagcccaagctggaatcaagattgccgggagaaatatcaataacctcagatatgcagatgataccacccttatggcggaaagtgaagaggaactaaagcgccttttgatgaaagtgaaagaggaaagtgaaaaagttggcttgaagctcaacattcagaaaactaagatcatagcatctggtcccatcacttcatggcaaatagatggagaaacagtggaaacagtgtcagactttattttttttggttccagaatcactgcagatggtgattgcagccatgaaattaaaagacggttactccttggaaggaaagttatgaccaacctagatagcatattcaaaagtagagacattactttgtcaacaaaggtccatctagtcaaggctatggtttttccggtggtcatgtatggatgtcagagttggactgtgaagaaagctgagtgccgaaaaattgatgcttttgaactgtggtgttggagaagactcttgagtcccttggactgaaaggagatccaaccagtccatcctaaaggagatcagtcctgggtgttcattggaaggactgattttgaagctgaaactccaatactttggccacctgatgcaaagagctgacttatttgaaaagaccctgatgctgggaaagattgaaggctggaggagaaggggatgacagaggatgagatggttggatggcatcaccaactcgatggacatgggtttgggtggactccaggagttggtgatggacagggaggcctggtgtgctacagttcatggggttgcagagttggacacaactgagcggctgaactgaactgaaacacatctagaggaaaaagaggctaacagtgaaaaggtctggcttgctcctgtgaccctcagttagtctcagtggctcagtttctttggatattaaagagtcacagtaccagtttactccagtccacccagatgtggttagaccccttccaagaccaacatacagaaaactgatttgtgaggacattactgtaagtaaattgtaggaattctaagttgataatttaactatcttagttttgtctacagcactgaaagggatttctggccttcgattttcatctctcatgtgtctgtttaatgtcaagtcagggagatttagccatcagcattctccttatttttcagagcggtcaggtattttttaatgggtgggatgaggcaaagacgcagtgtaacaaatagacttcaggcttgtccctgaatcttttccctctcaatgttaagagagcccgctggcacacattctttaggtctctcatcctccagcttcccaagtcagcgcacacccagggcagtgctgtgctgcagagagaagctgcagagggttaatgattaaggcaacgtgggccgcgttactgattgcccctttcagatcttccagcacccattaacataaatatcatctcaccattttggacaggggacaccacccttatgcacgttagtaatgcgatgctaatgcttaaccctttcattgccctctctgcttcaaaggccttgccctatgtccattgatagggaggctgtttgaacctcaaggcaaaaatgaaagggttaactccttttggttttgccctttgaccacttcaggatgagaggcatgatgaaatacttttcaaagtcttcagtggaaacacatcatgcataattggagcaaaaaagaataaatcaaagcttcccccccccccttttagtctggtaattcataattgaaaccacattgaggtaagggttgataacttcaaaacaaccatgaaaactgccctttcgatcaaggtagggataggtaaggctgctcataaccaggggcttgaaagcttctgtataacaagcctgtcctctccttttcctttctcctctgacttctcacttttctgttctggcaaaataccttcatttaaatcaaatgtgggttgactcaaatatctgcttaattctcttcttgtcacaggacgttatacatagtgacataaaaaaaaaaaaacctctgctataataggtgttaaattttggatcatcctgctttgaatgtgattccttctgtgtgggttttgcctagtgtaggttgagagagtcctttcgttgatattagaatgtgtattttataatcctcctgtagttccagcaagaccctttcagaatggctctagcagtttgcccaccagcttcctccactttctgtaggtaggtaactttggccaggatggttttggttaaggctcgcgtaggactcccatgacctttcaaccaaggaagtaaaacaggattgcaggaagggcccagacaacttccctgtgtgtgagcctgcccaaggcacaccagttagttgagttggaatcccctgcttgaaggagaagacccctttgaactggtggtgggctttaagcctgatctcccaaggcttctctttcaagagatcctgccacccgaacccacaagtccgtctcgctggctggtctgttcgctgttaattcattcaccagggcgctccaggataagagatgcggtagaacttctcgaaagtgggcgtggtgatgtgacagttgtcatggctgtcgtggattaagttaacagtctgagtttctcagcttccctgaagaatgtttgttctgagtgaggagtgcagtggggggagggtgagtggtggaggaaggaggtatttggcctcaacgcgccgtgcacacactcctcctctggtatcacctcccatcttggagtgaagagtttcaccgagcataccggaaagttccttgagagccagggttccgggctgcagctgcggtagcagggctcagccttctcctctggcccctcttttgctgggtgcctggtttcgtgccccatcgtgccatgattgagatttaatcaggccgccgagttaattgccagtgctgtttgccttttccctggcagaagtcagagtcttgagtaaattatggaggtttaaatttgcttgatttcctgcagaaagtggggtcaggaggtcccagactgtagacactctctttggtcagccagcagggggtcagtccttaacggtatttcagaggtgactaaacgctgtttttgaaatgtccttctccagtaagtttttttttcctctgggggttggtctgttgaccttaccctggcggagtggagtgtctgtatgaaggcacacaccccgttaaagctgcctttctcctccccatcaccgggctccaaaggcaaatggaatcatgtttgcggacggagaggcacccagcacagaagcccagctgcagatcagcatttcctgtttctgaccttttgagacacaatgtgaaggtagccgtgttaatttatcagtaaaacaatactctggacaagaaagcttcaaaacaaatcaacagaaaaccaatcatgagacttaaaaagaaacttctgtcgaatgggcttttcatgatgtggctttagtcttgagtctgaggtccgttgtctgtgctcccctccctccctcctcccgaccttggccctggcagagtctgcccctagccagcgaggaagcaggtctcagcacttgccactcagcttgccctgcggctgtttgattagctccgtcccctcctcacctcctccaggaagatggaggtgattctgtggatcttctgggcccacgggacttgaggaccttgttgtttgcgttataatttgtctttttacctagctgtcttccctaccagattctgaactgtttgaggagaaggaatttgtctttgcgtctttaaccctattcttggcttatagtttatgctcaggaaatgttgactgaatgaatgagcaccaaaaatcatactgatggaaagattgctgagctgctaatactgttgttttggcagatgagaatatgaattggagcgagctagttttttgtgtttacttacaactaccattcctagtttccctgatagctcagttcattcagggcttcttggatagctcagttggtagaatccgcctgcaatgcaggagaccttggttcaattcctgggtcaggaagattccctggagaagggacaggctgcccactccagtattcttgggcttcccttgtggctcagctggtaaagaatctgcccgcaatgcgggagacctgagttcgatccctgggttgggaagatcccctggagcatggaaaggctacccactccagtgttctggcctggagagttccgtggactgtatagtccacagggtcgcagagtcagacacgactgagcgcctttgactgtcacttcactttctgcatccctggagctctgtgttgagccctgggctgagggcgcacgtgcatcatctggagggaggtgctgtcaggctctccattttacaggtgaggaagctgagagtcacagaggaggttttccacaccacagtaaatgtcagcatccagggtcacactgtttattaaatatcctaatacacttcttcagaatacttagcacgataagattgtggatgcgtattttaaaatgaatgaactaagttggaaaccatttcagagctttgatgtcactcatttgaatgtacattagctgttcacacccatgcacagagaacagaaagaacacaatttaggcttcatcatttttaacaagtttttgttgagtcgcaccaagcactgtgctaggcgctgagtgttaatagcggttatgactctggttttcatgacgctttcaggatagctaaaatataatgaataggacagaaaagagaaaaactttgaaaataaaaagtgtatttggagaggaagaagaatccaggagaatccaggtaaaccaggaccccagagcatggtgaggtgaaggtagagaggaagaagggattcatttaagatcctagtggctggaagatagctcatgatcacttagccacctcccacctgcttttgagacctgcggaaagtgaatcccagggaaattgctgttcccataagaccatctgttggcttcctttatcgagtgttgctatgtgtcaggcactttacatttattagcttgttactgttattaacagcatctagattgttaagaaataataagtaagacatgttaataatttagttattgtgttaagtaacactacctagaaaagagcctccctcatagctcagtggtaaagaatctgcctgcagggcagaagacccgggttcgattcctgggtcgggaatagcccctagagaagggaatggctacccactccagtattcgtgcctgggaaatcccatggacagaggagcctggtgcccatggtgtcacaaagagtatgacacaactgagcgactagcacttgtacttgtgcctagataagttgtagttttaacgtgatcttatagattcagaaactgaggtgcaagagaggttaagcgctttgcccaggccacacagctggtaagtgaagatcctgggttctgattaaggacatgtgattctaaaactgacctccaatgtgctggctcactttttgaaggcatgtcctacagtaaagcgtcaggctcataacacggtagacccatgctcagcggtttctgtgtcgcctccatgttcatctcagcgcctgtgttttatgggaaagcgttggcaagatttcagagcttgttgcgcttctttacgtgccctgtctcccccagtatgctgccgtttctgcttctccagtcttcaagccctgttggcacacaagtgagcggtgtggaaaaattgagagccaagacaagttactgtccgtttcccacccatccttcatctcctcccacatcccataaccatccgcttcccgaagggagctggcataccagatgagacaggatctgctgtcttgccagagacttcgtttcttctcctggcagtgctgtgaaaccagagctgtgatgcctttcggggggtttaaattccaagggattcattttctgaaaccaagctttgcatctctcagggaattttccccctcttttgaatgtcatagaggcatgcgagtgtactggcgttccccacttctcccgaagaacagacctcattttgttggaggtttaatagtttttcctcctgtgttagaggtgtatttacctttttaaatttatgtgctccctttgtgtcagtgttatgtatttggtgtgtttttattccatgtaccctgtgaaatcatagtgtttaggtgggctcttacttattggcccagatttgatcatcaagaatcaagaaaaccttcagcatttctcttctcttctcttctcagaaaagagctacaagtaataagctctggagtttcccacagttccagagtctgtaaatggtgatgcttacctcaaactggggaaggaccatgaaacaccacatcctggtgttctggagtcagacagcctgggcgctttcctgctctgtcatgtataggttatatgaccatgaacatcattcactatttcttctttcctttctggactggtaaagcaaatgaatagacatcattcactatttgtaatcccacagtttcatcatctgtaaaatgagagtggtagccatctattttggggttgtcatgaggctcaaatgagataatatatgtacagcacagtgcctcacagtaaagtcattcagtctttactgaactctttggaaccccatggactatacagtccatggaattctccaggccaggatactggagcaagcagggattgaacccaggtctcccacatttcaggcggattctttaccagctgagccagaagggaagcccaagaatactggagtgggtagcctatcccttctccatcagatcttcctgacccaggaatcgaactggtgtctcctgcattgcaagcagattctttaccaactgagctgtcagggaaaccaaagtacttagtaaatcttgactattttgatatcacatattaaattattttacatacttctttaaaaactttttttgcttttccatttaaaatggtgattttcaagcacacaagttttgtgttgggaggggttgatgagtgggctgggttggtgagggcagagagaccacgaaccttagtgattaactgtgacttagtagcacagggctacttccgtttccatacaagtcaggaaactgatgagtaagataggagtagaaaatgaaaaggtgttcctgcaaaggtgatcggatgcagggtggaaacacgaagatccaggccccgtcctaaaaccacagcttaatgcccaagagtgatattgtggaaacaaaaagtcagccttaggaggatactctttcctgtggtgtctcttggttgtgaaatcttaattatggcctgttgtggacattttggcgttaatatcctgatgacattaggactccaggaatcccattcggtctcctggagttcccatccatgtgcattccatgtgcgtgtgtgcatttcctgccccacactgtcacatctggcaaacagctgctgtcatatacgtggtgatggctgtccgtcagtgatatggatgggggtgatgggggaaaccttggagatcccagagtatgaaaagactaaggagcgtgagcagccagaataacaggccatttcatattcagcctcctcctggtgaagctgaagttaggtcctaaagttaggcttttcatgtaaaagcacgtgaatgtaaatgtaactcatgccactgaaatgtacacttgagtttgaaatggcaggttttgtgttatattttaccagtttttaaaatttagcagtgtaatataccaaagccattgcataccttaaatagatgcattgtttagtacataaattattgggcttcccctgtggctcagctggtaaagaatctgcctgcaatgcggaagacctggttcagtccctgggttgggaagatcccctggagaagggatagcttacccaatccagtatcctggcctggagaattccatggactgtacagtccatggggtcgcaaagagtcggacgcagctgagcaactaaaccaccacccctcggcttcttctattgtctctgttttctctgacttcttgtgtttctttctgttcatttgttttctattgttcatgctgaagatgttcttgaaatgtctgattctagggtctgtggaagctctaaaaagctgatcaaaagctttttgtttaagagaagcttgttacctggagggctttgctgtgaggtgatggggcctcgctgtaaggtgatgcggcctccctgtgaggtggtggggcctcgctgtaaggtgatgcgggctccctgtaaggtggtggagtggcaagtaaaccacatggctgctagtgtttgggttgctagaaggggaggaagctgtagagctcatcattcaaaatttaggctctaactgcattttcctctcttcagtacctatgtttccatgtctagagctttaggtttttcagagaatcaacctcccttctgtagcgtgggtaaggaagaagcgtagttttctggctgagtgggcaggtggtgggaacgcttgtgccctgagcctgtcgctgctccttgcacgtatttcagctgttcctcctgtgttcgccctcctcctcttacctcgtgaggcccgccgagcacagcccctgggccgttgtggggctggcagtgggacttggctgccttgttgtctcgccaccgcctttgtagcacttgggttgcagctctcagccttctgctccgtcaccacttctccagcccctttcagtctttcagaagcatgttgctgctgctcatcagctgctgtctctgttcccatttcattgtctgggtagatttatattttttatctattaattgccattttagtgggtttggggagggaatggagataaacgcatgtgtttaatctgccatgtttaactagacatctcctcttagctgcaaagattctctgattctttttttaagggtatcagagccaaactcagttaaacagcatcagtacagattttgaggtggggaagactttgaacagttgcttgcctcttggattggtcatggtagaaactacatcgagtctcccgtgttccggtttattggtcatgctgagaaagagaccagcaccattgttactagatccttctctgacccatattaatctgttctcttgcagaagtggggcagtgtgagcaaccctctcttccttcccctcatccccccacaatctcaaggtttcacggccatcgtcctcacctacgaccgagtggagagcctcttccgggtcatcaccgaagtgtccaaggtgcccagcctatccaagctgctggtcgtctggaacaatcagaataaaaaccctcctgaaggtaagacgagcggagagacggtgtgtccaggaaaggcccggtttggacgttttgtgttcgtctcaatgggatcgagtttttttgatggacaaatgatttgcagcctttgtcttaaatgaactttcctgctttgtcaacagtaatgccattcctgaggcagcatgaccctggttttctcagtcatcttgttcttgttctagggtggccggtttaactcttagcccatggcatgctctgtagccacaagtgtttgagggcttaggagtcaataggatcaaaggccatcagccttgggaaaacagatctcagcttccctcaaccctctcagctgatttgtttacatgaaaagtcagtttaaaaggcattagttttctccttccttatctcggctctgatactgtgctcttcttcaaaaaggacggcctcacctctgatgggcagagtcagcaaagtgcgccctcccaggcttgaatcagctccttaaagtgacccctcactggaagttcccaccagctcatccgtaatcactctgtcccttgccttggccacccctgtaatctgctgactggcagctcgggctgagcctgtttgattcattggatttgtttttaattcaggacgcataatgtacgttccacctgcatttcgtttctcattcagtccgtctattatggatcctaatcccagggacttcaaagcccctgccctttatagcctattaaagccaggaagtttctgatggattagagcaaggaatgagtttgaagtttgtaatgtgcaccactatcaccgataaggtctagtttattgcctgacagtagtatctgcactttttatggctgttcccgtttaattggttatcctgaagtcgggatagtcaaccttggaaaacccagtagtagatccctagtctaggctgggagaagaatgtacttctgtctgtgaggcaaatcaggatttaaatgccctcttgagactttaaaattttttccttggtgggacttttgttctctgcatcagtacttgctttttagattcagggggccacagtttagtaaacaaagtagctcttttatgtgaaggcccatagtactccctgaggcacacggcgtttattatcgttgttgctattctcatcttttgtgccgggttgttttcctcccagcattcttggatttttttttgtttgtttgactctcaataaaggggacagaaaagggtagaagaaaaagaggatgttggcaccttctcagagggagagaggatgaaactgacgggaagggggaggaggaggaggccgacgggacagaacgggacagcagggaggctcttcagagccccgggtccacccagagtgagtggatcggaggcactgcattgctcatctgagtcttcattcataaaggatgggttgtgtttcagaaggaaggctgtgtgtcccttgttggagctggaacagctgtgtgtcgtatctgcagtcaccctgctgcatgccgaggccaccagcttccagggcgggagcaccccagctgcaccccaccctcctgcccagacaggcagtgctgctgctaaatgcagaagagacgtctccttagaaacccgggttagccactttgggcataaggtgacttatttaatgtgactttgagtcacttctttagagcaagtatcactgcaaactgtgaggaaagagaactagttttcttccttcttggtctaaggtcctgcctacattgctgtcactaaaagcaagagcacaaacctaggttctggagttgggtgtttagttttaatttttggtacaccctaccaaagtgtagtggtgaatgtcatgaccaagagtgtagtttatttttgaaatattttggctctagagaggaaagagccctcaactgatgaggcccaaattttgacacattcaggggctggaccagctttttttctaacagtggtttttaggtcagggctctggttctggcgttgtggttgtttggggagaacatgaactgtgtttaatcttctgtggggtgtttgttcagttggtaaatggggctgtcagctctggccctcgtgccagcaaaggcatctgatttttcatgtacggcattagtaacatatatattttttaaaagacaagtccaaggctttgaaatattagaggcaaaagtgatgtttttcactttctctacatttactaaaataaatattgtggttaacatgtctgctgctgggaggattagtgtaggcagaaatccctcccctctactttttttttttttcactctccaacccaaccaaatagtattttctttttggttacatttaagtctaaattgctgggtttgaagtttaagtaaatacttagactaggcaatagaaatttccagcgagctttcgtcacgttggttttgagctctgtcagacgaaacaggcgagccactgccctcctccgacctcagcttttcagtttcccggcttgatggatatagaggttgttaggcttggcccctggcctcagcctgaaggaagctgttacatcgtacatttcagatagatttaactcagccttctgttttaactgatttcagtttttaaatttgaattgatctcagggaactcccctatggccaccccagcttcgggttgctacttccacagttcttaataggcaggatgggattggctgagcgtttcagggcggtctgtcgggatggaggccatcgagaagcagaagcaggagtgcccttccttccagctgaattcagggaccgtggggagtgaggctctggggacccgctgttcaccaggcaggttgaggacttctggagggagctcaagatttgtacagagataacctgatggttgattttgtgaatctgtggttgtgtttgacagtgatccccaggatggacttggggtcatcgtcggacaacctgaacatttcgtagtacctttgccaagttggagaattctcctagctcacatggctcctggcatccaacttggagaaaactgttaatcaatacatgtttttaaaaaaatccttaacacaaggagttctttttctccttcaagttggactaaagctcagaactgtaaagttatcaggtcagttggctcagctgtttgtttttcataaatttggcaatgtttttgagaaatcttatttttcagactactttgtgtgaatggaaggtgacatttctgttttgccagctatagattcgcaggatgagacacaagctacaattcatgcagttccatttagaggttaaaagaataaggaaaagaccccacctgggccaagcagtggtggaagaacccgaggtttctagctgtgacactgtttccttctgcgtgctaaggagtcaccacctgttcttctgtctacaccgcttcaaaatgtcagaggcaaagtgaagtttgtaagaccttacaagaatccatttgtgaagatgcctgggtaaggaagatctcctggaggaagaaaatagcagcctattcccgtattctttcctgaaaaatctcatggacagaggagcctggcgggcgacagtccaaaggactgcaaagagtcagacatgactgaaccactacgcacatctgtgaaaatgagaatgatacagtgttgttacaacatgtatcgtgtaccttttatatccaaggaatgggaactgtgctgagtgctttacacgtattgtctctactgcttataacaaaactgaaagaagctgatattatccccatttacaaaagtgaaactcagattaactgagtaatttgccctagggctcccctggtggctctaacagtaaagaattcgccggcagtgcaaagacccgggttcaatccctgggttcggaagatccactggagaagtgcatggcagcccactccagtattgttgcctggagcattccatggacagaggagcctggtgggctacagcccatagcatcacacagagccggacacgactcaacaactaacacacacacgtcagaggtctgcagaaatgaacacaagtctctctggatccaagctcatgctctgtgccccgagccccactgtgtctattcagattgctgtttagcagcaaggagatcatgctttgtcaggtctctgatttgggattatgcagatcaaccaggttcttatcaggggctcgaagagaggcagaaaaataaatcttattcttttggaagtactaacggaaagaaaaatacaagtcttctgttctttctcacttacgtttttctttcctaccttgtattcctgtccttttttttttttttttcccaaactgtatcaaaatcctatatcagaattaccttctttaaggcagggagtaattatactgcttctttggctagccactttcttgttcccgtgttccacagaagggatacaaaagcccaaggaagttggatcccctccttagagccctgttctatttgaagctgtgaaaacaagcaactgggagttctacttggagtccttccttgtggtcacagtggttcatagggagctggaccgttgagctggagacttcttttaccaaagctgcctttggctctccaggcccaactcaggacaggaaaaggtaatcacctttccaagaaagccacgattcattctacatttccatctgtacccatttagctttcctcacttgctaaaacttccctatcaaagaccttatcaaggaaggttgtttttagacattgtcttgagtaattttctggctacacataggaaaagaatgtggaaaactctctgttgaagatgttgtgcacactccctgggtgagagggactctcatccagggtcgagaacattgacacatcatgcaagcagctcacctgttttctagcctctttctgtattgcagaaagagaggctgtttgttgccttgtgaaatttccttggcaagagttccatgtttcttgccaacggaactgttggcaagttaggttttttcagttctagaggattaatggcagggtttccgctagtctcaaagtgaactttgaagataaaagctgaggacatgccactagactgttggagtgatgtacgagatctaacgtgtgcctctttgccaccactacctagggttcatcctttggtctgtaaataggcacagacttgcaagggaaagcacagccaacgtttggtggacgttctgccacgtgctgggcactgagctgctaaaagcattcctgttcctcctcacgccttgccacaggtggagatggtgtcactatcccatcttcatagtcaaagaaacaggtacagaatcacggacttgttcaagatcacatagctgataaggaatacaaacacctgagatttgaagctctttccaaagactgttctctctgtgaaacgtgactggccatacagtcctcagaggtgtcttgtttggtccctgaagttggtacatttcacgtggaagagcacatagcgttgctgaaggcatttcgttccatcagaacattcttaccgactttgagcctgaatcggtttccctgtaactttcacccaggcgttggtcacgcttctagcttcagaagccatagagaagccattcagaagcctttcatctgggaacccttctgatagccctagtgtgttagtcaaggtccaggtgggagacagaaaccacacagcaacttgaccaggaaaagttcaagttttaactaggaattgttaaggaggactaggagattggtggctaaagggtgaagagagctcccgggatataggaaatcagcttggggattgagagcgcttccccaagctcggtctgagattcagacccactgcatggtgaagctgctcaggtgccacaggctgagtctggcaaacaggaaaccagcagcccctttggatacccatggaattcaatgggaagccatccggggggtgttattggactcagaggggtactggcgagggcttcagttcctgaggaaccacctgagggcagaacctcagctgagttgctgctgaaggtatcacagaacttgccaggaaactgcctgcagaggagctgccagactccttcgggagcctgttggggtgtctgtagaacctgctgggagcctcccttgggggtgccgtctacatgtgctggagaggggtgctgctgcctacctgctggccaagtaccgaaggagcaagtgaaaaaggcacaccagaaccaggaagcaaagccctcacctcccagcgtccctccagcacctgctgctgacgtgttaacatcaagtcagtgggcaaaggggaaatggtgatggggctggctgccagtatcacccccagggcagtgaagggtgaatttggagctgagaggcagtgcatggcatattcagtttaaagagatcctggatccgaggacctgtactgtcctcatcaactccgcctatctcccagtttgttggttcttctgtaagtgtttgtgtgctggtcgctcagtcatgtctgactctttgcgcccccacggcctgtagtcctccaggctcctctgccaatggaattccccaggcaaggattctggagtgggttgccattcccttctccagggaatcttcccgacccagggattgaaccctggtctccgacattgcaggcagattctttatcgtctgagccatgctgggcagctctattgcagtacccaaatctaaatacaaggtctcatacagaggttctcaaagtgcagtccggggactgctgagcatccctgaggtcctttcacagggttggtgaggttgaaattctttctctgagaatgctgagtcaccctgcgttcccacgcttatattttttacgtgtgcatagtggggttttccagggactgcccaacatgggatgacatcaccactctactggctgatggagggtatgctcgtgtgtcctgggttttaaagtttccttggttttcattcagaatataataaattgtgtgtgtgtgtcctacataaataaaacctctttggggatctcaacagtttttaaaagcacgaaggaatcctgacaccatttgagaaccactgatctaaagataaccactgatctagttagagctataacttccttcacttttagatattacaatttattcatgttttctaaggatttggatttatgacagggatttcttttacttaactggaagagggaatgggaagcagagaagaataaggatgattgtttttgttccagatccagagaaacatgacattttcaagctgcagtaaacggtgcaggtcctatttggtctttgatgctaaacagacttagattcaaatgctagctgtatcatttagccctaaccccagccaatgtacttaacctctctgagccttgtgaatcctactctgtaaataatttctgctctatagacttgtcaggaggatgaatgacagtgcacctgtaaagtatctggcatatactggttgcttaatgaaggttcccttctctctctccacttattcttaccctgagaataagggacctgcctaagaaccccatgggaaatgcagaactcaggtctccagtctcttagtcctgtgcctatccatttattctgaagacagtttttgattatgtcattctggaccaggttaagtattgattaaaagggactgacccattggatcatttctttggtctccagtcctttctcaaacatgcttttttttggagttgaacacatttttagaccctaagctttttcaggctgtgaaagtgttagtcgctcagttgtgtctgactttttgtgaccccatggactatagcctgtcaggctcctctgtccatggaattctccaggcaagaatactggaatgggtagccattcccttctccaggggatctttctgacccagggatcgaacctaggtctcctgcattgcaggaaaactctttacaatctgtgtcaccaggcactttaggccctaggcttatttagaacttaaggactatcttctccatatatatatatatatatatatatgtatgtatgtatgtataggaatatcttctttaattacagggcctcgcactttcacaggcctcttccaagaacctaggagcaccttacctggacacattgtaaattatgcagaagtaagatattttatatgatgtctgttagagcaaccttttttcctcctgattttccctcagtcatacttatccttcggtatgataacttcggagtggccacaggcattttggggcttcaagactcacaaaactagaccatcccacaattatgtgttgggtttctggaagcaatatgatacctttccgtgctcatctgacaaatgttgtgtgaagaacaaactgccctctgtaaagaaccctgaggtctatggacataggtcctgtaccaaagaagtgttaattttattctgtgctaaatgcatcactttaaaatgaggaacctgaggttgaattgcttcaaagtagaatgggaaaaatgtaaggcgagtcacaagttcgtgggtcttcagtataccagaaaatcccatgtaatcggacctagaaatttgatatttactctgtaaattggtcatgatgagaacccaaacccagtgagtcaggagagagcggctgttgagcggtgaggtgctgagttaggaaagctgaactcattcagggcaacggttttgtgagggtggcttcggcggccttgggagccgttggtggtggaagtgggcactctcatcttctttcagcaaatgtttcccagaggggctgcaggcaagtttggggctgggtgtacacacccggaaggattatgcagaggtggccaggtggtcgagtcctggtgttcaaaccatgggcctgagaatgaggcagacagggcccatgcctgctcctgccctgtccctgtgaccttcatcaagtcgttcggcaccccccacccccacccccggcctctcagttgtgccttggagcctgtttgtggattctggttaagacagcattgagcagcagcagtgttgtaaggatgtagagagaagagaagtatttatgatatattataaaaattcatctttaagaatgtcctcctgtagttgtgcatgcggttattgttttttccccatattcttatttatttttggctgtgctgggtcttcgttgctgtacggcctttttctccagttatggagagtgggggctgctctctagttgcagtgcgagggcccctccttccagtggcttcccttgtttcctagcgtgggctccagggtgcacgggacttcagtagttggggctcccgggctctagagcagcggctcagtagttgtggcacacgggtcagttgctctgtggcatgtgggatcttcctggatcagggattgaacccacatctcgtgtatcggcaggctgattctttaccactgagccaccggggaagccctgcgcctgtggatttcatgagagtcacagctttgagttgcttcttctgtcttatttgtgagacatggcttttaccagtttatcttttcaaagaagttagaagagtcataaataccttggtttttttttttttttttctttttccagattctctgtggcccaaaatccgggttccattaaaagttgtgagaacagctgaaaacaagttgagtaaccgcttcttcccttacgatgaaatcgagacagaggccgtcctggctattgatgatgatatcatcatgctgacctctgatgaactgcagttcggttatgaggtaaggaggtctcaaacagtgcgttttcatatttaatatttattgcccattattgcttgtcttgcctaatacagagggttatatttcattctctgaagttgtgatttgtaatagcacccaaaaaccgtgtttgggagcgtgggtcacgaagctgttctttagagcttcgtggaacccctattccaaaatgacaattgtgctggctgccagtttggggagctcccaaggagggtcccaggggacatcttcaaaagagcatctatgagatttaacaagcgctttactgatgtctccatgaagagcactgcccagactcaggcagctgggcatggccacaataaaaacatcagtaagtgaagaagctggcgacactgacaaacagaaagcagacccgctgctggcttgccttctgagtttgaagtgcagaggctggctgcctgtctttggggaggagcattctctatcaccgttcattcagggaggctgatggattaggccgggttcaagggttaagtatggagaccaggaaaggtctccccaggggaaagtgccagagccgaggcctggtgaaaagcactaggaattgatgtgtcctgtcagggggatgtccgcgagacgcgtgatggcgacaggcagaggcccggggctgcgggggcgagtcgggggtatcagagccaagcagaacagtgtgcacatcaattaaccacagccggcaccagggaaagtgggggcatttatccccaagactcatcctgctttggggagagaggtaaccatcttctttagttgactcaggatctgttaaatttgaggttctaagaaatcatgaaactttatttaatgaagccatatttggtacatgtgttatagaaatttattttctaagccttaaaagtggatttttctttcttattttttaaattattaaagtaagataacacgtttataggagacttagaaaatacagaacaaagttacatatagttctgctatatattacagttctttttttaaagtaggtaaattaagatttttagttggagtttcaatatcagagtttcaaaaattaatagaatgaatatacagaacagtagaaagatattgtagacctgaaaggcaccatgaaccaattcagcataattaagattcgtacaactttcacacaataataggatacaaattctgttcaagttcccatagactgtaaactaggaggcattggaacatagccagggacataaaacaaggtgcaaaaatctcatggtctaaaggtattgaaatcatagagtctgttctcttaccactgtggaattatgttagaaatcatatttttaaagttatgaaaataattcacctattttccactgcagcatgacatttaccaagagatatctttgatttagccattaaaagcctcaataaagttagaagactgaaaaacaatacagttattgcagagaagaaagcatttaaataagaacttaatatcaaagatactttagaaaacctcatgtatttggaaattcaatgtccactgaaagtggattttaaagggaagaaataggaatcccccctgtgaccaccagcaccaaaaattagaagcttatattcccccaggtgtaatcccatggaccgtagcttaccaggctcctacgtccatggaattttccaggcaagagtactggaatggattgccatttccatctccaggggattttcccgaccgagggattgaacccaggtcttccacattgcaggcaaacgctttaccctctgagccaccagcgaagtcctttttatatatagacataaataatttaacatatatggaaatatatataaataattatatataatgcatattatatataaataactatatatcagttcagcgctcagtcgtgtctgactctttgtgaccccatggattgcaacatgccaggcctccctgtccatcaccaactcctggagcttactcaaactcatttccattgagttggtgatgccatccaaccatctcatcctctgtcatcacctcctcctcccaccctcagtcttcccagcatcagagtcttttcaaaggagtcagctcttcacatcaggtggccaaagtattggagtttcagcttcaaaatcagtccttccaatgaacacccaggactgatctcctttaggatagactggttggatctccttgcagtccaagggactctcaagagtcttctccaacaccacagttcaaaagcatcaattcttcagcactcaggtttctttgtagtccaactctcacatccacacatgactactggaaaaatcatagctttgactagacggacctttgttggcaaagtagtatctctgctttttaatatgctatctaggttagtcttaacttttttttcaagaagcaagcgtcttataatttcatggcagcaatcaccatctgcagtgattttggagcccaaaaccataaagtctgccactgtttccactgtttctccatctatttgccatgaattgatgggaccagatgccatgatctttgttttcggaatgttgagctttaagctcaacattttataattatacataatgtatattatatattaaaatatatttatatatccattgagtataaaagaaattgttgtgaaacaggctggtatgtgaataccctggtcgacatcattactggccagtgaaaggcatgaatgtagctagtacatagactcaccttcctgggtttatttccattcatagagatgtgtttcctatcatcacacctaaggtgttggggatttttcatacactatctttccctttttttaaaaaaagcaaattacttatttctatagccaactcatggcaaacagacctggtttttttcttgccaatgttttctctctccattatatataatggcttcaaactatatccttgtgtttgtttttgtggtaaaaggaaggtgggaagtaggaacatttcaatgaggaaaggtagccaagatggacagagtgctgcctctttgctatttgcagtcggtctaaatgaagtctgataaaagaaaggtaccctggaagaattcctttcaaccatggcttaaaatcttgggatctcaggaatattgcagcctgaaatctctttcaaccttgagttcatttagagcgacaaaaagtgtattgatgagaggaagtgcaaaacccagagggactgagcttaaatcttcatctcttccctctgaggccctgtgtttactccacgtgacctctgaagcagtcaactgtggatcaagaggtaggggcagggacacccttcatctctagtaatgggggtcttctccagatgctaagacaggagtaacttccctaaccaccatctatgtgctgcttactgtgttgctaccggtttgctggctgtttacatacaccagtggagaccagtcctgggtgttcactggaaggactgatgctgaagccgaaactccaatactttggccacctgatgagaatagctgactcatttgaaaagaccctgatgctgggaaagattgagggcaggaggagaaggggacgacagaggatgagacggttggttagcattactgactcagtggccatgagtttgggtaaactccgggagttggtgatggtcggggacgcctggcgtgctgtggttcatggggttgcaaagagtcagacatgaccgagcgactgaactgaactgaacgatcttttcccaaacctcctaggcagttattgggttttgttgggttttggtttttgtttttgttttcccctttctttactgagactaagcaagattagatgacttccctacagtcacacagctaaataagtgggagagttgagatctgaatccaagtttgggtgacactatattgtcttccttgaggattttaccatgtcagagactactggtcctcaaaagtagtcctcagaaccatcttctatcacttgtccatcactaaattaccttggtactaaagaaaacaaatttatttctcaggcatccctaccactggtgctctagggtgctgttgtaaagttacctcttgcataataattaatactatgaaaaaagttagtgctggacccaagtttctgttctgtcaccgtggacgacttatgtaatctctctgggttgtagtttattcttggacagaatggtgctgacagtgtctgcttcgctgggttctcatgtgcattagataagattgtgtagcggagaaggcaatggcaccccactccagtactcttgcctggaaaatcccatggatggaggagcctagtgggctgcagtccatggggtcgctaagagtcggacacgactgagcaacttcactttcacttttcactttcatgcattggagaaggaaatggcaacccactccagtgttctcgcctggagaatcccagagacgggggagcctggtaggctgccgtctctggggtagcacagagtcggacacgactgaagcgacttagcagcagcagcagcgccagtgcacgggcagcgattagaaagtgcatgttctggctggtgggctgcgtgctctgctctttatgagatgcttcatcagctgctctaccatgactgctgccgtccttctcgtgatccagtgattcatggggagggtttgtggttgttttcatcattcagaagggatagtgctcttctggggagggtacccagttttgtgttaaaatcagaagacagaagaggatggtactcctaaatcctgttttttttatgccaaagcctgggattattttcatgtagacttgatggattaagagaataaacaaatcagttaataaaaatttatgtcctaggggggttatgttggagcccagaactacttctttgtcacagttaccactgtgtgttaggattatatctatcttgttgactgtttttcgtccttcagcactcagatcaaaagaagctttcacaaagtctccagactggagcactcaacctctttgtaagcaaataattgtgaagtgtgttgtttagtgtctgcttttcttctagaatggaagccccatagggacagaaatatggccatcttattcattgctgtatcctctgtgcctaacccagctcagtccattcagcagttagtaatatttgataaatgaacaaattttcattaccagcatgcacatgaaagataggagacagatgattctggcccgcatgcctttccactttactgggatccataacctagatgcaaccgtttcagcaaacagtttggtgtttaaaatgttgactgtaaatctacagtgtgaccccaccgttcttcccacagagatttaccctagagaaatgagagcatatgtatacataaatagttgtacgctaatgtaagttttagtgtttgtatttgcagtgtttatttgtaaaagcaaaaaactggaggaaaaaaatcttatcagaagttgaatgaatgaactaattgtagtgtctccatacagtagagcactacttagtaataagtagatgagctattgatatgtacaatatagacgtatttaaacatgattatgctgagtgaatgaagcaaggtgaaaaagagaacatattttatgatttcatttataggaaacacaaactaaatcttcagtgattgaaaggtcagtggctgcctggagaagggtagggcaagtcagcttgaaaactttagtaaaacgtgtgccccatcggttcaatgaaaactaggaaacattgctcaaagaaattgaacagcttcataaatggaaagacgtatcatacatttgaatccgaaggttcacaattattaaggggtaagttctccccaaactgatttatagattcaaagcacgcacagtcaaaatcccagaaggcttttttaaaaagttgaagttgacagcagttctaattgaaatggaaacacaacggacctgaaaaagccacaagccaaagttttaagaaagaaggacaaaattataagacggccttatgttaagacagataaagctaacgtaatcaagactctggtattggtgtaaacttagacgtgtagatcaatgaaacagaatataaagattctagaaataggctacatgtgtggtcagtcagttttctgcaaaagacccgagtcaattcagtagaggtggaataggcttttcactacatagtgctggagcaatttgatatccatatgtcagttcagaatagatcttgtcctaaatacaagaactaaaactataaaaacttcttcaaggtaacagtgaaaaaagttctagtgtgcctgagctaagcaatgattttttttttaaagtaggagacaaataacataatttataaaagaaatcattgctgctgctaagtcgcttcaatcgtgtccgactctgtgcgaccccatcgacagcagcccaccaggctccgtgtccccgggattctccaggcaagaacaatggagcgggttgccatttccttttccagtgcgtgagagtgaaaggtgaaagtgaagtcgctcagtcgtgtccaactcttcgagatcccatggactgcagcctaccaggctcctccgtccatgggattttccaagcaagcatactggagtggggtgccattgccttctctgaaaagaaatcactggatttcatcagaattagcaacacttaaaaagacacttctaagaaaataaaaaggcaatttatagactgggaaaaatattttcagtatatataaagaactattcacacctctctaatgagaaaacaagtcaattaaagatttgagcagttacttcataaaataaaatatacaggtatagactaacatatggaaaaactctccacctcatcgtcattggggcagttctacagtaaaagcacagagggtcatcaccatacacccactgctgtgactaaagaaaactgcaaatgctgacagtgctgcatgctggggggatgtggagcagcttagaactctcgtacgttgttggtaaaaatgagaagtggtacagccagtttgggaaaatgtttagcagattctcataacgtttaagaaaaaaaagtttcacttttctatgacccagattccaaaagaaattcatagcgtcactcataatatagaaaacctagatgaatcaacttgcaaatagagaaacagtatggaatacctgtgctgtgaaatatcattcagcaataaaaagaaataaactcctgatatatccagcaacctggataaatctcagaattgttatactgagagaatgaagccgtgtgcaaaagaccacttactgacatgttctgtgcataaaattctaggagaaactgtgttgccacgggcgagggcagacctgagaacaaaagagcagaaggaacttttcacaggatggaaatgttctgctctgattgggatggtggttataggactgtgtacttttatcaacacagactttgaactggtgatttttattgtaagtaaattattttcccaacctgaagaaaaattgtggaaagcagttcttaggggaaatttataacaagtaaacgcatataataggagagaagaaaaggttaaatgcagtcacctaaactttgttctattttaaggagctaaaaaagaagagtaagttaaactcagagcaagtagaaggaaagaataaagataaatgctaataaaatctaggaaacagaacagaaaattaatagaccccaaaactgactttttgaaaaaattaacaaaattgataaatccctagccaaaccactcaaaacaaaggaacacaacttatcatcattaggaatgaaaaggaattaaggaaaggttgtcatagatcctacaggcatttaaagagtattataacaaagaagggcttccctggtggctcagatggttaagaatctgcctacaacacaggagacctgggttcaatccctgagtggagaaaatcccctagagaaggaaatggctacccactccagtattcttgcctggagaattctgaaagatgactgccagaaaagtagagctcatcaatgaatttggtaaagttgcaggatacaaaattaatgcacagaaatctcttgaattcctatgcactaacaacagaagatcagaaagagaaattaaggaaacaatcccgtttaccatcacatcaaaaagaataaaatgcctcagaataaacccaccaaaggaggcgaaagacctgtacttagaaaacagtaagacacacttgaaagaagtcaaaggtgacaccaacagatggagagagagaccatgttcttggattggaagaatattgtgaaaatgactgtactactgaaagcagtcaacagagtcagtgcgattcctatcaaattgccagtagtatttttcacagaattagaacaaaaattcttacaatttgtatggagacacaaaagatcccaaatagccaacgcaatcttaaggaagaagaatggagctggaagaatcgagctccctgacttcagactataccacaaagagatggtcatcacaacaatatggtactggcacaaaaacaaatatggatcagttgaacagaacagaaagtccagagatgatccaggtacctatggtcacctgatctatgacaaagaaggcaaaatataccgtggaggaaagacaagctcttcagtaagtggagctcggaaaactggcctgcttcatgtaaaagactgaaattagaacattccctaacaccatacacaaaaataagctcaaaaggattaaagacctaaatttaaggctggatactacaaagctcttagaggaagacataggcagaactctctctgacataaatcactgcaagatctttttcaatctactgcctcgagtaatgaaaataaaaataaacaaattggacttaattaaatttaaaagcttttgcacagcaaaggaaaccataaacaaaacaaaaagacaatccacagaatgagagaaaatatttgcaaaccaagtgaccaacaagggattaatctccagaatatacaaacagctcatgcacttcgatgtcagaaaaacaaacaactcaatcaaaaaagtgggcagaagatctaatcagacatttctccagagaagatgtacagatgatcgagaggcacataaaaacatgctcaacataactaagtaatccaaacatcactagaaaaatccaaatcaaaactacgatgagctatcacttcacattggtcagaatggccatcatgaaaaaattcagcaaacagtaagtgctggggaagatgtggagaaaaggaaacctcctgcactgttggtgggaatgtaaattggtacaaccattatggagaacagtatagaggttccttaaaaaactaaaaatagagctacccatgatccagcagtccgactcctgggcatgtatctggagaaaaacatgatctgaaaggatcgcaccccagcattcattgctgcactgtttacagtagccaagacatggaagcaacctaaatgcctattgtcggaggagtggataaagatgtggagagatatatatacacacacacatatatattttttttcccacatatttatgtggaggcagaggacgggatggttaaatagcatcacagactcaatggacataagtttgagcaaactctgggagatagtggaggacagaggagcctggcctgctacagtccctggggtctccaagagttggacacaacacagtgattaaacagcaaagtatcactgagttagtgagtcacaccaataaggacatgtagttcgtatcagtgcagttttccccataccaggccagctgtaaagatcatgtagttcgtatcagtgcagttttccccataccaggccagctgtaaagatcatgtagttcgtatcagggcagttttccccataccaggccagctgtaaagatcatgtagttcgtatcagtgcagttttccccataccaggccagctgtaaagatcatgtaattcgtatcagtgcagttttccccataccaggccagctgtaaagatcatgtaattcgtatcagtgcagttttccccataccaggccagctgtaaagatttcattgtgtaactattccatttttgcctctgtgccactcaaacccatttgttcagaacctctttttggtgaaggtaaaaacacctgaacattttgtttcatattttaatattcacaaataatccatatagtcaggaaaacagaatctatactaatggtttcaatagaatggatttaatgtatgaaaatttctaatacatggtattagagagttgagagggcaaaaagcgaatactgaaaatacaaagagttaacaattccagagtgtacccaccacccctaggatgggagaagcaaaggaagagagggaagaggttggggttgttagaacctggaagcatggaggagacgccccatcccactcccttcctgccagagctgggccttagacctctgagggggtgggccactggctgccattgctggtaattccacggggtcaagatgaagttggttctgccagaagaggttagaaccacctgccgacatgggtgaaatgccacgctgtcgggggaaagcaggcaaaacagaagtagtttccttctcctttgctccagttttctaacttctatctggtgcccctgttgacagaacctaacaggaagtcacctagcaaaggtgagatgtttgctgagtctcagcccccacatcacaaggcagaatctagggtctacgagctgcaagcccaccacccctttggctactccacagccccatgcacacctttctccgcatgtcgctacttccacgcagcagcaactttgttcttccactgagaagatgtaactatcctttatgcagagagaggcctcacgttcccccccagaaggggagaaacaaagtcccgatggtcataattatcaacgtctgtgtaattccttcaatgaaaagtctatgaattccttctgtaatttagtcacaggtctccttaacattttataacttaattaatacattccaggcttccaattctggacatgattgagcaagcacatcccaccctgtttctggaaaagaaagacagactggttagggtcctcaggacttaagagatgttccagcagttcccttggattttttttttcccttcctttcttcctccatttgtccctgcctctcatatacctcagcctcagagctagagatggctatggcacagggacaggtaacatgctaaacaaacatacagacaaccatcctccactttctcttgtaagggctgggaagaacccagagatgcagatggaacccttctgaccatacccgcttgacgccaggcaaacgccaccaaagaagccaaatgtacccctgccagctagaggctgtggttatggagattgtctacagagccctgccatccatccccatcctgcactaaccggagacaggagcagagacggtgctcctggcctgcctctgtccactctggggactgtgaatggagcccacctgaccatccatgtcctccatgaagcgaatgtcagtgaagaggcagctgcagagactgttgcggggtggggagggaaatccagcctgcaccagacaagcaggcagcaagaggcagcatcgcatcccctcccagctagagtggagggaaggtcgcgttagggaggggttctttaaatctgtgtgtgaagttccgagcacactggtgctcctctcccttcaaagcagcccctgcatgaaactgactcgaaccaacacaagaaaagctatgagaactaaactgtggtgtagaatactgcccaggctccagagtggccgctgggtggcatacaagctgggtagaccagaatagccccaatatagtctttggaaattaaattgatattcagcccacagcccacagaagtggatcaggacatgcattctgaacctaaactctgtgctaaaattgaacacatgcaaggcagcagcattgcatagtaacactcaacatgttcaggaaaaaatccaacattactcttcataccaagaaccagggaaattgcaaccttactgagaaaagataatcaatagttgccaacaccaagatgacacagatgttggaattcttgaccaggatattaaaaccgctatttaaaaaagaatcctctaataagcaattatgagcactgttaagtggaaaaatagaaagtttccactttcagcaaagaaacagaagatataaagaaaaaccagatggtaatttcagtactgaaaatttcagtaaccaaaatttaaaacttgatgggctcaatcagtgaatgaaagtaacaggaaagaattggtgaacttgaggatgaaacaataggaagtatccagtctaaatagcacagagaaaatagattgggaaaaaaaaaaaagtgaacagacactcagggactgttgggacagtcacaaaggatatgacattcatgtcatcagagttgcaggagaggagaaaaaatgtggggcagaaaaaatatttggtgaaataatggctgaaagcttgcaaaatttgtctgtaaggtcattaaacttatagactcaagaagctgaggaaaccacaaatatccaaagaataagttgtataaagtatgtaccttgtatgtaccgagtaaatcctggcagtaattcttgtatcaaatagtcatcggaagagaaaaggagacaaataaattgggatgtatatgcataaggaaagaagaaaggttcatggctgctactttcttgcttctgcagcttggtcctgagcagctacccatctctccctttgttctcagccagcactctagcaggtgctggaaactgggtttacagacccagtttcttcaatggctctggtactattcagactctccatttcttcttgtgttagtttttttgataagttgtgttttccaaggactccgttcatttcatctaaattgtaaaatgcattgctataaaatcatccataacattatcttgttattgtttataaattctatagattttgaaatgatgtgttctttttcattcttgataattttcattttctctccttttcttgatcagccgtggtgaaggaatgtcaatgtcatattctttgcaaagaatcaacttttgattttgttatacacttttctttagtgttgacttgttttatatttagtaactattcttttcttgattaatctcttcctcagtttcaggagtttaagttgctgttacttttctaactttttgacttgatagtttagtgatctcaatattcagttttctaatatatgcatttcaagctgtaaatttctccttaattagggctttaggtaccgttccaggttttgacttgttctatttttattatcaagtttaaaatattttctaatttctgttgtaatatcttctttgacccataaaagcattaatatattgcatacttttgtcagtttttaaaattatgttcttattgatttctgatttggttccactgtggtcagaaaatgtactgcatatgatttcattgttttgaaatttattgagtctctatggctcagtaaaaggtctattttggtaagtgttccgtaggcccctgaaaagagtagacattctgcagctgaagatttaatgaccttagtaggtcagttaggtaaggttaggtacattggcaccttgaatataatcaaaattttttatttgattatttttattcttctctagaataggcaacttcgtttcttctgatttggtttggttgtgtgtgtgtgtgtgtgtgtgtgtgtagcttttgtcatttctgtagtagttacaatcacaatcttcttttaaatgaaaaatggtttaaacagtactgaatatgtgggaatcaaaggggaaccatgcggcagacctttcttactgtgactccagagacagaggcgcctcccacttgggaatttttccgaaaggggcagccagcggccctccagcatccaaggtggttttactagtcctgcctggagccgaaaaataggccagatgacttcccccaatccctcagctgtaatgaatctaagagatcagaagtcaggcagatgtgtaaggtggggtttcatgtgaaatagtatccaatgctatgcgtaggaagtgtcagactccctctgaggacccaggggtatttggaggtggtgccagcagcacgttaggagtggaagggagaactggccgctgccttagcggaggagagtgtggtgaccggggcctgtttggatcatggttctgctgtccctcttgagacctaacacgcaggctcaggccccagagtctctcacaaaccacagggcaatctcacaaacaagcatgtctctcaccaaccggagggcattttaaagcagagctgctattggagggttggaagcatcactccacagagtggttttaaagcaccaggctccacgggttgcttaaggggcttggagcagaggctgaaggcagctctgtgatggcacactccctgcctgagctgtgagaaagaaaggcggccccatgcagggatgaaagagcagtgatgggggaagatctctgctacctgggctgttagcttgaaaccggtcaggacctctggaatcccttggcagttgttggagccacagcagtgctcagccagcctctgtgaaaccgtagctcacgaagggaatgttgaggctgtcccaggatccgccaacaaggctgctgcactgacctggttcagaggggatccgaacctctgtgcggcgggctccattcatagcgccccaggcagaaagctctctgtcttggtatcagaatgggaagggaaccttggagaccattcagttacttcatgctttattgctgagaaaactgagatgtgaagaggttaagttacttctctaaggtgttaaaagtacgttgaatataaccctttaagggacaccctagcacgttcttgggactgaaaggccagatcctgtaagaaaggcttttttttttttccttccagtgtctgtgacttgagtacacagacatttggattaggcaatggcaccccactccagtactcttgcctggaaaatcccatgggcggaggagcctgttgggccgcagtccatggggtcgctaagagttggacatgactgagctacttcactttcacttttcactttcctgcattggagaaggaaatagcaacccactccagtgttcttgcctggagaatcccagggacaggggagcctggcgggttgccgtctatggggtctcacagagtcggacacaactgaagtgacttagcagcagcagcagcagcagcctgggtatcattattcatttaattggaagaaaacacaggggtcatttgcctgtttaaatgggtcgtattccctgttggaaggcaaagagctttgggaaatgtcacattctctagccagccccgaaatcagaagcaaaagagtgatcttctctggccctgcatgtttctgacttgtttgacaagctctagtgagctaccagatgtggaaattcttttcagcctgggataatttatttatctgtttatgtattttaccaggaaagtatctattgagtatcttgatactcatttgcaaggagctcctgggggaacagtagcagtgtatagtgttactcgaatgtttgattccccccaccccccgccacgtccgtctgcttttatttcaaacctaaagagctgagaaataaaatgcaacttgttcctcctcccctcccccgcctttcagagaggccgtttaaaacatgtggcagaaaatgagccatgcagacttggctggctgtatgaacccgggaatttatttttcctttcgtactttgatcctcatttccaaccataaaaattattagtagcacttaagtgttgaacatttttcacctgagagatgtgaaacactcctggccaataacactggttataaagtcagtcgttcacgtggggctgagaattgactatgagcagccatgtactgttttgtgcacaaagtgctgatcccttcctctgctcctctgcatcccataggtggcagcctctgggtaaacagcttgggcgctaataaattatgagaatttatctgtgttattttgtccacaactccacaaggtgatggcaagtaaagaatcaggagaagatagcaaaatagtgtggaaggccccactttttttttttcttgaggacacgaaaccatcccgagtgggttttcctgctgtagaagcaccaagagggaaagcacctaagttagaacacgctgggtgagtgtgggcgcatgggggactccaacgtcgattgacgtggtggggacggagcttgcgtttgggttgacgtcggaagctgggttggttcacccacgtagggccaggtgatggacaggctaagaggtcagctgaaaagtcgaaggcagtaaagaggtgttgtaggtttgcaaagaggtatctggtccaggcagtgagatcacaggatgaattagaggtgacaggttgagacaggaaacgccagttagataatggtcgtggtagcttaatgttcatctgggttgaagaacgaagggagaagggaaagacgggcgccgccagggctcgaatttgattgctgatgagggagcttggctagttggagaattccagagctgagcctctagtatctaacagttacattttaaatagaatcagttaactttaaaggtctctatatattcatccagggaaagatggtatattctgggattctaagtaaactatcccactttttctcttctcactagaagaacagaaagatggaaacaacctatcatagacttcataataacctttctaaaaagctggtgggtaacttttaaagaggaagaaagtgtcactgtcctcctgtgtgcccccgctgagccgcctgcagcgtcaagaccgcagacaccgcgtcttgggctgggtctcacagacctgtggctgtggtccttccgacggaattaactgctgtccttgctagacttcagccctagagtcctaaatctgaaactgtgctcaagagttgttgggaatccaaggagggttttctttacggtacttggagccacctgtctcatcctgccgtgggccctacagcaggccccaccacaccctgtggtctcacctagggcaccaagaagctgggttcttacagcccgcacggatgaaaacaaggcctccctgaaggagagcccagatgcagataacagcacgcagtgggagcctgcgccacgggttcccatcccttggcacatggtctgacccagtggctgcctggaaggactgggggcccgggtgttccttctcctctccggatgatgggctgcagcggcagcgccccgtggagcccgtgcaggagtcggtccacacggctgctcaggcgtcccagcgagccaggcctgtccctgtctgggacatgggcaaaatcgaaccttgctagagcggttctggatcagcattcatttgtactgggtctacactaaagctttacaatagctagatccattttgccaaaactgagctttttttttggaatgtaattgctctacagtgttgtgatactttacagtgttgtgttagtttctgctataccacaacatgaaccagccatacacacacacacacacacacacacacaccctcctaagagcctccctcctacccaccccgtcccacccctctgggtcctcatggagcaagaagctgagctccctgtgctattcagcagcttcctgcagaaagctgagctttttgaaaacgagaagctgtctgggacttgcagtgacttctctatttagggtctgaagaagctggtgactggaagctgtctgtgagccttgtgtgttacatccgagtgggggacagttctccccggagtgcctttcttgggcctggccgccctgcaggtcaatagaatgcggactgttgaaaggacatctgtctgtccttttcctttccttttattcacgtgaagcgttgcctttatagagactgttgtctcgagacaagagaacggggtggaatggaaggacccactggtgagggagtgactcgttctgtgagggtccctgcccaccaggttcagtcaggagacccgaggaagactggggttggagggagtcgtcctccctagagcatgacctccttggggtcctcgccacatcgcttcaggctcctggtctccagcctgtaggagccctgaatcctctgttaggcctatgctgtctccagcctgcactgatttgcacccggactctcggggcctggagagaggctagttctcaagtgctgcgctgtgcacgagctgctgaaagcagaccccaagcagcaggcccgggcccacggaggtgctgagagaagctgccatgaggtttgacctcttggagctttcctaaagcacccagtccccatcggagagtaagtcaaacaaatattaagggaaggaaatcgaatatgtctgctgaactttgaaataacatctgatggtttaaaaaaaaaaaaaagcacccaattgggaattacaatagagtgacactttttccttccccactgagcagagtaaaacagcaggcccagaatttgtttgcctagccgttaatgaaacagtaaatggaggactcgggccccttatcccgaagaggagggcggtgctgcccactggcacgtccaaccaccgaagggcgtctcctcgcagtctcccatcccacagaggtgatttcgcggttgtccgtggagctctgtgccggtgagggaagagctcgctgacctgcccactccctgccctctgtgccccccgcgcgccccccccccaacccccacctcaccatcttttctgtccgcagcactggcaggcccacctggctctgcagttctctgcctcaaaaacgaatgcttgtaatgtggaatcgttattttaaagaggtgttggtccacttgatggtttaaagaatcctttccctccccctgttctaaaggagataactgtcatgatattccagccctgagaggatccatatgtttgcgggttattgttccaaaacctgcctgttgtttctttgggattgattacagaccttggctgccccaggaatccagagcttcagaatttggtttatgatttggggatagggggaatttgtcttgcatgggctgcttctctgccttgattttgatggagatgcttagaggaggtgtgtgggcaggacttggcatgggaccaggggaaatgaaacccaccacagctctctcaggagggcagtgcccgcagcctccgagcaggattctgtggtgtggttcctgagtgtgaagctacaccattcatcgccagttataaagctgcttgcaaagtcaggacttggaaacagtggttggtgggtgtcattgcattagacagagcaattcctggttttgaaagaatagaaagaaagaaaactttaaaggaatggaatactcccctaatcttaaacatgtcacacctgggagaaggaatctcaagaaagcaaggtggccagtgggtgtgatatatacaattcacttctcagatcagatttatttctcggtgctccagaattggtgtgtacggttcacgtacggttcacacacgtataatcgtgattataactgctactgtctgttggctgctttagagtaagagttggcagcctttaaaagtaagatttctaggatcccatctagacctttgggttgggctgtagatttgagaggtggggcagaggtcctgatcacatgtggacactacccctagagaaggaaggaaggggctaacttcttataaccaaggtcactaattacatccacgatatagatctgggctagactgacaaatttcccttgagcacggcccgaccacacaggcctgtccgcctgctttgctggtgtctgcctagagcttgacacatagcgggtgctcaggaaatacttattgaccgagtggacttaatgtgattggacattgaattttcttttatatagtttctgtgagtgaaattataaggtgtttttcaaagttaagtgattccatattttgtattcagggggagatatctggtcttctctgtgttaacctgctaaatagcaaagagtaaaactttgtactaattatttatctgattgagggaagtggagatctcagctgaactttgtaatttaataactttcttttcctttctccccttaaaattcatgaagctttttccttcttcccgctttaccacccccgacttaactcttttttttgttgttttttgtactttttcctgcatgcttaccttatttcatcttcagttttttaatgtatatgtcattagtttttctaaatagtaagctccttagtagcaagatatatgtgttatttttgtattcactctttgaagtgtaattcagtccctaaaaatagttgggggcagctgatttttgcccaaaggtgcaaatgcaatttggtggaagaaagacagctctgttacatgatgctggacaaactagtcatccgtaggtaagactttaaccttgacctaagcctcacatgaaattaaaagacgcttactcctcggaacgaaagttatgaccaacctagatagcatattcaaaagcagagacattactttgccaacaaaggtccatctagtcaaggctatggtttttccagtagtcatgtatggatgtgagagttggactatgaagaaagctaagcaccaaagaattgatgcctttgaactgtggtgttggagaagactccggagagtcccttggactgcaaggagatccaaccagtccattctaaaggagatcagtcctgggtgttctttggaagaaatgatgctaaagctgaaactccagtactttctttggctacctcatgcaaagagttgactcattggaaaagaccctgatgctgggagggattgggggcaggaggagaaggggatgatagaggatgagatggctggatggcatcattgactggatggacgtgagtctgagtgaactccggtagatggtgatggacagggaggcctggtgtgctgcgattcatggggtcgcaaagagtcggacatgattgagcgactgaactgaagcctcacaccttatagaaaaattaactggaagtgaatcatggacttaaatgtaaaacataaaattgtgtaacgtttgggaaaaaagttgagaaaaatctttaggatctaggactaaacaaagagaaacatgattcataaaaggaagagttggtaaattagacctcatcagattttaaaacttttgggctatgaaaggccttgtgaagaggattaaaagagaagctgcagattgagacaaaatatttgcaaacgatatatctaacaaaagactaagcatctaggatatataactctcaaaattcggtagaagaattcagtagattaacagtagaagacgaacagtccaggtggaaaatggacaaaggacaggagcagtgatttcactgaagaggatatgcagatagcaaataagcccatgaaaagaggtttggcatccttaaccattaaggaaatgcagattaaaatgagaattatgtaacacctgtcagaatggctaaaattttgttttaaatagtgactacagataaagatgcagagaaacctattgaagacgctgagaaactggatctctcgtacattactggtatgaatgtaaaatggtacagccactggaaaaataatttgttccacttcttataaaaccaagcatgcaactaccatacgactctgcagttgcactcttgggcatttatcccagaggcagtaaaatttatgcctacagagtgggcacaatgaatcttcataataacttcatttgtagtgccaaaaaatgaatcagcccaggtatctttcgtgaatgaatggtaaaccaactgtggtacacccacaccgtggcataccactcaccagtgagaaggcacaaaccgctgacatggtacatccacaccgtggcataccactcaccagtgaaaaggcacaaaccgctgacatggtacatccacaccgtggcataccactcaccagtgaaaaggcacaaaccgctgacatggtacatccacaccgtggcataccactcaccgatgaaaaggcacaaactgctgacatggtacacccacaccgtggcataccactcaccagtgaaaaggcacaaaccgctgacacacagccacttaatcccaaaagggcatatgctgtgtggttccactgatacacagccttcttgaaattatgaaattgtagagacagagaacagattagcaattgtcagagggaatctggatggggaggatctataaaaggtcaataggagggaccctcggtgatggcagtgctctgtatcctgactgtatcagtgtcaatatcatgtggttaagtcacactgtagttttgcaagatgttaccattgggggaaactagggaaagatccagagaaggcaatggcaccccactccagtactcttgcctggaaaatcccatgggcagaggagcctggtaggctgcagtccatggggtcgataagagtcagacacaactgagcgacttcacctccacttttcactttcatgcattggagaaggaaatggcaacccactccagtgttcttgcctggagaatcccagggacgggggagcctgatgggctgccgtctatggggtcacacagagtcagacacgactgaagcaacttagcagcagcagcagcagggaaagatcatacaggatctctctgcattatttcttacagctacttttgaatctacaattatctcaaagcaaaatgttgaattaaaaaatcgtgaaaactcagtaagtatccaagggttaaatgtgtaaatgaattaataaataggaacactcaaatttcttcatccctatactatgacttgactttgctgtgggattgcctttctagcacttcatagcaggattaatatctccaagatgggtggaataccatcttacaattcttgaatttttaaagaaaagctgttgagggcatcatctgttttgatggtttggactttattctagtttttgccttttgtagtaaactgtcccaaatcatttttggggatgtagatggggtcgaaataaataaggaatttaaaagagttcttcggcttcttgattctccaccaagatattacggagtttaggcacataaggaggggactatggtggattaaagaatcagttcagaaccacatttatatttcagagtcagccaggattatagtttgtggtcaatgccgtttccttcctttgcacaagaagaagacaacaagcctggccagttccaccgtgtcttctgtgacaggagagtgatctgagtccccagggcagtggcttgaagtcacgagagtttctcttgctggatggagctggtgtctttagtccccagtcagtcaaagcaagcctcaccaagatcaacttgaaattttgaaggctgggtaatggttatttacattagaaatagaagctggagtgtctgtttatacctcttgcaggatttaaagagaatagccatttggcttatgatcattgttctccttttagcttcattcagtaatcccctgggactagacttagaaacagtatgacccccactggagttgaggttatggaaataaaaatgatttcacgtttagtagatcttaaggagcgcaaaatttgaaaacccatgacccaacgctgacccgtgtcttcctcctcccatcccctcaaactcacatcacatctcccctgcctttctgtattctttctcaagagattcattgcctcatgaaaaatacgcattaagtttaatcttccgttcagctgctctttgcttgtccttccctcccggccccagagtctctgggtcccagggttccagtaggtgctcaggggtcctgcgtggcatctcactcacttgctctgcaggctcatgcgagttccttctctcagtgggtgagcctctggctttctctgctgaatggaaaccgcgtttgcgacgagcctcaggctatcagaagacagcgctgaggggagcagaaggtcttgttgttttccccgccacctctctctccctgttctgttccctccctgctcattccgcatgcatgtgggacccgtgaccatctctgcccccagctgagcacattgacactggtgtgacctcagacgccgtcacctggcttccttgacaagtgagggtctgagctggggaagaggaggtgctttttagaaccacgagagcaggcgaccacaagactttaagagcagaggccggacgggaagttaggcgggaccatgtggtgtgacctcgggaccgccgcctcctctccagccctcatttcccatctgtccagggaggggataccggaccaagtgacctctgccgtcctggtgacccgcgagttagtgtggacggggctccacagctcagactgctggagcgtgagtctctccttagcatccaccaaaggcctgtgggccctccgtccggggagagagtgactgttggagccccctcctcagtgtgtatgcatgagactcactcagtcacgtctgactctttgcaaccctgtgaaccgtgtgtgtgtgtgtgtgtgtgtgtgtgtatcactcagtcgtgtccgactctttgcgacgctatggactgtagcccaccaggctcctctgtccatgggattctccaggcaatgatactggagtgggttgctgtttcctcctctgcctctccaatgatgccctcctaacagtggtagtggtgaaaggtttactggcacacagtcctcttaccagctgtggccagccctggtctgtctccttagcttcgccactgccccaggcctgatggaagggcttgagaggaatggtgatcatgtaccccagcgccagagcactgcgtccagccagactggggtgaatagggctgctgccaaaacccagaagcctcatggtgctgctcttgctttggcttattttaagcagaaccccaccttgggttggaaaggtctctggggcagactgaagtgatctgtctaataacaggtaaagcttaatacacaacgcagcatgtacatgagtttgtcaaaacccatcagcattagggcactccagatgacagcttcagggctcaccggctctaggtgcccagcatgaacatttaatggtgttggtgatggacagggaggcctggcgtgctgtgattcatggggtcgcaaagagtcggacacaactgagcaactgaactgaaccgaataaccacccttgacgaaaagtaccatccgccattgtaattaaaagcatgaactgaaaacaaggccagtcagtccctccattccacttcatctcaggaaattccaaatataaaaggtgagctttgttcccagagggccgagagtgagtccaataaccagctcctctgtggaaaccagctagaaattggaaccccggcagcctccccctctcccatctggggagcacgcaagacaagcccgacgggaaggatggccactaactccgaggtgttcgcacgtcgggttcctcgcagcgctgccctgactcgcctgcgcacgtggccgcagggtgtgggctgggtcatagcgttctgttccgctttgctggtcagggactgacacaatcaaggggatgtgtgcaaggagaggaatgaatcctagccccgcttggggtaccttgggcccaccgtcccccgtttccctcaccacttcttagtccgtctcttggttcacgagtcccagtacacgtggcgttcttctcacgcagtggcagtatccctgtggagctctgcctttgaaagtgtgatgactgcagcggccacttcagagagtccctttccccctggagctaacccgtgagctcctcagcagacatcccagatgggtgccacctgttgttataccttccccacccccccacctcccgacccctgggctgtttcctgaatctgactgtggttagcacacaccctgagtttaaaatgcactcgcttagctggcttagaatatagtgcattatcctcttttcaggaacatttgttgggtacctgtatgtgtatggcattttaaaaacaaagttaaataatgcagaccagcaactgatcactgttgctgaattagtttccttggaaataggttagtggggaagaagatataagaatttaggcctgaaagtgcattagaggtcatttaatccaacccctcattttgcagagggcagaaagaaatcagggcaacttgaataggtctggggcacaactcatccatggcagaactgttcctaggtatacactaaccatgtcctgcggtgcgttttctataggcggtagtcagggctgcctcctttgccttttcaaaaaaggtagaacatcttagttttatcctctaggagtgggaagccacaaaagacctcaggaagcttacaaaagaaaaaatatatataagtaggtaactctgaaaacagacacatgagcacagctggcagtccagagaggagaggagatttccataatccaggtaagaagtggttgggaccaagattataaaaattagaacggaaggacataggatggcagtctgttgttaccatctttgttggaatagtgatattgtaagtaaaaagtgaaagtgaagtcgctcagtcgtgtctgactctttgcaaccccatggactgcagcctaccaggctcctctgtccatgggattttccaggcaagagtactggagtgggttgccattgccttctccaggagatctctccaacccagggattgaacccgggtctcccgcattgtaggcagacactttactgtctgagccaccagggaagccctctttgttggaagagcgatatggaataggcttcaaatgtaaggactgtttggatttccctagtggctcagatggtaaagaatctgtctgcagtgcacaagacccaggttcaatccctaggtcggaaagatcccctggagaaggaaatggcaacccactccagtattcttgcctggggaatcccatggacagtggagcctgaaagactgcagtctgtgagtcgcaaagagtaggacacgactgagcaacacacagacacagacacacacacacacacacacacgcatgcacgcacaaggactgttcagattactgccaagcccctgatcgaggtgtggggtttgttttaacagacatgcgcctctgtcttcaaagatgtgtgcaatttaaaatagatgacactttatacaaaagatgttgataggacaggcaggcagttgaatgatggctttgtgacttgaacatattaatagctctgggagcactattactccagctatcgggatgtaatgtgaagtttcaaatgcctgcactacaatatataaaggcatctgttagtaatgacaaaaggctgaccccagagcatttccccgaagtaaaaagaaaaaaagtggaattagggcgggaggtggggagcgatgagggaaagggggttgaagtcgggttcccgctctctggctgggaaaatgctttactgtatacctaatcgggcttcgtactacacccgggctctgggagagagggcccgtccccttccgttccgctccgttttgatcctgcagtcagctgcagagttatagatgaagtgacaaggatggtatcttgattgcatccagctgcctggagtaagtcaagggcagagaggccccgcctgaggagccgcgcttgccgaagcctccaaaggcgtctgggagccaggacaccgtggggctttcagagcggcgacattcagcgttctgtgacccagtgtcaccaaattaagtgtccccagagagaagcctggtaagtggcttgagctcacaggaagaggactgaaatcaaaacagccttcgaccctaaaactgagcctgtgttgttatcattgctgtcaactaggggcttgaaaagaagaaccccttgggaaggtgatttcaccttccttcacatggattagagagctgtgctgtgctgtgcttaatcgctcagtcgtgtccaactcttgcgaccccatggactgtagcctgccagcctcctctctccatggggattctccaggcaagaatgctggagtgggtagcctttcccttctccaggggatcttcccaacccaaggatcaaacccaggtctcctgaattgcaggctgattctttaccatccaagccaccagggaagcccaggaataccagagtgggtagcctgtcccttccccaggaggtcttcccaacccaggaattgaaccggggtctcctgcattgcagttggattctttaccaactgagctaccagaaagccccagattagagagttgactcaaatacatgtgggtggggggagatgggattcagcttctatccagcagaggtgccaactgaatggatgccccccaccctgccccttctcctctaccttgcctgttgctggtgctgagtgcagttgcttgtgctcgtagccctccagaaacctctttcatcccattgagacgaattgatgatggtggttgtgatgtggcttgaacaggcaggtggttagtgccaagcagagaaacctggctgtgcactttggtggggtctaccccagtctgcaggccagatagaagttaggacccacagcctgtgttgtggctccataatagtattatattaatctgctgaaatacaaaccccagagtagagagaacacagccgcagcattatacatctgaggaaatgtttgacactgattcatttgtctgcagctattatgatgacgcctgtggccgattagctcagttgattacaacatggtgttcatgaggccaaggctgtgggttggaggcctgttaggtgagctggttccacacaaaggaaacctctttccgccattccagaccacgttcaagatcccagtcagacatcccatgaaagcagatagacgatcacaaagactgggcaagagagtgtaatgactcagcaaatgtcccttacctgctcctggaaaagcgatttaaaacacgtgctttagcgatggtgagttgttactgtggtcttctcacacaaaggctatcacattattacgaacgatgactggtgatcagtactgttgcaggcctcctttctgaatgaagaagtgtactagctctggctgtgaagaaaacaggaaggggctttggtccccttctttcctgctcagctttttcctgctgaggttttgcctgtccgtctgtgccttcagcaggtgtgcgtggaaggctgtcatgtgctctgtacttggaatgccaccgtgaacctctgtcctcatggagtttaaactctgcaggaggaaaagagataataaaaaaaatagatgataaataaccagtgcagtttcatgaaatggaagtttgatgcagaagagcatggagtgaggggtctaatgaagaatggctggtcagggatggcgtttaccttggatgggagggtctggaaaggcctgtcagagtaggtgatagttaactagagacgtgactgatgggcaggaaccagccttttgaagagctaggggaacagcccacctgagaaatagcaggtgcagaggccttaaggtctagtcacggttgaaggacagggaagcccctgactcgtcgagaacgtggtaagagagaggctggagggaggaaggctgtttagaacatgcgtaggagttagcgtattattcttaagaggaagccatcttttgttttgttttatgggtggagcattttttaacgctcttgtcgaaggggatgttatggattgaacatgcctaaaaaaggtattcccttttttacctttcaccaaaagtcttccctggtggctcagacagtaaagcatctgcctccactgtgggagacccaggttcaatccctgggtggggaagatcctctggagaaaacagtggcaccccactccagtactcttgcctggaaaaccccatggacggaggagcctggtaggctgcagtccagggggtcgctaagagttggacatgactgagcaacttcacttcttcacttcaaaaagctgaaatcctaatccttagtaaccttactggaaatagggtcttttcagaggtaatcaagttaaagtgagattgttagggtgggccctaatccagtttatctggaatccttataaaaaggagaaattcggacacagaggcacacactcagagaagaagtccctgtgaagacagaggtttggagtggtgcgtctacaagccaaggaatgccggagattgacagccacccaccaggagccggggggaggcaggcaggatcctttccccgggtgagatggccccaccagcactttgacttctgatttctagatttctgttgttttaagccacccactttatgttttaaagcagccctgtgctcggttgctcagtcatgtctgactctttgcgaccccaaggactgtagcccaccaggctcctctgtccatggatttttccaggcaagaatactggagtgggttgccatttcctacttcaggggatcttcctgactcaggaatcaaacctgcatctcttgcgtctcctgcattggcaggtggattctttaccactgtaccacctgaaaggtgtgggcaggcggggggtcctagcatgctgactttgggaaagcagtgacgcatcctccctggtggggcaagcctctgggtgctgtagcatgcatgaccataagacttagatacatgtgggtagatagataatgaatatatatataaattaataggatggcacccgctggttgaattaccctgtaaagcataccctactcttcttttagaaatagtccttttggggatgctctgtaaatgatggtttggaagggcaggggtggaagcacacagattggaagcctgctgcagtaacccgggaatccacagtgacctggtaatgacggtggtttgagctggggacagtgtagcagaagcagtaggagggcgtcagaagtagagtcagcagcagctgctagaggactgggtgtggagagagaggaaagaaggaggaaaggctggcccagatagtttactgaggcaggaaatactgagaggatggcttttcccaggctgtggaatcaagagtcatgcttcggacatattaagtctgaggtgcctgttgttaatccagctgaaggtattaagtaagtagttggttgtatcagaaacattcagggccagacctgcatttagaggtcttcagagtctagatggtatttaaaaagactgaagctagatgttgcttgtggggagagtttaagagggtatagctctccgggactaggccctcaggaactccactgtgtagaggtctgggcgaggatgaggactccgtggtgctgagcagaggctccaggacagctggtaaattgtcagtattcactaatggttagtagtttaaaggctaaggaaattgtatggaaatacatggaggaaagtactggatgtaccccacccttgtataccaattctgtttaacaaaactatatctcagagcctttccaaatgtggaaataaaatttttgttttcagtaaaatcaggcaacatctctatgcctaaggcttccaaaagatcgtaaaaaatgtctttgtgctccagataggttttttttctttttcagaagcagagactttgtaagtttccacaatcatcaaaatttaccataaatcatggtttcatttatacttcatttgtgcgttcattaagagtggaacctatttcactaatcgcctgttgagagattacataccttggcgcaattcaagaaactgttagtctacattacttgaaatttccaaatacaaacccgaaagttttagaggaaattagaatcaacatttatctttgaatgttggcagaagaactagcacctgttggatcgaatttcttcagaagcccattctgctcttagtttgtgaagatttgaatgcagggattctgtcatttaaagagctgagcactgtacttgcttattgtctgaactggggattgattgctgatgtgtttctgtcctcaggtctggcgagaatttcctgaccggttggtgggttacccgggtcgcctgcatctctgggaccatgagatgaataagtggaagtatgagtctgagtggaccaatgaggtgtccatggtgctcacgggggcagccttttaccacaaggtaaggtggggacgagcctgaagcaagtggggccgagctgacgaacacgagagcgtgatactcgttttgtttcgccgaaactaaatttcgccaagtttagggaaaatgttccgttcagagagcttaggaaggccatgctgtgtgatttaaaaaaaaaaataaagttatttttctaattattaaagtattacatgctcaaatatggaacaaatttattgtataagaaaaatcacttaaagttctatcataggattgctgctgctactgctgctaagtcgcttcagtcgtgtccaactctgtgccaccccatggacagcagcccaccagactcccctgtccctgggattctccaggcaagaacactggagtgggttgccatttccttctccaatgcatgaaaggaaaagtgaaagttgccatttccttctccaatgcatgaaaggaaaagtgaaagtgaagtcgctcagtcgtgtccgactcttagcgaccccatggactgcagcctaccaggctcctctatccatgggattttccaggcaagagtactggagtggggtgccattgccttcttgatcatagaactaatggtagagaaactactgggttggccaaaaagtccattcatatttttctgtaaatcttatggaaaataatgaatgaactttttggccaacatagtattattatttaggtgttttttccaatttgtcttttttatttattgattcacacatacagtcataaatattctcattagcttttttgcatgtattatgagttttttccttatgtcattaacatttctttataaacaacctctacttaacataattgctaatataccattatattaatattccatagatcatatacagctgtttctctgttattggatatttcagttcagttcagtcgctcagttgtgtctgactctttgtgactccatgaatcacagcatgccaggcctccctgtccatcaccaactcccagatatttaggttatatctaatttttttttgctagataatgctttgtatcaaatccttaaataatgatcagtatccattttcataactgcctatacttatgatctttatgatagattcctagaagaattataaacattttggggaggctttgttgtatatatttgtaggttctcagtatcgtttgtgccagtttacattctcaccatactctttcttgcatgaaatacattttttaaaaaaatcaaacttagctattttgctaagtgaaaattgattttattgatttcactttgatcacgtggggtttgcgattccttttgtgtatttagccttttgtattccttctgagaattgttgctttatgtttttgactcttttttattaaagggagagattgaagggtatacattgggctttagaatattatgtttggtggtggagtgctccctgggagtgtgtggcttaattgaacagtaatgttcagttgtcagcagtggcataaggaattgatgtcaggagaaagatgggttcagcccagtgcagttaagttgacttcactgtcagttgtcagcagtggcataaggaattgatgtcaggagaaagatgggttcagcccagtgcagttaagttgacttcactgtatttatatcggctgcgtgttgtgatctcagcactgctgccctcatatttttcagggctctcgatccagaaatgagttttttttccttagcaaagcataaataaatatataaggtttataattcttggggctatgatagactgataaatgcctttttggcagatgtatttctgtattataccacaaatttctgtattatttcatacatgtaggttatggtagttttggaattgcctcatttttgttgtactttaacaaagaacatgtctgcaatgtcagtccaccatcaatatatctctgttgctatctgcagtccatgggaagaagataaaaaacggcggagcactgtgactcagagggaaggattcagtaaagctcagtatgtagtgtgtctctgaactatgtttattaaacacatctgttaatcagtccaaagggcctgcatcttacagggacttcatactgtaccgtatgttcctctgcttacaataggtgattttttaaagaatgacagtgttaaagacagttggtcacctgaccaaaagcattctaatacttcccttggcctttgtattgatccaacattgtgactcatcttatgagggaaagcttgtccccctgtcatggctcagttgctctttgtgtatcagccatagggaactgctatttgaatatgtctcctcttttgtctcacctggcagtattttaattacctgtatacctacaagatgcctggggacatcaagaactgggtggatgctcatatgaactgtgaagacattgccatgaatttcctggtggctaacgtcacagggaaagctgtcatcaaggtaggaggcatctatggccagctctggggcacatcttgggcacctttctgagtctccaaagtgttcatagctataaatgaaacttggaatagatgaactttcagctgccttttagctttaatattctatgatggatatttcacattgtgaaattgaagctcaacgtattttcttttgttatataaaattgctttagttatagttttttaagtaccaggatcagaaagtaagtctataatggttaatctacaaattgtgtttgtttcaaaaatgtaattttattctcaacactgatttggaatctagttcttgtaaagaatgcaattatgtctttcttaaggtagctgatttttttaaaataaatgttgaaaagatagtatactttgtccaattatactggtagttgtcctaaacactgttttaagacagtgattatcaatttttaaaagaacctttcccatagcatattaaaagcagagagtatgtaaagatcatgtggggatctttgtttctataatagatgtctattcctaaaatgtaggtcttgcccaaatcagggccctataaatgtagaaaaagtatacttctttatatcagtggagtcatcttgacctttttttctttctggattagattccacatgtaaaaatggccaatgcttctaggccagaatcctatactggaagcttatattggaaacgtggtatgacattccagaatgattgctcatgctattaatattaatcgatgagagaagccagattaaagcagtgactgagaacagaggaaaagtcactgtccagacttgatccagtctttacactatccctcggctctctaaatagctcagtgaacagctcactgcagggacaattaaagaatgtaaattgcaagacgagttttaaaagaactcctcttatctccaaattcattttttgacattcttcaagacattctttagctgtgcctttactctggacagtaattcctcgagtctgtatacttcttagccgagtctcagcttagccgggtcttagctttcttccccacccaccctctactgtttacttatcttcgctgggttacgatagcagccttccctgagtttgctagaaaaatattcgaagtttgaggtgtggggaaggtggtggggagagtcagagaaaaggattcagagggcaaaggacccctggatttttttcctgacttaaaggggttgcaaaataacctgacatcttcaagagcatggtgccgcccgtagcttttgccattagctggaaggagaaagagcgcctgttggaactaaggttacggtgaaagttatgggaagctgtatttcatcgcccttatggctgcaagaacaaatggtgtttatacaaggaccttggcagtgagaaaacagtcattaaacaggaattaaggagcttgtcatcaccacttctttccagttacagaaggcaaaagccctccaagcctcttttattgggcccttgtgagttctgccttggccgagccagacagaattgaacggaggagcggtgaggggtgtgtgtgtgcgcgtggagagggagcaggcatctcagcttacagaacagaagcatgcagagtggcatcacaagcatggttttattgtccttagcactgaccgccgggtatatttttgtgctcctctggcaggtaacaccacgaaagaaattcaagtgtcctgaatgcacagccattgatgggctttcgctcgaccagacgcacatggtggagaggtgagtgggcctctagacaaaagtctgccctggcctctgatccccatttcctgctttgggcctgtttatgggactttgttgcagatataaggacagcagctggtagccatagtcacctccttttgcacatgggaattgggttagttcaagcccaggtcacccaaagaattaatttggaatgctattcactcaatatgtaatggctggaagggtcttaaaaatatagctggccttaagctccagaagccagattctccatgtggactaagcagttaaccatccacagtcactcaactggaagtgagctaattccaaggaaatcctgggttgtttttcagggtagtatctctgtgttacagtaaaagttcgattagagagaattattaaaccaaacccaccagaggccagattacctttgtggacacactaaaacacctaccaagaacagaattgaaagcacagctctttacatttcagagagcgaagcagggccgctctgaaatacctgctttatattgattcggtcacttagccaatgagcacccatttctgtggatgtaatacagggccttgtaggtgagcatgaaggaagtgggagatgtctgtctgccctcaagaccgtctcacttgtgtcccaaaggccccaagcttcaaatctcctgaatcaaacctaattcttccaaaaggcagcctcttacataagacagcgagctctggcaaccaatggcatttttgtcaagggaccagaatgcattcagagaatgttgtgatagctgtcagagtgagatatctagttcgggtctccctgggtctcatgcgtctgcgtgcagcactcatttctgaaccctaccagctgcatcagggtccttcaagcctggaggacgttgtacagggggctgtttcagttggcctggacccttctataatgctttactccttcttacgcattctctctaaataatctctttcatatgtaaatatcttacccaaatgacttgacttttaccaactgtcaatatgaaattatagacattatcgatattattatatatcacaatatatattatatatattatcacaatatatatataattatatatcacaataatattgatacaattatcggtattattgtgactttgaaatatctgtcactggtatcaagtaatcacctagtcaaggacagtgacattcctgacacagctcatttggaagagaccccaattatagttctgtggctgctgctgctaagtcgcttcagttgtgtccgactctatgcaaccccatagacagcagcccaccaggctcccccgtccctgggattctccaggcaagaacactggagtgggttgccatttcctcctccaatgcgtgaaagtgaagtcgctcagtcgtgtccgactcttcgcgaccccatggactgcagcctaccaggcttctctgtccatgggattttccaggcaagagtgctggagtggggtgccatgttagagcacatgaaattgttacttaaacctacccccattctgtgttgagaatttcatagcattaagcaattagtttgattgggctgagttacatggtggggttttatggtctaagaagaattgtatttagaaaatttcacccaatactggccttaaagttcatgctaaagctgaaaaactgagataacaaatgcaatgagaaagatttaagttaggtataattattatttaacactgaagtaagtaactaagggaagtgtggaagcttcctctcgggaaaagggttttgaaaatagactaggcactgcccagatgttggtaggcttgattttatcagatttttataattaacaacagtagataacacacatgtgtatgtgtgtatcgaacctaatccgaagctatatgtgataagtcaaaggaaggctcagaatgttgtaactggtcagaggaaagaaaagtgacttcttactaggttagtcattatgaatgattttgtaagaggtggcacttgaagtgagccttccaagattgctaggattccagctccaggcaggaagaggagatcctcggcaggggaaaccggatggtccaaggtgcatttggcaagcagtcaagatagccagttttgttagagcagaacatcctggcctgtcagtgccaaagggcttttttctctaggtaagccgtggaaggtttttgaatagggaagtagtttgattagagtattgttttaagattgatttggaaggagtacatagaatgaaataagggtgagattgaagaaagaagaacagttagaaggcttgaactaagttgagtgtggagaggaagggttgaagtaaattacacggttttttccttttttctcccaaaggagactgtgctagcatttgatgtccagctgaatggtgtggggaggtcttggaggagttgactgagataggaagaatggtagtacctttaacggtggaaataatatgattaggagggatgctaagtaggggaggggaggacatggatgttgtagctttcacagtgtaattaatgaggccaggagattttaatggtgtgtcaagttggagagcaataaggcaggtgcgtttgcgctcttaccctctgtatatgcagatatagatatagagcaagcagttggaaatatagaacgaggacttaagaaaaagattaaaacaaagatggagatttggaaggcatccgtccagagatgacaactgattgaagaaaagctatgggtagaatcttgagaaattcagacagttaggagacagagtaattgaaaagagagaagcaggcactagagagaggccaggagagaaccagggtctccagtgccagcagcattggagactttggaaaaagtaatgcgtagggtgaggggctgaagggcggtagcgtgggagctgacagtgaggacgcagggtcagtgacagcaggctcccctttggagaaatgcgatggagaacagaaggaaatcggtcttgaattaattggaaactgcgagcaaaactatttggattaaggttaaatgactgaattattaaataagacacttaggttgggtgggaggggggtatgatttaagccctactggataggccaagagatggagttacaactgcagaattgttcacttgtttcgaaatagttccctttgcggaatctgcatttttgcacaaaacggtgagaagagttaaccgctctgtacgttctgatcttgtgttgagttgtgtgcttcagtaagcttatttattcttatgtttggggtctgggatcagaaatctcagctgtggaggagagtactttaactgcccttcctccatttccaggtcctgctattagttaatttaagaaaaagaagattttgtaacttctttttgcccagtagctttacccacgtcgagacaatatggcagacaaacaatgccgatttaagaaacaaacgttgtattttgattcaggtcagtctttgttcaaattatgaattagccatttattagctgtctgacttaaggaaattgacttaccttatcctgaacttccaatcatttcatctctgaaaatagatatattaactgtcatattactgtatccattcaggaattcaacaaatatttattgagcatttttgtatcagtgctaggaaagtctgtaccctggggcctggcaaggttagttcacttcccagtttccccagcactgaccctcaagctccttgttactttaggtaatagtatgagtttgaaaggaaaacagaaagccattagtcttttctccccagcccttgcagcacttcagagatagacattggctgttcttagcctatctgttggttagaaaaccaagctaggggaacagttactgacatagtagctggacccccaagagaacaactcagatagacccaccagcaaccagcctgactcataatctaccctaacttgttccccaccccaggtcccctgcatgaaatgacggtaggagacactgtcggtacctggcagtcgaggacttgtaccattcagcatggcagtcgcttctccagggtgggctgcgtggagagaaaggaggactcagtagcctgggtcagccaggggcagctgtcagcaagccgagctggcctgagccccagcggcctctcgcctgtggcccggcccggccggccggtctagttacagaggagatgttgtcctcggtgctccccaggagcccgtagagccaccagcccctgccaggagccgtgagtcttctttcatccatcctcgctggcaccctatgctggacacggctgggctttgctggccggagtccacaagcagaatcactggaaactacaagaatctgtccgtgtttataggcagaacaactgtcaaagtgagaccttggggagatttgacttagtctgccgctgtcttgaccccgtaatttaaggtgagaagagctgttggccagtgcttgcttcctatcattttattaaaacctctctcagtcttttccccagtctcctgtcattttggaacctgagactgcgacctcctgggtctcgaaagactgccaacaggaggggagctcagtgatctgtgggccattgtagaatgtgatagaaagacccaggcctctcttagcaacgcaagcctgggtttgaacccagctccaccaccttccggctcagggtccttgggaaagtcccttagcctctctcagcccaaatctctcaaatcacacaaaccttcgggcttatggagggggatacttaaaggaatcactgggtcgtggatgtaaattagctggcacttaataggaaatggcaacccattccagtactcttgcctggagaattccaagttcagaggagccagaagcctgaagggctacagtccatgggattgcaaagagtcggatacgactgtgcgaccagctttcactttttcagtaggtgcttaacagttgttaatttccagcttctctctttggaccctagaataatgataataaataaatgtgtctcctctcatagtgaagacttcagcctcagcaaccagagtgcctggatttggctctgctctttccggttatttggctttggtcctgtctctgagtctccctgtggtgtgcctctgtctccccagtatgccgtgggaaggataggaggcctgcctcgtgagcttgttgtgaggggagacgtgagtgactagtgcacgccaagcacttttaacacagcccggaacatagcgtaaatggtaggtgccgctgctctgtggctgtgggcacagtgatgttattagctggaagagctcacagtgagatggtaacaccttctgagcagcagaataagtgcaagaaaagaattgacaataactggcttctttattaattggtttcttgctgaagctcaaaggactcaaataaaagctgtggactgaaatagcttgaagcaaggcttacagtctttccgaggacagaagagccttgcagtctgtgtcttgaaattcggcccttccccgggaaccggttctcaggtgccatgggcctgactgaggagagagaatactgacctcgctgaaatccatccttgaggtccccttaacttcaaaagcggtctttgcgggctcgttttccccctttggtctgatgccgaaatgtcctcatcacccgctgtcacactcagttggcctgattgaccagagccctttcctgcccgtgtgtctgagccgctcccctctgagggggctgctcacgaggtgggggtgaggggctgcaccctcccctcaccctgcggtgctaagttgcttgttgctgttgattctaacacgagaacagctacaattatgaaagctgtccagtgtgaacttcagctctcacagaggagcttatggaagtggactgataattcccagagattgccactcacagccctcgatagatgttggttgaatgcctgaagtgtttgaaattgaggttcacggcccccgttgtctcctctgccttccatcgccccacatgtaagagccttccttctcccttttaagctcaagcgagctgaagccaagatccaaccctttgcctcctccctttgccttctcttcagctggcagggatggggttaaggcttgttggcaactaatccacctaggttagaggcctgcttgtaaggagctggaggaatgactggaatttgagggggaggggaacgatactctgacctctgcaggcccatggccttcgaacagctcaaacagcatccctggttccctttctccctctgaactaatagagtacccccgatcactttgttcctccacagcctcccagccgtgggaagaacccgggagcatgctggggaaccttgcgtttctctggccagtgttgaatacgatatatttttgctctcagctctcagcctcttgaacgttttctttgttccccacccctgtccctctcatctgccccactgccctccccaattccccaggtccgagtgcatcaacaagtttgcttccgtctttgggacaatgcctcttaaggtggtggagcaccgagccgaccctgtcctatacaaggacgacttccctgagaaactgaagagcttccccaacatcggcagcttatgaagcaggccgctggtggaggtctcaacacgaatgccggacagaggaagagaactcggccccccagccctctgacccccggatttcagagtggaagactggcacctcctcgcctgaagagcagaggccccaggaggacatctgagcacctctggcatcctctgatgctctcaatgggttttctgaaaactctaggtggaagcctgtggcaggctccaggggaaggccagatcaggctttctttgtctccagctccagtacagtgatctgagaggaactgtccctggctctaagactgctgagaagcctccagcagatctgtgatatcatggaagagctaccggactcactttttgttatttcacatcagtgggttcttcagaggaagagccacacccagaatttggtgcacgatccaagcatcttagtggcgtttgatgccttgggagcaccagctgctgagttgggacccgtaaattccatgaaactctggtttggcttttggatatgattaaacttattttttattccgtttcatactacatcttaaatattgactgtggaactttgtgcgtatgtaacttgcatcttctgacctcaactccagcctccttcccaggtctgggaagacagtcggccgggaggcgtgtctggtgccatgtgtatccagcagagaagaacctccaactacaaatattcttagcgtccccagacctacaaaggagtctctgtgcttgctgattagatctagatatccttgggggaaagcagagggcctctcacagccatacgctgagtcgctctgctggtaccacattgtaaaattgagcgagttgtgaccctcgtcccaaggggatgccaaaatttccctcattcttttggtataaacctaacgttagccagggaggctctggctaatgttaaatgctgctataacaactgctttgcaatagttgccggtatatttaaatcgttacatttcagcatttagtaatactgcacatgtgtgaattatacctctttaagctcagttgatgaacaaatctactctggcaaatgttagatgttaaggattcgaaacagatttatctgactctaatattaagattagccacagtttgggctttagccataacatatgtccccagaacacaaaatacataacaatttgcttggaatatggatataattactgaaacttagttgtgtgcccggtccaagtcactaaaccaccactcattgttctgttgagtgacacggaggtgagctggtctcatgctggtgttttcagacttgcagttttaaaagaatcacttcaaatgtgttcccatggactttgagaggccaaagaaaattttcagaagtagcacaattgaaagtggaaatcttgagtctgatctttcatatttaggcctggtgggatggcaggctgttcttggcagaagggataagataacatctccaggtcattcattttgcagttgcagacactggagcagtgatttattcatgtgttgctgctgtgaacactggtgcttaatcaatacttttgatttgaataattatatcctagccagcaggggagatgggaaggcattttggcctcatagtactgggggtgatacggtagaaaccaagagaaaggggttttctcttgtcacccagataccagtagctgacacggcgagtaggaacctctgatctttccaggggttcccttgcttgttctccaaggtttggggcaagtggaaggggaggaggacctcacagttaaattctagctaaaatctaagaactgtgctgacattgtctactgtgtgacgtgcgttgacgcacaccctcggcatgtattagtagcgctctgctcctttcagatgaccagctgcactgcattagtggctgctttctaagccacctccccctctcaaaagggccacctttcctcctcctccttcctgccctccttctctccctctctattattcctttgattctggaagagaaatgcttcagtagcaggtcattgggggcccaaggggacagagccagtggacctttatcaccttctcagaagacattggtcttctaagggcattaaaactccatttcgagtacctaactacccccagaaatattgggggttagattttgcaccatagtgtacaattttcttttattattattatagaataaatattacatcctaggcactgaaagcaaacctcaaagcttctgtcctagggaaagagagagggtacaggtcacagggaaaaattcaggacaggagtatagcaagtgctctgattcatgggttcagaggggactaggagctccaccctgtcctgtgtgggtgtatttctgagctgctcaccagcaagcctcactcataagcttgggccaggatcatctgcccttgggatgccataggttctggtttcagaagcggaacaataatgctaattaaaatgtcatattctggcctacaacagacaacagctggttttagattaagaaatcttttttaaggaattgcatcgtactggcctgcaccgacgatgaacctgagctggccctggccttgccacacatgtatgtactaagtcgctagcctttgccctcagggacctaactgggatgggccgggctgcgtggaatgccagtgttgactggtgcgtgctcagttgtgtccgactccatgaccccatggactgtggctcactaggctcctctgtccgtgggattatccagtgggttgccatttccttctccaaatgccaaggttggaaacccttttttgctagttaagtttaggtagctatacatattcttatctgatgtattttagcaaggctcacacatgtttaggcaaaaccctccttttctgggaaaattaaggaggaaaatatccacttttgtcctcagactctctggttccttcctgcaagtgatgctatcctgtctcttcttactcggcatcgtgaaggtcagaactctggcttctctgctgaaccttctatgcttccagcttgtgggttttaggagtttctctgctgcctctggagagagtggccatcatcctggcctgtgctgaccaggaccgggagctggaagaggccaccagcctctgtgctctcacttgccaaagggtggctggtagctctgtgatccggcttcctcctgggacagtccctgaggatccttgccactgaagggccgcaggctcctggccacgaattctcaaggtgtgattgctggactcaagtccaagaccccttggtggtagaattaagaagtcaatgaagcagagtccgggtcaggaagtggttcactcctggggggactcttcctgactctacagtcacagaacccggccaagagggtggaggtctcgaaggttccccattgtctagcttccctgagcctggtgggtttcctctgctctcccagtggcggctccggggtaagctgttttctccagttctgttggtcatcttctgctgcagcagctgaagccgcagtacgccattccccgaggagccctggctcggggaggcagaggcagaaaacacccaagtggccccacgccacttcctgaggagccatcctggtggaagtgccaaccggattggcaggcagcggccgtcgagggaggctctggaagagacttgagctttccactgagggcccggaggcgtgtgaaccctggaggctggctgctcacctcaccccagagctctgtcccaagccccagggctcctgtgctaggccagtgtttgcttctaaatgaaacgggggcagcttttggccccactctgccagccaggcccggtgagatgatctgaaccggtttcactgagctgctcctgacaggacagtgaggcaggccagattctgctctcacttgtgaggttttcctctctcctaagctaaggggttccctggtggctcagtggtaaagaatccacctgcagtgaaggaggtccaggttcaatccttgggtcaggaagatcccctggagaagggaattccaacccactccagtattcttgcctggagaattgcatggacagaggagcctggcgagctacagtccgtggggtcgcagagtcggacatgacttagcaactaaacaacagtgacatcatcctaaacgaagctcacagcctctctcccacctccattaaatagactgtcagggctccccagcctcaggctttgtgatgtccagcctctctcccacctccgttaaatagactgtcagggctccccagcctcaggctttgtgatgtccaggttctctcccacctccattaaatagactgtcaggcctccccagcctcaggctttgtgatgtccaggttctctcccacctccattaaatagactgtcggagctccccagcctcagactttgtgatgtccaggttccctcccacctccattaaatagactctcaggcctccccagcctcaggctttgtgatgtccaggttctctcccacctccattaaatagactgtcggagctccccagcctcagactttgtgatgtccaggttccctcccacctccattaaatagactctcaggcctccccagcctcaggctttgtgatgtccaggttccctgggacagggtgtgggctgtctaggcaacattgtgctacagctttttcatgtccagctcaccactgcttccaacacaaggcaggtgacggggcagcttgccccctctcccagctttgcaggcatctttccttttcaagaaattgttgagtcacccacctctgtgtctggcatctcaaatttaacagcctttgtagggtagagttttttcctttcttctgctcccaactggttgggcccatggttacacttctttgcagcctccctgaagggcagagctgtcaggcggagacgaggaaagggaaggatgtctgggtgccactcgagaggtactaggcagctatgtctgataagaggttttcccagcaaagcacctgctcacccacagcatcttgggagctgcctgtggagacagctgccaacggagctgggactggaatctgcgtgtcttggacccaagcctccagccactccatccacccttcagagccttcccttcacagagtatcaatgtgagctcttatttatagctgccaacttctatcattaactactatgcccaaagaatgtctggttttctggtaatcacagctgaagaacaaccaactcgttactgaccaagatgagggaataattcagtcattcggttttctaattgttcattcactcattaaaatgtactctttgctagaacctgtgctgcgtctgagacagagtggaataaaacacagtcttgacctccaggaacttgggttcccgtgagagttcttggctctgtaagatggtgtgagtccctcagaggcctagggctccacacacgtacgtttgagtcctgactgcgcaggaagagtccattacgtgaacaccaccttcgctggtgcccagaggacgcaagcattgtttgtttcccgttctcatagcgccttcctttgagttgagccctgggccttcctcggccttgagggattccactcccttgccttactttcctcaaccagaaattgaagatggcaagagtgtttcgacccgggtggctgagaggcagtgatgtatcagatctctcccctctctacccagagccctggccagattgtacctggtggtgctggcagccggccctcttccggcccctatttctgctcaccctgttaccagagagcctgggggtctggatcctatccggccccgtcagggtagattaccagatgagtggctcttttgccccagtgcctttcctgtgctataaataagccccgtgtttattttctaatgttattgaaatgagcacttgggattggggcctcttgactagtccggagagcgtccacccggtgcctggtgagggccctgtgtggctggctgctgtctgaagctatttggagtcctccccctgtgttgtggatgtggcttcatttcaatagtaagggctgtatgcagccctgtatctgctgattttcaggtttcagctttctgccagcctcactgcctgcttagaagtaaagctgtgtttctcattaaggggataacagccacaattgagataattaacgaaaattgtatattggtggcagcaggtcctataggatttccaatagtctacctagtagatcctgaggggctttaccttcatctcctcccttctgcctaccctgtgcccaatctctgttcttgttttctgggtatagtcccagtaatttctctccatacagccttgttcttgtcagcagtttggtttgatgtactgacaacttgacccaatgaagatttcgtcaactgcctcttctccaaggcacagaagatgcaaggatttccccccaccaccaccaccttcgcaaagtatctcccatttgctggttgtaccttggatataagatgaatatccattaataacagatagacgttccctttagtactatggctgcaccatctcagtgggacagtgctgagacagaatggaaacgtaacagcccagtgcaatgtcttatatgctatgtggttgtctaggaactaggaagtgttccgaattaggatttcctcaggagtggtttcggagaaggcaatggcaccccactccagtactcttgcctggagaatcccaggggcaggggagcctggtgggctgccgtctgtggggtcgcacagagtcgaacacgactgaagcgacttagcagcagcaggagtggtttctagctccatcaggctttgatgatgcatcagacatatccagtgagactgagcaggacacagcattgtctatcagaacttgagctaaagatcgttatctactgctcaggagggattattctgataagtcactcggtagagaaatgtttgcatgaacaaaccagaacctatagcctattcatgagcagttacatctaacaaccagatttaattcactttcagacatggaaccagaagaaagtgaatttttactcatgtgtttttagtctattgactcctaaagatccttttctcttaaaaagtaaaaagtcttttgtgatggattcatatttattgagcgcttattatgtgccagatactgttcacaattatgaagacgctgcattgaacaaagtgcctgctcctgctaagtgtacattctaagaaggaagacaaccaaaaaaagtaaatgaatacaatttcaaatggtaaaaaaatgctatgaaaagaacaaagcagaataaggggagatggttagtgacttgagcatgctgctgctgctgctgctaagtcgcttcagtcgtgtctgactctgtgcaactccatagacggcagcccaccaggctccaccgtccctgggatcctccaggcaagaacactggagtgggttgccatttccttctccaatgcgtggaagtgaaaagtgaaagtgaagtcgctcagtcgtgtccgactagtagtgaccctgtggactgcagcctaccaggctcctccgtccatgagattttctaggcaagagtactggagtggcttggcattgccttctccatgaggagatatttaagatgagttggtcagaagtggcatttaaacacagatctcagtgttgaggagtggagacagggggatgagctagtataaagtccctcagataggaaaaacctcagcttgttttaagaacagcaggaacccctgtgtggctgcagtgtattgggtatggagcgcagggtagagatgatgccaggaaaagtaagttaaggccaaattgtgttgggatcatgcaggacagaagggtcctggatttttattgtgagtgagatgggaagacactggaaggttttaagcaggggattggcatccgaatgggatttttgtttaaagagtcatttcagctgttatgtgaagaacaagaatggaagaaaggaaatagagaggctgcgttacagagagagatggcagaggcttaactagactaggatggtagcaacagagaaagtgaggagttccattgggaacagaatctgttggacttactcacgatttggttataggagatgaaaataggattcacagctaagtactaggtttttgttgtaaccgagtgacacagttcagtgaagtagagccgactgggcagggtaaggacagagaaatgtgagaggggatatcaagagctccactttggatgggttaagtttgagatgtcttttggtcatctagtggtactgctatttaagcagttagatatctagagcttaaggggaaagttaaaactgaatatataggtgggatgtaaaaccacaggatagatgagactacccagagagtcagaatatgtagagacaaagaggactgtggcctcatttacggggagagaagaaaagaggaaccagaaaaggagattgaggcattgccagtgagttaggaagaaaaccaggaagagatctgcatgacagactgtgatgaattagagatacacattgtaaaccctgaagcaatggctaaaaacagtttaaaaggaagcacagctaataagacaatattggagatagagtatgataaaaaatattcagtcagaaagaatggattgaagaaaaaagtaaactaagaatagacgggacacaaaaaagtaggagactagacttaaactctaacatgttaataatgacattaaatgtaaatgatctaaatgttataattaaaggaaagagactttcattctgaattaaacatcaagggcaatcatatgctatctctgagtaaagcactataaaatacagttgaagtaaaagagtagaagaaattatactagacaaatatgaatcacaaggaactggaagtgattttatcagtaaaggacaaaataaacttcaacacaggaacatttccagggagaaagaggaataattcataatgttaaaggtgtcagttcattaagaagacataacaggtaattctaagtaaggtaggcactaaataacaacacttaaaaatatatacagcaaaattgacaactgaaaataaaatagaaaaatccacagttatatttggatgtctcagcactcccctcttagtaattgatagaacaagtggatggaaaaatcagaaagagagagatgactggataatgctgtcaaccgctttggcctagttgacagctctggaacacttcacccaccaataactaagcagatattcttctcaggtacacagacaatattcagcacctgtgtgtggcaccataaaaagtctccgttttagaggaaatgaaaatacaagtatgttctctgatcacaatggaattaaatcaataactgatatctgggggaaatccctaatagaaattaaataacatatttctaaatgcatgggttaataaagacacaaagcaaattagaatgtaactttaggtgaatgaaaatgtgtctaaatgtgtcaggtggagtgctcagagatctgttcttgaagggctagtgttagaaaagcagaaaaaactaaaactaattatgtaagctgctactataaaaaaattgaaaacagcaaattaaatacaaagtaagtggaaggaagaataaagacaagcttggaaataaacagaagatggacaaacaatggagaagaatcaactaaacaaaacctggttctttgaaaagaccaacaaatactttagactgttccagggggaaaaaaaagagtatataaatttaccagtatcataaatcaaacggggagctttggtacagattctaaaaatattagaagtgaaatacatgagaaaactttatactgataaaatctacaacatagatgaagtagttgcaaactttgaaagacacaaagtgataaaaactacctcaagaagaaatagaaaaatctaaatagccatatccactaaagcagtcaaatttgtaatttaaaacttcacacaaagaaaaacctaagtccacatagtctactgacatatcaaataattaagaaacgacaccaatcttacacaaactcttttcagaaaatagaagacaaagagatgggtcccagctcattttatgtggccattattaccctgttatcaaaaccagacaaaaatcttacaagaaaagaaaactgcagacttaaagccctgctgagcatagacacaaaggtctttttaaaaattagcaagccaaatcaacatacaaaaaagataatacatgactaaagaatatggaaggaaagttatgaccaacctagatagtatgtatattcaaaagcagagacattactttgccaacaaaggtccgtctagtcaaggctatggtttttcctgtggtcatgtatggatgtgagggttggactgtgaagaaggctgagtgccaaagaattggtgcttttgaactgtggtgttggagaagactcttgagagtcccttggactgcaaggagatccaaccagtccattctgaaggacatcagccctgggatttctttggaaggaatgatgctgaagctgaaactccagtactttggccacctcatgcgaagagttgactcattggaaaagactctgatgctgggagggattgggggcaggaggagaaggggacgacagaggatgagatggctggatggcatcactgactcaatggacgtgagtctcagtgaactccgggagttggtgatggacagggaggcctggcatgctgcgattcatggggtcgcaaagagtcggacacgactgagtgactgatctgatctgatctgatctgaaagaatatgaagtttgaaaatgtattttatatttgaaaatgtaattcacaatagcaatagaataaaggagaatagtcatatgattatctcagtagtttttaaaaaaaaaaaaaaaaacagaaaacatacatccacacgaagacttgtatacagatgctctcggcaggtttgttcatgatagccaaaacctggaaaaaactcaaaggttaaacagcagatgagtgcatagataaattgtggtatatccatacaatggaatacatctaggcaataaagataataaactactgagacacacacaaggatgtgtttccaaaataggtgagcaaataagccggacgtgaaagagtgcatacttcacatacatagcttctatacatacagggattccctggtggtccagggaaaggtgaggttcaccctcaactgttcagtcttacgcagtagacaggacccttaatggcttctctcctgtattagtttgcctgggatgaggctcaagggaacttttgggagtgaggagaaggttttatattttgatcctggtgatggttacatatatatatatatatatatatatatatatatatatatatttgtctaaactcatccaactctacatgtaaagttggtgcgttcttatctaagtaatacctcaataaagatgattattttttcagtgatagttaaaaactgtcatctttcacagttcctttgctttgtcatggaccagcatcccttttccctcctttacctggaggtttgcttagggcctcagggtccaagctgcctccagttctgtttagtgttttctatcgtgttttgacatggaatgcagatttgccagccatccagctgatatttatcaagtgcttgctgtaatactctaccaaatattgcagataaacaatgaagccgtttctgccttataatcaaactaacacaggagataagtccctaatagccctgtctactctgtaagactgaacagtggagggtgaatgcacgcattccctgctgtggaaggagcatgtgatcgcttctgacttggtgaaggggtgagacttgaggtgtactttggagaattcattggtagagacaggcactgtgtggataggggtgtgctgggaggttgggcagtcactttgacctgaggggggtgaaaggaaacaagaagatgaggaagtgtactgataccctagatttgtaggagcactaactcgaggttgggactttacaaattatagagctggataggggtttagggctagacaaaagagctaaaatgtttgtgggcagtgtgaaggctttgcagatgattgttctggttgggagcgtgatcagagctagtcttaggcactcccactttggcatcagcacagatgagcgggtggagcagagagggaccaggaatgggacgtgaccacttagcagtagcctaggtgataagtgacaaggaaccgagcccagtggctgctggactcatgtgggagcttgctggacctgttgagatgctttgggatatgaagttgagaagagcagtcacttcacccatttaacacataccagccagtcttcttgttacagactaaattatatcctccgtaaaactcatatatagaagccccaacccccagtgtgacgggatgaggagatagggcctatgtagaggtagtcaagtttaagtgagatcataagggtgaggtcttaattcattaggactgatatcctcagaagaaaaggaagaaatagcagagctctgtctctgcatgagcaaagaccacgtaaggacacagtgggaaggtggccgtctacaagctagggagagaggcctcaccagaaaccagatctgcagacacactggtcggagcttctagcttcctgcagctgtgagacaataaatatctgtaatttaagccacccagtgtgtgctcttctgttacaacagcccagacagaccaatgcactgctcaccaccagattcctcaccacccgccttcccaattcccataaatagaatattgagggttcctggtgctttatattctgatgacatacttttgtgaaacagtaccccaatctctggcttccttctcttagccgttcgggtacaagtcaaccctgctggcccttcccaaccgtcagcgtgtgtaattcctcctgtctctacagctcttaattatttccaagtattttaactcgtttggatttcataaccacttgtctcacttcctatgacataagcaggcaagagttggtttgaccccattttacagatgaagaaattgaaaatcgggattttcgtggcttgtctaagataaccaggaggagttgcaaagtcagcccattaaatccagctctctcacttcaccacacagcttcctggcctgtgcttttctatgactttgtctggagagagctttctgttcaaacctgtcttggagtggagatgtgggctgcctttgggcttccttcttcccttccttcctttgtcctcccagcatgcacacacacacacacacacacacataaacacacacatttattttcgttctctctccatagtgtagcttggccttaatgtcataggaacaaggagctcctccttttcccgcagagcctaaacatgacagatcccctgtgggtagaggacaaagaagggatgggcaaggggcttacgatgcaaccaaaagagaaacctcggaaggcgtagcaagaacactgtagcccctgggctgtctgaaccgcatcctggccagggctctggggggcatctcctgcgggccccacagtgggtttgctcccagctaacagttaaggcagcaccttaacacatggtatgcacatggtcataaaccgaagctcctttcggaagtttgcccttcctcaaggctgttggtctgtgttgggctaaaaaaagaaaatctgtttggaatcacagaattcctgggaaaatgtcaactctcacaccccattgtctttggagctggcatgcatcttctcacttctgagccctgttttgaggggcggggctgtttgcggagggcctattgttcctgagcagctgagcatgcaagtctgggcctgtgtgtgtgtgtggagaaagaccaccactaccgtatcaccccccaccagccgaatcaaggatttctgctgaatcacagagcagcccgcttgtggcgccctgaggttggcaactgttcttgtagcaagacctgggacaatgggattggtgagaaagattcactggtggttgaggcagagcagaggtggggaaggaaggtttagagcatgagggttgtcgagggatgacatggggaaaagcaggctttgagcccaaggataaatgggaaaacctctgagatgagcactctcagattacaacacctagccatctcgaggagctggcctcatgccacccaaaccccaagaacactaaagaggttaggcttcctgtcctgaccttggctcattggggatgcatatgacccctgaaggaacaggatggacagagtccacagacaagcggaggctgtgagttcacaccaggctcatctctagccttcctcttcctatcctgaaccaaaaacagagcctctccgcattcctaccttcccattcattctcccagtgaattgcagttactcagtgtactaattttctgaaggattcattcagaaacagaattggcctgtcttttcttggcacttggaaggcagctggggcagtcagacagatacataagatactattataaacagttctgtgggagcatggagccgcaaccactggcctgggggtggaagttgggacggggcatagagttaaggaagaacttcacgtagcacattcttattaggaggaagccacagaggatgaccaggagtcctcctggtagagtgggctggtgacagggcgcaggcattctaaggaaacagatggcaagtctaccactgtagccaagtgaaaggccccagatggtttggggaagcaatgagtggtcctggcacccagacagggaagggctgagccacagctgatgttcaaggaagccctgctgacaggggagtgtcctccccagcgatgctccttggaggcacacatcccaggaatgggttacttgagtccccagggaggaccagatgacccggcagccatcctgccctttggtgggagcactcatctggactccagatactgttggggttgctaagcaaggatttcaggcaggcggcttcccatgcacaagaaatttctccctctgcttccattcccaccttgggacttggcagcgacttggtgtggagagtccaagagctgccatgaccctgtcagatggcaagggtcacttttaaaaacaagttcctagttaaagtcaacctaaagccatctgggtctatagtgaatgaaaacttaccctagaggtacggttgcagaaaaggggaccccttccagggcccaagagtgggctcttgtctaacactcggaaatgaactgtccaaggagacacatgtgctaccaaagcaagagacttgggacggggcacccaggtggagaatggcagggtaagggaacccaggagaacggctctgccatgcagctcagtctcgggttttatggtgacggggttagtttccaggttgtctttggccaatcgttctgactcaggtccttcctggtggcacctgcattgctcagccaagctggatgccagcgagaaggactctgggcggtggcgggacaggtggcagctccttttggcctttccagaactcttgcagttggtgatggcttgttagttccatgttccttactaggacctcctgtcgtaacgtaactctcacagatgattcctagccaggatgggtggtttcagtcagagtgcttcccctaacggaaccaaatctgagatgtggctccaggaaagcagcatttacatagtcagagaagatgaaaaagagtttcatgtcggactcagagctggtagccggagggtgatgtctttctcccgggaataatcatagcccttcatgtattgccacatcagcttacaaagcccaggcataggccatctcacgcatacctccccatgaactgggaaactgggcagagacaggcttcttcccctccttcagatgtgagagttgatggtcaggcaaacaaggatgcctccccttcagcttacataggggtccctgggagactaactgcttgagtcacctttaggctggaaagcaaggaagcacctcttgcatagcaaaatccaggcctccagcttggctttataattagtatattagtgctttgctttaaaaaatccccaggccttacctcctgcaagctgtctcagccactaagcgttagagctgggacagagacctgcctctcctggaagcctatcctgcaccatttgtcccaagcagccaggtctccctaccctgacctccaggccctagctggcccctcgctttcagaacagaccccggaaacccagatggtaccagtgatatcgaggggccgtgatgagcgcgctggagacgcaggggcggcccctgatttccctccactgcttgtctgccgtggaggcaggtcctttgggatgactggggggggggtgctccagcccctcgcaggccatgcgcccctctttgtgcaggagccctggccggccacctgttgttaatctagccctggtaatccattctgtactgtgtcagcagttcaaaagggcattgttagtattttttgccgacttcaattaacgtgagatttcagaggcccctctgatcacatttcatctgtcacaagttaggaacaaaacagacaggttccagttgaggggaggaaggagaaggagtttattgcaaacaacaaccaagcggacagtgtgggccgtgcccagacaagggcgctctgcgaacgcgcagcggcgcccacgctccacgcggccccacgcctggaccccgacgccccctcctcatcaacagtccagcagtcctcccctccccccaaagatgtacgtgcaataacttactttaaaaggcaagaacttttttttttaatattttgctataatgtagttacatggtggtataggcagtaaaactttatggaaccgacttcctttttttatacattttttttctgaatttttaatgtctttttcatatatacttttaatattccaccccaggccattaagctaaaggaaaagttgcatttatacagggttacaatatcttacaaggagaacagtcattattgattgaggttcatgttccttccagcactcagctctcttttcaacccactgcacaccaaacagactattagacattgaaaactgtccttcaaaatcagtagtataaaggcctagctctgtgtgtgtaagtgcaggggaagaagggacagggcagggcaggttaatgtttggttgactgggacacctttcctcccccccagcatctttacagacatcacgtgggctcccgggaacttgaggagtggagttttccttgtcttcccagttttctcttttgtggtcatggattattatcctgggggcccgtttgcccagccatcctcagtccccaccaggccgtcaagcagaccggaagccccagaactgtccagcccagggggagaaaagccgcagaagcaggattcatggtcgtttgcatggaggagttcctctcgtcgacccagccgaaagacggcagaggggacactggcgctgggcccgatgtggccgcggccacgtgtactagggacccacctcacgcttgctaccgccaccccctggcagtgtctttgaaccaacctggccgccatgcttccccacgctgggggcatggtagtgagagccgagaatcaagtatagtggaaagaattcaaggaaagttccaaagcccgtgtcctccaagtgccgtcgaagactcggggacaagcctctccgtgcgtccccggagccccctctccccacgctgccctgggagcagcgacggcatctgagggtggtagcagcaccgtagtgtcaagccttggccgccgagactggttcccagcaggattcggggggctggcagcgtgaggaggctgcccgggggcctgccagccacccattatttccggttttgtgtggccatctctgttgagattttaggcaaagagcaaaggaaaccccacctcttttagcttattggcttaacagtgaccaccggagccatccactctcccctgtacctgccttcatgagaccgcgctcctgaggatgccgccagcccagagagtcacacccaagcttgctttagtcatcacaggagaggctgcgggatacctcccaccccagcagctgagaaggcagaaggcctaggcccagccccccaccctggggagctccacgcagcatcagtcatcagagcccctctcttagctctgcctcaagtgaaatgtggctcaggatgatgaccttccccactggggactggggtcatctccgttgtgaagggattgggcaggctagaagttttctgatcccttcccgctcagcgattctgtgattgttggagtgtcaggcaggccctagggtctgggacaggccaagggcaggcctaggggcaggcagctgcccgaagggagatgcagaggtgtttcaggccgacattttaatcctaaatacttagctgcgttggtgggacgggcggtcagcgccaaagagccttcttctatcggaagatttttcttctcaaggtttggggtggaatttgcctgactgaggtctggcccaggtggaaagcagacacacagcggaactgatcgccagaagtcccttccagaaggttccacttaagagccgtgtctctcccttccctccccctcggtgtctatcctggttctctctctctgtctgattctcacccaggtgagaacatacctcccaggtctttccagccatgatggcatttaaggaggaactggggggactgtctccatcctgaagccatttcttccttccttttcctagattttgggagtcttgagttgtttcagccaagcttagaactagaaaagccttcctaagagttattcccagatcctcaaacatagaaaacacttaaagctaagtctccaagaacattcaattcagtgtccaagatgcttttcactttgagaagatcaactttagagctacgattcccaaagaggtctgactcttactagggggaggttgaggccagctccttccatagtcctgtttctggagcacatgaggacttgagacacagctggagatggccacgtccccaaaaacctaagcaggtcaaagggggagggtctcacccctccccacccacccaagaggctgtgctgagtccagtcccctgggcctggtagacctagctctcctcactgacaggattggggtagaagggataaaaagcagacccttctcctcggggatacctgcaagggggcctcccagcgctcaaaaggccggccttggaggatggctaccccatgccccgagcttagctgacccctgggccctggagctgaaactcaagctgcaggctgcatgtcagacggaaatgcagcccccctgagaaggaggccccttcctgcaggcagggagattggcttccagaaagcactgtgtccagaacagagcaagcgaccccaaggagctcagccccacatgagccctctggaggccagaacaagggaccggggtcacccatgccacagggcagaattgccagggcaggcgctctctgccctacgcccagaggactgcacagaaggctctgggcaggtgtgatgcaggaggccgctaaccgggaaggctgtctgaggccgcgtgggggccgacctggtgcccggaggccgggtgagtgtgccctcgtcgtctgccagaccccacaacccgctctggaccctctggcaccgtgcccccaggccctagcggctgtgtcaaggtgcgagtccttctgcatcaggacactggcccaacagctccagcaagtgaccaggatcttcagattccacctggaggggccagactggacagggtacaaagctacccagacctgccccctgccaccatcatcctgcctgaggtggggcgtcaaggtggccggagagccgacgtgtgtgcacgagcccctgcctccccaggacgcctccctgcagccctctcggctcctagaagcgtgcttccagcaccgtggggccgtgggcagcgggtctccttccctgcactgttcacccagatccggagcctcagggtccaagggggccagtgtggctgggtgacggcgggctccttcggctccagctcctctctctctgccctgtccctccgggtgcgtccagcacaggggacagtcagcaggacgggggctcctcacgtccagggtaaaggcgaggtgggtgccttggatctaggaggttggagtccatgggctttgagggagagcacaggagatgggatggcagaggctgactgggcagggcatctgttctccagacacggtcaccgggctggtgatcggggaccacacagcagagggaaggacggactttgacggaaccgtagccacagctggtccaggtgggaatctagcctcaactcaatggtcaaaccgggcagggcggaggagcaggagtggggagaaagggtcttcagcttataatggatgcaaagctgcaaaactcgtgccacctggctgcctccactgcctctggccgggagcgggggcacctggcctcggtgcccgctgcccccttccctccccacagtccccgggggcctcagacttggggcggctgaaagtgctgagggtcaggctcctgggcccaggccaggtgcctaagaggggagttgagtggggggcccgggggcctggggagcatgggcatggccagtgccacagggcaggggagggcgcagggatggggcggggtggccctgtcatgtggcccaggagatggccgcgctgtgctccttggccttcatgcggagggccgcgatgctcgaagtcttgcggtccggctccccgttgagctcatagccgttgaggcccgggctgaggcccgcagctccaaacaggctgcccatgtgcgtctggcccacgtggctgccagcccccgagacgctcaggaagtcggtgacgccgctggccccggagccgggggggtgggcgtgaggggacatgcaggcgggcaccgggtcacagggcaccacacaggccggcacgggcgaggcggccccattgttgccgatccaggacgggttctgaatctggggagaggggagggagacgcgtcacaggctggctggacacaggggtgctttcctgtcccacacgggggacctctcttcacttacaaacccatcctgatgataaaaccattcctgagtgtttacctgggaggctttccaaacctgcctggccacctagccccctaggagcctcggggttttatgctctggttcctccgttcccaggtccaggagagcagtggacagaaactccgttttaaggactgaactccgaattccatcaagagtccagtgaaagggtcgtcctggggcacagctcaccagctgagggcagcccctgattctgtttatttatttgaccacaccacttgcagatcttagttccctgaccagggatcgaacctgtgccccctgcggtcagagtcctcttcattggaccgccagggaattcctggcagccccggattttagacacagaggcagactctgtctctccatccatggacccatttagggtcagcacctgggcctcaccctgctccggctcctccccaggcccagcacgccctcgtgacttgatccatgggaggggacacgagctcagagagctctggcatggcaacgtgggctccgaggctgtccactctgagagtggacgtcgggggggcctcaattctctcagaagccaaggtcagtagtggaggtcctaggggccggtgcccttgaggcttcagaagtaactcagggtctgaaagggctgccttgagaggtggtaagtggacagatagagccaggcgggatgcccacaggctctaggctggggcagagacaggtggaggaggtggaaggtgctcgggcagcgggtgaagggttgaactggacactttgtcttgtatctgcacggctaaaatcctttccccctccccagcttccagctctagaccccactttgccccagcatttatgtggaggcagaactggggactcagccagaaatgcatctctcctgggtgatgggaggcaggggggttggtgggcaggatgggctgtgaggagatggtgcacctctcggccacgtgaaagggcttctcagtggtcctggagcagcatctgccggcctgccccctgcccccagcgtttggagtacattcagagaagtctgtaataaaaggagtgacaaggcggccatgcccccagatgccatggtgactgcagtgacaacaagaactgcttgcaggttcgggtgatggggaccccctctctcgaaaggcctaggggatgtggtgtgtggtgctgcatggaaaaaatgagcatgagcccctctggtgtgtgagctcgggcagcagaggtcaaggccagggagtccgggctggaggtccatcctcccagggacagagggaggctggctggggggacggagagaggagagaaagatatgggcgggcccaggcttggagcagaaggcggaggccacagcacaaggaggcctgagaggaaggaaccggctgcctttgcagggcaggttgaggggcgtcaggatggtactcagccccaaatgcacctctccaagggtcctggctgcctgacttcacctacatcgcttcccgtcccttgacatcccactgaggacccaagtggcaccaggaaggggactgaaggacagccgtagaagaagatgctctggaagggatgggaagaaatgggccagatgcctgggagagccacagacatacgaagcaaccttcctaaaaccatgtaattcacaaagcacttttgcacaatctaacgtagaccttccaacaaccttgtgaaagagctgaggcaggtgttttttttggccacatcgtgtgggatgtgggatcttagttccccgactaggggttgaacctgaggccccccgcccatggaaacacagagtcttaaccactggccagggaagtcctgaggcaggttcttttaaaagacccaattcctaggtgaagaaacttgaggctcagacatgtgacttgcctgaagggagaaagctctggagctgggatgacagcctaaatcctgggtctgagaatcccacgttcattccagccttgcctcactcacgtcctcgaacttcaggaaccccttcttctaggaaacactctgggtggctcccggtggcctggagggcccctggccttcagcattcctcggttccgatgcccgtcaccgaagtggccgaaggaaaagtcattctcaagcttctcccccagtctccccttccctaaagtccagccaagcctgggtcaacgcttatggggggcagagagaattctgggcctcccacatcctggagacacaagtgcactcctggaacgggcctgaagcggggacttagagccccgagctctctctccagggaaggtgttcctcctaagggccccagagcaggcagctgggggcagggactcacctgggcatagttttccgctcgggtgaggaggggcagctcgtaggctgtggagaagtgggtccgaacctgctgcatttgcccgaagcgttccctcttcctccacttggccctccggttctggaaccagacctgcaggacagagcagttgtcgccagggtcaggcccggagctggggcccccgtgttcgaggcagcgccaaccttgccctcgagcccctctggctgtagttataaggtcaagactgtggggctacagattacacatctgccttggtctccgtgctcaagagctgataatctggaaggatcgacgctgagagccgggaaggcagttcacaccccagtcgtaggcgtgacttcgggccaataggttcctttttctgaacttcagaacccagagcaggcaagtgtcacaagcacacggaacacgtcaccagtcatctcagcggtcaaataaccagccctgacatggggcggcccagtcctctggggccacagactacggcagaatctgatgacagccgtgaaagcttcaccagagagtcattctcctgggtggccaagtggtaaagaacccgcccgccaatgcaggagatataataagatgccagttccatccctggcccaggaagatcccctggaggagtgcatggcaacccactccaatgctcttgcctggagagtcccatggacagaggagcctggtgggctacagtccacggggtcacagagagtcggacaagactgagcgactaagcacacacagaggatcacacacgtgtggtttttccattgttttccacttcctgaatcccccatgcagccttttgtggaaagagtctggaccccctgggttacagcttctctgggagacagggctttagccaggctgttggaggacagtcagggacagctttgtggagggaaggtgggggctggagggacagaagtccaggctgagtttagaaacggcccggccacgccaccagctgggagagtgatgggaggggaaggatggtggaatcagatggtaggggccctcaaatgccaggtgggagggtcaccttggtgttgtgaagagaagctgctgcaatctcaggcccccaagagcagcgtgatgccagggaggaggggaggcccagctgtcactcagcacgggggtggcacattggcttgaccagggtgctctaggggagtgtggtctctgagtcaggtgtgtatgagccagggtcttgtttcatacttaggaaaaaaattgagaaccttccaacaaaagctattgggcttaatggaagcttcctttgcaaagagcgtttctggcaggctgagaac

SEQ ID NO: 2 - DNA, 85941 bp, Bos taurus
actccttcctcagcctcccagggttttctgggcaggaggggaggccagggcgataaagagaagcccactggaagcatctagatatacgactggactgctttcttcagggctgaggagaagcctagaaaaaagtgaggttgccactgtggtgggggccgcccccttgagtctctgtggactcaggtcctacagtgggagacacacagggctggcaaagggggcctgggggtgagggcaggagcagagcaggggtggtgggaaaggggggcgggggacaggagatggaatgccggcgggcggggcggagcggggtagaggggcgacctgggttatctgggaagacagatgagctgggggagaggcagctgagagcgctcggcctttgggaagggagtgaagagggtcaggcagaggggtttctttgcaggttaagatggagatcgtgggaggcggcacggctgtggggagcaggctggggggaacagaactgcccacctccagccccttcctccctggggcctcaccctgcagatgaggctggagcttttctgcccagataaggcttaagctccattagcttcctggtcttcactctcatcatgcataaacttgggtggtggcaggggtatcctcccgggccccatctgtgctcacattcatgggcccccaaggcttagagcatcctcccatttctgacccttggagcctggggtgctgacagctgcgcccctagatgccctgggcctcaggctctgcagcccataggcttgactccactgggcaaccttgggcaggagaagggacccagaggccccagccttcccttcagtgcgtcacagcagcccaggaccaccccgctcctggcccaccctggcctggacccgctcagcacgggcctctccccaaggaaccaactgtcttggcggaggcccctgctaaccgcagcccatctggggccagggcggggtggctcagggcggggaggcctctgccttattgtcttcagacacagctgcgtagattggaatcccagacagcacagaagaggcccctgcagccccctggagcggctccaaactccgggtttcttcccaggaaaggtccatctcgggggcgggggggcccgtccctgtccccaccgtcactctgtgccaggcacctggcttcccatcagctgagtatgggcccccaacaaactcatcagaggcaagcagggggttccatccccacctccccccatctccagggcactgccaagcgcaacccagaggtctgacgcacctgccaatcccaccaaccctgctgcctttatacaaagtaaagtctcagaacaattaggcacgtgttcctggtgaggcaaactacaggaaaagcactggaaagggctggaactcagggtccctcattctattctcagcacggccaccccatcacctttcctgagtcacctggtcacaggtgatggtttcctgcctctgggtctcttcctcctcgatagcgaatgagagaacgaggtggtcctctccagcactagccttctagaattctgcggtctccccctgagatgataagacaggcagcacatcacagttcccctttttgctttggcggctgggaagctcagcattgaaatttgcgctcagtgaaatctaggatccttagcttagcctaggtctttgacataattaccttcccatttcatggcactgttattgttcgaggatgaagataaatcttttaagtgtaagctgccccagctcctttttggaagtaggcataaatgtgtgtgtgtgtagagagaacactcctgagctgtactgtggtgcctgaagaatgttaaacatgctctggcgtcatttcgtgtgtggtaccagaaggatcatcctatcaatgctaagcgaattctactcctgggcacttctgtccatttgttagggacacacacacgtgtgtgcacacacgtgtacatgtgtatacctcacagaaaacggacagttcattttgcatcaacacagcacctacaggaacatcccatagccagctgctctctctccaccgactccttacgctcctctcaaatccactggctgagctgaggccagaggctggggttcctgtccagagtctggggcaccagaagtctcaggtgagacagtgggtgtgagaaggtttctggaaccacagtgaggcctcagctgcctgtcttccttccccaaacactcactgtgagctggagaggaataaggtgggaagcttgctttggggaatttatgaataaatgagctgaccgttcagagttccctggtgtttctggatggctgtgtattcactggcaactaccagtctcttttacactttagccattttagatgatctagtttctggaccatgctattaatacaaatgtctctgccttgttaaacaaagactggtgagccttttattgacaccgtaggggttccctggtggctcagatgataacaacttggcctgcaatgctgaagacttgggttcgatccctgggttgggaagatcccctggagaaggaaactgactactcactccagtattcttgcctggaagaaccacggacagagaagcctggcaggctacagttcaaggggtctcgaagagttggacacaactgagagattttttcactttaggaacactctactttcctggtagcttaggtggtaacgaatcctcctgcagtgtgggaaacctgggtttgatccctgggttgaaaaaatccactggagaagggaatggctacccactctagaattcttgcctggagaattccaccgacagaggatcctggcaggctacagtccatggtgtcacaagagtcaggcgtaactgagcgactttcacttcacttcagaatactctgcagaagattctttagtgaaaaaaggcgagaaggggcacccttggggtctgctgagtaacagggctctcagtcctggcaccgaaaggaccccagctgagcttgctgcttttttaaaaagactcctctttgctcttaagctgttgatttctatagcccacgggagagggaaacactgttatctgcaaacacttgatttgctatttcaacatgatttgggctgggaaggcaggccaaacgtggcgttcatttagtgttacgctgaagcctgagctttatcttgtcatctatggttggggtgcttagaggactttgcatcctctctggtttttcctttgttacaatttcatgcagggaggggagacatacctctctggacaaaggaggtcagttggtccacatatatttattttttgccttttctgatcttagttcagccttctctaattggtgggaattatagtgatgaagaatcagaccgtacctgccctcatcatattctagtgagatgagacaaacagatgacataatttaagccaatcataaattctaagataaaatagcacaactggtagaaggtgacctggtttgtttgtgttgtattaggttgagttgtgttgggttgggttgagctgtatggggttccactgtggtgcactgaattgtgttgggttgtgctgagctatgtcgggttaggttgcggggtgttgtgttgtgctgagctacgttgggttaggttatggtgtgttgtgttgtgctgagctacgttgggttaggttatggtgtgttgtgttgtgctgagctatggtggactctgctggcctgtgctgggattagtgttttggtggtcgggtgatggtagggggcagtagtcatggtgattgggaagacttctcagaggaggcatactgagctgggacctgaaagatggaggaggaggcagccagcttgggcaaagagaagagttggattgtcttggtttctgcccatggggtgccatggatgtcttgtaagttcatcctgtttcctttctaagaggtaggccttccagagcagagacccctcatcgtcatcacctccttcgcaagagagcctggactcacacaccacacagaactcagaagcatagctgacgggctgcctgccccttccctgggagccccgccctagcccctgccaaccagagcatgaagcaggatgggaacgtgaacaaaagcagacctgctctggttacggcaggggcgcgaggggcgagctccttgagctgaggtgaagaaagccctggtgcgcctggggacgggcaagcggccctgtctcgggccactggcggaaggggccagccccgctctccccgccttccctggcaccctcactgacctgcacgcgggcctcggtgaggtcggtcctcatggccagctgctcccgcgcgtacacatcggggtagtgggtcttctggaagaccttctccagctcctccaactggtagctggtgaaggtggttctgttccgccgcttcttgcccttgttgctctccgagtcggccttctccattgggctggggaggtcggcgctggcccggtcctggggccccttcaccccagcttccttcacactgaggtagctgctgtccatccccaccgtgtcagagtcgggtggcaactctggctcacccagggagctctctttggctgaagggggaggaaacaaagtcaaaaaagcaatggctgaacccagcccgagaggtaggagggacgggggagccattccctgcttttcccttctcggggaagcatgggatgcaaggtccggaggcctgggttctaaggtcattaatgtgctttgtgaccttggctaagtttctttccctctctgagcctcttcgccgttacgtcagcatgggccccttttagcaccgtgtcctgtgactctgggccagccaatctgacttctataacatttaccctccgcccccactgacccagggctcctccctctaggactccccttgccagaacttctcctttgggtgttgaggatgttgtctcagtccttcggactcaaaagctcagccagcagccatgtctggcccaactggccttagggtgaggaggagaggctcctccttgcctggcctgattctgtttggggttcagcaccctgccccctccaggtgggggctgtttactaaggaaatagggggtcctccccgtgacatgcccaggatgctgagctgggaggcccagcagatttacacaaagctcatggccccggggcccacagccaccgctgtctagcagaacgggactctgccggctgattcctggacgctcagagctggcaaggaccagttatccagtgcaggtcaaggtgaggagacagagggagggcagggagggcgagctcatccttgttcagatagccccatcagtgccctgggcagacacactcgccccagggcggaggttaggggacaggccctgcaactggcctcagggtgtgagtccccccccatgtctgactcctgagctgtgtgcttggacctttcgctgagcctctctgtgcctcagtttccccctctatgaaatgagggcacagatacttggatctccctcacagggacttccctggtggctcagacggtaaagcgtctgcctacaatgtgggagacctgggttcaatccctgggtcgggaagatctcctggagaaggaaatggcaacccactccagtattcttgcttggagaattcccatggacggaggagcctggtgggctacagtccatggggtcgcaaagagtcggacacgactgagcgacttcactttctttctttccttcttttctttcaaagggtagctgtgggattaaaatgtcttacaggtaaagttgctccctgttctggtctagacttgtgagcagcagaagagtctagccctggaagagtcctcccagtgggcaggccctccctggagtcccccagcccaggagtgggtgcggatggggtgcgagtccctctctgagccaaattccactggggactctgactttacttatagagtgaaataacatacgggataataagacatgtgtggggagcattggataaaacttttcatgtgtgaaagctctgatcataacaagcatttgtggtctgtatgggcttccaccattctgactcttggtctcctcctctgggaatagatctgagccccaggtgttaggtgtcaggacggtgcctctagccccggtcccttgatcctgaccagatcctggggccgaagcttcggatccgccctcctccctggcactctgtgtttactgggggctctcttattgctggggagctgagagggacaggctgcccaaggcggtgaggaaagggagtggaggtgaatgctcagagtgggcctggaagcagcttcaggaggctgttcctgagggccactggcagcatgggcgccagcaggctggggggccggcccttgggcaccacggatgacacccatgtgagtccaggcacacgcagaccgccacggctttggtgacagcgtcatgcaaactgcagctggcagctgggctgagagctttcagcaaagatatttatcttcctggcagacaaatacgttttgtgttttggtttttttttttttttaaagagaagaaatcccacccagtctcaccaaaaataaacatctctgttatgcaacctcctggaggccagactcggggagcaggcagtggctgcagcccctgccccttgtgcccaagccctgctgcccagaccccatcccatgaccccaaggacccttcccgtatcctctggcatccagcctgggctcaggaccaccccacacagcaaaaggggctgggccgagcaggggaaactccatgttataaatatcaagggacggtagggactaaactccttgtgttaattcaggggaaacagaagccagagagagtaagaaatgtgccaatagggacagggtgcagcctctcagctctgggtcagtccttctcttctaccccgtggtcttggggcctttcccagggagagagggaaaaccagaaggaatccccagggcctcacgtaatcaagaggtatggtctgcacatcaaggaggcagagaaccagtccccgaggatgagagagtgaggatgtggcccctgggagaagcccacccccgctgagctggttctaggctcactgcgggtggagctggctgaacacccagtcgtccaagtagagaagcttccccacctccatccacccactgaaagggtaggcatggggggacagcatggtgtggggcgcagcatgggagaccccaggcccaaggtgcggagatctgcctcgtcccagctctcccgaaatctgcctgtgacctcagaggacgccttccaagtgccagagcctcctgtgccgatgggagcaggcaagccctgtccgcagtggggagtgcaggcaggaatggcatctcggccctgtggggcagccctcccgcctcagtctccagtgggaccttccccttgggacctggagtggggagaggctgggggcatatctgtccaggcaaacgtcagctaactagaaaggcttcaagatttcaagacggaagggattctggcaaaccgaggcctctctggccctcgaagagggacgagaccttccaaatctcaagtcttgattattcttgctttaggtggcttaggcggtaaagcgtctgtctgtaatgcaggagacccaggttcgatccctgggttgggaagatcccttggagaaggaaatggtagcccactctagtactcttgcttggaaaatttcatggatagagcagcctggtaggctataggccatggggttgcaaagagtcggacacgactgagtgacttcactgacttcactttaggttctgaggcttggcccctaattgagacagcaaaggaaaattccccaaggttctgacccaacccattagaatgacaagccaaggtactcatccctaaagtgccaggcttgggggcatcagttcccacctcctgccaccctataagcatctgcacccagtgctgtcactcaagctttggtgacggagctcatcgcccccaggaccactggaccgtctgtccaaaggtccagcctgtgtctgattcctgcggtctggctcattggcatgggttccaaacctccatgggccttccccaaggcagccctgcggacatctggagatggctaccatgcctctgagtgtcccatcctcctgaataaatatagctctcactgctgttctgtaggaggccctgagtttaacaggcccgtgtgagacaggtgaatgccagggatgggatgggtggtggtgctgggctctgggcaaagccagcagatcactctccaactagaggatccagagaggctgggtagagacttggcaccatggcccgagacctgggcctggtcaggatccagcttgctccgtaaggccctcctcaacaagacggccgctgaaaacagagggcctgagactcaggctgactcactcctgaacaaaggaaaccactgagctccttgctctccctcacctcctcctctgtccacctgtctggaacgcccctcttttccagctgacctccaaggtccagtcaagcaccagctcctccaggaagccttcccaaactgcggcaatcatacagaccccctccatctagttgctgcctacgttttaatcaaaccctgaaggtccagcttctcctcagggaaccagttccctgagggctggaaccttgggccatttgtccctgctcagcccaggatggggcaggcaggaggttaccgaggaatcaatggccaatacaaacattatcgagctctcaccctgtccagctacctctgtgtactcactggcagtacctacagggtacgtgcagagacgtctgctgaacaaacacatcctcggagacaatggagaaagctggtgagggtgcatgaaagagaggcatgcagtgggtagtgcctccatcagaactgacagctgaaacaggaggaagaggtgagatcaccgaacgacaagaggggaagagggcagaccgaaaggaggcaggaggaggaagaacaggcagacacagatgatgtctccacacagtgagcaagggggacacgctgggggaggccacgcaggagcaccgaatgggggctctagaattgacgtttccagaaacccagtccaggatctgccaggaaccagctctgcatttactgcactgccacaccaggagacaggaaaggccccagctaacccctttgccgggcgcgccttcttgaggccacatggcatccgctgctggcatgaagctgaacgacagccctcactaggaccaggccacctgcctgcccggctggcttccccacatcctccctggagcagcccctcatggtctctggccccatccagagcgggaagggacccctgggcgggctttgtcccaggctttgggggcccaagacaatgtcctcgcgagctgcagcttgccccggaatgtcttgatctggaaatacatcccagactacccagaggaatcatgtattgtgcacagaacgtcgagaatccagagtgcctgcccccagggagggatgcacacccttcagagactcccatgaccacttctgatgagtgggcggcgtttggttcaaggcttactgttcatcaggagtcacagacacccttgagcctctgcctgttctcctcctggggaggaggcctcgcctcttctgagggccaactggtctgtgccggacagccttggctgttggattttaagagctacaagaaacggggtacctttcttcattctcttgtgtgcatgctttgtcactcagtcatgtctgactctttgcaaccgcatggactgtagcccaccagagtcctctgtccatggaattccctaggcaagaatactggagtgggttgccattccctcctccagggaccttcccgacccaaggatctaacccaagtctcctgcatttctggtggattctttactgtccgagccaccagggaagccccttagcttctatctactcatcctggttgaatcctagggtgttgctccaacctagtctaactttttttttttttgacatgaaaggccttgaaagatttccaaagttcaccttaatttttttttttttttccatgactgtgatttcaagagaattatttctgtgattgtgatttaaggagaatgagcttctgaagccatttggggagaggctgctggagccctttctgcgtgtatccctttcttgtgcacggatgtgcgccttcgggcatgtagcacaagcctgctgttgtgaatgggtgtgggcagcatatgtgtagttgcctgcggataggtctgcggagcgtgcaggcttgtgtggtcaagggcaggcacaccagtgggcgtgtgtgccagtggatgtgtgctgctgtgtgtgtctgtgagtgagcgtgagcctgcagacagccatgggtgggtctggacatgagttgtgtgtatgtgtgtgtgtgtgtgtgtgtgtagctgtgcacacactctgacgtatggagcgtctgtctaaggggctggagtaattttatcctccatggttttgattatatctatttttcctccaaacatctgctttgtgggggacacaccgctcgccctgttctcgggtgagccagcctcacacgccctgtggcagtgtgcacagcgccaatgcaatctccttaaagaaaacaggccagcgtgaggccctttcctgctgtttaaagggcaccgagtaaacccatcgctgtccaaacagttgtgcgggggtgggggcagaggttaaaggtgagccatcgatcacgtccccagcccaccttatctgccactgggctctgtcccgagttcagcttggatggcccgagttttaggtccaggcccaataaaatacaccaacgggggagcccatgctggggccacctttcagcagacacacgtggagcttatctgtgcgtaccgggccgagatggactgtgttcctgtccgggaggaactgccaggcggaagaggagaggtaagccagggactcagccagccacccacgaggtggggtaggcactcctgccaccaacacacacccacccaaggcccaaacgtgcttctccaaacaaggcaagtctgagaaactgtcacacgtcacaggaggctgagacatggaggactaaatgtgatgtggtttcccggacagggtcctggaacaacagtaaaaagcacattagcggaaaactagtgaaatgggaataaagcgtggcacttagcaggtagatgggtaaactatttgaatggacatttcacccaagaagatatacatctggtggttcagatggtgaagaatctgcctgcaatgcaggggacccaggttccatccctgggttgcaaagatcccctggagaagggaatggctaccccattcccttgcctggagaatttcatggacagaggagcctggggggctatcctgaactcacaggatcccaaagagtcagacaaggctgagcaactaacaaatataataaccagctcaatgtattaccaattagagattatttactagggatattcatgagatatggctacacaccaacagcgatggaaataaaactggatgatacccagagatatcaaggaactgaagggcgtgaactcgcttccatcagtggtgggaaaacagcgcagcagtgccttataccatgaaacacactctcatggctcgagccagacatcccactgctaggcattcgccctcaagagacaaacacttcttttcagggcaaacctgtatgcaaatatttatagtggctttgttcacaatcgcctcaaactggaaacagcccaaatgtcctttaaaccatggaacagccatgcaatgggatactactcagagataaaaggagaaaactagtgatgtgaactgtggatgactctcaaagacagcattactgtctgtaaaagaagccagtctccagaggtatatggtgcctggctccacttgtttattgcatgccattctggggaaggtaaccgactcagtggacgtgaatttgagcaaactccaggagacagtggaggacccaggagcctgacgggctacagtccatgaggtcacaaagagttggacacgacttagcaactgaacaacaaacaaacaaacagtcccaggacagagagcagatcgctggtggcctggggctgggctggggtggttttaaccccaaagggctggtgtatgggactatccgatcctgtatcccagctgtgattgtgagtacacggatctgtacaggaacgtgttcaaattcataaaatcacgctcctcagtttaaattttcagcatgctatttaaaaaataaaaaatatgcaaactacagggcttccctggtggctcaggggtaaagaatctgcctgccagtgcaggagacacgggttcgatccctggtctgggaagatcccacatgctgtggagcagctaagcccatctgtctgtctgtcagttgctcagtcgtgttcaactctgcaaccccataaactgcagccgaccaggctcctcggcccatggggttttccaggcaaggacgctggagtgggttgccaaactgcaacgtaactatcgagcctgcgctcttcgagcccgggaacctcaaacgctgagcccacgtcctgcaagtaccgaatccaaggtgcccttgagcctggactccacaaacagaggagccaccacggtgagaagcccgcacaccgcaacagagaaaagctcgtgcagtaaggaagatccggcacagccaaaaataaataaaaatattaaaaaaataaaaaatatataaaactatagagaaaatacaaaattgggtttaaatatagtaaagaaaaactaagcaggtaaaatgtttgtatgtcttcacccaaaaatctcacctctagggatttaaagacagaattatggatagacacaaagaattaactagagagaaagttcctcgtggcatttttatatgggaaaattgaaaataagcaaaatctctattaataggagattggtcaaataaaggatgatatttccataagtggaagatccttcctggttttctttattctacacactgtttctctcttggtatttaacacatattattttgtatttatttgttttttccttcatttattgtctctctctcccttccataaaacagggaatatatctgaatatatttgtttggcttagccttttggtttaatgcctagtattgcggctggtatgcagaaggggttcaataaacactgatgaaaaatgaaagaaactgatgtccaagaatagcattttattacctgaaagttgttcatgttacagtaagtaaaacttattttgaaaaaaggggaggtgaggatgcggtgtctgttcttagaaaattctagcagtttccagaagtaaagaatatcagtatctgggtttagttgcttttttgtttctagtctgacttaattttcttatttgtcctaccataaacaggaatttctttcatcattttaaaatgctatgtgaaagcagcagctagaatcctgaatggtgccggctcggtaggtttgggactgggctgtgtgatgggaggcggagggaagcgctgcagggaagcagaggaggatggcagtgatgcttcagagaaaggccccttccgaggtgggactagctaccgtcctcacaaactgggcaccgccccccacggctctgaggcctcgtgggtgctggagagtcccagagctctggcttacgttcagacctcaccggttcctctgccctcagatcaccacgctccactacaaccggagacagtgtctaagacgcacactgctggtctagggccaccctgtctaacccagagctctccttgttgttcagtcgctaaatcgcgcccgactctttgcaactccacggactgcagcacaccaggcttccctgtccttcactagctcccggagtttgctcaaactcatgtccattgagtcagggatgccacgtaaccatctcatcttctgccacctcctttccctcctgccctcagtctttcccagtatcaggggcttttccaatcaggatggtcagatagggtacagggtgctcagttaaattagaatttcagagaaacaggagtcactttttaagtaaaagcatgtcccaaatattgcatgggaaatacttatgcactaagtcccctacgaacaaaccttcaagttgcagactttcaaagatgcgaacgtgcattccctcagtgtcagacccgagtgaaactgcagcttggcccctaccatctcctatagctgacgatccttccgctctactgtctcccacctcctgtccctccctccagccagcaactcttcttgcctgtgtgctggaggccagctcctctatgccagctgttacactgtgctactgcacttttcaaggtactgtactggaagatgaaaaatgtttaattttttctgtttgtttttatgtattgatattatttgtgtgaaaagtatgataaacctatttccgtacagtactacatagccgattgtgctcgctgggtttctaggctaacttgcttggacttacgaaccaagtgaagttacgaactcgctctcggaatggaactcgtttgtatgtaagggacacaatgtactgaacattattcattgtttatttgaacttcaaattaaatggaacatactggttttgttgttagatttttattttatgtatttattttttttgctaaaaccgccaatgctcgagtcaggctgactatggcattcaggattccccaggcaggaccggccactgtccccctcacagacttacgtcttgccacacccgcccctccacctcgcttccggtcccgctgacctgcccgcacgcccccaacacactgctcgagcggcttcaggcttccccacccttgcatccttctcgctgggacgccctctgcccttcgtgcacctggctaactcctactctccctgggtatcttcctctgtaagcttcgtggctttcctccccacccccactacatacagttatctatgttctatatgaagacaagcccaaagaagaaaactataatacataaacagaaaagggaaacataattttttataaaagtcttttgtaaaacctatcactgccctgtacatctttccatcctgttctatattcttctgctaatacatcacttttaatggctgcatagtattccatcgtacagacaaactctatttcctttcctcagtccttactgttgggtatacggattgttgattgtttgaaataggaacaggcctgtgatgaatacctttatagttaagtcccttgacatttttgtggcagttacctttgcaaaagttccaagaactagaaatactgagtttggtcacagtcacaccttaagagctctgataagtgtgtctgctagaaatgtgcccattttttcagcatgcctgcctgcttcaggacaagtaccaatcattacactgtttataaaaagctttgccagtgtaatggataaaagtgaaatctcatttgaatttacatttaaaaaaaaaaaaacaaatttgggcttcccaggaggctccggagaaggcaatggcaccccactccagtactcttgcctagaaaatcccatggaaggaggagcctggtaggctgcagtccatggggtcgctagagtcagacatgactgagcgacttcactttcacttttcactttcatgcattggagaaggaaatggcaacccaatccagtgttcttgcctggagaatcccagggatggggaagcctggtgggctgccatctatggggtcgcatagagtcggacacgactgaagcgacttagcagcagcagcagcaggaggctcagtggtaaagagtccacctgacaatgcatgtgacatgagttcgatccctgggttgggaagatctcctggaggacgaaatggcaacccactccagtactctagctgaaaaaagtcccacggacagaggaacctggcagtctagagttcatgaggccacaaagagccggacgtgactgaggccgagcgtgctcacacacacacaggcacatattactagtgaagttaacctttaatgtgttgcatttcttcttttgtgaattgtttctactgagcttccaaggttcagtgcaaaggtgactccttcttcctctctgaccccagggtcgggcttctctcttaaaggctgaattccccactctgaagcacggcggtgtggtgtggcctcccgttcgaccgagagctgttgggaatagggctgccctgatgcacttcggggtcctgaactgggccccggctccttctgcctttggtagcaccaggcagctctgcgtgccctgctgagggcgacaagggcacccagaggaagggggcccaggaccatgaggctgggctccagagcagggtggggggctgcgatcccccagcacacactgctgtggggtggctgtggcatgggggtggaccttggtccttgtggggtgatggggctcagagaagccggccacggggcaggtcgtgtaggaccaagccccatgtagctccagacactgctatggcctcaggcaagggttttggcttttctgcttcttcacgtcagcaagccctgcctggtctcctgccagggagaggagaggagggttacagaggaggtgcagccctccccgggaagccaagactgggatgttctcctcacccacagggcaggtttgtgcgggcccctgtgtgaaggctctaggcacccccatcctgccagtcaggatcaaggctgcagtagctcacactggccaccaggaggcactgcagtcccaagtaccttcctcagggccagaagtgaggccccagggccccgccgcagatgcttctcccagtttggttaaagacccaggccccctcccggagggttggccaaacaagtggtgattcagccctactatgtgtctctgatatcccagccctccacacagtgctgggtgaggagggtccctgcccttctccacagagctctttcctcctccggctctgagggtattcacgttcttgacttgtatcagtccccgtcttgagcgtcctcattttacagatgggcaaactgaggctcagagagagggaatccactaaacgtcaccagccactgggcagccggcctgacccgagttgctcttattccccccaatctcttccctttgtggagacctgaggggcatcgctggccctccggatactgacaggtaagccatgcccaggctacccgaccctggttttgccaagtcggctgtccaggcagctcgtggatttgttgctggctcctctgtgggctcttccccagggaagcttcagcagtctcggggactgcaggctcttcttcccatcactccacactgaggggaggggtttcctgtgagccagaggatcgagggggctccccagaatcaggagggaaaaggactcaaccctgggctctcactacacaccaggagcttcatggtttactaacatgctctcactgctgctgtttacaacagccatgtgaaatatgcccattttacagctgcagagactgaggccagagggcgtaaaggacttgtctaagaatgcacagctgctgagaggcaggatttgaaccccaagcctggggcatcttcctctgggccctcctggagtggatacccagtgtggggctgggacagggcagccaggagacagagtcagggaagaaggggaaaaaaaagtgggagccacaggaaaattcctcctctctgatgcctctaaatctgcccaacatatccaaccttcagtgctggccctggtgaaactgtcctgatagaacttcatagaacatctgggacctccagtgtggaccaaggccttttctcgggaccttgctttcagggtcactccatcccacagtttacattcaactaattttttcttttcctcttcctgacactgggtctggatttatattcaattctctgctaaataccgaagtatttaaaagtaaaaaggcttatgtctgcgacttacccccaaacagttctgaaagaaataatatgtatatgtgcatctgaatatatattaatatatacatacagaaagaacacaaaagagagtgagggcaataatgcaaatatggtaaaactttaagatatggaggatcggtgtcaaagggatctgggaatcctttgtattctacatttaacttttcctggtgtctgaaaacatgtcaaatgaaaagcttttaaaaagaacaagaatgcagacaaatgggaagaaaccactttatccaggaatccaagttctgaaaccggcatttgttacagtttgaaacaggacccagactagagtctagtaagcaatctctctgcctccctctcaccagcaccatccacaacttctacaaacgggcttgcacgctgaaatcaaaatcacaaggcagatcaactcaagggacaaacgtatactccgtagtcctatggaaaccctcatttccatccccggaagcaaccggagaccccattaaaacatgcggttcttctaaggccgtaggaggcagtattttgttagctgaggtcgagctatccttgggacttagaagtacttggtactgtttgagcctattctaatttcagctcccttacagttagtccaagcgggtacggcttccttgcacccaaagagaagtggttaaaaaggaagcccgccgacaatagctggcacatgcaagtgaccgtgttcatcggtgatgaacctccacacagaggaggattattcaccctcagaaaggagggaaattctggcacctgctacagcacggatgagccttgaggacatcgtggtgaatgaaacaaagcagtcacaaaaaggcaaatactgcacgattccacgtacacgagtccctggagcggagacagaaagtagaacaatggctgccagcggccggggcccaggagggtgcggagttggtgtttcacggggacagggtttcggtttgacaagatgaaaagagttatgcggctggatggtggtgatggttgcacaacattatgaatgctcttcataccgctggaccgtacacttaaaaatggtcatgatggtaaattttatttttacttgtattttaaaattcttttaaaaattggagagaaagaaaagaaagtccactactggctagggtccctgcgggccttcatccggccttccttcagtccccacggtcctctccgtaccccagcttaggccccagattcttttctcttctcttctccctgacactgtggcttgggttgcgtctattcattgcactcattcgttagttgacttgtttgttcattcaccaagcacctccccagggcctccctttagacatggcctcaccaaagggacagacgacaccacggcctgacagcaagagccctgtgacctgggcaaggcaggctctatgggaggagtggccggtgctggctgaaaaggtcaggagaggctttagcaaggatgtgaacttgacctgagtactgacatctacagaggccttggcaaggcccggtaaccacattttatggcaaaacaggacacccagctttcagctgaactggtcctaaattactaagtgagaagccccaggggccctcccccagggctggtgacaacaatgagacaatcatacaattgcttgcgttgcagcagttctgacccactcactgtctcatctgatctcctcagatccctaagaggtccgtgatgaggaaactgaggtccagagaagcttgacttgctcaagatcacaaagcgaggaggggcagagctgggatttgaacccagatggtccgactccaagcctggtgtcccgtccacggcctctcacgtgtctcctggacacaggcaggcagtcttggggggtggggcgggcactggctgactcggtcttgtctttgagtgacacagttgagcctgaggagaaatttccatctcagaggaataacctcaacagagctctggggtcaccgcgtgctggttttggtggtgtagtcactcagtcgtgtccaactcttgcgaccccatggactgtagcctgcagggctcctctgtccctgggattctccaggcaagaatactggagtgggttgccatttccttctccaggggatcttcccgactcaggaatcgaacccagatctcctgcactgcaggcagattctctactaactgagctacaagggaagtccatgggtcaccatgagaagccccatttatggtctaacacttggacatcacctggtgctgttctctggggtgggctggggtcatgacctcagtcagggactctaactacccctgtgaacagccctcctcatggacactgaggtttcccagctccccgcttcctggcccagcccaggtctcatccccaagccctgcctcacgaggctggaggggatccagagacccagcaggggaaggggcagcggcccaggctgggtctactcccagagaaacagaaggaacctcccccattctcccttcagcccaaaaagaagccccagagctcaggggccaccctgggccacccctcctctttcaggcaggatacggagacccccgagtgatcacatcgcctgtcagtggaaggactgtcttctgatttcagatcagctcagtggtctgcctgccccttgtccccctctacctttacagaaccatgtggccttgggcacgtccttatctccttgatccctcagagcctccgtctctcctctgcttcagaaaatctgcattcccaagcatccccttggggacactgtaataccaccgttgtattcaagggaggcaagatggagaagctcagatcctgctctaggtgaaatgaatgaggagtccttggagcaggacggacttgggttgactcctgaaactgcccctgacttgctgcatgcattcattcagcagcatttctggagcgctagttatgtgcagggcaaagctccaggagatggaaacaaagtccaagaaagcatggcccctcccttacaaaagccctgcttcatgaaggaggcagtgattatggcggtggaggtggatacggcaggggcatctcggccagcctggcgcgggaagcagtgtttctggggcctctccagatctctaggcagcgatctggttggaggggtctgtcgaagctgtgtacagctagctctgtgcatacgatgaagaaatgtcagctctccccatcaaagaaatggggaaatttctcagggagattgtttaggaggcttccaagccactcatgccttggcaaagtgactttggcctctgtgtgactgggtttcctcatctgtaaagtaagggggaggggacccacctcccagcatgtttgtgggctctcagtgaggccaagggcgagaaggcacacctctgcctgacacccagcacgctgtctgtcaacgggaatcctccacgggagattaacacacttattatttggagggaagttaggccagggcgggaggagaccgcagaagagcccctcaagctgcagtgtctcatcctccccctgcccccgcccccgccccaggtatatgctcttcagctcaagtttccccagctccagctgcccagaacattccacagacctccgtcccaaggagctccccccaccccagtccccttccaatcaaggctcatttccacgtacatataacagagaaataaatggggtgaaacactgaaactattttgtttgtcatttgttatagatatttaaaagtgctagctgatgttcaagaaagccccttccatacatacattttgacccaggctgcaacttgtagtagaggcatgaaaggaggactctaattttaagcgctgcatcgaagacagtgtggcttatgcgccagttgacaaaggtcagagaggctgtgagctcctttcaaaaacctcgctcctgcttagacacttggtaacatctggacgactctcctatgcaggcacaccaatatttaacggctccagaagaattttctctgtgggggccttcgctgtcctccacccagcataccacccaaaagctatctgggggtggggagtgtcgagagggatcgttccagaaatgaaccgccactcccactggcgctgctgtctgctgtcctatagaaactaaccttggtgattcggggctatgtcagggccctttctgccagagaaacccaaatcacatttccaggcccgagtctgttaccacttcctctttgtagatttcctgccatgccctcctccaaactctccttcttcttctgtgaaagtcacttagtcgtgtccaactctttgtgaccccatggactatacagtccatggaattctccaggccagaatactggagtgggtagcctttcccttttccaggagatcttcccaacccggggatcaaacccaggtctcctgcactgcaggcagattctttactagctcttcacgagccacaagggaagcccccgaactactcctaataattcgactgcttttcacaaagcattgccaagtgtggatgcatggcaccctccaccattagacagtgtcttcatcacaggcagggaaaaagtcttgtcaaactctggtccgcacatctggcactcaaaaaatacctgctgaacaaccaactcaacagatacctgaattaaatgaatctggttaagtaactaaatgtctcagtctgtggaacgaccacatcggcctagagagaatgtttttcccaccaaaatcaagggttttcttcctccagatgtgttcatggtaaactaaacaatcatttacaagccccaaaagcaagaatatgttttctccccttggacaagaagggaaaaattatggctggatcaattgtgaggttgccagttaaagttttctgaggttgataaagatggataagattattcagggtttcccaggtggtggtagtggtaaagaacccacctgccaacacaggagacataagagatgcgggttcattccctgggtcgggaagatcctctggaagagagtatagcaacgcactccagtattcttgcctggagaatcctattgacagagctgcctggcaggctacgggtccacagagttgcagagtcagacacgactgaagagacagcacagtgcttgtcaggaaaatgactcatcccatgaaacagtgagaatgcaccttatggagcacaaaagtggcttcctgtaatcttatccgaggggctgagtcctgggtagctcattaggtaaacctggccctggatgtccactcaggcaaggcctctccaaggcagcccctgatccctggcacagaaccaatgccgagccaacgaggaggccgttccaggagcggtcatttcttgcgagtttaaggaagggagcctaaactccccagcagacatggcttgaggggtctgaggggcctaggactacccttctcccctccttgatccctccagcccccatgctgggcagacagccagcttatacccttttttcatcactcatctgcattttaaaatgctttgtcaaccacacaatatagactcagcaataatttgttgatttttcaattttatgcattcaagtcaaccataactgtctgtcaaagtttccggcctcacctactcaaagttccagcaacatcctattatcaacaataacaactaacatttattgagcacttctgctgccgtaagctctttacctatatcaccctcctcaatcctcacaacattccttcatggtagataggattcatatgcccactttacagacaaggaaaattgaggcaaggaggcaaagaggtcaactcatttgccgaggggcactcagctggacaggcccaaatctgtttccaggtcctggctatactggcctcactgaaccaaaggcacctgaagatgcaggcagccggtgcagcctgactgtagcatgaacacagtttctataagattccaaggcattagaagctgcgcctgttacttgcctttgggcttccccgaaggctcagaggataaagaatcgtctgccacgcaagagacacaggagacatgggagatgcaagttcaatcgctgggcagggaagatcccctggaggaggaaatggcaacccactccacctggccgggagaccaggcctgtgtttaaccaactgggctggggggtcccgaggcaggatacactggtctcatgagcgagccccctctggggctctcactgctccactggtagcggcagccacctgtccgttgccccctgcccctctggttacccaccaggggcgccgaatcctacctggtccctggctgagccactccctgggcagagcttgggatgaccagggttgggtacagggagcagcagatgcctctgatgaggaggaagcagaaaatcttcaggacattcacgggcccaggagtacagagcagggtggggtgcatggacttccctccaggggctcctcccagccctgtggggagagggctctggggtcatgttatggaagggagtcctctccgatctggtcctcttagggggtagtgtgaggaccctaagagcacatctaccccaaggccatatcctcatgggagactcagctgatggacacgggggctcccaagtatggccacaggcctagtcagaggccagggctggggcgtgggcaccctcgccactagctctgccctcccccacttctcatccgctctggctgctgaaaaacaaatgtgtcccagagccgaatctctttggcgggtcagtcgttggcctggaggtgaggagttgagcaggatgacttaatgaagttccttatattctagaactctggaccttcatctcaggaggccatgccacactggggacattgactagtggtacagaatagagagcccagaaataaattctcaaatacatggccaatggatttttgacaaggcagccaagaccattcaatggagaaagggcagtcttttcaacaaatgataccgggaaaagtggatatccgcatatgaaagaataaagattgatccttatcctataccatctacaaaaattaactcagcgtggttcaagacctaaacttaagagctacaactataaagctcttagaagaagacataaggaaaatcttcctgccactggagttagcaatgatttcttggctatgacaatgaaaacacaagcaaccaaggaaaaaattgataaattggattttttcaaaattaaaaaattttgtgcatcaaaaggacactatccggaaagtgagaagacaacaatagagaaaatgtttgcaaatcagatatctgacaagggattatcatatatatatatatatatatatatatatatatatatatagggctccctggtggctcagatggtaaagaatccacctataatctaggagaccagagttcgacccctaggtcaaaaaaatgccctggagaagagaatggcaacccagtccagtattcatgcctgggaaattccatgaacacaggagcctgacgggctacagtccatggggttgcaaagagttagacatgactgagcgactaacacacatatatagatatatccagaatatacataaaactcctacaactcaacagcaaaagccaagaatgcaattcaaaaatgggcaaatagatatttatttataaatatttgccccaaaatgggcaaataaatcttctttctccaaagaaagaagttgtcagaataaaggtcaaagataaataaataaagattgtcaaagaataaaggtcaacaatggtttgacaaaatggtcaaacacagaaggacaaatattgtgtaattcgacatctatgaggtgcctggggtaggaaaattcagagacagaaagtagaacagaggtttccagggaatgggaggagagggagatggggaattattgtttaatgggcccatttgggatgatgaaagggttctggaaatagtggtgatggtcacacaacaccacgaatgtacttgatgccaatgaattgttcattttaaatccttaaaatggtaaattttaagttacgtatatgttaccacgggcttcccaggtggcaccagaggtaaagaacctgcctgccaatgcaggagacgcaagagatgcgggtttaatccctgggtggggaagatcccctggaggagggcatggcaacccactccagtactcttgcctggagaatcccatggacagaggagtctgacgggctatggtccacagggttccaaagatccagacatgacttagcacagcacatattttaccacaataaaaattatccctaattaaaataaagatttgaatgttgaaagaggctgcactaagtctggggacgcagcctctcactgcactgctcagaggcttccagggggcttcccaaagtctgaacccctcgtcagaaagtgaatggccccctctgctttgcccacgccgttcttcaggctgattcttgcctctacccatcatgccctcgcaagtcacacacctgctctttcccaggcattccacgcactttcctgacctagttctctctgactctagagtgctgtttccagctcttccagctggagaactcctattcaacctccaaggcccacttccaaatgcccctacccactaagtcccagtccattcgcacttttcctgagaaccgcgatgtttgcacatttagctccccgtgggcaggaatcacctgactcgctgcccagtgttccacacccaacaacacagggcacaacccggcagcagcatgtgtcagatgtgaagggggagcctttggcagcacacctctcacagccccgtctatgcccaactccttctctacagagagacggggggagcccttccatagagtctcctccgcctttgttggccgccacctgtgagagctgactccaacccaggctcaggtctcagtgtttcttctcatctgggctgtgagctcaggacggcatataggcccgagcggaggggggcatcccctggcggggggaatctcagaaccctggggaggggcagggctgagtggaggggggaggacctagggaagagcgtcatcagaagaaccaggggccccaaactcactgcagggaaggggggagccctgaggagaggagggtgtgtatgggtgggtgagggggagacggcccaggagactgcagccacggtgctcctctaactgcaggagcctgagaggacaccaggcctgtgcagaacagagcaactcaaggtctccttctaccacgcttcagaggggtccccgcaggaaatatttttcgccagggtagaagagaactagaagtcgggttctcacccatgcccacgcctccttcaaaaggaggaggaagggaaggaggatgggaaggttccctccctcctccctgcctccctcccttcctccctacgttcccaaaattcactgagcgcctgccctgtacagcaagccctgctctaggctctgggaaaacagtttgaaacaaaaccgttttgtaagacaaaagcctcacaaggtctcacaaactccccaggaggcaggtatttcctcctgtggtcttcagctccaccctgccctgatcagaggttctggcacgatcctggaacctctgggacttaagatgcaatccctcacaacagttatgaggcaccgatgccacacctgaccccaacacagcaggacacttgaacagggaagaaactaggcccaaggacacagggtcaggtctagactcggacctaccggggctccaagcccatccttgacctcctccccagccaccgccaccccacccgccaccactgcacctccagacaggaggatgggaggagctctttggggccatgtttgtaaacccatgtcatctgagataggatgagctgtgtttagtcgctcagtcgtgtccaactctttgtgaccccatagacttcagcctgccaggctcctctggctcctctgtccatggggattctccagacaagaacactggagtgggtagcctttccctcctccaaggggatcttcccaacccagggatcgaacccaggtctcctgcattgagggcgaattcttcaccatctgaaccaccagggaaacccctggatatcagggaggcccacaaacccaggaaggccccagtgtttcctttcttaccctgaaacctgatcagaatacccccctggcccccaactgccaccaccattcaacataccttcatcatctgcaagatgaaatgagaaggcagcatcccttagtccagactttggccaccttgcaggataaccctgcaggcatatcagctctctgcatcctcacatttcctcccacagaggcgggactgccccacacagcccccgttttgcaaacgaagaaataaactaaagagataagcagaccatttcaaaaacacaggcaatttccagggaagagccaacacatgaacccagagctcttgatgtccagagaagccatggagtggggaaggagaaagcaaagaagcagctactcagccaccagagaagatccaggtggtctgtggcaactctaagccctccccttacggggtaaagctccacccacctgggagcgggctgcccttctccgcccagcctctccagcaccaacccattaactgcagtgaaccaagctactagtctgtctccccatcccgacctgaaggcagaggctgtgtccagggcctcacagaatcccccaggaggccgggaacagggacaggcaaagtctggtgaggagctggaattaggcatttcagtccccttctctgtgaaaactgggcatttgggccaggaggcgtctggcgtctcttccaacactgggcgggacacccatcccgacaccaggacccatcatgttggagggttgacttccgggcctcaaccaaggtccctgacctggcaggtggtcagcctcagaggaggaaataagtcatgtggcctcagctaacacccttgggctctcactgccgggtttccacacacttaggaaacaaaacccgggtcagggagcccagcagagagcaggagtccctgccccactgggtcacatttggggatgagttcctgcaatcccatcaggtgctcttctgttgccctggcaaccccaggagctcccagtggagcccatctcatttctgaaggagggggaaagggcaagcatctgctgtgcatgagacacagagtgtgtggccaactggatggattacagtgaaaggagacggtaggagaaggggcacaggtaccccagctccttcttccttctccaccctctgcagtccttccttccttcccactgacttgcctgtggggttccattcccctttaggcctcagtttgttgttgattagtggctaggtcgtgtccgactcttgcaaccctgtggactgtagcctgcctctgtccatgggattttcccaggcaagaatactggagggggttgccatttccttctccatgggggtcttcctgacccagggatcgagccccagcccgcgtctcctgcatcagcaggtgggttctttaccactgagccaccagagaagcccaggcctccgtttgccaagacggtgctcaggctggtttctcttcctcagaactgaggaaatgctcaagagctggaggtgggaggaggccccactgcggtgtgggtgggctgggtgagagctctaggaagcctctggggcttgttccacccccaagggtcccagggagactctgcctccccggggagccacaaccagcaaccgggaaggccacaggtcatgaagcacaggctcttagtcaccgcctcctcgcccaagactctggtggttgcacagaccacaagcatggtccatcccccggggagggggtcgcacaggatcccatgcaggttctcattctcacctgcacgctgctgactatactgggtgcatccttgcaagtgctagggggcacgcaaggcttccatataggcgtgcacatgaattggtacgcatgtatggtccctggaatcaccctgggacagacaccctggtccaggacagatgagtgtgagccgggaacaccaccccacagtcacgcgggactcgggctcacacccactcccaggcctccacccgcaccagtaaaggtattgggatgtgctcatagcttccttccggacaaaagaaaagtgaggagggtgaggaggaccaggaaattgggtgggcgggggagcggggagagtccaaattggtaccagcacagtaacccctagagggcctttggaaactgtccaaactcccggtttcgtagaaataaaatccggatccagaaggggaaggggacccaaggccgtgcggggagttaagaacagcgtggtggagacccgccaccatctcaacctggaccttagtgcctgccccaaaccttccacagcctctccatcttctctgccatagccttgccctcggacagcttgggaaacctgctgctctgccccgacccgggaccccggcagtccatcttcgaggtgcttggggctgatgcccaggttcctgaacccctagtttggaaggaaatccgctctccaggacaatctgctttaagaatccgatgaaatgcgcactgactccctctctttctaaaacgcacagctgcaacgcgcccacgcccagcagccccaattctgtggttcccaggacacgaccaaccgaggtgcggtcgtagggaaagcagggtagttggcaggtggtggcacctccgtcagtgaccctccacgcgggagggctatgatatctccccgtaaccctcctctcacacgccgtggtgaccctgctagctgcccccggagctgaattccgaaaccgcccgctgcccagtccccagcctttcgcagctccgagcctcctctttgcacgtctctttccctccccaccaagcctcagctccccttccgcggctccctctcttccttggagctgccggttgggggtgggacggggcagagaagagagtcttggctacggcggcagaatgtttcagggaggcgcggggtgcacggccgctggtgtgtctggggtctcgcgacccctccgtggacacactggaggctccggtcgcactcttgagtcagctttgctggagagaacaacgtccaggcagcacggcgtcggctgcccgcggccaccccagggaatggaggtgggggtgttaccgtccatctgtaggcgaaagaagagggcacaggctgcacctctgggtgggaggcccctgggagaccaccaggaactcggatggctgagaagcaccagcttatggctggaaggtttgttggccagacagaagtgtttctctaaaaaggccctgttttgaaggaaggccctcttctcaactctctagtctggaccagccgtctcggccagtggtgtccacagccccagaccaaggggtggggagcggggttgggggggggagggtgcctttcactctaaattcgctgggtggtatcatcccgcagctgagtacccccctgtctgggttttcgaatctgatgacccgccttgtgaccgtaatacctcctggggaaccgggtagcgagcgggagcatgtggccaggtggcacctgggtaagctgggtccagatacaggaccaccctcccaccgccagtgcctcagtccttcggtttcctggtggggagtggagggtcctgggagctgaggacgcggaggttgcctccagccaggcattctcccagatccatcccaccaactcccctttcccggccgcaaaaggcgcacccgctgcagctcgggcttcgcgggtctccaccccaagccctcggagccgctggggatcccggcagttttcctatcctttctcctgccgccgcttcgggcaccctggaccagaggtgaacggaaaagtccaagcccctgacaaacgccgggacccctccctccctccccccagcccccgtcgacgcgaaggtcgttatatttccattttatatttcaatttgtcaccgaaacaaagccgcacgcagatttgcgggaagagagaaaagggctgggaccaagggataaggtatgatcacggggcagcgtgcgcgcaactgctttctgaaacgaaagttctcatggagcatggcgacattttacgtttggtactgttaacttgttttcctgttgtggcccctctctgcagcgcaccaaactcggggcttcagcgacttcgggagagcctttggcggcaaggtttctggggcagccggcagttcccagcaagagtgaggactgcgcaaatgcccgacaggcaaggttattctctggagaaacgccactatcgggagaggggcaggttctctggctcccccgaagcctcttctgaaatttccttgaaacaactgaaaaaaaatcccccaccctagttttcgtttagggatacaatatgtaaatggtctgtatctccatccacgtgatagttatacctggagcaagtaattaacaattcttccaacgtttcattaaccgggtgcttcactgtatataaattataataaagacacatcgactgttttaaaataataggagacctttaatccaggtctgtttttctatttaagatgctatgtgtttaggctgaactgtttcagcagactgagggctatagaaattaacaaagtaaaaaattaaaagcatctttcttcttctcctccccacccgcaactgactggggatcctggacgtcacagctcctacgcttgagttttcccttcatcatcccacaatcatccatgggttcttaaggctaatgcgcgcccctcagtttctccctgtcttttggggggatccctcttacacaaagcacaccctggttctttggcttaatttgactatgacccacgtggagtttacatatttcgtggtgtgtggattgtgtcggtgctggtggggggaataaatatctctagcattcaatcactgcgtctaattcgacaaatcaaggccagcccctcggtggcgccccaggggtctccggccgggctcaggtctcccaagacctctgcgcggagagcactgccttcacgcgccgggatgggaaggtagcaatcggctagcaacgaaaacctgcgtgcaccaaatagaaagcgaaagagaaggaagtagcaagactgctttcggaagcgtcccggcgcgcctggccgaggcctggggtggcgagcgcggcttggagagttggcccccgtttgcgagcgagatgccagggtcccgactcctgcagggatgaggctctttgagcagctgaaaaacactgggtctccaacctggcaggtctcctggggtacaagccgccaagtatgggccaggatgggcggggactttggaagggcgacgtgtggccccagggaacctggtggggttgggattgcagaaggcaggatacggtggggctcttgcataaaatagtgaatgtgtatgcctgaagggaaccgtgactccacggttcgggaccctttcagtaccgacggggaagcagtggaggcgcagttaggggcaggaacttctgcagcctggattcgtgtctcccctccaagttccacatctccaagcagcaacccccccaactccccacaggaggctgcaggcggcttcctgctccaggctctcgggctgcggccccagtgcagcccccggacccacagctctcccgtcaaggagcgcttgtatccaagcactggctcccggcggaggagacctcagaccctcacttttgctccgagcagttacacagatggaggaactggcctctggcccccggaccaacggagctggaaaggtggttgccaggccgaagccccactgggtggcggcccgagccacagtcagcctggcaccaaagctgagtctgcctctgcctttcccaagctcctgggaacctccgaggctttttccctcctactcacttactttccccctttggggtacacactctcacgagtgtagggagagaaccatgtcggtgtgtgtgtgcttcagagtccctgcctgctgccagctccaaaggtcttctgcgcaaccgcctctctgggcggcagaacctcacctcttggtgcctcggttccctccttttgggcctaatgtgcctttccccccaacccccacctacaagagccagaaatctcctcctggccagagagacagcagctgctgagagaaggaaacgaataagcagagctgtccatagtttgcggccgcaccctgagcagtgcctccatcttggacagccggggagggcagtcctgttggtgttttccaagctgccgtttgtcccaacctgcggctttgggattttaccagcgcagggtcagcgcccgccctgtcctcacaagcgggtgcttacaggttcccagtgcagcacagaggccgacggaagaaacgtggagggggcaggctagactcctctgtctctctgggctgggggccgtgggggtgtgtgtgtgagacatttactacccagtgaggcctgactgtcatctccctgggatgcaggtgaggaaatggggctttagagaagctcctcacacgcaggtcagcggggagccccatggccactgcccatcgagggtgcattatggagactggcagtgggggcccagcagggctcagaggtggtgctcagaggggtctcacactttccccctttccctccaaagccgaaaacatttccaaatgagacattgggagccacgtagaatctctccttacctactttctcagacgccttgctggggaatttctctaaaacatgaaaaaccagtgcagagggagggaaaactgtccgtggggcgttccgtctgggatatacccacacctaacgttcccaaaggacaggaaccaggagaggcctggcgggggaggaagcctgagtcccagggggcagggggggcgtgcaggcaggggcacgtggacagggtgaaagcccctcgctgcctgagtgtggaagaagcatctgtgcggagatgcggctgtgattcacgccgcctgaactcattccaacaggaaggtggagaggcgaggtagaggaagtggggagagccccggagctctcagcctgctgttctctgggtttgagaggccacacgagtgtactaggcgtgtgtgtgagcctgctgctcctaaggaagaggttgtgtgtgtgtgtgtgtgtctgtgtagaatccaagtgtagacaagagtacgaccctgtgaatgtttgtgcgtgtcaacaaccacttagtggaccccttctgagcctgtggggagccagctccaggcagagggtaaaaaaaaaaaaaaaaaaagcatcggccgaagttgtttcctaacgttattccagtcgccaaggcctccaccccgtctcacctctgtccccggggcccggtgtctcatcccgcctggatcagtgcagccaaaccactgtactgtcagaaccagctcgtttccagggccctttgtgagggccgtggcccaagggggagcgtgtgaacccagcgctcctagcccagagattctcccatccgtctcagtttctctccctgccagactggagctcaccagtgccgatcctgtagcagaaaaggttctggccgcaggccttccttgagaagccctctcctcctctttggtcgcggcgctccggggcccaggcctgtgtcccgtggccgcgttcaagggggcttggaggtcatttaggcctcgagttcccgttgggcccaaatcaggacccagaaccttccctctggcccagcaccgcgccgcctggagctctgtggcttcctgttttcccagccgctcggtcctaaagcgtggttcagaggccggccgcctcccgggatcgcccaaggcgaagcgcttgggaactccgattcgctcgcctcgcctgctgcgagccaccccgattcgggtcatccgcggcctcgggcgtcttcgaacccgcacccagctggctcccctgcgccaccgcatccccggcgcgccccgccggctgcgcttcaggctcccggccgggctccgcacctgcgatgctcccacctgcagcgccgcccgaggagccttctagccggccaggagtcaggcctcagcggcccgggaacccggagcccaagcgttgtgctgcaactgtttcccgccgcacgccgggagccggtgctgcgaagcatccgcttcgaagccggcccggcaagcagcgcaaagcaagcggtttgcggaaacggcgaaaaggaggagaaacggactccgggttgagttttaacagccaacgctccgtgcccttggccgaaggatcccaagggggctggacggattcccccggtcccttcagagcggtcgatccccattttccgacccaaagtcacaaaccgctcggccccacgcctccccagcccgccaactctcgcctccagcaagttgatcgcgtttcgaaggtcccggagccccgggtccaggggtccccagccccgagggattcgcacatgcactcaccgtagcaggggacctgcaaggccgcgttgtggccgccgccgccttcctggagtttgaggctgccgtccgggggcgtcttgcaggcgcctcgttgcaagtagagatgcggctgcggcgcgggcggctgcggctgcggcgcgggcggctggggctggaacttgctgaaggagccccgcgccccggcgccgctctccagaggtgctgccgggtcctgctgccccgcgccgtagcgggcccggctcttggcgtccccgaatccctgccctttggcggcggctgacaggaaagttgtgctgaacttatcaccgccggggtatgccctaaaaggcgacgagccctcccggctctgcgacaccgggctgtagtaggcgtccatggcagcagccggcgactcgcagtaagagacgcaagtctcagcattcatgcctggctcgcgcgggcgacgggcgggggcgcgagcgggagcgcgaggacgccaccgcgcgccttggccgggagttaggagaggccaggaggcggtggctgtgcgctgcgcgcgggcctgctcgctccccctcctcccctccacgcctctctctctggctcctcaccccctcctttctctcccttctccctccccacagctggccaagggaaagaaccgaggactgtaaaaagattcagatgtttcggaaagttgaccagatctcccaaaccctcttaaggtttttgaaccggaaaaagagagtgctttttttttttttttcccccgactctttctttttcccttccgtctccctctcctcctctctgcctactccccttctccctcacccttaccccgtctcccttcctttcttttaaggaggtgctactaattcggtcgccactccgaggggattttacgcggagccgcccgaaggccttttcaagtcaaggcggggccagggaggtctctggactccccgggctcccgaggctaggtgggtccaagcttcggcctatgggaggggggcatgcacaactttagtgatggattaaaaaaacaaaaacgaaaaacaatccaaaaaatctgacagggtttgacatttggaggcagaggggccgctaacttctgggtgcaggcaagtcgttggggcacatccctgagaatttagcgcgcagctgctacggtaaatgtagcgcgcaactttcctgtcttcccaactggtgccttttgctctatttccaaccccttttccctattgtcggttcctccttgaaagtaacacagtcacacacacacacacccgcgcgctgcagtcaccctgcgcacggccactttcttgttaactgttttcctccctttaagggcacgggcgagggttgcggaggaatccatgttcactcagacactggagaaccagcaggctgctccagtttctgaagctttgcgcccagcccaggagattttctggctgggttaagggcgctttgagagtggggagttgagagacccgcacctcaccgctgcgcagtcacctgctcactgctcaggtgggggtgagacagtctaatctctggcctcacagtccccactagaaaagtaatggcctggatttgacgggttgtgtgcgcctcagggccctggaaccggccacagcaagggaacaggacttcactacagtgagccgcgtcccttctacacggctaatcactgattgcgcctcacagcaaccgtgcttggtggaaatcggggtccccaaattttccgatgagcaaaccgaggctcagagaggtatgagcccttgactgaggtcacacagctgggaagcagcagcgcctgccttgaaacccacgctgaggctcgtccctccgccgatctgggatgacgttgctgacagcgaaaacgaaaccacgcagaacggaggaaacggagctctgactgtaggatgactcgggtttggaggagcccattaagagcagtcctggataaactgcagagaaaaggatagaagacatatctcaagggaggctgggagtggggtgaaaggtgaaggggcaaagtaaaatgagagagggcctgagcgccctgaacccaggaatgtaggtcagaagtcaatcttctgcacctaaaaggtctccctaagaagaaatatcgggggggggggggcgtcatgcttctacccccaaccttatcctagctaatcctcacccccacccctctcccttggtatctctgctccttgctctgccgccgactgcgcgatcccacatctggtttccatcgcggcttaccttgaagtgctcgttaataaagatattctgcaaaccgtggccaggggtgcgcaggcctgaacccctcccccactgcccatagggacccgcacctctctggctcacggcccaagccaagtgaggatctcatatatactgagtccttgttgtgccagaagccatccacaggttgtctcttggggctaccccagcaatctggggatgctggtcgtctcctcatacccattttccagatgggtagactgaaactcaaagacaccaactaattttccaacctcagttagccaaaagtgagtgagcctgcatcatcccaaattctcctgttcagccctagtttcttttagttcttagactctgccaagttctggaattcccgtctcatccctgtgctttttgtcttgctaataacttggtattcagcaaagagtgtctcctccaggaagtcttcccatctttcccagtcgaagtgcatctctgttttggacttcaggtttgtctggttgccggttcagtgttcatctccatcgcactagtctgaggttgcaagagggcagcttctgcgctgcctggcgccccacaaatatttacagaataaaatattagagcctgggttccttccactgctcagcgaggcagttcccagcgtatttggtttcctctagccaaggacgaccggctgctcgccctccccctcttcatgaatcaagcgacaaaattaagagtacgctggcctctccctggccgcacaagaacagtgagttctctcagagaaggggccaggcctgtcttgttcactgcagggcttggcacatcctaggtgctcataaaatgcgcattgaatgaatgaatgaaaacactgagcgatccagtctgagatcttaaaggtgcattttcgctaaggtgttccaagctgagaaggctactccggtcccctgtgccccaaaactggctactagaacccagatgatgtctgcgatccttctacttctctcccctcattttccaattccaggagtagacacaagggggaaactaagagaagtgagctatatggggtggggaatggtgactagacaggtagaatggcaatcaccatattctctagtaacttctgagctgctctggctgtcaatgcaaaaagacaaaaccaaaaaacaaccctgctgcgatccagaaagctttctttgcattggtggtatagtggttagcatagctgccttccagaaagctgtctttgagtgctgcctcctgccacctacccaccccacgatcttatcaccagtttcatcactactccattatttaaccttttcctggccaccccccacccctcacccctcacccagggaggaatttcagcctcccagccctccaggcacagactctggcgtcctcagtcagaagccctcctggtgcactgaggtcatggttcttggaaccagcaggggggccttctacctttcaaggtaccttagaggtgaactcagaaagagtgagcatctgcctgggcctggaatgtgcatgtgggtttgcctgtgaccagtgagaacattctgggcatgttgttttccccatgaaaacctccacagaccccctgcccaagtcccttcaaacacgtcttttctaggagaatgagaagcatagctggttatttctgtgtgcgttaagtcatttgcaggttgatcagcccatgcctccctatctccctttatctctccctccatctctccatccatcaatctatccatccatccctctgccatagctgaaagccaaggctctcccacccagttctcctgcaagggtgcggggcatctttccagccagaagtgaggtggatgactctgaagcctggttctggtctttgctctccccttgcattatttcttcctcggctaaggctcctccggcttgctgattgtccctccatcactgatgtgtctggcagtatctctacacttctgatcactttaatgtgccaggaatagtgggtgcagccagtccctctgcttcaagtcctgattcctgtccccagccctggcagacacaccccttggccatcccttctctctcccagtggcactgtcccacttcaccctcttggaagcggcttcccccttcgatgggctccgcacacagcctgggaagggggagtggtgacaaagtctttgtcaaggccaagtgctcagtgtcacctcctcttggaggccttccttcaccaccctagcatctgcctggttatttccttgatcgtctctattctgacctgacattttctcccacatttatcaacctacttgttttattgtctgtcttcctaccagaacgtgagcccatagctacactcgccccaggaggaaaatacagggcttggcacatagcaccactcagcactcgataaatatctattgagcgagtgactaaatgagtgaatgaaccagtaagttgaatgaacagatgaagaatactggaggagaaatgcagtaactgcccagtccctatggctcagggcgggagctcttgaggctggggtgtagaactgagttggagaacggggaggcatggactggaagttctttcttttaaaggggaggacggattcggagccaccccctttggtctgagctccctccactccatccggcgctgcagtctccacctcctacagacagtggagctggggaggggagcagccctgcttatctgctacttgacttctcctgacagtgccccaagtcttggcccccagtgtgggtaaaaccgggaccatacacacctcagcgagtcacgtactacttggcttgcacttgccactttgtaaatgccaggaggcagcaaagattgctcgaaaggttggggctgctgaatagacattttggacatctgcagaggggcaggaagagtcaggatatgaagggggaaggccagggatcttacaaatcttgggcagaaatgcttctgctctgcagacccgggcacacagacctcacattcccagcaagacataaattagcaactgtgttttggacaactgttaaaagtcagctcttgcacacagatcttagcagtccttgaaacggcccggtgatggtactattatcctcgttttacaatgattaaaaaaatatatatcgaagcacagagaagtgaagcgactgcttacagtcacacagcaaagcagacgacttgggattcgaattcaagtgacttgatctcagaacccacgctcttaaccactgcgcgttctctgagatctctgcggcgacgcgggtggaaaggttcccgagtcctctcggcctaccgggcgctcagagaagctcccccttcgccagtgccgcgggcataggggcaaggggctgggggcgctcgccagcctcggccgcacgcgcggaccctggtcctgtggcggaggaccaggctatcgccccgaggtctgtgccagcgttcgccaccggcgtccagccttccagcgtctgcccgatctcccaggaatgcagacacctagtcaccttcctgcattcggctccagcccccgcgcagcccccgggacagccgcgcctgctgtgggatggagcccgggagggaggcactccccaccaacattctccgaagactccaaggccacgcggcgtgggcgggcgcacccccggcagtccgcaccctagtgcgctaggctgcccggggcaagaagcgagggtctcttaggcgttttgagccgagggaggacctcgcgaggggccaccacgctccgagagcgccgggtcgcctgcgcttcctcctggtccacggcccctatctctcacccggaccggtactccccgtttaggtgtttagcgttcgcggagtctggcccctggtcggctccctggtggcgcacgataggggattcagcgcggggagaggccccagaaggacctcctcttccccattctccgctttctaaggccggggagcgagggtccaaaaggaggtctgcgttcagggacgtatcttcattcacatgggaaaaacactaactcccgaaatgcgggtaaacggggcgttctcgtgggttctagacgcttgcacaactgttcggtcgggagggtcaatgaaatacaaagctggagggatgaaggtttttaaaatgaccaggcgcgacgccccgacccgcaggaacgcatccctccaccgcccttccccgcccccacggtgcgtttctgggacctgccttcccagtcgccctggtacttttcagcgtgggactggggctcctgtctgaacgcgcggtccaggcgcacgacagaacctggttccctgctccccacttatccgcaggggcagagcaaagagctcagggcagggagagagaactgagagggccacagctggcgaaactgcagtctggcgaaccccccaggaaaaccgacttggctggaaagctcgtgaggggaggcctggacatcgccggggcgaccgccttaatcctggatccggagaggaagttggaaccggaagtcctcctacgggggtccttgcaccccttgcatatcggttacagcccaggagaacacagatcattcacctccgcagataattcccaaacgtcattagccaaaaccccagccaggtgttgggactggagccccgtgtcccctctttgtcttctctgtcccttctctcctggaagccattcctctagggtttcaaaaagatgggccgtgggaaggtggaggccagaacacaccagctcggttatacaggagtcctgtggcgccttgtgggcagaaatgaggtcttatttcctgggggtgcttgctgggtggagaggatctgcccttccccgctcagccttagttctagggaagtatatttcgaaactttctacccatttccccgctggggagatggaggcttcagtgatggagggaagggagtgtggatggccagaggggcctctgtgccgtgcgcgctgggactgctgagctgacagcccacaggtctcttactgtaaggggtggtcttcccaatctaccgctcagataactgagctggagccagggagggggagaggtgaatgggacaggctaggcttcggattctccttctttctctctgagatccttggggtgagccctggggggccctttccctccgccctcccttctccactccgctcctgcccaccaatccctggagcctgctggcaccacaccctctcagtgccctgaccacactctgctgccatcactgggaaggggcggggagcacggcccctaattcccttttctcctctcccacaaccccagagtccttctcctctaaggtttcgtttgtccctttgtcccctccctcagccccaagaattcgtgggcagccagcagcagttgtgatgactctaattcctgcagtgcccccagcgctggtctcatggatggtgggaggggctactggactggaggacagagctgggctcaggagccctctgtggcaggcctctggggctgggaaaaaagtgggggagggtggttcagggaggaggcagaattagtgattccattagtggtcaagaggccagctgtagattcaagtttgaatcccatttctgccactttttcgctgtgtaacacaggctatgtgacttcgctcatgggcctcagtttcctctcctgtaaagtggggagattagtagcacctatttcattgaacagctgccaagatgaaataagatcatgctcacttaaggagttttctagtatcagcaatggtaatctcctttctgtctggaacactctgctcttttaaacttcacagcccagacttaaggaaccagaaatgaccagttggagttcctgcccttcaaccatcgtgacggccggtcatatttatctttcagaccacagctcctccctggtcagggtcttttaaagcaccaggagtgaaaaggccaaaggatttttcaggctctgcggtccagccattcctgatagttggtgcacataagatggaggcggtgcgttgtgttttgcagaggggtagctgcgcccaggctcagccccacctcagcgctttcagcagcagcgccttcacctctcttgctgctgctgctgctgcaaagtcgcttcagtcgtgtccgactctgtgtgaccccatagacggagcccaccaggctcccccgtccctgagattctccaggcaagaacaatggagtgggttgccatttccttctccaatgcctgaaagtgaaaagtgaaagtgaagtcgctcagtcgtgtctgactcttagcgaccccatggactgcagcctaccaggctcctccatccatgggattttccaggcacgagtactggagtggggtgccattgccttctcttcccttgcttctctgtaaactcatgactgtcctgtggtggagggtttatgtcatcgctttacaggcagggaaatggacgccccttgcctacagcacactttgagcaagaggtcaggatgaccctcaaatacggggctcctggctcccaggcctggtatgtttgtcccagcgtccagagcgcgcaggggcctcagagacagggccccggggtctggccacagcgcctcctgcctgttcctcctccccccaccgcctgcctccaggcggtgaggtctgggccccagcacctgtcgaaggagccagccggagcgcaccagctgatccgggtaatccggggctttgtgattggcagggaggagccctggaggcgggagggtggggaggaggggaggatgaaggagaatgcgggagacaagcacttgttgagcaaccctggatgcccgagacgaaaggagagactgataaccaagggcgcttgactcagtgcctggcacatagtaggtgcttagtaaatgatgatggttattctatttctatcgcctcctagtcttactagatgtctgccaacttacctaaacccctccatctgaggatgtcatgccgtaacgttcaccctctctcacctaccccaccaggggtccttccctccttgggaccagtggccgcactccccgcttgcctggagcccggtccagagcgcacagctgcggccaggctgtgaacacttctggagtcaacctctccgcccagagcccaggggtggggggtaggggtggaggtgggagtggggtcggggtggggtgggggtggggagagcggccttgaccgaggagcgaaggacggaaaagcagtgcagccttaagtcttcagggacgctatggcgagtgctggcaggtcgccccaggccggagggaattgacttggaaaacgaaaaaaaggcaactgataaaagaaacaaccaaatctactcccccctccaggcttgagcagccgccagaggccagaggcgaggccccaggaacccgcccgcggacatccttaggaggtggcgtttttgctgcatttattcggtgtcaattcagggcccctcgcacctctgctgatccgcgcaacatccctcccgccatagccctcacccaggggctgcgaggctgaaggggacacggccgaacaccccgggaaagggcaacccggccggagtcggcgctggaacgaactttgactggagagccgggccctgcgttctcaggcctccgcgcctttacgcgctgggggcttggacacccaggttcttgcctcagcctcttctccccagatgccttgccatcctcgggagcgcggaaacgcctagctgctttctccaagtagaattcgtttccaggtcgtagtggaatttttcaacgggtagttgagaacggtcagcttctggaggcaggcatctgggcgctagccctgatccgggctggtgcaagcttgggcgatccattctcctccctgtgcttcagtgtgcttatctgtgaaatggcagtgactcttctcatctcctgggaaagctgtggggagcagagatcacctgggtaggtgctcccttaactccagttgttaatcttttgggggatagttgcagatcttgggggacttggaggaacactcagttcttgtggccaccccgacctcctgtccagttcagatcacccctcgccaggattccaggattctgggattcctgagtgcttcacccgggaccatgtgactagctctttgatggcccatagagtcctttcgtggtgggtcttctttaaaaaaaatttcactggtcttgccctcacccttgccaagctaactggactcactttcagtttcttggggatgaaggtagcagacaaaatgagcattgccaaatagcaatcgatgcatttttttcttttcctgtcttaaggtgaaatccacataacttaaaattaaccatttaaggcttccctggtggctcagacagtaaaagaatatgcctgcaatgcaagagacccaaattcaatccctgggttgggaagatcccctggagaagagagtagctatgcgctctagtgatcttgtctggagaatcccatagatagaggagcctggcaggtccatgaggtcacacagtcagacacgactgagcgactaacatataaccatctaaaagtgagcgattcagtggcatttagcacatttgcagtgttgtacaaccactgtctctgtctagctccaaaacatttccatcaccccaaaaggacctatttttctttttcaacactttctccagaactgagctgtgtgctggcggagagcgagaggtcagcacttcatgccctttcaacttatcttttcaatgcctttaacaggagctctgtttacaaatgacttgcaaagttgagctcaggaaaccagggagccccggagatgggagtagggtgtttattcagaatccatggggggaccagtgtgtgtctgagagtgtgggctgggcccagggaattattggacagggagctggagaaccaggtccagagagagggtctttaatggccctgcccatctaaaagctatagataaaattctcatcctgcaggagtgaggactctatttcacagacattttaaatcacaggctctcagtctctttaggaaagctacttctttgatcatccgatgaaagctgaggaccctcttcttggacagatgcagcgaaatgctgccttagtttccaagggtttgtaacaaatgactgcacacttggtggtttgcaagaacagacatttattctgtctcagctctggaggccagaagtctgaaatcaaggcgtcccggggccaagttcataggaggggacttgcttgcgattcccagcttctggtggctccaggctttccccttggcttcacttttcccatcactaccttcatcttcatgaacttctgtcttctgctctcccgcctcttataaagacgctcatcatcagaattagagactacctgctggagagcccaagatcaggtcttcttgaggtcctttacttaattaaactgtaaatatcctttctgtagggcttcccaggtggttcagtggtaaagagccctcctgccaatgcaagagacacaggagacccggctttgatgcctgggtcaggaagatcccctggagaaggaaatggcaacccactccagtattcttgcctggagagtcccatggcaggctaaagagtcaaacatgactgtgtgactgagcacacatcttttctgtaaataagctcggtctcacaggtactgtggagaaggcagtggcaccccactccagtactcttgcctggaaaaccccatggacagaggagcctggtgggctgcagtccatggggtcgctaagagtcagacacgactgagcaacttcactttcacttttcactttcaggcattggagaaggaaatggcaacccactccagtgttcttgcctggagaatcccagggacgggggagcctggtgggctgccgtctatggggtcgcacagagtcggacacgactgaggcgacttggcagcagcagcagcacgggcactgtggccactgtcttgggggactaccctttaacctcagtacaggtgcatacagccagcttcatactcaacctcaggggctctcaggctcctgtcaagtcagaaatgtctgattgaatgtcacaaagcgaagtgaagtcgctcagtcgtgtccgattctttgcgaccccatggactgtagcctactacgctcctccgtccatgggattttccaggcaagagtactggagtggggtgccatttccttctccaggggatcttcctgacccagggactgaacccaggtctcccgccctgtaggcaggcgctttactgtctagccaccgggcaagtccaggaacgtcacaaaggaatccttaactgccaccttcagaaaggttggagagagtagaaaccacccttctctttaaggaaggacattcccacttgcggttaaaaggagaaaatgaacccaccatcctgcctcggtgataccatctgccctcttgaggaaggacattccagaacattctagaggcagaaaccagttgcctttttacaaagaagcccttgaaaaatgcaagcatcagccagaatctcaatttgtgaaaatggaccgacaggaaatttcttcaccctgtcagacaaacttcataaaggctctgtaggggccctaccactccgagaagccttttaagaaaattgcaacttttagtcagaccctgagggaagtctcacaaaggtcttcacacccctttggctcctctttgtggggtggtgagggaagagcaaggtttttttttccaggggaggagagaggtgagggatcagggctggggagaggctgctctgaggcagcgaacaggatggctccacagatagaggtgacagtttcctcaactgagtccccgggtcgccacctgtgtccttcctgcagattggatttatagagtgtaacattttaataaaaaacacacacaccttcccattttggtgcattaagagatttctaacaaaacactgtaatgttgctataaatttttctcccagcaccctcacaagtaataactcagccccggaattttgcatttaatgtttattttgatggctacattccagtcagagtgggggctcagggtggggagggagaaagcaaaaaaaaaaaaaaaaaaaggctcctctttcctagccattaactgtgtgtttttaataaaaaaaaaaaaaaaggtccccacaaattggggagccaacacttggaaaggaaggagggaaatcatgacaccctatgaggaagcgccagtggcctcagctctaggaagttgacagggtgagccctggccgggtcctgccaaggtgggtctccagagggtttctggaatgtcgcctttgcccatgccatgcattcagcctgagatgcctttttctcttccctcagagcttctgagcctcagaagtctcatcttctggaattttccccacatcctttctccacgagagttatgggcccttcccctgaacagtattcattctattcactcaacacttgtagtggaatctgtggctggaacgggctcagatgaaagcgtattgagtgaatgaacaagtgaactgagtaggttggagaattctattttattccatctgaattgcttccgcgaaggcagctattttggaatggttaatgattttctccctaaaaaagaatctaaatagttcaaaatcatgtggagatcagatcacaaccccttttccttcaaaagtgctatattttagttgtcttatcccccagcgtcagaagggaagcttctagtttattcttttgcctccaggtagaaacaaaagatgggatgctggtccagactgtcacagatgggtggagaccacttcctggaggctgaggctgaagagtcccactgcctgatagctgattaatgctcatcttccctgtgccttggctgtggggctgatacccttctcacaacaccctctccttctaagaacatcacattcccccagcatgtcttcccatggggccactgatcaaagcctgggttctggggttgccctgttcccgaggcaggggataacactcaaattgtcggtggggataatgaatagagaaaaatgcttgtaaactatggagtgcctggaacacagagggtggcccctaacagcgtgtatgtctcagtgactgtgaggcacgtaagtggggggatcagaccccaaagtcagatgatttgcgcttattggtgtaagacctcagttttctcatctgtaagctggggacaacttcagcagttggatcctgggaggtgggagaaggggaggattatttgagaaagttgttcatggcatgtgcctgactcactgttcaggttgggaggggacctggccctacccctgccactcactgacctatgggcaccgtcagctggctgggttggagcccctcgctttccttacctccaccgcacagccttccccagaaaggaaggacagcctgttccaagctgctttactttgccttctccatggcgaaaaactgttttttcatgaagggtttcaaatcctgcctcttccacttatttgctggttgacctcggatcaattgcttaacctctctgggcctcttatttgcttatctgataaatgaggatgataatgctggccttggtgcattgccctgaggattgaagacgataaggaagcctggtacacagcaggcacttgataaatgttctccctttccctaaggagatgagaagcagatactggatgtgatgctccgggtacctgtcctaggacctacgcacaaacagtgcctactaactgctctggaggtatgggccggcaggaagctgcattgcccataatggggagctctcaggaagaccccctcactcatcatgctctgtgaggacaccatacattcttcccttttacaaactctcagcctggcccattgcattttaaagctgaataaatagttccagggcaaagccagggatcagggctgctcttggctctttccaaagtatttctgagcctttcagcaggatccgagtccccaccctgcaacccctctaactttaatgggtgctgatcactttacaccctgtgaaaccctcagcaaatggtcctgatctctgatgtgccgttcaggctggcagcagggcgctggcactcagactctgcccgaggaaatgaaagcctgatggggctggagggggaggtggcgaaggagcttccaggccatcatggcagctggaggaagttttcccctagctgagccgtcctgcaccctgccatctctcccttctcaaacacggcagccaggcctgctccgtctggggcctcacaaaggcccttcctgagcaagctgtgtagatgtctaccctacagaagtaataatcgacaggaccatttatcgagcgcctattatgggtcagacccaattcttgacttatcccatgaataccctcttctgcggccccctggccaccccgccagtcaaaattagctccctcttcggggactcactgtccctcacctgctttaattcttcagaaccatttgtcactagctgtaatgatctggttcatttatctttttcctttgtctactatacatctctcctattacaatgacgggaaccttgtctgtctgttcatacaccatggcccggagaagcgctcagtgcagaataggtgttcggaaaatattttatttttcatctgaatgaatcttcattcatcttcagagtgatcttatgggaggggactatgattatccccatgttccagataggaaaactgaggcagagaaggggccaggagttttgacaaagggcacacaggaagggcagagctgggattctgaccctggaatgtttcactgaaatctgtgctgttctgccccacacaacctaaccccacctacctggcttccgatagagtgagtgctctgttggaaatagaagatcttgtcagaaatggaaacttgaggcccctgtgtctgaataaatacttcccgaatatttatactcatgatcctacaatattttattttctgtagttttcacagaaacccattacattgttatctttctataccattttataacgaagaagtcaaaactcagagaggttgtgtgcttgcctaaagacacacagcgaggaagtggctgagtggccttgggtctggactgggatggaaaggctgtgctcatctcacttctcagatgcatggcctcctggggaatccgagtgacccctctggactcacacagtcgggaagtgacaaagcagatacagggccaggtcttgtgatgctctgcccaaatctcccctatttcagtattgcagcctggaaaccaggcacacagaaggaaggcatgagggtcctgaccgagggcatgatgtggttgccttgagggctgggaattggcaaggaaactcacaaggtcatctgcatgtgtgtgtgtgtgtaaaacacaaagatctgaccctgccctaccctactgcgtggatcagtaactgtggagggaagacaaggggcagagaagctggacaaactagccagatttatatgggaaaacctgcagagacataggaagtaggagaacatcgagaaagggtgaagctgcagcctatggaggagagagggaggtttggggggtctggagtcaaagaagggaggcttcggaggaagctgccaggtctaggagttgggggagcagagggggagagagagtcctcaaggtatttttccacctgataccttgggaatatgatcttccttttctggtgagtctgccatcaacattttaatagaattttaagttacctgtggatgctggctgacattttgtggattgagactccgtgtcgagagagaggcatgagccagcctcgtttgcgagggttcatgtgtctatggggttctctacatgtgtgcacacgtctgcacatgtatgtattcacgtgggtgggggcgtttgctctaactgaaggcctgtgtgcatgtgtacgtgcgtgcctggggtaccgatggcctgtccatcccccctgccctgccccagattgtgaacacagcccccacccaggagtggagtcagacgactggtgtggggagaggggggttggagggtctgaattgggaggagtgccccacctccattgtgggaaagctggcatttgtgggggctaaaacagggtgtggggggatggggatgggcttgcaggggcacaagacacagaattcctccagggggatttagcagagagaggggaagagagcagagagggaggggcccaggcaaggggggggtggtccttggcgggggtgggggggagtggggacactctagaggcggtgagggacatccagcaagactgtccccatccctgcctttccctgccagtgatgctggggcctctgtgaaccgatggccacgtgcaggggctcagcagccaaggaaccacgcacagggggagcttttgaacttagacccgagaaaccgggttctcagcctagctcagctcatcccaacaggctgtgcaacctgggcagatcacctgggctctctcatcctagcttcctcactgtggcgccacctggggggctggaggcctgcatgggcgggtgggtgggacggggctgggtgggctctggagtgccggtgtgtgaggcctgggtttcttactgggatcgccagttttggccctgccctgggctcccaagaatttctggacctggtgttcagcaagcagggtacctattgggttgtctccaaaaaaatgttctcagccttcattttgttgtatttaatcttcttgaatgtgaccaaatcagtctgttttaggaggcgggggccctcgtgcaggtccacctgccccgtgatcaccctccccgtgccaggggggtgactgatgactactccctgccctttttggtgttaactttaaaaggtgttaactgcccttggtatcccttcagacccagctctcgcctctccaggagggatcacctgcctgggtgatcgctgaatacacagaagcagccttggcagttggcatgagtgttgccactgactggccagggaacccagattcttgaccctcggcgagtaagcactggagccagcaggccggactcaaggccttttgtctcctagcccggtttggccacagactgtaatactatgttgatgacgaacttatttgtcaaccactttcctctttgcaagtatcagtcatcccccacatcgctgaatgtgtttgttaacacttggtttttctggccgtgccgacggacacacactgttctgaaacgtgtcatgtgatggctacacctgagacaagccttgaacaaatggtttctacgactgctgccttgcaggtgtgtgtataatggtaccctccttgccctgatccctttctatttttttctacgctattccctcccctctccgatcacacttttccttgagcctccctctcccccacattccctctgtaccctcttgcaacctggtctgcccccgtcctctctctctcccccagccccgtctctggagccagagccttggccaggatgaaagccactggccaggcccataaatcagccggcctgtgccccttaaccttccccattaacctctcacacgggcaggcattaggtgttacacttggctcccgtaaattacttgccaatgaatttatgagtgctcacccctcagaggcctgcttcctcatcctcccccactttccctcagggtttccagacatcgagccccagcctagcagtcagtgcctggttctggcccgcgactccctgagtgatgctctgcacctgatccccccaccccgtttcacacatggactagaagattgaagaggggaaggggggggcgggtgtgcgtgacagtcaccccttggctccgctgtctagaatccccttccccagaggccaccccctggagcttccacacgtttaccttggaaggtgagaggaccctgcataagaaccgtcaccactgtttattgtgtttctgccacgtgtttctaccacgtgccagatgatcatgctcattgtttctaattctttgaacaattcttcggaggtatgaattgttatcccgttttacagatgaggaatcagaggtgcaggcaagtgatgtgacttgggcaggaccgggcatcactggtgctgagagttagtggatttcgttgtatcctgcgtgcctgctcagtcgctcattgtgtctgactctgcggttccttggactatagcccaccaggcttctcagtccatgggctctcccaggcaaggttactagagtaggtggccattgccttctccagggaaacttcctgacccaggggtcaaacccaggtctcctgtattcgcaggcagattctcctaccaactgagctcctactctatacaaatccaaatctgcaaaaacaaggccacgtatggaaaccgtgataagcccgtgacccagaatctctctgcctcactccctcatcttgctgcatgtcggtttacaaggaaaaatagtagtgacgatgttcatcacgagaatggctgacccccgtggatgtttactaagcacctgttattatgccaagcgatttatgtgcagaaacttgcttaattatcatccctgtgtaaggttgatgctatagttatgcctgttttacagaagaggacataggcaaaaagaggtcagttaatttgttgacggtcacccagtgaggaagggccgggtccagcggtcggaccccaagagtcagcctgcagaacgcccaacacttcaccatgagtagtgacactaatgacaaatgtgatgccaccacagatagagcgacagttgcgtaggcgccccaggggctcagtgctgttagccccgcgtccctgcggggagcccctggagagttctccgtgggtgacccaggggcgcatctcacctggttttccttctttcgcttttcccaggtgacagcaggggcggagctacaaactttccccagatgagcccaggacctctctgggtgcctgtccctggctgctgctccagctctgatccctgatccccaaagctatgaggtctccacctcagcttcctgagtgtgatcctggggagacgggagcacatgtctgggatggcagggagcagctggagggggatgaggagggatgcgaggagagcacgctgtgttccggggacctgcagggacagcagaccgaagctggacggcaggactccaggaggatgtttctgggccagctgtcaccaagggccctcccctccctgtgcccccaccccccgaacttcggcccatgtgaatgcctcctgtgcaattcaccaagggccctcctctccctgtcccccccctgaacttcggcccatgtgaatgcctcctgtgcagttcagtcaccactgcaaaggaggggcagacagctgcttgtgttcctctgggttgtgtgtgaccctgacttagagggaaaaggaaaggtcagccagagggtgctgccacaaaggcaggacgcgggccctgttatactgggagggcaggaaagcaagagccatacagacctggctcaagtcccaggggaacagggctgaatagggtcccctcccaattcacgcccacctggaacttgtggatgtgattttgttcggacaaaggtgtaactgcaggtgccgtgagttaagatgagatcaacctggattagggcgggcctggcatccttgtaagaggtgggaatctagacacagacacgcaggggcgaaggccctgtgagatggaggcagagatggagtgtcagatccacaagccaaagaatctcagcggttgccggcgctccccaggagtgacaagaggcaaagaaggatcctttccttagaagagagtatggccctgctgacaccttgagtttggactcctggcccccagaactggacgagagtaagcttctgttgtttcaagccactcagcgtgtggtactttgttatggcttcttgttaagaaagctgacaagcgagctccaatcacttatgagcaggatggcctggcatcagtcactggtctctccaggccttgctgagcatccttgtagtgagtgtgacactagttcagagggtgctgtgaggatgggatggaatgatgcctccaaggctaaaacggttcagtaaagacagtggatttggctctgggattcctgggtcaggcagatctgctggagaagggataggccgcccactccagtattcttgcgcttcccttgtggctcagctggcaaagaatccgcctgcaatgcaggagacctgggttcgatccctgggttgggaagacccccttgaaaagggaaagtctacccactcccgtgttccggcctggagaaattccatggactgtatagtccatgcgatcacaaagagtcagacaaaactgagtgactttcactgtcatgtctaaatacagcgattcttcagagctgggcttcctagttccttctggaaggactgagagccccgtgttatttttttatggttaattttggaaattccaagttaacagttagggtacaaggtgatcacatttatgtaatctttatcagaagtctcacaagttgtatattcaacattttgaagaaaaaaaaaaagcaactctagtttttactctgtacatccagattacatgcttggaccaactgcacgaacagttagtttataccagatgccagctgactgcctgatgccaactggaaataaagttatgtgcgtatgcacactcatataaactgatcgctaaccttgtatacaaaaccatatgtgtctatctccctatgcatattggatgcttacatattcataacagatgcgtacatatatctgctcaagaaatccatccagcaaatatttattcagtgttcaccttgtgcagggtgtgtgcgtacatgtgggtgtgcagatagagaggaatacgtgttcatacataaataatgtatacacgtagctgattagaccagaagccaaccaaatatctagatgccagtgtgggagcatggctgttggaaagccatatacttcaaattccctggctgggaacaggccggagcctggaggtgagctgagcccccaaatgagagaggggtcagcacagggcagacagctggggcgggaggaaggccttgggccccctctaatgccatcggggtgaggcttggggctcccagccccttctggtcagttgtccccaagctgtcactctctctgtctttgtggggacttggaacggaaacccattttgagaagagtagcatctaggggcacggcgacctgttcctgccttctcccactctgagcatcctcagagtgggatgtatcctctctggatacaggggcgctggatgctttgctggccccagcaccactttggagacctgctgtgtgcttctcaatgccaccccctccctgatggcacttctctccgtgaaggggccaggccggggaagggctgctctgctgtcacgcttcctttcgtaactgaagtgatacaatctccgcctgccccgcctgctggactccaagggtgctgttgggacatggctgctcttcctccacagcccagcccagagtctgcttcctaggaggcccttcgtaaaatctggggcactcaggggtttagccaggtgaaggggtgtgtccagggccgccaggcagtggagggaggcttatgcaaacacgccctgactcagcagactccctgctccagaaatgctgcagctcaccacccgccttggaccaggacctccctgtgccacggaccttgtctgaccctgccaaccggagaggggaatgcctggggtggcccttgtatggcactttcatatctgaatttccagtaaccagcatgccaacctgcatacagtaggcaatcagctggtgtttgctgaaagaattaatgacttcaaggcctgggtatacagctctgggccctcgggactgtgagggctcagaggagcagtgtttttgccctggtagacggcgaattctctgctaagaccctaaagccaatgtggcgtcttaggggagaggtgggtgggcagtcagcatggggcaggaattggctgctgggttaacagaaaatgaacgagatttgcctgtgtcagaggggctctggtcaaggacacttcctgtaataagtaaacattcctgaaagggtgagagcttaaacacagtaagtgatgctaaatggaggagagcagttcaaggagggagattctttccaggtgaggcttggaaggggagggaaggaaacgatctttctttaagctcctgtgaagactgatgctagagactgcacacatgttcttcctggaaaagcaggtcactggaacgatctctgtttaacagatgaggacactgaggttcagagaggtaaagcacctcgctcaaggtcacacagccaggatgtggcaacatcggtccgactcaactcctgtactggtctggctgctgaatctaggtaatagctatgtattgcctgcttgaccttccctttttgttctttttctgtgaggatttagttattctctagagcaaaaagaatatgttttgggggtattcaagctttggggatttttttttccgtcctctggtatattgttaggagggcttccgtggtgacttggtggtaaagaatctgcttgcaatgcaggagacttggattcaatccttgggtggggaagatcccctggaaaaagaaatggcaacctactctagtattcttacctggagaatcccatggatagaggagcctggtgtgctacagtccatggggtagcaaagagttagacatgactgagcgatcaaacaacaaccgcaacaaataccgtttggaagggtatcagttctggcctcttccaaaggctccatcaaagataacgtgggaggctttcctggtggctcagtggtaaggaatccttctgctagtgcaggagacacgggttcaatccctgatccaggaaagtcctgtgagccacgactattgagtctgtgctctagagccccggagctgcagctactgactccgtgcactgcaggtattgaagtccacgcccctcgagcccgagctccacagcaggagaagccatctcaatgagaagcctgtacatggcgactagagagtagcccctgctcctcgcaactggagaacaagccctcgcagcaactaagacccagcgaaggaaaaataaataaataaatgaaattattaaaaaaaaaaaagacaacatgggcttcctgcaaggtgcccctccaccataaacctatccttcctgctccttcaagagcgatccctggatgctgtagtgagaaggatgtgtttttgagggggcctcttcccaggtggtgctagaggtaaagaacccacctgccaatgcaggaggcataagagacgccagttcgatccctgggccgggaagatcccctggaggagggcatggccacccactccagtatttttgcctggagaatcccgtggacagagaagcctggtgggctacacaatccatgaggtcccaaagagccagacacgactaaagcgacttagaacagcacagctcatccctgtcaactggtataaaagcaaattgtttgcaaacttagatgctggagagatcaatgaaagaactaaaagtgtttccgcgctgatcccataatcaacacacgtctggggactgatccgaccgagcctgtgctagtggcatgtgttcgactatccccaggacgaggcccctgcaggtcacaccggtgtccccttcccccagaatcccaggtaagtggcagagagccttggcagacaaacctggtcgaaaacacttcctgtaataactaagccagtctgtttatctagcttcttggctactaaacaggcattgtgagggagggggcccttcgtcactaaatccccacgcccctttgtagcgagtgatgtcctttgtaagttgctgggtgtccctgaacaagttactccagccctctcttcctcatctgtttaaaaggggattatattaccacctcctacgtgctgtgaagttttagcccaagcacttagcacagagccgggcacctgggaagctctgaatgaacattagctgttaggaccacagttagcatcatctgccgggcagggctgatggaagagctggtgagtggaggtgagtgtccctgtgtgagggtgggagggtgtgtgtgacccctgcctctgctctggggacactgaggccctagggcagactctggagggatatggataagtctcctgtcttgcttgggcagactctggaggcatacagatgagtctcccgtctttgctccagcgaggaggaactttgtcctcccgcacaggctgcctcctgcagactctgtcctctggacccgctgcctgtcccttccctggcgagggcggggaggaggcccagccacggggccctggcccccggggccgctggcccagcaggccaaggactctgtgtcctgggaggggagcagccctgcgccgcagctgctcctcctgggaggggctggggcagcaatctcatctctcagcagagacctctcaacccagggggcagtgccccagaaagggcacaaccccgagacccgctcagagctgcttcctcctcctgttcatgtgtcccagaaacctgagaaaatgagccaagtcctggggagagggggctccagctgggggacccggtcctccttgacccccacttactgccgcttgggggtggggagagcggtcgtccgccctccaccctccggaggtcccctccgcgtcccgccctccctctcaggccacgggccggttttccagacttctccctttcccacactctttccccgaagcctccttcacacacccagttcttcttaaaggggttctaagaagaacgaatttcctatgaacagaacaatgccctattaatcttacgagggatttgaacttggccacatttcagcttttgatttaaatatcattttggtaatgggggagaaaaaaaagaaggctgactcttggacagccccgagtttccaataccttctgagcatgagacgatttcaaatataaaattaaatccaactcccttctgcctcccccctccccccaccgtccccttcccccgctttcactcagacaaaaaagctcacgtcccacatacttcatggggatatttcaagttaaagttttgtttaccttatagaaaagttaattagttccttttgatctcacggggaaaaccacacaggaaatttttcttcagaaagtgtacacacgaaggcatgctttaccctcataaccagagatattcattaaaacaattcgtttcgatttttaatttaaagaaaacattttacattttcctgatttattattaagagagtagttgtccctggggcagagcgtgaagggggtcggctgaagtgtttttgctgtgctggcagccagcattgggcagtttgttttgctactttggataacaagagacttctgcagagaggcaaaccttcaaacacgccagatttcagtcgccaaaatattaaaaatccagggtttgggagattgggactgtatggtttaaataaagatatttaagttctgaaatataaatattcgagatcctgaaaaaacatatcattgcattaaaatctgaaacaggcaatgggtgaatcgaggggagaaaatgtttacttttttgaggtttatctaatgaaatataataaatagtacatcatgctcttttttttttttaacccctagagatatttaagcaatacctggatttgtacaataagctgcttaggaaaagtctagaaagagaaacggttggattttcgaaatgcgattttcaaccttaagcacattttccacatcttgtattgttatgggatgagggatgcagaggaccctgtaaattgtggaactcatttccagcccagaccaattgaaaaaagaaatagtatggcttccccgaggttgaaagaacaaattaaaagatattgctgatggagcagccaaaagctgtggtccccattactgcaaggaaaaaaatcttaaatagatatggtatggacaaaaagcctgtggaagagatgtttttactaaagcaaaaccgctaaagcaaaacagctctaaagagtggtgttgacatcagtatacgattttgagggtatttttgggtgcgtatccctttcctgcccctgacacaaatatttggagattgggactgtcattgttgataaaaattaaattgatgccgcatacaaaagatatataaaaaccatgttaacatatggggcgactttgatgatgttctgtacacagcaaaagtataaaaatctgatgacattttattgtcaccaatttgctttttaatctgtcccaaccactgcttttaaaaacagaactcttaatatagatttctttatcaaaagtttgcacctgaagataaattttctgatttaaatgtattccactctcctattttttatttttgttttttggtaaatggtgaaggtgtgtgacagtccgtttgtgaagttagggaagaatgcatttattttgaagagggatttaaaaatgctggctgtaggagcagattcctacaaatgaagccagttcttaaggcaagtttccaaaatctttctgttcaaatacagagtctgaaaatctttttaaaaaattgtccttctacaacacaattagttttatttcctgcctaagatacctttgtattttcttctgaagaaggatttaccttttttttagagctggaaatattcccttttttcttcttttcctggaggaaaatggtttaatgtaaagtttaagggagggagagagatgtatggaaatgaatggaaaacagaagcttcaaacggctacccaattccctctttggatttgccaaataacaactcaaaaatcagatacttcccataagaccattgatgggagaccttcccaataaatatgcatctattcctgttttcatcaaaacaaataattttctctgctgattaggggcccttccttctccagtacccatcattgggaactttgagatttcactaaacatatatacatatatatatatatatatatatatatatatataattttcctttccgtgaatttctacgaagttggctttctttctccaggaggctggagtagagtcgggaccctgggtagacacttcattggtctgaatgcaagagggaaaggggtgacactctaaagggaaagccacgaccccaaaggagaaagcacggacagataaaaagaacaagacaagtaaatattttcctagcgcgtttccaagttaaaaccgatcaagctacccggttaaagaaaggcaaaaacaaaattcgtccctacctttgtcagatgcaaagtaatgaggggttgttcccagagtggttatgacctacacagagcttcttaccttaaatctatacattttgaacaaaaacagcttggagcaattctctccaaacaaccccaaatgttaaccattccaactcaagatcgggtttctccttcagcctagacggtgatatcagttgggttttcttttcttttctttctttctttctttttttttaaaatatgtattcttatgtttccaatcctcaaacatacttttttcatctttgttaaggctccttctggctgctggatgggctgggagtcgcaggagtccttctcttcgggacaattagtctctcacttgatcacttgaaaacggaatgatttgtcgtggtcactgagtggggctctctccctctttcctctaatattgttgcagatacgtaaagtttactgcgtgctgtaaatatgtcagcttttgccagggagtttgctaataaagaacctagaggtattattgatggaaaagataatgttttcaataagactggcccgggctaaagtctgagacattccacacccagccaagcataggcgtttgagacgccgaccccctggccgaggtgccgggctggtttaccggccacaggccggccgtgctgcctgcttctccgtggtccccgtgactgttttattcagcgcagttttaaacgcccgactggttttaggagctctgtcctttctgaaaaaatggccttcctgaccgtcaggaataggtgaacttatcagagaatcatgagatacgcttgcccagattgatctctttggggatttatcagctcacccatgctgagagcagaggaaaaaaaagaagtccacccttttctgtttgatgctcactcacattttcatttgtcgtgacaacttctgagtgtaaattggattcattttttttcctggatgttcgtttgtggaggattgtttggcttccctctcttaaaggaaaaaaaaaaaagaaatcgcagaaagcttggcgtccgtctgccccacggccctggtcccctgatttcagtctcctggtctcggtgttttgtcacatcacccacatgacagtttcatctggtgctaatgtggccgtcacatgggttggtcgtcagaattccatgctcatttaccattaatcaaatgcatcattatgaattgtgtatgcattttagggcagacccaaaccccggcttgataggaaagtgtttttgtttttttccacttaacgttcggcaacggcagatgaatggcaaggccaaagtgacactctttgtgtttgcttagcccagaaatcaacaaccctaaattacaggttggtaagctatgtccaggtagcattagcttctctgggtgattattattacttaagggcggccacagtcagcgccagcaaactgaagctggccagagggagggcagacaaggcgagcagagagcagggtctcggcttccaaggggcccggtgcctccttttgactggggggattttagtcttagattgcattttcctggaaatagggactggcccttctaatttggaagaatgtgaggtatttgcagggcgagttcggatgagtgggggtggggacgggttaccactggatgcttttggcaatttgtggactctacttccccaggactcacttgccagcttggcctctgtgggaacattccaagttctggaaccttccttggaaggcctgactcctgttggttggtggggacctggcgtctcctgggcgtataaagacggttttcattgggagttttcagtgattaaagtctttcctgaggacgggggactgtgtttcaggtaaaaaccctttcctgagctgtcacaatagttctgggagatggggggcctggtgcctgagaccggagggccccaacctgacccctctcccccagcgagccttgggagacagatctgacctgggcttagctcccattcgttgtctaactgccagccgtcccaggccagatcgggaacagggaaggaggttggtcccgaaaagcgatcagagacggggtacaggggctgcggtgctggcttatttgatggcgcaattttttttttccttcccagcagacagctgggcctggttggtggcttggaggcctctcccagggaagcggcggagcactcttgcccccaggaatgtctcttatttgtgaggctgtcctgggctacttgggagctcaggatgttaggaaagcctgttaaaagataaataccttttcagaataagctgctcaggatccaccttagcctgcggtgggggaggctccaggagaggcagagagtgcagtgctggggggccagggtctctcatgaggcctcctgggctggctttgggaatgttcctcccttcaaagccccacttcagcctggaagggctgagccgcttccccagagggcagtccagggccgcggcacacagacttcagcccttctcccctcttctcctggtcagcagggagagatggggctcccgagagctgggtcgggtggcagctgctggcagaggcctcagaggtgggccctatgcatagggctggggccaggggagtgtgtgagtgtgggtgaaggggtgtgtgaggggcaggtgtgacctcaggaaggagggctactggggtcgcattgcttctcctcccagcccgtgaccttcggggcctggaaccaggcagtagcatccagcctctgagcccaggctctgagcccaggctccgagcggctttgaagaggcctgtgggtcagcgctgagggtgctgttttttgaggccttaggctccactagccgggtctgtgtcttcatcccagaatgagctgtggcttcaccaaggggctcagaacggttttccactcattcaaggtcacctcaagcccagggctttctcatctgaatcttgaacctagcctctgtgaaggatgtcctgagctgtcaccctgctgacaaaaatgctgggccctgagaccctgaaatagagcttctcagacaaagatttgagcaacagtctaactgaactccttatgaaaatacagaggtctggtgccaggccgagtgggcggccttgggagtttaaggaaataggtctggttccatggactttttttttttaaattagggtttgggtcattttggtttgaagttcaacaaatcagcatggcttgtctggctttttatctgccacctggtttggttcaaggacagagaaacgtttctgtttataaaacatgtgcagacgtggctggccaatgctggtgtcatttctcctggcttctgctccttttcaagggagctggtaaagctgatcttctccaccctgggctggtcatttagccgggcccggctgaggcccctgtgtaatggacaactgcagccggctggtagaggtgatgggtgtgtgcgtgcgtactcagttgtgcccgactctttgtgaccccctgcactgtagcctgccaggcccctctgtccatggggttttccaggcaagaatactcgagtgggttgccagtcccttctccaggggatcttcctgacccagggattgcacccgtgctcctatgtctcctgcaacggcagacagattctttaccactgatccacctggtagtatgtaagatagatattatttgatgtggagagatgttcactgcaagcactaatatagactcatttttgtaactgcaaaaataacacataaatctggtaagaaatgaaagctgtacagaagtatttttaaagtaaatgaaataccttaatatttgtacagtgcttagaagaacagtacctggcatgtaataaacactagacacacttaataaataagagtatatggaactagaagtcatttttcctcccaatttggatctgtagtctccctctccagaagcaaccaaattaatattttcttgcatattcttccagaaattttatgtgcttatgaaagcgtgtcagcacacataataattttacatcgtaaagagaaaagggtaggttaccaagtagctttaaagagcataattctatttttaaaacaaacaaacaaacatgcatagaaaataatctggaatcagatatatcaatcaggtaaaactatgattatcttcaaaaggtgggatcatgggtggattttctttgtctttctgttcactccttctataattctaaaacttgattagagaaaaacgcatatgaagaaacacatgcatggatgggcacatggaccctggtcctcttatataaacgtgtttaagagggctgacatccatgccaggccctgggatcccatcacagaaccagcctggacaacctcccctttgctccctggggccagctgctctgttgctgacactgaaccctgatctgcgtttggctttgcccagtggctccaggactctcctggggacccctggactgatgatcgaatgaggataagatttgagggactgtccaacacatgcatccccctttaactgtttgttttgctgcttctatatccctctgccagttgtggggaagaccccttcctccctctgccagcctgggcctgggggtggcatgcctagttgaccctgatctcccatcaaggaacaaaggtgagccccgtttgaacttacatgtccatcctgagcccccgagtctcatggcctgggtcctttggacctatcagaagccccctctgatgtgcctctgagatgcagacaggcaggccggtgcagcactccagcttccccacctgacttcctagaagccagtctggtccctgtaatgaccagtggagcaggggtctcggcggtggtggtcctgagtaggcacagtggactcctcttgactctgtagacaagaatggggaatcaaacagacctgggccatgaaccagctgctgttgctgtaggacctggggcagatcagttcacctttctgagcctcagtttcctcatctccaaaatgggtgcactgagggtaccttcctcctggggctcatatatcttcattctcaattcattcctttaacacagatttttctgagagccttgtgtgccaagctctttactggactcacaaaggctcacatataagcttctacataagataccagagagaacaacagcaacaaaaatgttttccagaggtaaaagcatggggaaagaaggagaggcttcttgaaggatgcataggagttctgacacggtgagggttgcagttgggaaagaaagatgaggggtttccacagtccttgcatgtgtatgattttgcatatttctcacaatactcttagaatttgaattttctgagggtaggctgtcattgttgctggttgctaagtcttatctgattcttttgcgacctccgtggacctccatggcccaccagcctcctctgtccgtgggacttcctgagcaagaatgcgaatgctggagtgagttgccatttccttctccaagagatcttcctaacccagggatcaaacccatatctcctgtgttggcaggtggatcctttaccatctgagccacctagaaaacttagactggattcagctagattgaatcgtaagcagagggtaatgtgctagacaaaaccaaataatatcgaagagtccaatctcctgtctcctctgctctcatcctggggaattggttgcctcttcatttttgagtcagggagaggtttccccaggagacctatcttccattttcctattgctgttgttctcagggctggaaagaataaggtctttagacaggctggcctccatccctgactctgtcacatgtgggctggaggtctccagcaagcctcagtttcctcatcagcaaaatgggcacagtggcaatgtccaccttgccaaactgctgtgagacttcagatggggcaaagcacccacttaatgctgggcacatgccaggatgcctgagacagggctgagtcctctgccagcagggcccagtgtctggactcaaggcatccttctctttattacaaagccacacattgagtctgagctcatctggggcccatgtggaccccggggagtccagagatgtttcatccctctgtttggcttccattttccccaccctggctgaatgcctccagccacctgccacacctccagatccccatcaggcagagaaattggtgacccgtaagagcagagggagaaggaagctgtgaggtagaaaccagaagagatgtgtttgcagatgtgagtcgcaggggtgattaggaccccaggcaccgtttctgcaggatgggaccttctgggcgacagagccgaccgtggaatctaccctcctggtcccaacagttcccaaatcgagaaggcaaagaaggattggcttttccccctccgcccctccctcctctcatctgtaaaattgagacattacagttccaaaatgggctttgagcctgtagaaattaatctggtctggcaggaggtctacaaataaacatattgtttcgattggtgttgacatttgaattgtgagaaggtttgaaaatgtaactgtagttgggattttccagcccatctaagtttttttccatggaggggctcagtgaatgccaactgggaacccagcaactgccctctttaaaaggacttgggcgctgggcgaggctgcggatgtcagggctcagagcctgtgctgaggcggcagcccttttgttggggggtgggcaaggatttcaggaggcccagtgcctctgggcatctggggcaaccagccactccaggagtgacctgaaacgtttagagcaactcctgtgtgcccggaaccttgtatgtgaaacctcactcaagtcctcacaccagtgctctctgttgtccagttgctaaattgtgtccgactctttgtgaccccatatactgtagcaccaggcttcccagttcttcaccagcttctggagtttgctcaaactcatgtctattgagtcagtgatgccacccaacccccttatcctctgtcgttcccttctcctcctgccctcaatctttcccagcatcagggtcttttccaatgagtcggctggtctcatcagatagtcaaaggattggggcttcagcttcagcatcagtcgttccaatgaatattcagggttggttttctttaggatcgactggtttggtctccttgcagtccaaaggattctaaagagtcttctccagcaccacagttggaaagcaccagtttttagtggtcagccttctttgtggcccaactcttacatctgtacatggctactgggaaaaccatagctttgactatatggatctttgtcagcaaagtgatgtctttcttttttaacacgctgtctaggcttatcatagctttccttccaagaagcaagtgtcttttaattgctcttattatccccattttacagactggaacactgaggaaaagagaggttaatttgctaaaaccacataggagtaaaatgcagacttagacacagccttctctggctcttggtgtcctgccctatacaatctactttatgccagaattttccagaatgttgtctcaccaagagaaaggctcattgtcccagaaaaaagactgagccttgactgaggtgatctggttgttgagaacaccagctccattctatgtctggaacccttgaggatttaagccccacccccttatcttcctgccaccaaggaaggggtctgtccttctccctccctccccacctccacgctctggatctcatccttcagtggcctctgagtccccactgcctgcccatcccacctggctccagcctccccccaccggccccacagggatgcagctagttccctgtgggaggggcagctctgagacagccccctactcaggggtgaggtgcacatggctttcaaccacgaactcagagtgctggccggttagggcagacacctcgttttggggaaaggcttggtgatttctgtacctggcaacttctgtgtgtgtcaggcatcatctcctggccccttagctccagagatgcgaagatccatcttcagggtcttcagtcacctgctaccatctcccttctgcgcttccacacctacccccacagagctgagcatggccgggaaacacacatgggaactatgctgcctgctccccctttatcctcatcattgggagcatctaaggggcccatctggagaagccttacaaatagctgtgaaaagaagagaagtgaaaagcaaaggagaaaaggaaagatacaagcatctgaatgcagaattccaaagaatagcaaggagagataagaaagccttcctcagtgatcaatgcaaagaaatagaggaaaataacagaatgggaaagactagagatctcttcaagaaaattagagataccaagggaacttttcatacaaagatgggctcgataaaggacagaaatggtatggacctaacagaagcagaagatattaagaagaggtggcaagaatacacagaagaactgtaaaaagatctccacgaccaaaataatcacaatggtgtgatcactcgcctagagccagacatcctggaatgtgaagtcaagtgggcctttgaaatcatccctacgaacaaagctagtggaggtgatggaattccagttgagctatttcaaatcctgaaagatgatgctgtgaaagtgccgcactcaatgtgccagcaaatttggaaaactcagtagtggtcacaggactggaaaaggtcagttttcattccaatcccaaagaaaggcaatgccaaagaatgctcaaactaccgcacaattgcactcatctcacacactagtaaagtaatgctcaaaattctccaagccaggcttcagcaatacgtgaactgtgaacttccagatgttcaagctggttttagaaaaggcagaggaaccagagatagaaaaggcagaggaaccagagatcaaattgccaacatccgctggatcatggaaaaagcaagagagttccagaaaaacgtctgtttctgctttattgactatgccaaagcctttgactgtgtggatcacaataaactgtggaaaattctttaagagatgggaataccagaccacctgacctgcctcttgagaaacctatatgcaggtcaggaagcaacagttagaactggacatggaacaacagactggttccaaataggaaaaggagtacgtcaaggctgtatattgtcaccctgcttatttaacttatatgcagggtacatcacgagaaacgctgggctggaagaagcacaagctggaatcaagattgccgggagaaatatcagtaacctcagatatgcagatgacatcatccttatggcagaaagtgaagaggaactcaaaagcctcttgatgaaagtgaaagaggagagtgaaaaagttggcttaaagctcaacattcagaaaacgaagatcatggcacctggtcccatcacttcatgggaaatagatggggaaacagtggaaacagtgtcagactttatttattttttggctccaaaatcactgcagatggtgattgcagacatgaaattcaaagatgcttactccatggaaggaaagttataaccaacctagacagcatattcaaaagcagagacattactttgccaacaaaggtccatctagtcaaggctatggtttttccagtggtcatgtatggatgtgagagttggactgtgaagaaagctgagcgccaaagaactgatgcttttgaactgtggtgttggagaagactcttgagagtcccttggactgcaaggagatccaaccagtccattctaaaggagatcagccctgggtgttctttggaaggaatgatgctacagctgaaactccagtactttggccacctcatgcgaagagttgactcattggaaaagactctgatgctgggagggattgggggcaggaagagaaggggacgacagaggaggagatggctggatggcatcaccgactcaatggacatgagtttgagtgaactcggggggctggtgatggaccagggaggcctgacgtgctgtgattcatggagtcacaaagagtcggacacaactaagcgactgaaccgaactgaactgacacttgctcccccactgcctgagtcctgtcctctcctatctccccaacaactatttacgcaggggtccaaattaggccagatgcgggaggaaggagtcctcaagggcaggacctctatgccacctaggatgaaggctccccacgcccatacctcccatccctttgatgcctggagggacaggaagcagggtggcaagatggtgcctctggtctagtccacaccccacacccctggtttggtggtgaggtggccccatgcctgaccaggaagatgagtccttggccacagactggcctcgctgcatcctctgctccctacctctcctcctgaggtccctgggggtggggggaattggtggcctcctaggaaagaacccacttcatctgtactacagataccacccccgaccctgagaccacgagaggccttgtctttgcaccttaacacacctcgtctttgctcctctgtctgccctgcccccagcctccattggtttgactcctcagtgccttcctcctcatattcaaaagctcgaaaccatgaccgaaagtttggggacactgggtttgtcctgacacatctgggagcacatgttcaggcttggatccgtctgtctggatgttttccgtttgttggctggttttgcctgcaagaggtgtgaacctctgattggctttgaggttctggaaatcaggaaggtttgtgttcatttcaaagttcagaaaaatcactgtggttttgggtcacggttgggtttgcctgcttgatggcagcggtggtctggggggtatctgagctaggtctctgtggggtccccatggggtctgagtgtgagacgctcagggggtggccctctgggtggggctgatcctggactcagtgatggggtgagtggccaagggcggaggcaggggtagggcgctgtgctcgcttaatgaggtctggatgctctggctttccagctatggtttaaaatcctcagtgccactgtcagcctagtggccttggattttggtctgtaaatgccaatattggccttgacttactgacttcctcaactttctgggcactgacttaatcctcactgctgctgtggtatggcatgatagcactgtcctcattttatagatgggaaaatggaggctcagagaggtcaataacctgcccaagatcacacagcgtatagtggcagagctgggacacaaagccaggtctctggccccatggtggactgtctgttctgccccagacacagtgcacagattggaacgagggagagctggtgtccagagctgctctcccttcaaacttactaccagcacacctggatgtgatttttcgtggacacagagaggtggtttgtggtcaaagcgtgatgagagggcggggctcagagtctgagggccccagacacggtggtgtgagcaggcagtcaggggtcttctctggggtctgccgatgacggggctgctcctcacttttctgaaccccaggttcctcctgccgcttcctaaggcggttctgaggctgcatggggataatgttgatagaaactcacagaggaggtcagaacatatccgatttcatgagctctgcaaagacggctgtgcctcgttagtcatgttcttcccaagttagcgcaatgcttggccctgcgctcgctcagtaagccctcttgaagccacgtggggtctggcctcacagtcaccgctgtcagttttagagataagtctgtttccctgaccggcactctgggttggagtcccggatggccccgacttgttctggaaggagccccactgtctgggcacagcagtcttcctgcaacagacacgagcctttggttgcctcccaggggtgtttcccctgcctgctaaggcctgtcacctcctgcctctgaactgactctatgcctttccttcttcccagggctggaggagcctgacttccctcccctgtccaaggagttcccacagtctttgggttttcaattggaacaatgtgctagagcttgaggtgccttgaaatttaaaacagctacagtttaaaatcctcagtgccaaggtccctgtgtcagcctagtagccttgaatttgggtttgtaaatagcaatattggcctcaacttactgacttaactttctattctaggctcctagttaatcctcacggctgcctcggtgggtggtgttatagcattgtccccattttatagatgagtttgaagaggtccagagaggtcagtaatgtacccaaggtcacacagcttgccatggcagggctgggacacaaaaccggctctctggccctgtggccagccatctcttctgccccaaacacagcacacagatcggaataatgtgaagctggtgtccagcggacccctctgcctctgacttggtcggtgactcagagtctccccggatcccttccccatctggaacagggaaggcgaggaatgtgcccagccttgggagacatgaatggctggctgcttcgtcgggctgagtcgtcacagcctgttcagaaggtgtgacagtcatgaaaggtggcctgttctaaacagcactgcccagggctgtggcgggggctgccttccccacactgcatgctgattagctggtcatggcccccctcactccccaccccgcccagatctgttctgccaccaaaaccacgagtctcaggtcggcagccccacaccctgtcccagccatcctggccttcctcctgcttccctggggacgagcggcccaggccccccgtcaatgctgcagtgttccgggtggcattgcccacgtcaggattgctttgccctcttcaaaaaggatcggctgctacccccacgggggccttaccgggcttcggggttccctgggtgggagcacagaaggccttttgtggctgatgtgaaaaggcgctgccaccttggaggataagaagcgacctctgatgagagagtcacgttggcagacagaaggacacaatttaacttgtcgaaaaagatttttgtttcccttcaccccgtggggcgggagtggaatgaggcaggaaggttctgtcccccttcgcatgctctcctagctttcttctccggcgtctccctttcctttttcttgtccggcctccctctttgctctgctgagatgtccccctgtgtgtaccgcctctcaaaacgcacccctgtctgtctgcttcaggcccgtggatagcatctctctctgagatgcgcctgctctgcgggcttccactcctgctctccttggatcctcacaggggccaggatgaccttgaaaagcaccggtctgattccattggcctcaactacctcctgggtcgtctcctcccctcttgcccttcagtttctgctctggggttctccaggcaggcttcaggctcagaggctttgcacatgtggttccttctgcctggaatgctcttccccccagtaaccgcgtggttcccttatctcatctaggttttcccgaggtcatcttctcagaccctgctggtctaccccatttcaagtggcagccccctttgccaacaccactgatctccctctgcccccaaccccttgcctttcttcatcacgtgaccactgtctggctagaggtggagtttctaagaagtcaatgatgcttaagcttcagagcccctcatcagtaggttccaaggcccgacacctgaattgatactgataatttttcattcttttttcataaggaatccactctccccaccaaactgtataagcttcaggtcccacagaagcctggatcgacctggtcctatgtctgacaacgcatctgttttcattctactgtgtctctccctctactgttaaaaaacaaactccaagagggcagagggttttttaaaattatttattcgatttttggggttgtgccgggtcttcactgctgtgtctgggctttctctagttgtggagagcgggggctgttctctagtttcagttcacgagcttctcgttgtggcggcttctctcgtcgcggagcacaggctctagggcacgcgggctcagttgtcgcaacatgccagcttagctgcttcctgccatgtggaatcgtcccagaccagggatcgaacgcatgtgcccggcactggcaggtggattcttagccactctacctccagggaagtctagaaggtaggtgtttttgtctttttttgtaaaactattttatctccagggtccagaacagtggctatgcacagtaggagcttaataaatgttgctgtatgtacgtatatatgacaactccaggttcatacttctttcagggcgtcacatttctcttaggataaaggtcagcctccttaacacaccccacaaggccctgcccttctgcctgctcccagatgtgctaggattcagactcagggcctttgcacatgctccttcctctgtctgatgttctttctctcccctgcctctgcctccatcaggtctctacagcgggcagtctgcatcagtgggcactccacatatgtgggctccacagcctcagatatcgatggctgacagtaccacaccatcttagctgagcgacttgaacatccacggatttcagtatctgcagaggtcctggaaccaaccccctgtggagactgagggacgactggacttgaatgctgcttctctgtctgtgcccagcctggccctggggatacgtgttctgggaggatcttggagcttctgttgatagcacttgctgccaccctcacgtggtatccatgccattgctgggttagtacttatttccctccaggactgtcagctccatcagggcaggaccgcctcttgtgtgctcctgtatccccagcgtaatgcctgacatgtgctcgtttttactgactcagtcaagagctgctgaggagatgtgcaagataataatactttaaaaaaaaaaaaagggagggaaagacagaaatcaggcacagttaatatgaaaatacacaaaatcacagactctctggggcttccctggtggaacagacagtaaagaatctgcctgcagtgcaggggccctggatttgatgcctggctcaggaagatcccctggagaaggaaatggcaacccattccagtattcttgcctggagaatcccatggacagaggagcatggtgggctgctgtccatggggttgcagagtcatacatgactgaacgacttagcagcagcagtagcatgtcaagtcagagtcgggagggcatttgtccacagcttgtagactgtggttcctggtgccctggggatgcgcctctgggaattctgggaattctatggagtggtccctacagtccactgctgtgaacgtgcaagtgtgtgtgttcacatgtgcacatatgcgagtgtgtgttcacgtgtgtgtgctcatgtacacgtatgcaagcgtgtgtgcacacacacaggtgttgtgactgggggtcagggaagtagaacgagctgcatacggacagatctcagagccccattcccacttcagacagagcagttctgcttttacctgttgaggtattgaacttctgcttctgatgttgtttgaaaagaaataagctttccatccaaaatcaaatttggaaatttctgatctgatttacggagaaggcaatggcaccccactccagtactcttgcctggaaaatcccatggaaggaggagcctggtaggctgcagtccatggggtcgccagagtcggtcacgactgagtgacttcactttcacttttcactttcctgcattggagaaggaaatggcaacccactccagtgttcttgcctggagaatcccagggacggggaagcctggtgggctgccgtctatggggtcgcacagagttggacacgactgaagtgacttagcagatctgatttaacgccccctttccattttagggatgagtaaactgagatctagagatggaggtccttgtcctagatcacagagtgaaaggagaacaggactgggaccaaccatcaacccccctgccccaccccgaggccctgcccacggcctgttccctgatgcccagccccctctttctggttttggtacagcgctgccgcctctcagctgtgaccaggcggccgaacacctggatgtcctggagctgatggaaaagctccagggatccccatgctttaatcccatcatgtaaatcaggaatcattttacttgcagaagacatgacttcctacatgtgtgctggaaaattcacattgtctggtacacctactcagggggaaaatcccaacacacaaaactaactttcctttgagtctttcagttgatatccactggctacctgttcatttatattattgtctgaaagtgctcctagcagacacccagcagcaagcactgaaggtttagaaaaatctgtgggagcaaaaagcaaatgattccatccactagtttcatggggggtggggggtgtaggtgtgaagatatgaaaaaaaattaaaaggatcatttcccatatactttgatgctggaacaaatacctctggccaatgtataggggagagaaaaaccattatttttcattctttaaaaataagtcttcaattcattctctgattacaaaagtacatcataggtacttctggagaagtagaacttctagaaatgtacatgacttagaaagtaaatatcagtattattcctagccccctcccaccaaccacagctaattccaatttgctttcaaacctactaagtgtcaggttctattctaggcactggaggggcaacagtgaacaaacatagatgacagttgtgccctgatgggactcacagctgaatggagaagagggggaaatggtcacacaattgtaaaaagtcaactgtgttttgctttgaaaagaagagatatgaaagcataaaatctggggagaggacctttcatgaggatttctggcgaagctttgtgagggagtcaagatagagttgggaatctcccacgtttgtgaagatgggaagaaagagaggtcaagggagaaaaaaaacgtccatgcaaaggcctgtggcaggagggcaaagagcgacatgaccagaggaactggttcaggcgaagcggcagaggctcctggggttttgtgaagaccacggtaagatttttgggtctttgttctaagagcaaagaaagctataaactgtttaaagtaacggttgctgtctagtcactaagtcgggtccagtgctgttgcaatcttatgaaatacggttctccaggctcctctgtccgtggggtttcccaggctagaatactggagtgggctgccattctcttctccagggatcttcccaacccagggatggaacatgcacctcctgtattggcaggtggattctttactgctgagccaccagggaagccccttaaataaggatgcgtatgattatatttgcatttgatatatattcattctgatttttcttgatgcatacatgcatcagctaatgttgtagcagaatggcttaagcagaagagattctgaactggctcttgtggctgcagcttgatctggggtctcaaacagtgtctccagtttctctccaattttcatccttttcatctgccctcctgactcttccaagccccatggagggcctcctgtggcaccaggcgttcatacactatctttcagcctgtacctcagtgaaaacgactaacctgcctccgctggcatggcagttctagccctgctcatcttcagccaacccctaaaccaatcactgtgtttagaggaatgcgatgctctgattggctaggcctgagtcacatgtttcacaagggggcgtggaataaactctacccagagcatatggcctggtagagggaaaatccttatgatgaaattacttgtagaattagggctgagattgaccaggagagggcgcagggtgtgttctaggagggttggaaatgttctatattcctgatctgggggtagttaggcaggtatgtgtgtgtgtatatgtgtgtgtgtgtctgtgtgagtgtatgctaagtcacttcagtcgtgtccaactctatgtgattctatggagtataggccaccaggttcctctgaccatggagttctccaggccagaatactggagtgggcagcctttgccttctccaggggatcttcccaacccacggatcgaactcaagtctcttgaattgcaggcgtattctttaccatctgagctaccagggaagcctaagaatactggagtgggtgatctatcccttctttaggggaacttcctgacccaggaattgaacccaggtctcctacattgcagacagattctttaccagctgagctcccagggaagcccatgtccagctataccaagattttaaaaaatacctcactggagagtgggaaagaggaaaatcgggatgttgttaccaagaggatggtgagtgggtgttcattataatacattaatatgctgtgtatatacatttgctgtgtctatacacagggtttcccagttggccctagtggcagagaatctgcctgccaatgcaggagatgcaaaagatgtgggttcgattcctgagttgggaagatccccagagtaggaaatggcaacctccttgagtattcttgcctggaaaataccatggacagaggagcctgttgttgagtcgagtctctgagtcccatggactgcagcacaccaggcttccctgtccttcactattttctggagtttgctcaaactcatgtctgttgtgtcaatgatgccatctcaccccataatctcatcctctgtcgcccccttctcctcctgccttcaatctttcctagcatcaggatctttcccaatgagttggctcttcacagcaggtggccaaaatattggagcttcagcttcagcatcagtccttccaaagagtattcagggttgatttcctttaggatttgagggctacagtccatggggctgcagagagtcggacatgactgagagattaagccctcacacacacgtatgtacgtacataatatatacatgtatgttatgtatcagttcagttcagtcgctcagtcgtgtccaactctttgcgaccccatggactgaagtatgccaggcttccctgtccatcaccaactcctggagtttactcaaactcatgtccattgggtcgatgatgccatccagccatctcatcctctgtcatctgcttctccttctgccttcaatctttcccagcatcagggtcttttccaatgagtcagttcttcacattaggtggccaaagtattggagtctcagcttcagcatcagtccttccaatgaatattcaggactgattcctttaggatggactggttggatctccttgcagtccaagggactctctagagtcttcaagaacaccacagttcaaaagcatcagttctttggcactcagctttctttatagttcaactcttacatccatacatgactactggaaaaaccatagccttgactagacggacctttgttggcaaagtaatgtctctgcttttgaatatgctgtctaggttggtcataactttccttccaaggagtaagcgtcttttaattccatggctgcaatcaccatctgcagtgattttggagcccccaaaataaagtctgacactgtttctactgtttcccatctatttgtcatgaagtgatgggaccagatgccatgatcttagttttggttttcagctttactgaggtataattggcaaataaaattataatacaataagtcccctatacatgaatcttcaagttgcagactttcaaagatgcaaacgtgtgttccatcaccgtcaggtgtgagtgaaactgcggcttgcccttcatctcctattgttgacgatccttcagctctactgtctcccacctcctctcccttctccagtcagtaactcttcttgactgttcactcagtgccagcccctgtgtgccagctgttgtactgtagtactgtacttttcaagattctgtactgtgagatttaaaatgttttctttatgtttgtttttttaaaatgtattatttgtgtgaaaagtattataaacctattacagtacagtactatatagctggggcttcccaatgtctcaatgggtaaagaatctgcctgcaatgcaggagacacaggagactgtgggttcaatctctgggtcgggaagatcccttggaggaggaaaatggcaacccagcccagtatttttgcctggaaaatcccatgggcagagaagcccagcaggctgtagtccaaagggtcacaaaaagtcagacacaatataactgactgtgttagttgggaacctaggctaacttttttggacttacaaacaaactgaacataggaacacactcttggaatggacctcattcgtatgcagagaatgtccaacgtgatggtttgatatatggctgcattatgaaaggattcccactacccagttaattagtgcatctgtaagctcacgtttcccttttttgttttgttttcggtgagaacactcaagttctactctcttagcaaattttggcgttacggtacaggattttcaactatggacaccgtattgtatgtagatagacatttggtgagaagtgctgtagcaca

SEQ ID NO: 3 - DNA, 1686 bp, Bos taurus
gtttaacgaattcgcccttcaggcaatttccagggaagagccaacacatgaacccagagctcttgatgtccagagaagccatggagtggggaaggagaaagcaaagaagcagctactcagccaccagagaagatccaggtggtctgtggcaactctaagccctccccttacggggtaaagctccacccacctgggagcaggctgcccttctccgcccagcctctccagcaccaatccattaactgcagtgaaccaagctactagtctgtctccccatcccgacctgaaggcagaggctgtgtccagggcctcacagaatcccccaggaggccaggaacagggacaggcaaagtctggtgaggagctggaattaggcatttcagtccccttctctgtgaaaactgggcatttgggccaggaggcgtctggcgtctcttccaacactgggcgggacacccatcccgacaccaggacccatcatgttggagggttgacttccgggcctcaaccaaggtccccgacctggcaggtggtcagcctcagaggaggaaataagtcatgtggcctcagctaacaccccttgggctctcactgccgggtttccacacacttaggaaacaaaaccccgggtcagggagcccagcagagagcaggagtccctgccccactgggtcacatttggggatgagttcctgcaatcccatcaggtgctcttctgttgccctggcaaccccaggagctcccagtggagcccatctcatttctgaaggagggggaaagggcaagcatctgctgtgcatgagacacagagtgtgtggccaactggatggattacagtgaaaggagacggtaggagaaggggcacaggtaccccagctccttcttccttctccaccctctgcagtccttccttccttcccactgacttgcctgtggggttccattcccctttaggcctcagtttgttgttgattagtggctaggtcgtgtccgactcttgcaaccctgtggactgtagcctgcctctgtccatgggattttcccaggcaagaatactggagggggttgccatttccttctccatgggggtcttcctgacccagggatcgagccccagcccgcgtctcctgcatcagcgggtgggttctttaccactgagccaccagagaagcccaggcctccgtttgccaagacggtgctcaggctggtttctcttcctcagaactgaggaaatgctcaagagctggaggtgggaggaggccccactgcggtcctcaagggcaggacctctatgccacctaggatgaaggctccccacgcccacacctcccatccctttgatgcctggagggacaggaagcagggtggcaagatggtgcctctggtctagtccacaccccacacccctggtttggtggtgaggtggccccatgcctgaccaggaagatgactccttggccacagactggcctcgctgcatcctctgctccctacctctcctcctgaggtccctgggggtggggggaattggtggcctcctaggaaagaacccacttcatctgtactacagataccacccccgaccctgagaccacgagaggccttgtctttgcaccttaacacacctcgtctttgctcctctgtctgccctgcccccagcctccattggtttgactcctcagtgcc

SEQ ID NO: 4 - DNA, 35035 bp, Bos taurus
  misc_feature (1822)..(1871): n is a, c, g, or t
aggaccatggagcctccccacacctggagctgctgagccctatcccacgtgctagacagagtaaggggtgctgggggatcctagcgaagggaagtcctcctcttcaccagcctctggcccacttgtgggtaaagacgggaaggcaggaattggcccccacgtccccacacagacatgcagactttttgtttgaagcaggagcccaggaggtgagcccaaagtcaccaggtgggtgtcagggtcaggcaggacaccaagaagagacctctgaagcagcatcagccagttgcctcttggtcaccatggacaccaggctgcaaagaaggttgatgtgcccactggggacaggtcagtgtggccctgggggcaggttgcagggtttgtgctgggaatggcagggctgatgggaacaggcctctctgggcagggcagtcagctctgtggttcccagcacggagcattgcccatgcctgtttttgtttgaaatctggggccccagaacccccaacaccatgttctgatacagtgtgccccctcagttactgaaacagaaatggccctaagccctgcccacaagccacagggatggggcctgggctgtggctggtcagcccaggatgggaatggaaggggacagggggctggcctgtccctgcacccgcagctggcactcacaggggaactgcttcccaatggacatgagtccagtctgctctgctcatcaccaccactgcaagggctgtttctgggccttgctccacccatcctcttccacaaccccatcatcaagtgggcacgtgatgctggaggccctgggtgaccatgggcacattagttgtatattgctattgttacttacaataactttaattttacataaaacattgttaattatagctgtacaacaaattaacctacaatttatccgtttaaaacaacaaacacattatctcacacaatttctgagggataggagtcctggaactgctcagctgggtggtcccggctctcagtctcttcggattgctactgagatgtcggcagcagctggggtcccagctgaaggctggagaccagcttataaaccccacactctgttggccgctccatagcaaaggacagctggcctcctccaagtggaggatgttagagacagagagagacaggccaggatgcccctgcgacctcatcttctacaggaccttactgctgatgcaggtcaacctcagtacattcagaggtagagtaccaggtcggggctcttgggggggctgcctcctgggaggctgtcaaccccagggcctgtttccttgcccagtggtgcctcccaggataggtatggcccctagagcttcaaggggcagagagcagccagacacggctccagaaccctctgggctcagcttctttcttgggggaaaggggagcaggtcctggagcctagaggaggctgttggggcctggagataatcaggtgatcacaggagctctggttgggaagctaagggctcacctttcaaaagtcaaggctcccaggagccccaggtcctaccccatttcaaactcccaagtacctggagttttctgggcctggcgaggccgactgctgtcaccgttaggaccaactctttctccagatttcaaacacctatgatttgctgtcattgttagtccccaagccctgacctcacaggcaagaaagagggacccagagtcacagggttccacagggcgagacacccacagaggcaggcagagcacctgactacacacaggaattcagcaaacactcattgacccttggtccccaccaggaactggnnnnnnnnnnnnnnnnnnnnnnnnnnnnnnnnnnnnnnnnnnnnnnnnnnggattgcggtgcagaggaattccccagagcccctctgaggcaggggagggacaggcaatgggtggaagagtggggagggtgggaggaagaagagtccaccccagctctccacagaccaggcaggggtccaggctgagtgtggaaagggccatggctccacccccagcagacgccaggatactgccctcagctgcccaggggcagcctggggaggggcccagcctcagctgtgcttcctcagactcccctaatctaatcaagggtgacactcctttcacccaagctaattaatgaggcctgtatggcctgagcgtctttctgttccaagcaggtactggggataggagtgagtgacacagacagggccctggcttccagagcccttggagcctggggtggctatctcaggtgggaaagtggggagagaacctcggggggcagggagggggctgagtagggaagggccaaagggggcagggaagggacagagacacgaggaacttgcctgtctgaggaacagtgtggttccctcagagccggagaggtccctggagggctgacaggggcctcagcacaaacacctctttggcgacagcgtcttgaggacagatgaccaggaacaggaagtaaggaagtaaagggaagcacagcccaggaacagggggatctggtttgacactgtgattgccccctccctgaggagtgatgacccgagctccatgtgaccttcctgggcagccctcctccctctggccaccaggtggggagatccctcttattctttcaggccacgtggaacccagctctgaggaggccttgtccacccctctacccacccaaccacctggcttcatatcccaaagtcccttgccttcccctcctgcactgagcctccaccccaaacagctcccctttcccaaaaaagctgaacttctggctcctgaaatggaccctctgcaactcttctgctgggagggctcccaggctggtcagcagtaatgcccccacagtcctgctggtgacccagctctctgagcctccccagggcctggaccagtgagtgtgtgcaggtcccagggtgtggctcccggcccacatggctgccactgccccaaaccactggcccagaccctggacacccagggagatgggccaggcagccagagagcaggaggggactggcttggtgggacttggagggccctgcttggtgtgcacggctgagtcctggagggcagatgaaccttggtcaggaactgttggggtcattggaggaggagataagcctctagcagtgactaaccctcctcctccctgatgctccctggccaggcaggtagctggcaaccatctgatgaaattggctgtgggtgggaaacccatcttgccttgacctcagagggctcaggatgagacactgcagtgtcagccacatgactcaaccacctcctcactgcccctggagcacagcgtccagcagcagcctgaggagacctaggaccacaggacagtggctccccggccaatgccccacgctggctaggatcactgtattgtggggtcagcataaagccagtgttcaagacaggagcataggcctgccaatgaacccccaattccttcctactgtcgggggactccctctcaggggaggactgggctctggcgggaggtgagccccagtacaaagtgccctttgtcaggccggttgggggccctgcctctaggactcagatgccctcttctgctggcccccatctgctgggcagagactggcttggggcaggcttgacccacaggtgccaagagtagttctgtgctcccccgggagaaggcaggcggtgcaccaggccagaacccaagcttcagcccacctcagccccagagacaacagcgtgcacacgtctctgccttccaggggcctcaggaggggttgcggggagagaactgtgccccaggaggatgcacgacctcttctcagcactgggagacgctctgggaaagtggtaactgttccagtcctgcctgagccccccagggacatctcacacacaccctcctccctcgaggcggtcggcttcagcgcagagggacacagccaaactcaaggccgggcgaacccctgcgcggcgcaccggccggcccaggtgggagctgaataaatccccacggcccggcccggcccggcccgccccgcccgttcatgagaagacatcaaccgaccccggcgctgggcctcggccaaactggacccttcacgaagacgacaaaacaaaattccctacccgatctctggaaataaccattttctggcacagtctcagtcggttgtagggccgcggtctcaccgcagccacggcctccagggcgcgccctggccctgagccccggccccgggggcggcgagggccagcccgggcaggaagatgggtgccaaggccgccccggggcgcggcaaggggcaagggccgtcaaaggctggcggcggcgggcggcctgcgtagatctcgggctcctgggcttcagtggctggggccagtggaagcgggcggcgcgcgggccccagccgggccaggcaggctgccagatggcagagcagacgagaccgcacgtccgcagggacgccctcgcagccagccaggaagcgattcacttcggccagacactcatggaagccggcgcgatacttgcccaggatagcggggtccgagcggagggcagctgcgggggtggcagggacaggcggtcggcgcccgcggcctcagccccggcccctgagccctcgccgccgccctcgcctcacctgtcacctgcacgcgccgcaggctctgcaggtgcctcacggtcagctccaggatgtccgccttctccagcttcgagcggcgggagctctgcgggcggggccggtgagggcggcgggccgcccaggaaggccgggattccagactccccagtcttgtccctacccccaactcacctctttcctgagggcgtccaggaggaggctctgcagctgagccagactctcgttgatgcgcgctcggcgccgcttctccatgaccggcttggaggactgtggagccggcagccactgagtcctgcaggcatcatctcccctctccccttcgcgcccccccccccccccccgccccgctcctgccaacccacgcccggccaggtccccaccttccggtgctcggccacgctccggggctggtctggggtccggctggcgccggccgccgctcccgcccgcggcgaggccctcggcttccccgggatgtccgcaggcatggtgcgccccttcgccacccccagccgagggcaggctgggcgcgcggtttcccaggcacttctcccgcgggtcccaggctcagctaccaagcgcgtgtctgcagcagcccggctatttaaggcagcgcggctgcggggcgtgggaatccctctccgcgttctttcccacactcgagccagccaatgagccgccggcgccgggcagcccgcccccggccgctgcccccgccggctgtcagtcacgagtcagctcccggcccacagaccccgctggcaacaaaggctagccggggcaccccgcccgcgcctgcctcggaccacgccggccaggcgggaaaatcgccgcgccccggtccccagttccgagcgatgcgccgggagggggcctctccgggagggcggaggcgcgaggctcaccttcaggccgagcggagaccgcgagaagtctgaaaggaaaattcgcgaggggcccttatgcgctcacgaagaaccggcgagcctccgccttccgcggatcccgcgggggccttggacctccgcgcagcctgcgcatctgaccctcgccgccagcagcacctgcacgatgccggggacacagtgaacatggaacaccagccgctggacaggagagaagctgactggccaaggtcgtgagggaagcagggtggcatcacagggccaaagaaagtaatgccgcagagcagatagtgacgtcatcacaggcaggatgttgtcatggtatggaatgtaaggatgttactggtcaggctggtgatgtctccaagcaatcataggtgatatctcagggttaaggtagatgatgacacaagccagatgtaatgatgtatggtcactggtgatgtcactctcttctggattacccaaaagtgatgagccaagcagcagccaagaaagtagaactcacaaaaaatgaaggataattgttacatttctgtttgttatttcaaagaagcacgtggaggaaagagggctaagcttattttcgtgtttgatgttgttttcactttgaattcccttgtggggcacaatcatgttttgagttttggggatgccagcccatggtggcctgggcagtcttgtctgcatcccacaaacctctctggaggctcactgtaggcctgactgttcttggggctggggaggcctctcctgaactctgaactgatgtgggaggaaaaggcaaatgagcaaaataaataatgacatggtttccagagacagaaagaaatgtgtaggttttggggggagccgaaagccttctttccactgagtggtctggatggtatttttgcagtgagctctgctggagaaggcaatggcaacccactccagtacactcgcctggaaaatcccatggatggaggaacctggtaggctgcagtccatggggtcattgagagttggacaagactgagcgacttcactttcacttttcactttcatccattggagaaggaaatggcaacccactccagtgttcttgcctggagaatcctagggacgggggaacctagcaggcggccatttatggagtcgcacagagtcggacatgactgaatcgacttagcagcagctagttgcaagtccagaggaggaagtggcattcccagaatggaaaggaaccacagggccaagggaggaggggccgggacaggtgggaaccaggctctagtggcttcaggtccccagaggggtctaagagagtcatgcttggcttttctgagcagcagagtggcccaactggcagctcggaagggccacctcccacactacattctacctggtgggtggcgatgctggctggcccccacctcaataccctttcccaggggtccagtctggggcccagtgcagacgcctgtgatgcaggtttgatcactgggtgggggaagacaccctgccccctccccggaggaggaaatggcaacccactccagtattcttgcctgggaaatcttatggacagcctggagggctacagccaaaagggtcacaaagagtcagacacaactgagcaactgaaggaaggaccctcctgtacctgtttcctcacctgtaaaatgggctaccttttcatttagccctggactatagtccatggggtcgcaaagagtcgaacatgactgagcaactaacactttcacactttgactgagaagataaatgagatctgattttgaaagcacccagcacatgcaagcgggacagagtcacctggattcacctctgccacaagccccactgtatcctctaccctctgagacaccctctccctacctggggtcttcagaagcctgtttcttggcccttccagccccaacaaggagccttctctcacccagagcagagtgagtcccaggggtgtcatctacatccctgtgtccttcccactgggctgcacccccactctctctctgggccctcctacctctggctgtcccctccctgccttttacatctggatttgatctctggtaggacctgggactcctggggaccgtaggggctcccttgctctgagtagagagggagagtcctaggctgcaggggggaaactgtgctgagaaatactgacgtggaacactgcattttccaaagatggctacaatacatccaccccaaatactctacctgtgtgtccttggtgagattttgtgatgacattaaccagcaaagacaggggcagtggtgatctatgaattctgaggcttgacttgaaaaacgtgcaccttgctgtcctgggaaactcactcctggaaaccactgacccctctgtgaggaaacccgaactggtctcatggaaaggcctcatggagaggccttgtataagtgttgagctaggaacctagaaatctcagcttccagacaggtgatcccaacctgccgcttcagagcccccactcacactttcccagccagctctgtgtgctctgttccactcttgattcacatgactggtgttactgtgacactttggggtcattcgttacgaaacaatagtaactggaacagctaagatggcccaagccctggacccacccccctccccagccccaccccccaaaccttgttccttcctttctctggcattcacagagtcacaggagcttcccgcactggctgtccatctgacgaggtgacttttgcagctatccaaggccaactatctgtcctcctagatccaggcttgagttggccaagtcccagtaggctgatggtcccaggggcatctctagggaccactgttggccactgggcaggaaaagtagtgggggagtctgtgtgggaagataagtgtattttccagtccagacactgggctgcagccttggtggggggtggggggatggtctttctcacgtctggctcccatgacagcattccacaacattcagacagtgtccctccggggcctgccaggtgctgtctgtgcacaggcccctcccaacctctgacaggggggtggcacctgcacttgcaccctgcccctcagcattccacaggcacatgggtccctgaatatcagagacaccatagaccatgaccagcaaccccatcctcctgttccctcctcttctgccaagggacaacaggctgtggactcaggcagccaggaccagagttgccatgtgaccaggctagagtcattgcagggtctctcctggagcatgggagttgggtcctgagatgataaagtggcagaggctagggctggaggaacccaggattcagtatagagaagaaggaagcagatgaccaggaccctaatcttaaccctgagggggcatctttcatttttacaggccagcggaccaagacaccttccttcggctcttggtgctgggcagttggagcatccccccaacacctgatgcatgacagcacctcccccgaggtctgggcattgtcaacatctttctccattattgaatgttgacacctctccacgctggggaagccatgtctccccaattctgtgtggtaacagagcttcccaccctgtctgcctgtgatgccacctccctgtgggcttccatgggtgcttctctctccagtctccttggaccactactctctggagaggtatgctttgctctcctgctggtcacatccagactagtttcttcttcaccaaaaaaagtcccagctttgaacctcatcttattaacagatctcatcttgtgaagtccactgcaaccctgcggggggggggcctccccataccttctgaaggttcactcctgattccctgtcactttctccagcggctcctgtcacagtccttgttgacttccaaattcacacggttgatcttctggacttcctggcctccatggggcatcctagtgtagagttggcaactaagcttccaatcctaagtgctgggtgtagactgtagccgtaatattcccatccaccctggaggacacgcccaccaggaagccccgcccacagggcacaggtcagcatctgcagtacaatatcctgctgctttcccagtgtaacctggggtcctgcctcctccctccacactctcagcctcttgtctcaggttctgcctcccgcttctccttgtgcctttcagtcctgtggaacttggggaacaagtcttcactctaatctttctatccacacaactggagcactctctgcccagtggtggagcctgactgaggcaccagcctcctctgaatctgactattctcagttctcagaccagattctgccatcgtcctcgaccctcagagttcactcccagtgctgctctggcaggtcctacagtggctctatctccctgatgtatacaaacacctacttctgacctattgcttttccaccactggctgcaagccgccaccagcttcggcctggaattttgtagttctttgccatggcaaggcagccagtcttggagccgagtgctcaatgggggcaggtggacaggagaggagattagagggtcagcagagtggtcagaagaacaagcctaagctcctatcttctctcggaagcttatgggaagccatgggagagaaggttgtgtgtagatcactctggcccttggctccaccacagattatggggtcaggatagaagcacagccactcggctgtatccatagcagcctcagtggacgagggcgcatagatataagtgaggtcatggatattcttgggagttttttgttgttgctactgctgttagatggctgaagggcttaaaaataaaagcaagtgtattaaacgatatagatactgttttaaaagttttaggaagtttaagttagtttgtaaatgaaaccaaatattaaccaaagacccctttataagtagccattgtaacttcataaataggatattcaggtttttgtctagaacatatgtattttgtgatcatgttgttaggtgcatacaaattaaaaataatttcattttatcagtgagctcaaccttttattgttatgcaaccttctgtatctagtaatgctttgactataaggtttctttgttaatctgttagtgaaactgttaatacggtcacatctgcttccctttagttaacattacgcagtaggtttcttttctccttttactttcagtttcctgtacctttatatttcatattatttatgtcttctgtaagctgtgtaaaatcagattatttttaatcaggctgacaatcctggtcatttagtccccctggatttcagacctctgctctttagaactgtgagggaatacatttctgttgttttaagccacccagtttgtggtggcttaaaagcctccaagacacagagacatgcttccagggctatcgttttgcctgccgaaagcctttgcatctagttcctcttgtccaaagcaggtgatattcaataaagtaataaaagaaaataaaggttctattactatatataccatatttttgttatctttttaaaattttttattacttaaaatttttttgaattgttgtttcattcccttatctttgtgccatatttaataatggtcacctagggactttcctggtggtccagtggttaagacttagtgcttccaatgcaaggggcatgggtttgacccctaatcagggaactaagatcctgctggctgcatgccatgcccgaaaaaaacaaaacaaaacaaaacaaaaaacagaatcaaaataatggttttttgaagaagacagggattttcacattctgtaatatttgttttgtttgtggctttctttttataatactaatattatttatgcaaggaatgagtcaagttagaaatttagaataatataaacaatatttaaactgttttatgcatgttaacaactttggtgccttatgattatttggggggaattgatggaatgattaattcatatgaaaacaatttggttttccccattccttttatttatttaaaatttgtttatttagtttaaaatttatttttaaaatatttaattatttttaggtatttatttttattttatttatttattttggctccatcttgtgggatcttagtttcccaaccagggatggaacctgtgctcctgaagtgaaagtatgaagtcctaaccactggactgccagggaattccctaagtatttatttttatttttagctaacatatgcctgagatgtgctgtttgtaatagtaaaaatgtgggaacaaccaatatttatttaaaaatatgagactggttaactaaattacactttattctataatggactttgttgctatgggcaacaaatgcacatatgcagccattaaaacaacttgcataaaaatatgagggcttacaaatagctgagaaaagaagagaagctaaaggcaaaggagaaaaggaaagatatacccatctgaatgcagagttccagcgactatcaaggagagataagaaagccttcctcagtgatcaatgcaaagaaaacaatagaataagaaagactagagatctcttcaagaaaattagagatatcaagggaatatgtcatgcaaagatgggcacaataaagggcagaaatggtatggatctaacagaagcagcaaaagaggtggcaagaatacagagaagaactatataaaaaagatcttcatgacccagataaccacgatgatggtgtgatcactcacttagagccagagatcctggaatgcaaagtcaagtgggccttaggaagcatcactatgaacaaagctagtgtaggcgatggaattccagttcagctatttcaaatcctaagacatgatgctgtgaaagtgctgcactcaatatgccagcaaatttggaaaactcagtagtggccacaaaactggaaaaggtcagttttcattccaatcccaaagaaaggcaatgccaaagaacgctcaaactaccacacatttgcactcattttctcacacactagcaaaggaatggtcaaaattctccaagccaggcatcaacagtacaggaatcaagaacttccagatgttccaggagttggtgatggacagggaggcctggcgtggtgcagtctttgagtcatggggtctcaaagagtccaacacgactgagcgactgaactgaactgtgaatgaagcagggtacaaaatgttgagagtagcaggaggattttttttttcaattttcttttttattgaggtgaagctcatgtaacataaacttaaccattttatagtatataatccagtggcatttagtatatttagtccaaccatcacctctatctagttatgaaacatttttattattcccaaaggaggccttagctccatgaagtgtctctcctcatgcctcctccccgcagctcttggcaattactaaggtattttcagtctctatggacttgcctcttgtggacatttcatatcagtcgaatcacatgctttgtgtcctgtgtctgttatttgtcactgagctccattcacaccgcagcctgtgtcagtgctccatccttggttatggctgaggagcattctactataaggaggcaccacactttgtgcctccagctgctgatgggtttggttccaccccccatttcaaccattctatgactagagctgctgttaacatttgggtgtgagcatttgtttgaacacctgttgtcagttctgtgggcataccctagttgtggaattgctgggtcatatgaaaattctatttaaccttttgaggaatgactagactgttttccgcagtggctacatcatttcacaatcccatcagcaatgtatgaagattccatttttttgacatcttcaccaacacttattattcactttttaattattattatattcatgctagggggtgtgacacagtacatctggttttgatttgcatttccctggttcccatcatttccaaagatgttgaacatcttttcatgtggctcttggccatttatgtattttctattttaaaatgtctatttaagtcatttgcccaacttttaattgagttgcttatcttttagttgttaagttgtaaaagttccttatatgttgcagatactagactcttatctgacacatggattgcaaatattttcccccattttgtgtagtattttcactttctcaagagtgtccatgagttcatttttatagaaaagatacattctttttcattaaaacaatctgaacatatattgtcataaacacaaagtgattatctctgaatagattgtcaatgttttgttattttggaattaaccctggcaatatttttcaaggttagtaatttcacaaattggtaaatggaaagtgaaagtttctaattcttagctcatgttattatgatgtgtgtgcatcttggaaaggacccaaggtgtggaacatgcagggatttctgaacactcaatcctagaggtgggagtgctatcacagtcccagacccagctggtcaggtggaaagcccccctgcccagcccacctcagttcctcgggtcatcatcatcggcattgttttgtttctgtttctcactctggggctgtcctgggaaaaggaaaccgaaactgaagctgaattagcctatgtaactctgacgtggtttgctcaggctattaaagggctgagctgccagggctgaagagagccgagagcagagaggcagccgcagccaaccagtgtctgcagagagcctggcaccagaacccacggccatggtgagtggtgaggcgggtttgacaggtggggctctgtattcccctgcctggtggcatctcccaggggaagaatgcctactatgcacaggcaggtagagctactcaaagcatccggttccctaagtgtgaggggagggacagggctctttctaaaatcagggtctgggtcctgaaaggtggcttctgcccagccgccaactctgagatcccttgggatcagcacagggcaggggggtattttattggagagttgacatgaagattgtgggtagtagctcttctgctcccttgcactctccagtgcctggggtccccatctccgtgggtgggaagggtggagaggagggagctgcatccccggggtctgacactcgtcccactgccagtgctacacgtgcagtagtcccaggacttctgaatctgtgtgatgcccacagggcggggacctgacggtgaagatgctagggggccaggagatcctggtgcctctgagggactccatgacggtatccgagctgaagcagttcatcgcccagaagatcaatgtgcctgctttccagcagcgcctggcccaccttgacagcagggaggtgctgcaggaaggggtgccccttgtcctccagggcctgagagctggcagcaccgtcctgctggtggtgcagaactgcatctccatcctggtgaggaacgacaagggtcgcagcagcccctatgaggtccagctgaagcagactgtggctgagctcaagcagcaggtgtgccaaaaggagcgtgtacaagcggaccagttctggctgtcttttgaagggaggcccatggatgatgagcacccgctggaggaatacggcctcatgaaggggtgcaccgtgttcatgaatctacgtcttcggggtgggtagggaagggccaggagggccttagggagggctccccatgcagcgcagtgaataaagttgtagcaaagccaaatgtgaagtgttcattccaccctggccagcaccccatgtcatcagccctccattcggcacccttctggtgagattgggggaagggtggaggagggagtgggtgaggggactcagggtcactgagtggtcaccaggctagggtggccaagttaagggacaaaccctgtgggatcaaaatggtgaccctctggagaaactgaagatgtgtccccacctctttgggaaattaacttctagagaaataaaaagaggcaggtctgttgaagcccatccctgtcttccggttgagcctcagtcagccctgcacaactggtctccaccttcacagctgggtcagcagatagcagagggagggaaaaggcctggctgggtcccaaaggccacacagtgagtgtccctggaagcggtgaggggctgagtccctagggcctgccttggccagtggtaagggaggttcccccacaccagctggaggctgagggcctccaggacccatcagaatacactgtccagccttcattggtctggagggaattggggactgggccttgggcacacacatcaccattagggtgagctgctccttcagagttcacatcatggaagggccatagccagcaggtgagggggcacccaggatgggggttcctgttgttcacatggatgggaggggaacaaactacacctgcttccacactagatgcttctttctgtgccagtttcccaagtgagtcagccttggacagaatgcggtgggcagggagggggacacagaacctatacctcttgggccaaagtccatgactctggggacactcagatctcagctttcggactcttcaggggcatttggctggagacccagaccccttctttctgatctgtgggagtttgcatctgagctgggggacccccaggtctggcttgcctgtgacacttatgtagacacgcaggtgtgtgtgtgtgtgtgtgtgtgtgtgtgtgtgtgtgtgtagggggactcctggctctagcctcatttcctgggcccccaccctcacctctttgtcatactgtaacttaacaagtgtgagcacgccctctcggtgacaccctcctttcttgaatcccctcagcagggtctgggcgaggtagggtccttctccaagatgcctctgggctgctcacccttcatggcctctggctcagagatctgtggttggcttggatgagggggtgtgcagcagactcagccctggaggaacagaagggatctcctctcctcccattcccaccatcttcagctaagtcgggctctaatttgcacactctggatctcagtttaccccctggacccagcgggaatggttggagaagtagcgggaatggttggagaagagccttgggggagctgggaagggggcgtggctggccgtccatcgctgctgctattcccacctcagtcaacagggggagcccgcagccctgcagagggccgggagccgacccgggatcgcgaggtgggcggggctgcgcttcgctgcgaccaatcgccgcagctcccagcccgcattggtccccggtggccggtggtcccgggcggccgagcttccgtccccagttcctgtgaggggctaggatcccccgtcaggttcctctcgccgggggaggggcgcagccctttagggcgcgaggggccgcagggcctcggatctggcagccccttcttgcggccagggagcccccgccgccccagccctggcgcagcctggagccccttggaagtcccgtcacgtcgtctccagattatgcattaacccgatttcagccgcatttcctggcgggggggcgggggggtggaacgtccgcgcgaacgggggaggggcgtccgtgggagtgcgccacagcagggagagcaccctcgagcccgccccaaccccgcccctcggcctggccaggcccaccacccaccttggccaaacctagaccggccccctccctcccccagccaggcctccgcccggcgccgctttcccggcgctttgttcgcggctgccgcgggtcgcaggacgccgggagggccgggggcgggcgggccgggggggtggggggtgggggctggaagggggtggtgctgccccaaggcccgcccccgcaccgcccgcgctcccgccacctcccccgagctgcgtcccgtcctgtccagtccagtccccggcgcggcccggtccgtgcgcttgctccggccgccttccgccgtcctctgcccgcgccatggccagctttcggccgttcagccgggccctgctgcagcctttgctgctgctcctggtggtggccgtgcgcgccctgcccagcgccgacgggacgtgccccgaacgcgcgctggagcggcgcgaggaggaggcgaacgtggtgctcaccggcaccgtggaggagatcctcaacgtggacccggtgcagcacacatactcgtgcaaggtgggccccctggggacccccgggaccctgagccccttcagacccttctagaagccctggttgggggcccttcccccgcgatcccggacccctagcccggaggtcttcccgtcgcgatgccggccgcgcgctgagacccccgtcccgggacgtgggaacgagccccagacgttccgcagcaccagctccggctcctgctcccttagcgaccgccgacccccggtgggaggggtggggggcgggtgcgggtctgagaaaactcgcgagcgccggagggaaagttcctgcggtgccgctgcagtcccgctcgcggtgcccgggccctggagacccgatagtgtctgctccttcaccccaaacccactcccttactggaaacaagtgcgccccgccccccggagaatgttcggctaccgcgccccctccccaccggccaaaggaaaggggacgaggggaggtgacgctttctgcttgggatggaggtggtgatttggcctgtggtcagtgctgtcgccctctccgcagaggtgagaatgggtggtccggagagagaggaggggtgcacacctaatctcccaaccgcgggggtcctggtggggcttgctgcagtgatgggtgtggggccagagcagagaagtgggcggggccgttgctgagcctcaagtgcggggggtgcagggcaggtgccccatccgctcctgggagagctgtatctcccctggccttagggagaggggccttgcctggcctctccttcagttgaatctgggagctcccggggtaggtctggtggtttttttcccacaatctgttcctagggaggactgaagggacccaggcccctgcccctctctggcccctaaaaccacctccctgaccagaaatcggatcattcctcttccttggagatggggaggggtggcagctcctttcagactggcctgtgctgactacagctgggatcccccgccaggccccctcccctccaccacagccagggcactttgccaagtccctgcaggattttcccgacttcctccccgctgctcctgggtgtggctggggtggggggaggcgatgaaagccgcccagctctcccctgctggttattggccttccagagcctactctgtatctaggcatggggggcgggggaagagctggcagccccaccatccctgggccactggagggtggagggagtccctgtagattttgcagacccaaggctcccagcaggggtggccaggtggacgggccactgttgtctttcactcctctttccagaagtcccggcccccatagatcctgaagaaaggggtgttttttctcagggacagcattcccgggcagtccccatgggcctattcctttagggcagctcattctgcctgcagtgcctgtggccctcctgcccccactgccagtagtctgtgggcacccccagggcttggtttgtctgagcaggtgccctcagggccccaagagcagagtgtggagggtggggctgccctgtgctgcccaaacttgattttgtggttcacaggctgccttggcagcactcactgtaggagccttgcaggaggaggggtctttcacctcctggctggaccgtgtcttagcccacccacagggctgcatggagccaagctggacaggagccttgagctcagagcccgtcaccttgaaacttcatcatccatgttagttgctcctgactctttccagaggtgggcccaagtactgggtgggttgacagggagagtaaggtgtggaaggctgtgaggtgtgacatttagaactggggtggctggggtcatctaaacctgagggttagggagctgcttcttgagggaaggggtgtgttctcaggccaggtatcctggagtataacaagggcagtggcgtctgtcatgtgtgtgtgcttccctgcagcttcctcctggggggttggggcactgactccacatatctgcccagggtttgggtatactccggacatctggcctgagcaaatgaggtgggaaaagaaggaaaagtccaggcaccagtctggtgtctgcctctggcctctgtggccatctcccccgctccacccagttagaagagaaccgccgacaattgaacaccccctcatgtccaccctaggttcgggtctggcggtacctgaagggcaaagatgtggtggcccaggaaagcctgctggacggaggcaacaaagtggtgattggcggcttcggagaccccctcatctgtgacaaccaggtgtccactggagacaccaggatcttctttgtgaaccctgccccgccatacctgtggcccgcccacaagaatgagctgatgctaaactccagcctcatgcgcatcaccctgcggaacctggaggaggtcgagcactgtgtggaaggtgtgtgtggggccgagcagagcaggtgccttgggggcagaagggagcttgtcctaggctagggcacatggcatcttgtggtccgacctccttttctgggtcctggctccctggtttcctggccacctcagctccagactcttaacataagcagcactccccaccctggccctagtgttgccactatctgtcatcttggagtctcagacctgcaaagggataggcctcccattcttccctccgcctccccagctctgcacgccccttcctggagttccctgttctaggaaagaggctggaaccaggtgaacgaacaccagttgtttgtgttctgcagaagcccgaacacatgtctagacggggcccagatgttgagcctttggggtggggcttaaagattgtgctggaggggaggctaccctgttatgtgttcccagaggcctgccatttctgggaagggacagttatgtggagtgagctgcttctaggagtttgaggggttgtgggatcccctgacagaggtggagcatctttccttgtctgctgtccccatgtgtctattctgaatggctcaagagcaaccctccctcacatgatggctcacccatccccttcaccccaggagaatcccagttgggggctgagatccttggctgaggaatgtagcagggagaagtaggaactgcctgtcgccctcacatcctgtctgtcccctcatggctgcctggggagggatgtatgggaggaggtggagagatcgcttcctcccagggcagcaggaagggtccctgacttcctgtcctgggaaggcgaccctgtccctgtgacttgctctgttggccggtagggaagttccatcttctacccaacttaactcctgtggtgctggcttgcttttgtgggatcctggatggtggactcatctggcctggggcagactgtgtgtgtgtgtggcgaggtgtggaacctgaagtggagagccctgtagcatcctgctcggcctgcagtcatttgctcctgggattgcatctatggtcctgatgcaacatcgcacagagatgccagcagggctgtgtccctggtcccttcccagcactagcatagctgaggggtcccagcatagccctcccaccaggtgaggactggagaccctgactgtgctttggaagctttactctaggggaaatatgggagagtggcatggagaggaggcaggttgtagggtgtgatccccagttttggaggatgagttggagactaatgagaggtgggaagaggggtcaggctgcagaaaatctgtttctgcaagatcaagaagacaagtcattaactccatagctggaagggtctagttcgagggtctgtggggtcagagggcatgtggcaggggggccaccctaggcagaagagggtctgccatcttgccaggtcctattctacccaggccaggccctggctctggatatgtcagcctgggcacaggacccctggaaaggagggtctgcagagagagcccccagtgagacttcatctgctgtctgctggctaccccggatagggaccagtctaggcccccagctccagctgggccagtgtgggcatggtggtggaggggaggcccccagggagggagggacccagaatgagaagcttggggtccactgtaaggctctggggagccctggaagttttgagccagtgacacggatcccttctttggaaggaaagcagtgccataaggggcagggggccagggagagggtagattagatggggtgcctccctctaaggatccccaaagcctgaaggacaaggctcccaggagttggtgccaggcttgaggaagacacagccctcttcccacctcccacagcagggagcaggcaggagaagggcctgcctcccattctgtaagcgatacctcccattctgtaagagataaagacctctgaccccaccccaggctctccagggagccaagcctcaccttagcatggggggtagggttagggatgggccgagcttgagctacaatgacctctcatctgaccctgacccttttctcttccttggggccccatagctgcccccgtccctcagggccccgagtgaaggctgtgaagctactgattgtggaggtcaccccaagacctgtgtctctatcacaaatgtcccctgcccctaaaaggcccacaggagccaaggtcaggggacacatcctggaggcccacccccctagatgcccccacctcatccccacctggaccctgcttcctgcaggggtacctgttgctgggaggcatacctggtccccagctccactgggctcgccttcctcagtttctcccttagggcctgcagggtcttcctggctctgggccccttccccaacctccagtccccaggaagctcctccccaatcctctgcctggggagccacattccgagcataactgagacaggtatgtgtccctccctctgcaccacttgtgggtgaaggagactgcgggtttgctccaggaaggaaggagaccctcacgccctgctttgtcagggaagtggcctggcctcttccagtgccagctgcccacgcatcatgagctgcccaggctggcagctgagcgggcatctcctccagcctttggccactataccctggtctggacttgtacacttgtacactcccaccaccctcctctcccccacctaataaagactggtccttgggcccccctgaattccctaggagggatgcaggcttggggtccttccccaggcctttccctcccgaactccgagccaagagcagagaaaggaagtgttgtctgccctctccgggcctggcacaggaacagggtcggagagtctggcattagtccagcccagccctgcccagtcctgaagccgggggtcctggtggccacagcctagactggcctgcctgattccagtgagcaccctcccttcctccctgccacgctagcctttgggccttcacgccagtcttgggcctttggggaaggtagtcctaggctatatctcaggctagagagatgctgggtcccgggccgccggccttggccttggtgcaggccctgtggcctgggagccggatgtggacacagacacccccagacagggactggctccgcagagcaaagcggggcaggtaggagcccagggcaggttccagggctggcttcctcagggctagagctggctccctctgctgtctggcatttagcttctgtacccccaagtcctgagcccgccctactcccccacattcctccctgccgaggggggaggtggcttcttcctgtccctgtttccctcttccagacgtaagttctcaccccaccccctccagacccgccccagtgccgtcctcagcccctcccccaggcaggtccttggagctggggcaggcccaggctggaggctgcgcgcctgggctttctccttccttccagccactgggggcggggcatctgctcaggtggagcgtggcctgctcctgcactgtcctctctcacctagggtccccggtaggaagtctccgagagcatgggcttgtgggaccacagcgggctggctgataccctcactggggcggccccaggagcttggggcgtggtggggggtggggagggggttgctgctgaaatcaaagggcattggatccatcacttcaaggcagtgcgctcctttcccgcaggcctgagctggtggaaacagctcctgactttttccagaccccaccccatcaccctgctccccacccagagaagagcccccgcctccatactccccagcagattggaaatggaaaaaaaaaaaagttgttttgaaactgtcctctcccctgctgtctgtacctgccttccgcagggctctggacagagtagattccagtggggcctgcctgtggaaggaaggcagagccatggaggtgctcgcagcctctctggggtgctgggaagaactggatcgcctggtcagggctagctggtctggatctggggctctagccccctgccccaccaatcctttcagactgaggtagggagagtgcagagggtgggatggggctgccccaggtaaggttagcaggggcgtgagcccagacttcagggttgtagatctagccaactgttgcttgccctgcacagcctcctccagcctgttttgcttccttcttaaagggttaatcccccaatcccctgagaatcaaagcctgcccccaccccatcaccacagtggggtggaagacagctgctgaaattgacctctctcctgcccctgccccccaagtcctttgccttcaccagaaggcccaggccagctgccctccctcccagcccagaggcctgaactgctctgctggccccagcaaccccctcagcattgggataggggaactgtggctccccacacaggcccctgaggcccagcccagcaagctgggggctgcaggcacaatccctgaagtagccccaatagcacttaacactccagagtcccaggaagcgcccacctctcacaaagagaaaaagggatctcagaaatgagagttagtgacagctaccctgatgagattgacccagctcatcagagagtactgtgtccccataggctacagggggctcaggagggatctgggccctattctaggctgtggctgggaaccggggaggggtgacctggcctctggtggagcttccaatggggagggaagctcccctcaaaggggcctctccctccagagcagatcctgagaacttcctctgaggaaggccccctgctaggggctgcttacagctggacctgggaaggggaagatggtccctcccacctcccctcccactgaggcatgggaggggcaagctctctttcctcacaggcagccggctgtttccagctgtgtggcctgcacatgtgtgtgtgtgtacatttgtatgcacgtgtgcctgcaacctcaggtgtaccaaccaccacacaacagttctggttgaaactccagctggtggcctgtggttcctgccacccttgtatcgcaggactcagcctggggaggggaccctgcagatgctctgccaatgggatggtagccggcaaccttggcttgtgtcccagagttctgggaacagggctgtgggccctctgtgggtgccgtctcctgcctgctgccccagtgtccagctgtgctcccctcccaccaccccatccccggcagagagctgggcccctgctcctgtgggctctgccgctgggtttctcaatgccagagatggaagggctgcattgaacaggaagagtccctgggatcacttgggttccctgtcccctcacgcctgctggaccctgtgtctggctaagttagggagcagggcaggtcaggagaggtgggtctgcctttgatgtgaggtgggcctgaagctccctacctctcctcttagcaacttcctctgcactgccccctctgcataccaggcccagaagcaggcaacctgttgtggctccctgaggcacctgtccctctggcaacaggtacatgatttctatgtacattggggagaaataaaggcacaacatctgggccagattatggcctgtcctgacccacaagtggggaaaggatgtatgtccacccctgttcctagagggggggaagtgaagcttgctagagtccactcgtgaatccagccatcagttccttcctcacccacccgtccacgcaggacctagcagcaggccctcacaggaccactagaagggctgctgcagctacccacgcggctctctcctgtctggtcggtctttctgtccctttgttcccagcttctcttggttccaatgttgtagccatgcgttctgggaaacacactcactcagaagcacaatgcaaataacggagtgcagtttattacaccggcgggcccaaggcagagtctcctcttagccaaggaccccgaccagtttttctgaaaaccttatataccctaagtgtacgtgcccaaacccacctccccaaattccctgaaactagtctgaacagaggaaaagaaagatacagtcaaagttaacctgtgattcatatgccttaagcctaggtagttaacagtggaccattattaataggcctgtgatcataccccaataagcataatagaatttatgattctatgcggttacacagataattagggtattctttaggtaacagagagtctaggaacgagccctggggctcttccatccggaggggtctggttttccagttggtgtgtcgtttccatagatactgggcatatagctcaaagtccacagtccggcccaagatggagtcctgctttcaagatggagcctggtctgtctgtttcctccttcatccctcctcttcatgctcttaactcatagtatgagcatcattcatagggatttattgcaccctgactctccgacctccaatttgggagaacgacatcaacctttgggttacagagttcataatacattgtaaaataaagggtccacaaagcagaactatgaaggcaactataacaatggtaaatatagttttccaccaatcacccttcacccaacataggaccgaagtccaaaaaggaagatgtatcagacataacttttacctgacctttcatgtcatctagggtagctgatctatagccagataaatcaggaatatacacacaacattcaactttaattatagcacaggtccctctttgtactgaaactgtggtcaatctcaagatgatagcagcaagtccttgtagcagcagttgattgtagagcttcatctctgtttcttctttaagatgatcttggtttcatggggatcagaggggtccctctgcacagtccactcggcgtcctccgggtctgcatggtatgctctcttcagcctcatgtggaggatccagggagtgacacctgcaactttaactgcagtagggctggttagaacaacagcatatggacccttccaatgtggggccaaggagtcgtgtttccagtccttgaccacacccgatccctgggcacaaattcgtgaatctgttccccaagggggaacggcaccttttcttgtacaaacttagttacctgatttattaccttacccagttgttccatctgctgtgaaatctcatctccccttacctgaggcaaatttgttgacacctgttttattatgggagggggcctcccatacacaattttgtacagagacgattcatgggactgtggggtcatcctgagtctgagcagacccgtcagaaacaagtccacccaggaaccgtcagtctccaagatccacttggagagtgtctctaagtgtccggctggttccttccaccatcccagaactctggggcctatatgctgtatgtaatttccacttgatgtttaaagttttgcttacttggttatactaaaacagctacaaaagccgggccattgtccgatccaatgctggtaggaaatccaaatctgggaactatctccctaagcaggcaccgggctacttttgatgctctttcagtgagggtaggaaaagcttctacccatcctgagaacgtacatactgtgaccagcaggtaatggtagtgtccgtgaggtttcatttcagggaagtccacttccaggtgttcaaagggcagcgtgccttttacctgaatccctgcaggtttctgtctgtgccaacaggcagcattggcctgtaagcaggcagtgcagttctgagattttgtcctgcatggggaagagaggaggggaacaaagaaatagtttcaaattatctcttccagtttatcatggcatagatgggtcgcttggtgtgtttggcttaccagagtgggtgccagctcctccggtacccataatttgccacttggcaattcccactatcccttttcagtcttgatggccccttctgctttggctagttggttttgagcttcagtgtattttggagagtctagtgttagctcaggtagctccgccaatatgaaagctttaacaggggcttcacttgtcacccccaagcccttggctgcttgtttagcggtcttatctgccagtctgttccctgagcccggggagtatcctctttttgacgttctcagcagtgtatgactgcaatcctttctggttcccaggcagcatctaatagggtcttaatttcttccttattttttatctttttcactagctgtcaaaggcctctctccttatacagagccctgtagtgtggcaaaagcatacctggagtctgtgtcaaggtttgtcttcttaccttttgacagctggagggcctggattagagcatatagttcagcctgttgaatggagcagtgtggtggcagagagggagcctcaacaatggtttcttctgtgaccactgcatatcctgacagtcattgtccttgtttcaccaggctggtgctattggtgtacacgaccgaatctgggtccgggattggctggtctctcaagtcaggtctgctggcatatttccttgcgatcatgtgagggcccaccttctcccacaggaaggagagtggccagattcagggcctgacaaggctcagtagtaacatgggggttctcacataacagtccctggtactgagtaatctgggatgttgacagccatttatggaggtccccttgcaggagagtgttgacctcatgtgggacttttacaaacaaatcttggcccaaagtcagcttggttgcatcctggaccagtaaggcaactgcctgtaagcatcccagccacccagtagcaacactgtccagctgattagaggtaagtcacaggtctgtcccatgtccccatagtctgggacaacactcctatagccaccttgtccttttcagtcatgtaaagagtaaatggcttagcaaggtctggcaggcccaaggcgggtgctgatgtaactgtactgggtttgagttttattaccactgggacttgttttgcaagcctgggggggttgtcttctgcccagacctcagggaattgttgagttaactctctctcttgaccattgagcctgtccggtttcccttctgggagatcgtgcaatctcccttcatcttgaggggttactgagaggaagagtaaataggtggttaagcccactcgaacagtgggtctttcttggggagacagtgacttgttcccccaatttagacagcaagtctcttcctaacaaaggtactgggcattcagggatgtataaaaactcatgagtcacttggtgtccccccatctgacattttcagggtaggctagagacacaaggctgcatttatcaccatctgagacccagcagcctctggctgggtctattggcgtataaagtctataggcctcgcatagtctttcgcagaactcagggtgattcgctttccctttgaatcacttcagagggttttgtgatacttatagcttttcgggcccccctcttgagaccttgtaaaatagccacccgataactctccaggtggcccctcccttcctctgtgttacagtcccagttgggcctctcatccggggtggctagttctgcccaccgctgcgggtttgcggtaccctcaggtgccatttcggagaaggcaatggcaccccactccagtactcttgcctggaaaatcccatggacggaggagcctggtgggctacagtccatggggtcgctaagagtcggacacgactgagcgacttcactttcacttttcactttcatgcattggagaaggaaatggcaacccactccagtgttcttgcctgcagaatcccagggacgggggagcctggtgggctgccgtctctggggtcgcacagagttggacacgactgaagcgacttagcagcagtagcagcagcagcaggtgctatttttcttaaccattttctggcctcagttaggatcctgtgtctttcctcagtgctgaaaagggagactagtagttggattatgtcatcctatgtagggcggtgggttcgaaaaatagtctctaatagcctaatcacggcttgtgctccccggagtatggtggagtgtgtctctgccagtttaatatattcgtagaggaaaatggctagtaataataggctacaggggctgatggtagtgccccatgtgtcctgaactggaggctgttgtagctttctaaggggcatctgtagtggggttctttccccctgttccttggtggagtgcagcctctgtctaattgctgtgttttgttcccccttcccatcagtactcaccgggagaggtggatacaatctaggaggtccagctgaggtggaattctggggacattgaccagaggtctccattgctgggattggagcctgccgaaacggcagctctacgaactccgggagagctggtggaagagcagtggctgctgcaggaacttcacctggccctggatggggcagtaaggtggcctccggtcctggtggagcactgggaggcaggcgtgtcattatccagtatgggggaggggtcaggtcatttccgtccaaatcctgtaaaatttccttttttatcatcagtcaatttttgtgccattaatattttcccctttcccttctggatacagaaccttgtccaagtaggagggtcttgagctaaccctagccatgagtcaatatatggatattgatccaggtgtccgggctctcctgtgattactgtatagacggcttccactatttttaagttcatggtgttctctggtggccatcctactcccatagggggccattcgacctcacagagtatgtggaggcggttaggcttcatcttcaccccgttcagttcagttcagttgctcagtcgtgtccaactctttgcgaccccatgaattgcagcatgccaggcctccctgtccatcaccaactcctggagttcactcaaactcacctccatcaagttggtgatgccatccagccatctcatcctctgtcatccccttctcctcctgcccccagtccctcccagcatcagagtcttttccagtgagtgaactctttgcatgaggtggccagtctcctccaaatcccttctttaaatttttaatcatgcactccaatacagttgccttagattcacttcccctcatcttgcttaccttctcggaccttctacttttcctttcgttctgtctacgaagttctcagtaccctatgtacttctgatatttccactcaatgacacttaaattgccattctgccaccttcctaataggggtgagaaaagccttacctgccagatcccagagggagaagggggattggcatgtcttcacctgccggtcagcacaaccaaaccagaccaccacgtgatccaagattgtttctcccttaacttttaaagtccattctgcctcggtgggcttgatcaggtgcggatgaaaatacagatgggttaaatatcccatgtcccaggccatctctggaaaagccaattcgtactcacttatgtatccccctcctatcctgagaattccgagggggtcaagaatttcatagcagatgggacatctccttggggagggcagaatacgagcacacagaaaccaagtttcttctcctaaaaatcccctggcaattgcttggtaataattttccccaagatggcatggacaccacctcctaaatgaccttcacaagtttccttcctaagccctaacacactcatcaacctgtgtaccaagcaatcgttgccacctgcctattccaggctcctgtgggtctgtgccccttctagtccctcccagggcggtgatcaggccccctcttccaccttgctgggtgggttcctcctcacctgagcactcagttcccctgctgccatccactacctgctaacatgaaaggtccgggcattgagaagcagaatccttccagaagggcgaggtgccttcccccctctagaagatttgagccacaaggcctcagagtagtcccaaatgggacttgtctcctcaaagtgaggagtttcccggccaacgcaccaaatgttgtagccacacgttctgggaaacaaactcactcagaaggacaatgcagatagtggagtgcagtttattacaccagcgggcccaaggcagagtctcctcttagccaaggaccccgaccagtttttctgaaaaccttatataccctgggtcgggaagatcccctggagaaggaaatggcaacccactccagtactcttgcctggagaattccatggagggaggatcctggtaggctacagtccatagggtcgcaaagagtcagacacaactgagcgacttcactatataccctaagtgtatgtgcccaaacccacctcctcaaattccctgaaactagtctgaacaaaggaaaagaaagatacaatcaaagttaacccttgattcatatgccttaagcctaggtagttaacagtggacaattatcaatagccctgtggcataccccaataagcataatagaatttatgattttatttggttacacagataattagggtattttaggtaacagagagtctaggaacgagccctgggactcttccatccggggggtctggttttccagttggtgtgtcgtttccatagatactgggcatatagctcagagtccacagtccagcccaagatggagtcctgctttcaagatggagcctggtctgtctgtttcctccttcaccaataggtgtctgtctgccactcctcttcatcttgacatccagctggtcaatacctccaagaagcaccgccccccaactcagacctttgtgctggactgtgcatctctcttggcctggccatggccctcagcaggaatacagggtctcctgactcaccggcactgaccacacctgggtgccaccctggctcctccagctttctgtttcctcctagacatgggcaggacctggccgaaagagatggatttggggctgtcctagaggggagggacagaaggtatggatcagctagctccaccccatcccctacctcaggggtccccaaccctgaggccatggaccgctactggtcagtggcctgttaggattcagaccacacagcaagaggtgaggggtcggagtgctggtgaacaagcgaaatttcatctgtgtttacagctgctccccattttggcattaccagctgagctttgcctcctgtgagatcagtggcggcataatgaacgtaatgcgtctgaatcatccccaagcctctccccacccaactccaaattcgtggaaaaactgtcttccacaaaactggtctctggtaccaaaaaggttggggacagctgccctacctttgatttccctggtggctcagacgtatctgcctacaatgtggcagtcctgggttcgatccctgggtcgggaagatcccctggagaaggaaatggcaacccattccagtacccttgcctggagaatcccatggatggaggagcctggtgggctctaccttttctccaggtgtctactaccccctcctctgttcggctggcgccccctggtggctgaagcctgtcttggcaggcgtcccggccctgggttccccaggcctgcgggtctggccttggcccttgtttccctgagacctcaccaggctggcttctcctggtctcgtgcagtctcctgagcttgtttttactgggcctctgtagttggctctgcctggatgtgcaaatccctcgaccccatccttcgggtctctgtacagtcatcccccacgtagatgctgcgatctagggcaatctctgctacagggtaaccttccaggggcaaagagctcacccgactcggcacctgcctcctgcactgagcccacccccggaggagtttggccggggccgggcaggatgaatgctgagtaatgaaggaggaggggtgccggggtcagcggccgctgccctgcgacccagccccaattcttccacaagctctcacttaatgccacctcccgacagcgcgggctaaccagcagtgtgggccctgcccggggctcgtgggtgggcatacagcccgcggactcaagtgggcccctcccggccttagcctcacctgagggggcgatcggggccgggctgcacgccgcctccttcccagcggttggccccagtccaacagcgacgtcccgctgtgaaacgcccattgtctgctgaggccgagggggaggggatggccctgagacccgactctaggaggctgggggagggggccgccctgccccatcctcttcccctcccccgccagggcggccagacacctgtggctggaggccgcctggcactcgactgttggatacactttacacactagcggggtaccctcgagggcccaggagtggagactggcggaggtccccacagcaaaggtgtgtgcgcatgcgtagagtgtgtctgctgccgggagggcggtgccaccccactcccaccccatccttttggagcgcgccggcccctcccctacagagcgaaagggatcagagggaggccagtaggtgggacttcatattctctgtggacttcgttcccggtcttcctcgcagggcaagggaagcgtctggaggggcccaaccacacgcgtgtatgtgggggtacacccgatgtgtgctgtgtggatgaggggcaaattttgcattgcatgaaggtgtgcagtgtgtatgcacacaggtgtgtgtgtgactgcacagtgcctgtgcagacttgagtgagtggttgtacgtatgtccgcatatgggagacctggaacatggagtg

SEQ ID NO: 5 - DNA, 809 bp, Bos taurus
aagcagcagccaagaaagtagaactcacaaaaaatgaaggataattgttacatttctgtttgttatttcaaagaagcacgtggaggaaagagggctaagcttattttcgtgtttgatgttgttttcactttgaattcccttgtggggcacaatcatgttttgagttttggggatgccagcccatggtggcctgggcagtcttgtctgcatcccacaaacctctctggaggctcactgtaggcctgactgttcttggggctggggaggcctctcctgaactctgaactgatgtgggaggaaaaggcaaatgagcaaaataaataatgacatggtttccagagacagaaagaaatgtgtaggttttggggggagccgaaagccttctttccactgagtggtctggatggtatttttgcagtgagctctgctggagaaggcaatggcaccccactccagtactcttgcctggaaaatcccatggacggaggagcctggtgggctacagtccatggggtcgctaagagtcggacacgactgagcgacttcactttcacttttcactttcatgcattggagaaggaaatggcaacccactccagtgttcttgcctgcagaatcccagggacgggggagcctggtgggctgccgtctctggggtcgcacagagttggacacgactgaagcgacttagcagcagtagcagcagcagcaggtgctatttttcttaaccattttctggcctcagttaggatcctgtgtctttcctcagtgctgaaaagggagactagtagttggattatgtcatccta

SEQ ID NO: 6 - DNA, 2050 bp, Ovis aries
ggtgacagacgctccatcctcgggggatgatgaagatggggacgacgaggctgaggacgcagcaggggccccttactggacgcggcccgagcggatggacaagaagctgctagcggtgccggccgccaacacggttcgcttccgctgcccggctgccggcaaccccacgccatccatcacctggctgaagaacggcaaggagttccggggcgagcaccgcatcggagggatcaagctgcggcaccagcagtggagcctggtcatggagagcgtggtgccctcggaccgcggcaactacacgtgcgtcgtggagaacaagtttggcagaatccagcagacctacaccctggacgtgctggagcgctctccgcaccggcccatcctccaggcggggctgcccgccaaccagaccgccgtcgtgggcagcgacgtggagttccactgcaaggtgtacagcgatgcccagccccacatccagtggctcaagcacgtggaggtgaacggcagcaaggtggggcccgacggcacgccctacgtcaccgtgctcaagacggcgggcgctaacaccaccgacaaggagctagaggttctgtccttgcgcaatgtcacctttgaggaacgcgggggagtacacgtgtctggcgggcaattctatcgggttttcccatcactctgcgtggctggtggtgctgccagccgaggaggagctggtggaagctggtgaggctggcagtgtgttcgcgggcgtcctcagctacgggctgggcttcctcctcttcatcctggctgtggccgccgttacgctctaccgcctgcggagcccccccaagaagggcctgggctcgcccgcggtgcacaaggtctcccgcttcccgctcaagcgacaggtgtccttggagtccagttcatccatgagctccaacacgccactggtacgcatcgcccggctgtcctcgggcgagggccccaccctggccaacgtctctgagctcgagctgcccgccgaccccaagtgggagctgtcccgggcccggctgaccctgggcaagcctcttggggagggctgcttcggccaggtggtcatggcagaggccattggcatcgacaaggaccgagctgccaagcccgtcacggtggcggtgaagatgctgaaagatgacgccacggacaaggacttgtcggacctggtgtccgagatggagatgatgaagatgatcggaaaacataagaacattatcaacttgctaggcgcctgcacgcagggcgggcccctgtacgtgctggtggagtacgcggccaagggcaacctacgggagtacctgcgggcgcggcggcccccaggcactgactactcctkcgacacctgccggctgcccgaggagcagctcaccttcaaagacctggtgtcctgcgcctaccaggtggcgcggggcatggagtacctggcctcgcagaagtgcatccatagggacctggcggcccgcaacgtgctggtgaccgaggagaacgtgatgaaaatcgccgacttcggcctggcccgtgatgtgcacaacctcgactactacaagaagacaacaaacggccgcctgcccgtgaagtggatggcacccgaggccttgtttgaccgcgtctacacccaccaaagtgatgtgtggtccttcggggtcctgctctgggagatcttcacgctggggggctcgccgtaccctggcatccccgwggaggagctcttcaagctgctgaaggaaggccaccgcatggacaagccggccaactgcacgcatgacctgtacatgatcaggcgtgagtgctggcacgccgcgccctcgcagaggcccaccttcaagcagctggtggaggacctggaccgtgtgctcactgtgacttccactgacgagtacctggacctgtcagtgcccttcgagcagtactcaccgggcggccaggacacccccagctccggctcctctggggacgactccgtgttcgctcacgacctgctgcccccggcccctggtagcggaggctctcggacgtgaagggccactat